(19) United States
(12) Patent Application Publication
(10) Pub. No.: US 2007/0167488 A1
(43) Pub. Date: Jul. 19, 2007

(54) Novel therapeutic use

(75) Inventors: Laetitia Maud Elysa Bouerat Duvold, Frederiksberg (DK); Jef Fensholdt, Stenlose (DK); Simon Feldbaek Nielsen, Herlev (DK); Xifu Liang, Clostrup (DK); Sophie Elisabeth Havez, Valby (DK); Ellen Christina Anderson, Virum (DK); Lene Jensen, Malov (DK); Jens Rainer Hansen, Stenlose (DK)

Correspondence Address: BIRCH STEWART KOLASCH & BIRCH, PO BOX 747, FALLS CHURCH, VA, 22040-0747, US

(73) Assignee: Leo Pharma A/S, Ballerup (DK)

(21) Appl. No.: 10/582,935

(22) PCT Filed: Dec. 16, 2004
(86) PCT No.: PCT/DK04/00875

§ 371 Date: Jan. 3, 2007

Related U.S. Application Data
(60) Provisional application No. 60/529,630, filed on Dec. 16, 2003.

(51) Int. Cl. A61K 31/454; A61K 31/4439; A61K 31/404

(52) U.S. Cl. 514/323; 514/337; 514/414

(57) ABSTRACT

Certain oxindole compounds have been found to be effective in experimentally induced autoimmune encephalitis and are therefore suggested for the preparation of a medicament for the prevention, treatment or amelioration of multiple sclerosis, or to delay the onset of or reduce the relapse rate in multiple sclerosis.

### FIELD OF INVENTION

The present invention relates to the use of certain indolinone compounds in the prevention, treatment or amelioration of multiple sclerosis.

### BACKGROUND OF THE INVENTION

Multiple sclerosis is an auto-immune inflammatory disease of the central nervous system characterised by T-cell infiltration, demyelination of white matter and axonal injury. The disease mostly affects young adults with an onset at 20-40 years of age and affects twice as many women as men (A. Compton and A. Coles, The Lancet 359, 6 April 2002, pp. 1221-1231). Multiple sclerosis is more common in temperate climate zones and thus has a prevalence of 50-130 out of 100,000 in northern Europe and North America (N. Hellings et al., Immunologic Research 25(1), 2002, pp. 27-51). While the higher incidence and prevalence of multiple sclerosis in certain European populations has not been adequately explained, it is believed that increased genetic susceptibility in these populations is partly responsible. The presence of a genetic element in the etiology of the disease is supported by family studies showing that first-degree relatives of multiple sclerosis patients have a 20-40 times increased risk of developing the disease relative to the general population (J. H. Noseworthy et al., New England Journal of Medicine 343(13), 2000, pp. 938-952). Furthermore, it has been recognised that populations with a high frequency of for instance the HLA-DR2 allele have a significantly higher risk of developing multiple sclerosis (Hellings et al., supra; Noseworthy et al., supra). However, no single major susceptibility gene for multiple sclerosis has been identified so far, and the results of genome screens conducted to identify susceptibility genes rather point to multiple genes exerting a moderate effect (Hellings et al., supra).

Based on these studies, it would appear that genetic susceptibility is not enough in itself to provoke multiple sclerosis. This theory is given credence by the fact that the rate of prevalence of multiple sclerosis among people of European descent living outside Europe is half of that persisting in parts of northern Europe and that the low frequency of multiple sclerosis in Africans increases significantly among first-generation decendants living in Europe (Compton and Coles, supra). Environmental factors have therefore also been proposed as contributing to the development of multiple sclerosis.

In particular, it is believed that certain antigens present on pathogenic organisms such as viral or bacterial epitopes which structurally resemble autoantigenic epitopes of, for instance, myelin basic protein, proteolipid protein, myelin-associated glycoprotein or oligodendrocyte glycoprotein, which are all components of the myelin sheath, may lead to activation of T-cells that are reactive with such antigenic epitopes and initiating the inflammatory process eventually resulting in clinical manifestations of multiple sclerosis. This phenomenon is generally referred to as molecular mimicry (Hellings et al., supra; A. Bar-Or et al., J. Neuroimmunol. 100, 1999, pp. 252-259; A. Karni and H. L. Weiner, “Organ-Specific Inflammatory Diseases” Chapter 77 in Clinical Immunology; Principles and Practice, 2^{nd} Ed. (R. R. Rich et al., Eds.), Mosby, London, 2001).

Multiple sclerosis is usually defined as either a relapsing-remitting or a progressive disease. The relapsing-remitting form with which 80% of the patients are initially afflicted (Compton and Coles, supra) is characterised by discrete attacks with full or partial recovery between relapses. In 40-50% of the patients, the disease eventually becomes progressive (secondary progressive stage). The disease may also be progressive from the outset (primary progressive form) characterised by a gradual decline in neurological function with no periods of remission. The clinical symptoms of the relapsing-remitting form of multiple sclerosis may vary widely from one patient to the other, but commonly affected individuals initially experience some degree of visual and sensory impairment, limb paresthesias, limb weakness, clumsiness, fatigue and gait ataxia, while in the later stages cognitive impairment, progressive quadriparesis, sensory loss, ataxic tremors, pain and spasticity are more common (Noseworthy et al., supra). The primary progressive form may initially manifest as one or more of these symptoms, gradually declining into quadriparesis, cognitive decline, visual loss, brainstem syndromes and cerebellar, bowel and bladder dysfunction (Noseworthy et al., supra).

Pathologically, multiple sclerosis is characterised by the presence of demyelinated plaques or sclerotic lesions where the myelin sheath surrounding the axons is destroyed. The inflammatory infiltrate in the lesions is composed of T-cells, B-cells, microglia and macrophages which interact with the myelin sheath and participate in the demyelinating process by local production of immune-related molecules such as adhesion molecules, cytokines and chemokines as well as demyelinating antibodies, oxygen free radicals and nitric oxide (Karni and Weiner, supra). While axonal destruction is not pronounced in the early stages of the disease (although more pronounced in patients suffering from the primary progressive form), demyelination of the axons results in slowing and blocking conductivity (Noseworthy et al., supra).

Regression of the symptoms may be associated with partial remyelination after the initial inflammation has subsided showing that oligodendrocytes (myelin-producing cells) are present in the lesions (Karni and Weiner, supra). In later stages, irreversible axonal injury, gliotic scarring and gradual loss of oligodendrocyte progenitor cells may result from repeated episodes of inflammatory attack and leads to permanent loss of neurological function (Noseworthy et al., supra).

While the immunopathogenesis of multiple sclerosis is still largely unknown, it has been shown that autoreactive T-cells specific for myelin basic protein and other antigens of the central nervous system exist in the periphery of healthy individuals as well as individuals who later develop multiple sclerosis (Bar-Or et al., supra; O'Connor et al., J. Clin. Immunol. 21(2), 2001, pp. 81-93). Thus, the presence of myelin-reactive T-cells in the periphery is not enough in itself to explain the development of multiple sclerosis. In multiple sclerosis patients, these T-cells become activated, possibly by cross-reactivity with bacterial or viral antigens that structurally resemble myelin antigens (i.e. the phenomenon known as molecular mimicry) and/or by bacterial superantigens, and persist in an enhanced state of activation (Hellings et al., supra). It has been found that the autoreactive T-cells are predominantly CD4+ T helper cells type 1 (Th1) producing interleukin-2 (IL-2), interferon-γ (IFN-γ) and tumour necrosis factor (TNF-α) (B. Gran and A. Rostami, Current Neurology and Neuroscience Reports 1, 2001, pp. 263-270). In order for such proinflammatory T-cells to migrate to the central neurvous system, they express chemokine receptors, adhesion molecules and matrix metalloproteinases that enable them to cross the blood-brain barrier. Thus, it has been found that expression levels of the chemokines which are chemotactic for Th1 cells, IP-10 and RANTES, and their corresponding receptors, CXCR3 and CCR5, are elevated in sclerotic lesions and cerebrospinal fluid of multiple sclerosis patients (Bar-Or et al., supra). Altered levels of the adhesion molecules ICAM-1 and VCAM-1 have been identified on endothelial cells of multiple sclerosis lesions (O'Connor et al., supra). ICAM-1 and VCAM-1 are important for endothelial-leukocyte interactions and leukocyte extravasation. Matrix metalloproteinases expressed by activated T-cells, monocytes and astrocytes may disrupt the basement membrane of the blood-brain barrier and facilitate transmigration of T-cells and breakdown of the extracellular matrix (O'Connor et al., supra).

Once the T-cells have entered the central nervous system they become reactivated on encountering the autoantigen, e.g. myelin basic protein, presented by MHC class II expressing antigen presenting cells (microglia and dendritic cells), and the Th1 cells respond by producing proinflammatory cytokines such as TNF-α, IFN-γ and IL-2, while the Th2 cells produce anti-inflammatory cytokines such as IL-4, IL-5 and IL-10 (Bar-Or et al., supra). In turn, the inflammatory process leads to up-regulation of MHC class II expression and adhesion molecules on the blood-brain barrier endothelium, facilitating a further influx of T-cells, B-cells and macrophages and hence an amplification of the inflammatory response (Hellings et al., supra). This theory is supported by the finding that myelin basic protein reactive T-cell clones from multiple sclerosis patients were found to secrete increased amounts of different cytokines such as TNF-α, IL-2 and IL-10 (Hellings et al., supra). Demyelination (myelin destruction) is believed to be brought about by the combined effects of cytotoxic cells (macrophages and T-cells), oxygen free radicals, demyelinating autoantibodies and cytokine-induced toxicity (Hellings et al., supra).

Traditionally, corticosteroids such as prednisolone have been administered intravenously to multiple sclerosis patients during acute relapses in order to attenuate the inflammatory response. It has been found that treatment with corticosteroids during relapses reduces the duration of relapses and their short-term morbidity, but not the permanent disabilities resulting from repeated relapses (Compton and Coles, supra). Furthermore, treatment with potent corticosteroids at high doses has serious side effects, notably osteoporosis, aseptic bone necrosis, skin atrophy, striae cutis, insomnia, myopathy, posterior and capsular cataract and glaucoma as well as reactivation of the disease upon cessation of treatment. More recently, interferon-β (IFN-β) was introduced as a treatment of relapsing-remitting multiple sclerosis and was found to decrease the rate of relapse, increase the proportion of patients who were relapse free and reduce the number of patients who had moderate to severe relapses. On the other hand, INF-β treatment is extremely costly and its long-term efficacy has not been established. There is concern that the treatment may induce the formation of neutralising antibodies that may reduce the activity of IFN-β (Noseworthy et al., supra). Most of the patients initially experience flu-like symptoms when treated with IFN-β. Glatiramer acetate is another recent treatment based on a mixture of random synthetic peptides intended to mimic myelin basic protein. In a double-blind trial of relapsing-remitting multiple sclerosis, glatiramer acetate was found to decrease the rate of relapse. Glatiramer acetate is believed to be most effective for mildly disabled patients with a recent diagnosis of multiple sclerosis. Fewer treatment options exist for patients in the progressive phase of the disease. Immunosuppressive therapy, e.g. with cyclophosphamide or methotrexate, is frequently attempted, but it is generally recognised that once the disease enters the progressive stage treatment is very difficult. IFN-β has been in clinical trials for secondary progressive multiple sclerosis but the results did not show that the treatment slowed progression of disability and the benefits of this treatment in secondary progressive disease are controversial.

Thus, it would appear that there is a continued medical need for effective treatment of multiple sclerosis. It would also be beneficial if a medicament suitable for oral administration were to be developed.

### SUMMARY OF THE INVENTION

In the course of research leading to the present invention, it was surprisingly found that certain indolinone compounds which have previously been shown to be inhibitors of receptor tyrosine kinases and suggested for the treatment of cancer exhibit a substantial level of activity in experimentally induced autoimmune encephalomyelitis (EAE) which is generally recognised as an animal model of multiple sclerosis. EAE may be induced by injection of antigenic peptides of myelin such as myelin basic protein, proteolipid protein and myelin oligodendrocyte glycoprotein. EAE is an inflammatory condition of the central nervous system characterised by T-cell infiltration and focal demyelination. EAE can also be induced by transfer of myelin reactive T-cells to normal individuals.

Accordingly, the present invention relates to the use of a compound of general formula I 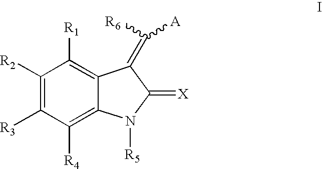
wherein

R₁, R₂, R₃ and R₄ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂;

X is O or S;

R₅ is hydrogen, hydroxy, C₁₋₆ alkyl, C₂₋₆ alkenyl, C₂₋₆ alkynyl, cycloalkyl, heterocyclyl, aryl, heteroaryl, C₁₋₆alkoxy, carbonyl, carboxy, amido, thioamido, guanyl, guanidinyl, ureidyl, sulfonyl, trihalomethanesulfonyl, —C(O)OR₁₄, —C(O)R₁₄, wherein R₁₄ is hydrogen, C₁₋₆alkyl, C₂₋₆ alkenyl, C₂₋₆ alkynyl, cycloalkyl or aryl;

R₆ is hydrogen, C₁₋₆ alkyl, cycloalkyl, aryl, heteroaryl, heterocyclyl, halogen, —OR₇, —C(O)R₇, —C(O)OR₇, —NR₇R₈, S(O)₂NR₇R₈, wherein R₇ and R₈ are independently hydrogen, C₁₋₆ alkyl, aryl or heterocyclyl, said C₁₋₆ alkyl or heterocyclyl being optionally substituted by heterocyclyl, —OR₇, —C(O)R₇ or C(O)OR₇, the zigzag line indicating that the group denoted R₆ is present as the E- or Z-isomer;

A is phenyl or a monocyclic or bicyclic heteroaryl ring, optionally substituted at one or more positions with hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₁₆-alkyl, C₂₋₁₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; the zigzag line indicating that the group denoted A is present as the E- or Z-isomer;

or pharmaceutically acceptable salts thereof, for the preparation of a medicament for the prevention, treatment or amelioration of multiple sclerosis, or to delay of the onset of or reduce the relapse rate in multiple sclerosis.

In another aspect, the invention relates to a method of preventing, treating or ameliorating multiple sclerosis, or delaying the onset of or reducing the relapse rate in multiple sclerosis, the method comprising administering, to a patient in need thereof, a pharmacologically effective amount of a compound of general formula I as shown above.

Compounds of formula I are disclosed in, e.g., WO 96/40116 in which they are indicated to be inhibitors of tyrosine kinases and as such useful in the treatment of cancer, blood vessel proliferative disorders, fibrotic disorders, mesangial cell proliferative disorders and metabolic diseases. There is no suggestion that compounds within this group might have any utility in the treatment of multiple sclerosis.

### BRIEF DESCRIPTION OF THE DRAWINGS

FIG. 1 shows inhibition of EAE with compound 226. Mice were immunized on day 0 with the PLP₁₃₉₋₁₅₃ peptide. Compound 226 was dosed daily i.p. from day 0 as follows; compound 226-50 (50 mg/kg); compound 226-25 (25 mg/kg); compound 226-10 (10 mg/kg); compound 226-4 (4 mg/kg). Control groups were given either suspension vehicle i.p. from day 0 or dexamethasone (1 mg/kg) p.o. from day 1. The experiment was terminated on day 21 p.i. A) The frequency of animals with EAE symptoms. Mice dead or sacrificed during the experiment are included. B) The average disease score in each group. Mice which died/were sacrificed during the experiment were given the same score for the rest of the experiment C) The average weight gain or loss for each group. Weights are compared with the weight on day 0. D) The mortality in each group. Only mice dead with EAE symptoms were included.

FIG. 2 shows inhibition of EAE with compound 226. Mice were immunized on day 0 with the PLP₁₃₉₋₁₅₃ peptide. Compound 226 was dosed as follows; compound 226-50 (50 mg/kg); compound 226-25 (25 mg/kg). Control groups were given either suspension vehicle i.p. from day 0 or dexamethasone (1 mg/kg) p.o. from day 1. The experiment was terminated on day 28 p.i. A) The frequency of animals with EAE symptoms. Mice dead or sacrificed during the experiment are included. B) The average disease score in each group. Mice which died/were sacrificed during the experiment were given the same score for the rest of the experiment C) The average weight gain or loss for each group. The weights are compared with the weight on day 0. D) The mortality in each group. Only mice dead with EAE symptoms were included.

FIG. 3 is a graph showing IL-2 production by spleen cells of mice immunized with PLP₁₃₉₋₁₅₃ peptide and dosed daily with compound 226 (n=6) or suspension vehicle (n=6). The spleen cells were collected on day 10 and restimulated in vitro with different concentrations of the PLP₁₃₉₋₁₅₃ peptide with or without compound 226 present (FIGS. 3A and 3B, respectively). After 3 days of culture, the supernatants were tested for production of IL-2 using a time-resolved fluorometer. The average for each group is plotted together with the standard deviation.

FIG. 4 is a graph showing cytokine production by spleen cells of mice immunized with PLP₁₃₉₋₁₅₃ peptide and dosed daily with compound 226 (n=4), compound 57 (n=4) or suspension vehicle (n=4). On day 10 spleen cells were collected and restimulated in vitro with different concentrations of the PLP₁₃₉₋₁₅₃ peptide. After 3 days culture the supernatants in each group were pooled and tested in duplicates for production of cytokines using a time-resolved fluorometer. The average stimulation index (SI) for each group is plotted together with the standard deviation. SI=(counts with peptide (0.5 mg/ml)/counts without peptide). FIG. 4A shows the results for production of IL-2, FIG. 4B shows the results for production of IL-6, FIG. 4C shows the results for production of IFN-γ, and FIG. 4D shows the results for production of IL-17.

### DETAILED DESCRIPTION OF THE INVENTION

Definitions

In the present context, the term “C₁₋₁₂-alkyl” is intended to mean a linear or branched hydrocarbon group having 1 to 12 carbon atoms, such as methyl, ethyl, propyl, iso-propyl, cyclopropyl, butyl, tert-butyl, iso-butyl, cyclobutyl, pentyl, cyclopentyl, hexyl, cyclohexyl, etc. Analogously, the term “C₁₋₁₀ alkyl” and “C₁₋₆-alkyl” is intended to mean a linear, cyclic or branched hydrocarbon group having 1 to 10 or 1 to 6 carbon atoms, respectively, such as methyl, ethyl, propyl, iso-propyl, pentyl, cyclopentyl, hexyl, cyclohexyl, and the term “C₁₋₄-alkyl” is intended to cover linear or branched hydrocarbon groups having 1 to 4 carbon atoms, e.g. methyl, ethyl, propyl, iso-propyl, butyl, iso-butyl, tert-butyl.

Similarly, the terms “C₂₋₁₂-alkenyl”, “C₄₋₁₂-alkadienyl”, and “C₆₋₁₂-alkatrienyl” are intended to cover linear, cyclic or branched hydrocarbon groups having 2 to 12, 4 to 12, and 6 to 12, carbon atoms, respectively, and comprising one, two, and three unsaturated bonds, respectively. Examples of alkenyl groups are vinyl, allyl, butenyl, pentenyl, hexenyl, heptenyl, octenyl, heptadecaenyl. Examples of alkadienyl groups are butadienyl, pentadienyl, hexadienyl, heptadienyl, heptadecadienyl. Examples of alkatrienyl groups are hexatrienyl, heptatrienyl, octatrienyl, and heptadecatrienyl. Preferred examples of alkenyl are vinyl, allyl, butenyl, especially allyl.

Similarly, the term “C₂₋₁₂-alkynyl” is intended to mean a linear or branched hydrocarbon group having 2 to 12 carbon atoms and comprising a triple bond. Examples hereof are ethynyl, propynyl, butynyl, octynyl, and dodecaynyl.

“Halogen” includes fluoro, chloro, bromo, and iodo.

In the present context the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system, usually a mono- or bicyclic ring system comprising 5-12 ring atoms, where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are oxazolyl, isoxazolyl, oxadiazolyl, oxatriazolyl, thiazolyl, isothiazolyl, thiadiazolyl, thiatriazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular pyrrolyl, imidazolyl, pyridinyl, pyrimidinyl, thienyl, quinolyl, tetrazolyl, and isoquinolyl.

The term “carbocyclyl” is intended to indicate a cyclic hydrocarbon radical, which may be a saturated or unsaturated, non-aromatic, mono- or bicyclic ring comprising 5-12 ring atoms, such as C₃₋₈ cycloalkyl, e.g. cyclopropyl, cyclopentyl, cyclohexyl or cyclooctyl, or a C₃₋₈ cycloalkylene radical, e.g. cycloprop-2-enyl, cyclobut-2-enyl, cyclopent-2-enyl, cyclohex-3-enyl, cycloocta-4-enyl or cyclohex-3,5-dienyl.

The term “heterocyclyl” is intended to mean a non-aromatic ring or ring system, usually a mono- or bicyclic ring system comprising 5-12 ring atoms, where one or more of the carbon atoms have been replaced with heteroatoms, e.g. nitrogen (═N— or —NH—), sulphur, and/or oxygen atoms. Examples of such heterocyclyl groups are imidazolidine, piperazine, hexahydropyridazine, hexahydropyrimidine, diazepane, diazocane, pyrrolidine, piperidine, azepane, azocane, aziridine, azirine, azetidine, pyroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular imidazolidine, piperazine, hexahydropyridazine, hexahydropyrimidine, diazepane, pyrrolidine, piperidine, azepane, oxazinane (morpholine), and thiazinane.

The term “EAE” or “EAE model” is used herein to denote experimentally induced autoimmune encephalomyelitis (EAE) which is generally recognised as an animal model of multiple sclerosis. EAE may be induced by injection of antigenic peptides of myelin such as myelin basic protein, proteolipid protein and myelin oligodendrocyte glycoprotein. EAE is an inflammatory condition of the central nervous system characterised by T-cell infiltration and focal demyelination. EAE can also be induced by transfer of myelin reactive T-cells to normal individuals.

The term “alkoxy” is intended to indicate a radical of formula OR*, wherein R* is alkyl as defined above, e.g. methoxy, ethoxy, propoxy, butoxy, etc.

The term “alkylaryl” is intended to indicate an alkyl group covalently joined to an aryl group.

The term “sugar residue” is intended to indicate a glucuronide, e.g. hydroxyl or acyl glucuronide.

The term “halogen” is inteded to indicate fluoro, chloro, bromo or iodo.

The term “pharmaceutically acceptable salt” is intended to indicate salts prepared by reacting a compound of formula I with a suitable inorganic or organic acid, e.g. hydrochloric, hydrobromic, hydroiodic, sulfuric, nitric, acetic, phosphoric, lactic, maleic, phthalic, citric, propionic, benzoic, glutaric, gluconic, methanesulfonic, salicylic, succinic, tartaric, toluenesulfonic, sulfamic or fumaric acid.

The term “indolinone compound” (used synonymously with “oxindole compound” herein) is intended to include compounds of formula I, II, III or IV as shown herein as well as other, structurally related compounds, such as the compounds disclosed in WO 96/40116, U.S. Pat. No. 6,316,635, U.S. Pat. No. 6,225,335, WO 99/48868, WO 99/61422, WO 01/60814, WO 00/56709, WO 01/83450, EP 934 931, U.S. Pat. No. 5,834,504, WO 98/07695, WO 02/02551, WO 00/08202, WO 98/50356, WO 96/22976, WO 01/45689, WO 02/055517 and WO 01/94312 which are hereby incorporated by reference in their entirety. Methods of preparing the compounds are also disclosed in these publications.

The term “KDR” is understood to indicate a receptor tyrosine kinase which binds selectively to vascular endothelial growth factor (VEGF). The DNA and amino acid sequence of KDR as well as its proposed use to identify agonists and antagonists of VEGF action are disclosed in WO 92/14748.

The term “ameliorate” is intended to mean reducing the severity of the neurological symptoms during relapses of multiple sclerosis by administering an effective amount of an active compound whereby it may be possible to reduce or delay permanent disability resulting from neurological damage sustained during relapse, in particular demyelination.

The term “delay the onset of multiple sclerosis” is used to indicate a prophylactic administration of an effective amount of an active compound to prolong the period where no symptoms, or at least no severe symptoms, of multiple sclerosis are observed in susceptible individuals, e.g. in first-degree relatives of multiple sclerosis patients.

The term “reduce the relapse rate in multiple sclerosis” is intended to mean reducing the frequency with which relapses occur or, in other words, prolong the periods of remission. This may make it possible to reduce or delay the accumulation of disabilities resulting from the neurological damage sustained during each relapse, in particular demyelination which eventually leads to increasingly severe disability.

Preferred Embodiments of the Invention

In one embodiment, the invention relates to the use of a compound of general formula II 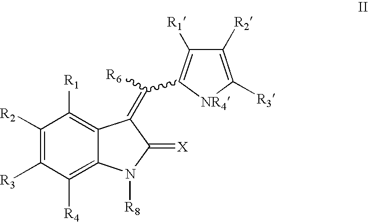
wherein R₁, R₂, R₃, R₄, R₆ and X are as indicated for formula I,

R₈ and R₄′ are independently hydrogen, hydroxy, C₁₋₆alkyl, C₂₋₆alkenyl, C₂₋₆alkynyl, cycloalkyl, heterocyclyl, aryl, heteroaryl, C₁₋₆alkoxy, carbonyl, carboxy, amido, thioamido, guanyl, guanidinyl, ureidyl, sulfonyl, trihalomethanesulfonyl, —PO(OR)(OR′), wherein R and R′ are independently selected from hydrogen or C₁₋₆ alkyl, —OR₁₀, —C(O)R₁₀, —C(O)OR₁₀, OC(O)R₁₀, OC(O)OR₁₀, —NR₁₀R₁₁, —C(O)NR₁₀R₁₁, —OC(O)NR₁₀R₁₁, —NHC(O)R₁₀, —SR₁₀, —S(O)R₁₀, —S(O)₂R₁₀, —S(O)₂NR₁₀R₁₁, —S(O)OR₁₀ and CH₂-aryl-OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, OC(O)OR₁₂, —NR₁₂R₁₃, —C(O)NR₁₂R₁₃, —OC(O)N₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂, —S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; —C(R₂₄R₂₅)—OR₁₆ or —OC(O)R₁₆, wherein R₁₆ is hydrogen, C₁₋₆ alkyl, aralkyl, acyl or —PO(OR)(OR′), —C(R₂₄R₂₅)—NR₂₆R₂₇, wherein R₂₄ is hydrogen, C₁₋₆ alkyl or aryl, R₂₅ is hydrogen, and R₂₆ and R₂₇ are independently hydrogen or C₁₋₆ alkyl or, together with the nitrogen atom to which they are attached, form a heteroaryl or heteroaryl ring optionally substituted with hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂ or —S(O)NH₂; —NR₂₀R₂₁, —O(CH₂)ₘNR₂₀R₂1, —N(CH₂)ₘNR₂₀R₂₁, —O(CH₂)ₘC(O)R₂₂, —N(CH₂)ₘC(O)R₂₂, wherein m is 0, 1, 2 or 3, R₂₀ and R₂₁ are the same or different and independently selected from the group consisting of hydrogen, C₁₋₆ alkyl, cycloalkyl, aryl, carbonyl, acetyl, trihalomethylcarbonyl, carboxy, sulfonyl or trihalomethanesulfonyl, or R₂₀ and R₂₁ together with the nitrogen atom to which they are attached form a heterocyclic or heteroaryl ring, and R₂₂ is hydroxy, C₁₋₆ alkoxy, aryloxy, amino, hydroxylamino, carboxy or —NR₂₀R₂₁, wherein R₂₀ and R₂₁ are as indicated above; and

R₁′, R₂′ and R₃′ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₁₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄alkyl, C₁₋₄alkoxy, nitro, cyano, amino, oxo, halogen, trihalomethyl, C₁₋₄alkylthio, C₁₋₄alkylamino, C₁₋₄alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; and

pharmaceutically acceptable salts thereof, for the preparation of a medicament for the prevention, treatment or amelioration of multiple sclerosis, or to delay the onset of or reduce the relapse rate in multiple sclerosis.

Compounds of formula II are disclosed in, e.g. WO 99/61422 in which they are indicated to be inhibitors of protein tyrosine kinases and as such useful in the treatment of cancer, diabetes, hepatic cirrhosis, cardiovascular disease, such as atherosclerosis and angiogenesis, immune disease such as autoimmune disease and renal disease. There is no suggestion that compounds within this group might have any utility in the treatment of multiple sclerosis.

In another embodiment, the invention relates to the use of compounds of formula III 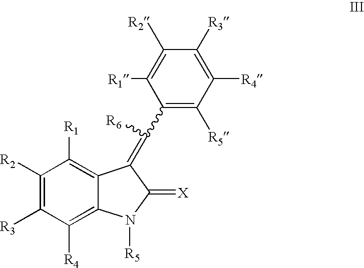
wherein R₁, R₂, R₃, R₄, R₅, R₆ and X are as indicated for formula I, and

R₁″, R₂″, R₃″, R₄″ and R₅″ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein OR₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, halogen, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₁₆-alkyl, C₂₋₁₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, —S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂;

and pharmaceutically acceptable salts thereof, for the preparation of a medicament for the prevention, treatment or amelioration of multiple sclerosis, or to delay the onset of or reduce the relapse rate in multiple sclerosis.

Compounds of formula III are disclosed in WO 96/40116 in which they are indicated to be inhibitors of tyrosine kinases and as such useful in the treatment of cancer, blood vessel proliferative disorders, fibrotic disorders, mesangial cell proliferative disorders and metabolic diseases. There is no suggestion that compounds within this group might have any utility in the treatment of multiple sclerosis.

In a further embodiment, the invention relates to the use of compounds of formula IV 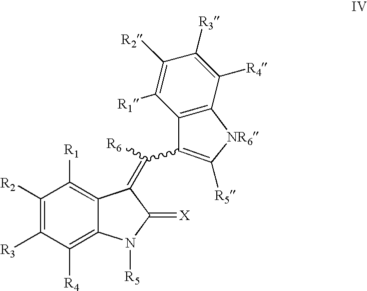
wherein R₁, R₂, R₃, R₄, R₅, R₆ and X are as indicated for formula I,

R₁″, R₂″, R₃″, R₄″ and R₅″ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₁₆-alkyl, C₂₋₁₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, —S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; and

R₆″ is hydrogen, heterocyclyl, heteroaryl, —C(O)R₂₃, —S(O)₂R₂₃, —C(O)OR₂₃ or C₁₋₆ alkyl optionally substituted with heterocyclyl, heteroaryl or —C(O)OR₂₃, wherein R₂₃ is hydrogen, C₁₋₆ alkyl, aryl, heteroaryl or heterocyclyl;

and pharmaceutically acceptable salts thereof, for the preparation of a medicament for the prevention, treatment or amelioration of multiple sclerosis, or to delay the onset of or reduce the relapse rate in multiple sclerosis.

Compounds of formula IV are disclosed in WO 98/07695 in which they are indicated to be inhibitors of tyrosine kinases and as such useful in the treatment of cell proliferative diseases such as cancer, atherosclerosis, arthritis and restenosis, and metabolic diseases such as diabetes. There is no suggestion that compounds within this group might have any utility in the treatment of multiple sclerosis.

In the compound of formula I,

X may be O or S;

R₁, R₂, R₃ and R₄ may be the same or different and independently selected from the group consisting of hydrogen, C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, S(O)R₁₈, S(O)₂R₁₈, S(O)₂NR₁₈R₁₉, S(O)₃R₁₈, SR₁₈, NO₂, NR₁₈R₁₉, OH, CN, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆ alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆ alkyl or aryl, and n is 0-3;

A may be phenyl or a monoclyclic or bicyclic heteroaryl ring selected from the group consisting of pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,3,4-thiatriazole, 1,2,3,5-thiatriazole, tetrazole and indole, optionally substituted at one or more positions with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OH, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈, R₁₉ and n are as indicated above;

R₅ may be hydrogen or C₁₋₆ alkyl; and

R₆ may be hydrogen.

More specifically, in the compound of formula I, R₅ is preferably hydrogen; X is preferably oxygen; R₁, R₂, R₃ and R₄ are preferably the same or different and independently selected from hydrogen and C₁₋₆ alkyl, R₆ is preferably hydrogen or COOH; and/or A is pyrrole, phenyl or indole, said pyrrole, phenyl or indole being optionally substituted at one or more positions with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OH, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈, R₁₉ and n are as indicated above. In particular, A is pyrrole substituted at position 3 and 5 with C₁₋₆ alkyl, or or at position 3 with C₁₋₆ alkyl and at position 5 with CH₂OH, COOH or a sugar residue, or at position 3 and 5 with C₁₋₆alkyl and at position 4 with halogen, or at position 5 with C(O)O—C₁₋₆ alkyl, and at position 5 with C₁₋₆ alkyl. Alternatively, A is phenyl substituted at position 2 and 5 with C₁₋₆ alkyl, C₁₋₆ alkoxy, halogen, C₁₋₆alkyl-NR₂₆R₂₇, NH—C₁₋₆alkyl-NR₂₆R₂₇ or O—C₁₋₆alkyl-NR₂₆R₂₇, wherein R₂₆ and R₂₇ are as indicated above.

In the compound of formula II, R₁, R₂, R₃, R₄, R₆ and X are preferably as indicated above, and R₁′, R₂′ and R₃′ are preferably the same or different and independently selected from the group consisting of with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆ alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆ alkyl or aryl, and n is 0-3.

In a currently favoured embodiment of the compound of formula II, R₈ and R₄′ are independently hydrogen, hydroxy, —PO(OR)(OR′), —OR₁₀, —C(O)OR₁₀, —C(O)NR₁₀R₁₁, —C(O)R₁₄, —C(R₂₄R₂₅)OR₁₆, —OC(O)R₁₆ or —C(R₂₄R₂₅)NR₂₆R₂₇, wherein R, R′, R₁₀, R₁₁, R₁₄, R₁₆, R₂₄, R₂₅, R₂₆, R₂₇ are as indicated above.

More specifically, in the compound of formula II, R₁, R₂, R₃ and R₄ are preferably the same or different and independently selected from hydrogen, halogen and C₁₋₆ alkyl, or R₂ may be hydroxy or heteroaryl, such as pyridyl, or a group C(O)R₂₀, wherein R₂₀ is heteroaryl, such as pyridyl or thienyl, and R₁, R₃ and R₄ are hydrogen. Furthermore, in the compound of formula II, R₁′, R₂′ and R₃′ are preferably the same or different and independently selected from hydrogen, halogen, C₁₋₆ alkyl, C₁₋₆ alkoxy, CH₂OH or C(O)OR₁₈ or C(O)NR₁₈R₁₉, wherein R₁₈ and R₁₉ are as defined above. By way of example, in the compound of formula II, R₁′ and R₃′ may both be C₁₋₆ alkyl, in particular methyl, and R₂′ may be hydrogen, or R₁′ may be C₁₋₆ alkyl and R₃′ may be C₁₋₆ alkoxy, CH₂OH, C(O)OR₁₈ or C(O)NR₁₈R₁₉, or R₁′ and R₃′ may both be C₁₋₆ alkyl, in particular methyl, and R₂′ may be halogen, in particular chloro or bromo, or R₁′ may be C₁₋₆ alkyl and R₃′ may be C(O)O—C₁₋₆ alkyl, or R₁′ may be C₁₋₆ alkyl and R₃′ may be C(O)NH—C₁₋₆ alkyl substituted with hydroxy.

Examples of compounds of formula II are selected from the group consisting of
3-(3,5-Dimethyl-1H-pyrrol-2-ylmethylene)-1,3-dihydro-indol-2-one (Compound 226)
4-methyl-5-(2-oxo-1,2-dihydro-indol-3-ylidenemethyl)-1H-pyrrole-2-carboxylic acid ethyl ester (Compound 01)
4-methyl-5-(2-oxo-1,2-dihydro-indol-3-ylidenemethyl)-1H-pyrrole-2-carboxylic acid (2-hydroxy-ethyl)-amide (Compound 02)
3-(5-hydroxymethyl-3-methyl-1H-pyrrol-2-ylmethylene)-1,3-dihydro-indol-2-one (Compound 03)
1-[4-methyl-5-(2-oxo-1,2-dihydro-indol-3-ylidenemethyl)-1H-pyrrol-2-ylmethyl)-pyrridinum; chloride (Compound 04)
4-methyl-5-(2-oxo-1,2-dihydro-indol-3-yfidenemethyl)-1H-pyrrole-2-carboxylic acid (Compound 05)
4-methyl-5-(2-oxo-1,2-dihydro-indol-3-ylidenemethyl)-1H-pyrrole-2-carboxylic acid (2-diethylamino-ethyl)-amide (Compound 06)
4-methyl-5-(2-oxo-1,2-dihydro-indol-3-ylidenemethyl)-1H-pyrrole-2-carboxylic acid (2-methoxy-ethyl)-amide (Compound 07)
4-methyl-5-(2-oxo-1,2-dihydro-indol-3-ylidenemethyl)-1H-pyrrole-2-carboxylic acid [3-(1-formyl-piperidin-4-yl)-propyl]-amide (Compound 08)
4-{[4-methyl-5-(2-oxo-1,2-dihydro-indol-3-ylidenemethyl)-1H-pyrrole-2-carbonyl]-amino}-butyric acid methyl ester (Compound 09)
4-methyl-5-(2-oxo-1,2-dihydro-indol-3-ylidenemethyl)-1H-pyrrole-2-carboxylic acid (6-hydroxy-hexyl)-amide (Compound 10)
4-methyl-5-(2-oxo-1,2-dihydro-indol-3-ylidenemethyl)-1H-pyrrole-2-carboxylic acid cyclohexylmethyl-amide (Compound 11)
4-methyl-5-(2-oxo-1,2-dihydro-indol-3-ylidenemethyl)-1H-pyrrole-2-carboxylic acid (4-hydroxy-butyl)-amide (Compound 12)
6-{[4-methyl-5-(2-oxo-1,2-dihydro-indol-3-ylidenemethyl)-1H-pyrrole-2-carbonyl]-amino}-hexanoic acid ethyl ester (Compound 13)
4-methyl-5-(2-oxo-1,2-dihydro-indol-3-ylidenemethyl)-1H-pyrrole-2-carboxylic acid (tetrahydro-furan-2-ylmethyl)-amide (Compound 14)
4-methyl-5-(2-oxo-1,2-dihydro-indol-3-ylidenemethyl)-1H-pyrrole-2-carboxylic acid [2-(1H-indol-3-yl)-ethyl]-amide (Compound 15)
4-methyl-5-(2-oxo-1,2-dihydro-indol-3-ylidenemethyl)-1H-pyrrole-2-carboxylic acid (3-phenyl-propyl)-amide (Compound 16)
4-methyl-5-(2-oxo-1,2-dihydro-indol-3-ylidenemethyl)-1H-pyrrole-2-carboxylic acid (4-phenyl-butyl)-amide (Compound 17)
4-methyl-5-(2-oxo-1,2-dihydro-indol-3-ylidenemethyl)-1H-pyrrole-2-carboxylic acid (5-hydroxy-pentyl)-amide (Compound 18)
4-{[4-methyl-5-(2-oxo-1,2-dihydro-indol-3-ylidenemethyl)-1H-pyrrole-2-carbonyl]-amino}-butyric acid ethyl ester (Compound 19)
4-methyl-5-(2-oxo-1,2-dihydro-indol-3-ylidenemethyl)-1H-pyrrole-2-carboxylic acid [1-(4-chloro-phenyl)-cyclopropylmethyl]-amide (Compound 20)
4-methyl-5-(2-oxo-1,2-dihydro-indol-3-ylidenemethyl)-1H-pyrrole-2-carboxylic acid benzyl ester (Compound 21)
3-(4-bromo-3,5-dimethyl-1H-pyrrol-2-ylmethylene)-1,3-dihydro-indol-2-one (Compound 22)
3-(4-chloro-3,5-dimethyl-1H-pyrrol-2-ylmethylene)-1,3-dihydro-indol-2-one (Compound 23)
3-(3,5-dimethyl-1H-pyrrol-2-ylmethylene)-1-(4-methoxy-benzyl)-1,3-dihydro-indol-2-one (Compound 41)
3-(3,5-Dimethyl-1H-pyrrol-2-ylmethylene)-i-methyl-1,3-dihydro-indol-2-one (Compound 42)
acetic acid 3-(3,5-dimethyl-1H-pyrrol-2-ylmethylene)-2-oxo-2,3-dihydro-indol-1-ylmethyl ester (Compound 43)
3-(3,5-dimethyl-1H-pyrrol-2-ylmethylene)-1-hydroxy-1,3-dihydro-indol-2-one (Compound 45)
3-(4-bromo-3,5-dimethyl-1H-pyrrol-2-ylmethylene)-1-hydroxy-1,3-dihydro-indol-2-one (Compound 46)
3-(3,5-dimethyl-1H-pyrrol-2-ylmethylene)-1-methoxy-1,3-dihydro-indol-2-one (Compound 49)
acetic acid 3-(3,5-dimethyl-1H-pyrrol-2-ylmethylene)-2-oxo-2,3-dihydro-indol-1-yl ester (Compound 51)
2-{3-[3-(3,5-dimethyl-1H-pyrrol-2-ylmethylene)-2-oxo-2,3-dihydro-indol-1-yloxy]-propyl}-isoindole-1,3-dione (Compound 52)
2,4-Dimethyl-5-(2-oxo-1,2-dihydro-indol-3-ylidenemethyl)-1H-pyrrole-3-carboxylic acid (2-diethylamino-ethyl)-amide (Compound 227)
5-(5-Fluoro-2-oxo-1,2-dihydro-indol-3-ylidenemethyl)-2,4-dimethyl-1H-pyrrole-3-carboxylic acid (2-diethylamino-ethyl)-amide (Compound 228)
(3,5-dimethyl-1H-pyrrol-2-yl)-(2-oxo-1,2-dihydro-indol-3-ylidene)-acetic acid (Compound 229)
3-[2,4-Dimethyl-5-(2-oxo-1,2-dihydro-indol-3-ylidenemethyl)-1H-pyrrol-3-yl]-propionic acid (Compound 230)
3-(3,5-dimethyl-1H-pyrrol-2-ylmethylene)-4-iodo-1,3-dihydro-indol-2-one (Compound 231)
3-(3,5-dimethyl-1H-pyrrol-2-ylmethylene)-5-methoxy-1,3-dihydro-indol-2-one (Compound 232)
5-chloro-3-(3-methoxy-1H-pyrrol-2-ylmethylene)-1,3-dihydro-indol-2-one (Compound 233)
3-(3-methoxy-1H-pyrrol-2-ylmethylene)-1,3-dihydro-indol-2-one (Compound 234)
3-[5-(4-chloro-2-oxo-1,2-dihydro-indol-3-ylidenemethyl)-2,4-dimethyl-1H-pyrrol-3-yl]-propionic acid (Compound 235)
4-chloro-3-(3,5-dimethyl-1H-pyrrol-2-ylmethylene)-1,3-dihydro-indol-2-one (Compound 236)
4-chloro-3-(3-methoxy-1H-pyrrol-2-ylmethylene)-1,3-dihydro-indol-2-one (Compound 237)
3-(3,5-dimethyl-1H-pyrrol-2-ylmethylene)-2-oxo-2,3-dihydro-1H-indole-4-carboxylic acid (Compound 238)
3-(3,5-dimethyl-1H-pyrrol-2-ylmethylene)-5-pyridin-3-yl-1,3-dihydro-indol-2-one (Compound 239)
3-(3,5-dimethyl-1H-pyrrol-2-ylmethylene)-5-pyridin-3-yl-1,3-dihydro-indol-2-one; methanesulfonic acid (Compound 240)
5-pyridin-3-yl-3-(1H-pyrrol-2-ylmethylene)-1,3-dihydro-indol-2-one (Compound 241)
5-pyridin-3-yl-3-(1H-pyrrol-2-ylmethylene)-1,3-dihydro-indol-2-one; methanesulfonic acid (Compound 242)
3-(3,5-dimethyl-1H-pyrrol-2-ylmethylene)-5-hydroxy-1,3-dihydro-indol-2-one (Compound 243)
3-(3,5-dimethyl-1H-pyrrol-2-ylmethylene)-5-fluoro-1,3-dihydro-indol-2-one (Compound 244)
3-(1-methyl-1H-indol-2-ylmethylene)-1,3-dihydro-indol-2-one (Compound 245)
2,4-dimethyl-5-(2-oxo-1,2-dihydro-indol-3-ylidenemethyl)-1H-pyrrole-3-carboxylic acid ethyl ester (Compound 246)
4-methyl-5-(2-oxo-1,2-dihydro-indol-3-ylidenemethyl)-1H-pyrrole-2-carboxylic acid pyridin-4-ylmethyl ester (Compound 263)
(3,5-dimethyl-1H-pyrrol-2-yl)-(2-oxo-1,2-dihydro-indol-3-ylidene)-acetic acid benzyl ester (Compound 264)
3-(3,5-dimethyl-1H-pyrrol-2-ylmethylene)-1-pyrrolidin-1-ylmethyl-1,3-dihydro-indol-2-one (Compound 266)
3-(3,5-dimethyl-1H-pyrrol-2-ylmethylene)-1-(4-methyl-piperazin-1-ylmethyl)-1,3-dihydro-indol-2-one (Compound 267) and
3-(3,5-dimethyl-1H-pyrrol-2-ylmethylene)-1-piperidin-1-ylmethyl-1,3-dihydro-indol-2-one (Compound 268)

In the compound of formula III, R₂, R₃, R₄, R₅, R₆ and X may be as indicated above, and R₁″, R₂″, R₃″, R₄″ and R₅″ are preferably the same or different and independently selected from the group consisting of with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆ alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆ alkyl or aryl, and n is 0-3.

More specifically, in the compound of formula III, R₂″ and R₅″ are preferably the same or different and independently are C₁₋₆ alkyl, in particular methyl, or C₁₋₆alkoxy, in particular methoxy, or halogen, in particular chloro or bromo. By way of example, in the compound of formula III, R₅ may be hydrogen, hydroxy or C(O)R₁₄ or C(O)OR₁₄, wherein R₁₄ is as defined above.

Examples of compounds of formula III are selected from the group consisting of
3-(2,5-dimethoxy-benzylidene)-1,3-dihydro-indol-2-one (Compound 110)
3-(5-dimethylaminomethyl-2-methoxy-benzyidene)-1,3-dihydro-indol-2-one (Compound 32)
3-{2-[(2-dimethylamino-ethyl)-methyl-amino]-5-methoxy-benzylidene}-1,3-dihydro-indol-2-one (Compound 33)
3-{4-[(2-dimethylamino-ethyl)-methyl-amino]-3′,5′-dimethyl-biphenyl-3-ylmethylene}-1,3-dihydro-indol-2-one (Compound 34)
3-(2-dimethylaminomethyl-benzylidene)-1,3-dihydro-indol-2-one (Compound 35)
3-[2-(2-diethylamino-ethoxy)-5-methoxy-benzylidene]-1,3-dihydro-indol-2-one (Compound 36)
3-[2-(2-diethylamino-ethoxy)-5-methoxy-benzylidene]-1,3-dihydro-indol-2-one; hydrochloride (Compound 37)
3-[5-methoxy-2-(2-morpholin-4-yl-ethoxy)-benzylidene]-1,3-dihydro-indol-2-one (Compound 38)
3-[5-methoxy-2-(2-piperidin-1-yl-ethoxy)-benzylidene]-1,3-dihydro-indol-2-one (Compound 39)
1-acetyl-3-(2,5-dimethoxy-benzylidene)-1,3-dihydro-indol-2-one (Compound 44)
3-(2,5-dimethoxy-benzylidene)-1-hydroxy-1,3-dihydro-indol-2-one (Compound 48)
3-(2,5-dimethoxy-benzylidene)-1-methoxy-1,3-dihydro-indol-2-one (Compound 50)
3-(phenyl-4-tolyl-methylene)-1,3-dihydro-indol-2-one (Compound 53)
3-[bis-(4-methoxy-phenyl)-methylene]-1,3-dihydro-indol-2-one (Compound 54)
3-[1-(2,5-dimethoxy-phenyl)-ethylidene]-1,3-dihydro-indol-2-one (Compound 55)
3-(4-hydroxy-3,5-dimethyl-benzylidene)-1,3-dihydro-indol-2-one (Compound 95)
3-(3,5-di-tert-butyl-4-hydroxy-benzylidene)-1,3-dihydro-indol-2-one (Compound 96)
3-(4-bromo-benzylidene)-1,3-dihydro-indol-2-one (Compound 97)
3-(2-methyl-benzylidene)-1,3-dihydro-indol-2-one (Compound 98)
3-(2,4-dimethyl-benzyidene)-1,3-dihydro-indol-2-one (Compound 99)
3-(2,6-dichloro-benzylidene)-1,3-dihydro-indol-2-one (Compound 100)
3-(3,4-dichloro-benzylidene)-1,3-dihydro-indol-2-one (Compound 101)
3-(4-chloro-benzylidene)-1,3-dihydro-indol-2-one (Compound 102)
3-(2,4-dimethyl-benzylidene)-1,3-dihydro-indol-2-one (Compound 103)
3-(2,5-dimethyl-benzylidene)-1,3-dihydro-indol-2-one (Compound 104)
3-(2,6-dimethoxy-benzylidene)-1,3-dihydro-indol-2-one (Compound 105)
3-benzylidene-1,3-dihydro-indol-2-one (Compound 106)
3-(4-methoxy-benzylidene)-1,3-dihydro-indol-2-one (Compound 107)
3-(2,4-dimethoxy-benzylidene)-1,3-dihydro-indol-2-one (Compound 108)
3-(3,5-dimethoxy-benzylidene)-1,3-dihydro-indol-2-one (Compound 109)
3-(3,4-dimethyl-benzylidene)-1,3-dihydro-indol-2-one (Compound 111)
3-naphtalen-2-ylmethylene-1,3-dihydro-indol-2-one (Compound 112)
3-naphtalen-1-ylmethylene-1,3-dihydro-indol-2-one (Compound 113)
3-(2,3-dimethoxy-benzylidene)-1,3-dihydro-indol-2-one (Compound 114)
3-(3-nitro-benzylidene)-1,3-dihydro-indol-2-one (Compound 115)
3-(2-fluoro-benzylidene)-1,3-dihydro-indol-2-one (Compound 116)
3-(3-methyl-benzylidene)-1,3-dihydro-indol-2-one (Compound 117)
3-(3-fluoro-benzylidene)-1,3-dihydro-indol-2-one (Compound 118)
3-(4-fluoro-benzylidene)-1,3-dihydro-indol-2-one (Compound 119)
3-anthracen-9-ylmethylene-1,3-dihydro-indol-2-one (Compound 120)
3-(5-bromo-2-hydroxy-benzylidene)-1,3-dihydro-indol-2-one (Compound 121)
3-(2-hydroxy-benzylidene)-1,3-dihydro-indol-2-one (Compound 122)
5-chloro-3-(4-isopropyl-benzylidene)-1,3-dihydro-indol-2-one (Compound 123)
5-chloro-3-(4-dimethylamino-benzylidene)-1,3-dihydro-indol-2-one (Compound 124)
5-chloro-3-(3,4-dichloro-benzylidene)-1,3-dihydro-indol-2-one (Compound 125)
5-chloro-3-(2,5-dimethoxy-benzylidene)-1,3-dihydro-indol-2-one (Compound 126)
5-Chloro-3-(2-chloro-benzylidene)-1,3-dihydro-indol-2-one (Compound 127)
5-chloro-3-(2,3-dimethoxy-benzylidene)-1,3-dihydro-indol-2-one (Compound 128)
5-Chloro-3-(2,6-dichloro-benzylidene)-1,3-dihydro-indol-2-one (Compound 129)
5-Chloro-3-(2-hydroxy-benzylidene)-1,3-dihydro-indol-2-one (Compound 130)
5-chloro-3-(4-methoxy-benzylidene)-1,3-dihydro-indol-2-one (Compound 131)
5-chloro-3-(4-chloro-benzylidene)-1,3-dihydro-indol-2-one (Compound 132)
5-chloro-3-naphtalen-1-ylmethylene-1,3-dihydro-indol-2-one (Compound 133)
5-chloro-3-(4-bromo-benzylidene)-1,3-dihydro-indol-2-one (Compound 134)
5-chloro-3-(4-methyl-benzylidene)-1,3-dihydro-indol-2-one (Compound 135)
3-anthracen-9-ylmethylene-5-chloro-1,3-dihydro-indol-2-one (Compound 136)
5-chloro-3-naphtalen-2-ylmethylene-1,3-dihydro-indol-2-one (Compound 137)
5-chloro-3-(2,4-dimethoxy-benzylidene)-1,3-dihydro-indol-2-one (Compound 138)
5-chloro-3-(2,4-dichloro-benzylidene)-1,3-dihydro-indol-2-one (Compound 139)
5-chloro-3-(3,4-dimethoxy-benzylidene)-1,3-dihydro-indol-2-one (Compound 140)
5-Chloro-3-(3,5-dimethoxy-benzylidene)-1,3-dihydro-indol-2-one (Compound 141)
5-chloro-3-(3,5-di-tert-butyl-4-hydroxy-benzylidene)-1,3-dihydro-indol-2-one (Compound 142)
5-chloro-3-(3,4,5-trimethoxy-benzylidene)-1,3-dihydro-indol-2-one (Compound 143)
3-benzylidene-5-Chloro-1,3-dihydro-indol-2-one (Compound 144)
5-chloro-3-(3-hydroxy-benzylidene)-1,3-dihydro-indol-2-one (Compound 145)
5-chloro-3-(2-methoxy-benzylidene)-1,3-dihydro-indol-2-one (Compound 146)
5-chloro-3-(2-methyl-benzylidene)-1,3-dihydro-indol-2-one (Compound 147)
3-(3,5-dibromo-4-hydroxy-benzylidene)-1,3-dihydro-indol-2-one (Compound 148)
3-(3,4-difluoro-benzylidene)-1,3-dihydro-indol-2-one (Compound 149)
3-(2-hydroxy-naphtalen-1-ylmethylene)-1,3-dihydro-indol-2-one (Compound 150)
3-(4-methyl-benzylidene)-1,3-dihydro-indol-2-one (Compound 151)
3-(3,4-dimethoxy-benzylidene)-1,3-dihydro-indol-2-one (Compound 152)
3-(3-methoxy-benzylidene)-1,3-dihydro-indol-2-one (Compound 153)
3-(2-chloro-benzylidene)-1,3-dihydro-indol-2-one (Compound 154)
3-(3-chloro-benzylidene)-1,3-dihydro-indol-2-one (Compound 155)
3-(2,4-dichloro-benzylidene)-1,3-dihydro-indol-2-one (Compound 156)
3-(3,4-dichloro-benzylidene)-1,3-dihydro-indol-2-one (Compound 157)
3-(3-bromo-benzylidene)-1,3-dihydro-indol-2-one (Compound 158)
3-(4-hydroxy-benzylidene)-1,3-dihydro-indol-2-one (Compound 159)
3-(3-hydroxy-benzylidene)-1,3-dihydro-indol-2-one (Compound 160)
3-(2,4-dihydroxy-benzylidene)-1,3-dihydro-indol-2-one (Compound 161)
5-chloro-3-(2,5-dimethoxy-benzylidene)-1,3-dihydro-indol-2-one (Compound 162)
3-(3,4,5-trimethoxy-benzylidene)-1,3-dihydro-indol-2-one (Compound 163)
3-(3,5-dimethoxy-benzylidene)-1,3-dihydro-indol-2-one (Compound 164)
3-(2,3-dimethoxy-benzylidene)-1,3-dihydro-indol-2-one (Compound 165)
3-(2-methoxy-naphtalen-1-ylmethylene)-1,3-dihydro-indol-2-one (Compound 166)
3-(2-methoxy-benzylidene)-1,3-dihydro-indol-2-one (Compound 167)
3-(4-hydroxy-3-methoxy-benzylidene)-1,3-dihydro-indol-2-one (Compound 168)
3-(3-hydroxy-4-methoxy-benzylidene)-1,3-dihydro-indol-2-one (Compound 169)
5-bromo-3-(2,5-dimethoxy-benzylidene)-1,3-dihydro-indol-2-one (Compound 170)
6-bromo-3-(2,5-dimethoxy-benzylidene)-1,3-dihydro-indol-2-one (Compound 171)
7-bromo-3-(2,5-dimethoxy-benzylidene)-1,3-dihydro-indol-2-one (Compound 172)
3-(2,5-dimethoxy-benzylidene)-6-fluoro-1,3-dihydro-indol-2-one (Compound 173)
3-(2,5-dimethoxy-benzylidene)-5-trifluoromethyl-1,3-dihydro-indol-2-one (Compound 174)
5-amino-3-(2,5-dimethoxy-benzylidene)-1,3-dihydro-indol-2-one (Compound 175)
6-chloro-5-(2-chloro-acetyl)-3-(2,5-dimethoxy-benzylidene)-1,3-dihydro-indol-2-one (Compound 176)
3-(2,5-dimethoxy-benzylidene)-5-hydroxy-1,3-dihydro-indol-2-one (Compound 177)
3-(2,5-dimethoxy-benzylidene)-2-oxo-2,3-dihydro-1H-indole-5-carboxylic acid methyl ester (Compound 178)
3-(9-ethyl-9H-carbazol-3-ylmethylene)-1,3-dihydro-indol-2-one (Compound 179)
3-(2-hydroxy-3-methoxy-benzylidene)-1,3-dihydro-indol-2-one (Compound 180)
3-(2,5-dimethoxy-benzylidene)-4,5-difluoro-1,3-dihydro-indol-2-one (Compound 181)
3-(3,5-dichloro-2-hydroxy-benzylidene)-1,3-dihydro-indol-2-one (Compound 182)
3-(2,5-diethoxy-benzylidene)-1,3-dihydro-indol-2-one (Compound 183)
3-(2,5-dihydroxy-benzylidene)-1,3-dihydro-indol-2-one (Compound 184)
3-(2,4,5-trimethoxy-benzylidene)-1,3-dihydro-indol-2-one (Compound 185)
3-(9-methyl-9H-carbazol-3-ylmethylene)-1,3-dihydro-indol-2-one (Compound 186)
3-(2-hydroxy-5-trifluoromethoxy-benzylidene)-1,3-dihydro-indol-2-one (Compound 187)
3-(1H-indol-5-ylmethylene)-1,3-dihydro-indol-2-one (Compound 188)
3-(1H-indol-4-ylmethylene)-1,3-dihydro-indol-2-one (Compound 189)
3-(1H-indol-7-ylmethylene)-1,3-dihydro-indol-2-one (Compound 190)
3-(1,4-dimethyl-9H-carbazol-2-ylmethylene)-1,3-dihydro-indol-2-one (Compound 191)
3-(2-benzyloxy-4,5-dimethoxy-benzylidene)-1,3-dihydro-indol-2-one (Compound 192)
3-(2,5-dichloro-benzylidene)-1,3-dihydro-indol-2-one (Compound 193)
3-(2,5-dimethoxy-benzylidene)-2-oxo-2,3-dihydro-1H-indole-7-carbonitrile (Compound 194)
3-(2,5-dimethoxy-benzylidene)-6-methanesulfonyl-1,3-dihydro-indol-2-one (Compound 195)
3-(2,5-dimethoxy-benzylidene)-2-oxo-2,3-dihydro-1H-indole-5-carbonitrile (Compound 196)
3-(2,5-dimethoxy-benzylidene)-6-trifluoromethyl-1,3-dihydro-indol-2-one (Compound 197)
3-(2,5-dimethoxy-benzylidene)-7-fluoro-1,3-dihydro-indol-2-one (Compound 198)
3-(2,5-dimethoxy-benzylidene)-2-oxo-2,3-dihydro-1H-indole-6-carbonitrile (Compound 199)
6-chloro-3-(2,5-dimethoxy-benzylidene)-1,3-dihydro-indol-2-one (Compound 200)
3-(2,5-dibromo-benzylidene)-1,3-dihydro-indol-2-one (Compound 201)
3-(5-bromo-2-ethoxy-benzylidene)-1,3-dihydro-indol-2-one (Compound 202)
3-(5-bromo-2-methoxy-benzylidene)-1,3-dihydro-indol-2-one (Compound 203)
3-(2-fluoro-5-methoxy-benzylidene)-1,3-dihydro-indol-2-one (Compound 204)
3-(2,5-difluoro-benzylidene)-1,3-dihydro-indol-2-one (Compound 205)
3-(2-chloro-5-nitro-benzylidene)-1,3-dihydro-indol-2-one (Compound 206)
3-(2,5-bis-trifluoromethyl-benzylidene)-1,3-dihydro-indol-2-one (Compound 207)
3-(2,4-dichloro-benzylidene)-1,3-dihydro-indol-2-one (Compound 208)
3-(2-hydroxy-5-methoxy-benzylidene)-1,3-dihydro-indol-2-one (Compound 209)
3-(1H-indol-6-ylmethylene)-1,3-dihydro-indol-2-one (Compound 210)
3-(2,5-dimethoxy-benzylidene)-5-fluoro-1,3-dihydro-indol-2-one (Compound 211)
3-[4-(quinolin-2-ylmethoxy)-benzylidene]-1,3-dihydro-indol-2-one (Compound 212)
3-[4-(naphthalen-2-ylmethoxy)-benzylidene]-1,3-dihydro-indol-2-one (Compound 213)
3-[3,5-dichloro-2-(quinolin-2-ylmethoxy)-benzylidene]-1,3-dihydro-indol-2-one (Compound 214)
2-[4-(2-oxo-1,2-dihydro-indol-3-ylidenemethyl)-phenoxy]-propionic acid (Compound 215)
2-benzyl-3-butylamino-5-(2-oxo-1,2-dihydro-indol-3-ylidenemethyl)-benzenesulfonamide (Compound 216)
2-benzyl-3-benzylamino-5-(2-oxo-1,2-dihydro-indol-3-ylidenemethyl)-benzenesulfonamide (Compound 217)
3-[(furan-2-ylmethyl)-amino]-5-(2-oxo-1,2-dihydro-indol-3-ylidenemethyl)-2-phenoxy-benzenesulfonamide (Compound 218)
3-methylamino-5-(2-oxo-1,2-dihydro-indol-3-ylidenemethyl)-2-phenoxy-benzenesulfonamide (Compound 219)
2-benzyl-3-ethoxy-5-(2-oxo-1,2-dihydro-indol-3-ylidenemethyl)-benzenesulfonamide (Compound 220)
[2-(2-oxo-1,2-dihydro-indol-3-ylidenemethyl)-phenoxy]-acetic acid (Compound 221)
3-(4-(6-methyl-pyridin-2-ylmethoxy)-benzylidene]-1,3-dihydro-indol-2-one (Compound 222)
4-[4-(5-chloro-2-oxo-1,2-dihydro-indol-3-ylidenemethyl)-phenyl]-piperazine-1-carbaldehyde (Compound 223)
5-chloro-3-(4-isopropyl-benzylidene)-1,3-dihydro-indol-2-one (Compound 224)
4-[4-(2-oxo-1,2-dihydro-indol-3-ylidenemethyl)-phenyl]-piperazine-1-carbaldehyde (Compound 225)
3-[5-methoxy-2-(2-morpholin-4-yl-ethoxy)-benzylidene]-1,3-dihydro-indol-2-one; hydrochloride (Compound 258)
3-[5-methoxy-2-(2-piperidin-1-yl-ethoxy)-benzylidene]-1,3-dihydro-indol-2-one; hydrochloride (Compound 259)
3-(2,5-dimethoxy-benzylidene)-5,7-difluoro-1,3-dihydro-indol-2-one (Compound 260)
3-[4-(1-quinolin-4-yl-ethoxy)-benzylidene]-1,3-dihydro-indol-2-one (Compound 261)
3-[4-(pyridin-4-ylmethoxy)-benzylidene]-1,3-dihydro-indol-2-one (Compound 262) and
5-amino-3-(2,5-dimethoxy-benzylidene)-1,3-dihydro-indol-2-one; methanesulfonic acid (Compound 265)

In the compound of formula IV, R₂, R₃, R₄, R₅, R₆ and X are preferably as indicated above, and R₁″, R₂″, R₃″, R₄″ and R₅″ are preferably the same or different and independently selected from the group consisting of with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆ alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆ alkyl or aryl, and n is 0-3; and R₆″ is hydrogen, C₁₋₆ alkyl, heteroaryl, heteroaryl-C₁₋₆ alkyl, C(O)R₁₈, C(O)OR₁₈ or S(O)₂R₁₈, wherein R₁₈ is as indicated above.

More specifically, in the compound of formula IV, R₅″ may be hydrogen or C₁₋₆ alkyl and/or R₆″ may be hydrogen or C₁₋₆ alkyl.

Examples of compounds of formula IV are selected from the group consisting of
3-(1H-indol-3-ylmethylene)-1,3-dihydro-indol-2-one (Compound 57)
[3-(2-oxo-1,2-dihydro-indol-3-ylidenemethyl)-indol-1-yl]-acetic acid methyl ester (Compound 24)
[3-(2-oxo-1,2-dihydro-indol-3-ylidenemethyl)-indol-1-yl]-acetic acid ethyl ester (Compound 25)
[3-(2-oxo-1,2-dihydro-indol-3-ylidenemethyl)-indol-1-yl]-acetic acid (Compound 26)
3-[3-(2-oxo-1,2-dihydro-indol-3-ylidenemethyl)-indol-1-yl]-propionic acid ethyl ester (Compound 27)
3-[3-(2-oxo-1,2-dihydro-indol-3-ylidenemethyl)-indol-1-yl]-propionic acid (Compound 28)
3-[l-(2-chloro-thiazol-5-ylmethyl)-1H-indol-3-ylmethylene]-1,3-dihydro-indol-2-one (Compound 29)
3-(1-methyl-1H-indol-3-ylmethylene)-1,3-dihydro-indol-2-one (Compound 30)
3-(1-propyl-1H-indol-3-ylmethylene)-1,3-dihydro-indol-2-one (Compound 31)
3-(1H-indol-3-ylmethylene)-2-oxo-2,3-dihydro-indole-1-carboxylic acid tert-butyl ester (Compound 40)
1-hydroxy-3-(1H-indol-3-ylmethylene)-1,3-dihydro-indol-2-one (Compound 47)
(1-Methyl-1H-indol-3-yl)-(2-oxo-1,2-dihydro-indol-3-ylidene)-acetic acid (Compound 56)
3-(2-phenyl-1H-indol-3-ylmethylene)-1,3-dihydro-indol-2-one (Compound 58)
3-(1-methyl-2-phenyl-1H-indol-3-ylmethylene)-1,3-dihydro-indol-2-one (Compound 59)
3-[2-(4-chloro-phenyl)-1H-indol-3-ylmethylene]-1,3-dihydro-indol-2-one (Compound 60)
3-(2-naphthalen-2-yl-1H-indol-3-ylmethylene)-1,3-dihydro-indol-2-one (Compound 61)
5-chloro-3-(1H-indol-3-ylmethylene)-1,3-dihydro-indol-2-one (Compound 62)
3-(5-methyl-1H-indol-3-ylmethylene)-1,3-dihydro-indol-2-one (Compound 63)
5,7-difluoro-3-(1H-indol-3-ylmethylene)-1,3-dihydro-indol-2-one (Compound 64)
5-bromo-3-(1H-indol-3-ylmethylene)-1,3-dihydro-indol-2-one (Compound 65)
6-fluoro-3-(1H-indol-3-ylmethylene)-1,3-dihydro-indol-2-one (Compound 66)
6-bromo-3-(1H-indol-3-ylmethylene)-1,3-dihydro-indol-2-one (Compound 67)
5-hydroxy-3-(1H-indol-3-ylmethylene)-1,3-dihydro-indol-2-one (Compound 68)
3-(4,5,6,7-tetrafluoro-1H-indol-3-ylmethylene)-1,3-dihydro-indol-2-one (Compound 69)
3-(6-fluoro-1H-indol-3-ylmethylene)-1,3-dihydro-indol-2-one (Compound 70)
3-[2-(4-chloro-phenyl)-5-nitro-1H-indol-3-ylmethylene)-1,3-dihydro-indol-2-one (Compound 71)
7-bromo-3-(1H-indol-3-ylmethylene)-1,3-dihydro-indol-2-one. (Compound 72)
3-(6-methyl-1H-indol-3-ylmethylene)-1,3-dihydro-indol-2-one (Compound 73)
3-(7-methyl-1H-indol-3-ylmethylene)-1,3-dihydro-indol-2-one (Compound 74)
3-(2-methyl-1H-indol-3-ylmethylene)-1,3-dihydro-indol-2-one (Compound 75)
3-(5-fluoro-1-methyl-1H-indol-3-ylmethylene)-1,3-dihydro-indol-2-one (Compound 76)
3-(5-fluoro-1H-indol-3-ylmethylene)-1,3-dihydro-indol-2-one (Compound 77)
3-(5-methoxy-1H-indol-3-ylmethylene)-1,3-dihydro-indol-2-one (Compound 78)
3-(5-benzyloxy-1H-indol-3-ylmethylene)-1,3-dihydro-indol-2-one (Compound 79)
3-(6-methoxy-1H-indol-3-ylmethylene)-1,3-dihydro-indol-2-one (Compound 80)
3-(5-methoxy-1-methyl-1H-indol-3-ylmethylene)-1,3-dihydro-indol-2-one (Compound 81)
3-(6-methoxy-1-methyl-1H-indol-3-ylmethylene)-1,3-dihydro-indol-2-one (Compound 82)
3-(4-benzyloxy-1H-indol-3-ylmethylene)-1,3-dihydro-indol-2-one (Compound 83)
3-(1H-indol-3-ylmethylene)-2-oxo-2,3-dihydro-1H-indol-6-carbonitrile (Compound 84)
3-(1H-indol-3-ylmethylene)-2-oxo-2,3-dihydro-1H-indol-7-carbonitrile (Compound 85)
3-(1H-indol-3-ylmethylene)-2-oxo-2,3-dihydro-1H-indol-5-carbonitrile (Compound 86)
7-fluoro-3-(1H-indol-3-ylmethylene)-1,3-dihydro-indol-2-one (Compound 87)
3-(1H-indol-3-ylmethylene)-6-trifluoromethyl-1,3-dihydro-indol-2-one (Compound 88)
3-(1H-indol-3-ylmethylene)-6-methanesulfonyl-1,3-dihydro-indol-2-one (Compound 89)
3-(1H-indol-3-ylmethylene)-5-trifluoromethyl-1,3-dihydro-indol-2-one (Compound 90)
3-(1H-indol-3-ylmethylene)-5,6-dimethoxy-1,3-dihydro-indol-2-one (Compound 91)
4,5-difluoro-3-(1H-indol-3-ylmethylene)-1,3-dihydro-indol-2-one (Compound 92)
3-(1H-indol-3-ylmethylene)-5-methoxy-1,3-dihydro-indol-2-one (Compound 92A)
6-chloro-3-(1H-indol-3-ylmethylene)-1,3-dihydro-indol-2-one (Compound 93) and
3-[1-Methyl-2-(4-methyl-piperazin-1-yl)-1H-indol-3-ylmethylene]-1,3-dihydro-indol-2-one (Compound 94)

Compounds of formula I are generally lipophilic in nature which makes the compounds sparingly soluble in water and consequently difficult to formulate in, for instance, parenteral, injectable compositions where isotonic saline is used as the solvent. To provide an adequate solubility of the compounds, they may advantageously be provided in the form of prodrugs. The term “prodrug” is intended to indicate a derivative of an active compound which does not, or does not necessarily, exhibit the physiological activity of the active compound, but which may be subjected to enzymatic cleavage such as hydrolysis in vivo so as to release the active compound on administration of the prodrug. In this particular instance, the prodrug comprises the active compound which in itself is highly lipophilic provided with a side chain with predominantly hydrophilic properties imparting improved solubility characteristics to the prodrug, thereby making it more suitable for parenteral administration in the form of a solution or for oral administration to obtain an improved bioavailability. Examples of prodrugs are compounds of formula II wherein R₈, apart from being hydrogen or alkyl, is a group —PO(OR)(OR′), wherein R and R′ are independently selected from hydrogen or C₁₋₆ alkyl, —C(O)OR₁₀, —C(O)NR₁₀R₁₁, wherein R₁₀ and R₁₁ are as defined above, —CH₂-aryl-OR₁₄, —C(O)R₁₄, wherein R₁₄ is hydrogen, C₁₋₆ alkyl, C₂₋₆ alkenyl, C₂₋₆ alkynyl, cycloalkyl or aryl, —C(R₂₄R₂₅)—OR₁₆ or —OC(O)R₁₆, wherein R₁₆ and R₁₇ are the same or different and independently selected from the group consisting of hydrogen, C₁₋₆ alkyl, aralkyl, acyl or —PO(OR)(OR′), or wherein R₁₆ and R₁₇ together with the nitrogen atom to which they are attached form a heterocyclic or heteroaryl ring, or —C(R₂₄R₂₅)—NR₂₆R₂₇, wherein R₂₄ is hydrogen, C₁₋₆ alkyl or aryl, R₂₅ is hydrogen, and R₂₆ and R₂₇ are independently hydrogen or C₁₋₆ alkyl or, together with the nitrogen atom to which they are attached, form a heteroaryl or heterocyclic ring. Such prodrugs and other, similar prodrugs may suitably be prepared by a procedure described in WO 01/90068, WO 01/90103, WO 01/90104 and WO 02/081466, the disclosures of which are hereby incorporated by reference in their entirety.

Currently favoured compounds of formula I are 3-(3,5-dimethyl-1H-pyrrol-2-yl-methylene)-1,3-dihydro-indol-2-one (Compound 226), 3-(1H-indol-3-ylmethylene)-1,3-dihydroindol-2-one (Compound 57) and 3-(2,5-dimethoxy-benzylidene)-1,3-dihydroindol-2-one (Compound 110) as well as derivatives thereof, e.g. the compounds shown in Table 4 or Table 7 below. These compounds have surprisingly been found to exhibit certain properties making them particularly favourable for the present purpose: they are highly lipophilic (logP 4.7) which might enable them to cross the blood-brain barrier, and when tested on a number of different cytokines, they have been found to be potent inhibitors of the proinflammatory cytokines IL-2, IL-6, INF-γ and IL-17 (at a concentration of 10⁻¹⁰ M) while not inhibiting cytokines such as TNF-α. In case of the latter, it is interesting to note that a trial where anti-TNF antibodies were used in the treatment of multiple sclerosis actually led to an exacerbation of the symptoms (Karni and Weiner, supra). It would therefore appear to be an advantage to provide a treatment of multiple sclerosis which does not involve inhibition of TNF-α.

As indicated above, compounds of formula I have been described as tyrosine kinase inhibitors and, in particular, the compound 3-(3,5-dimethyl-1H-pyrrol-2-yl-methylene)-1,3-dihydro-indol-2-one (Compound 226) has been in development as an inhibitor of the vascular endothelial growth factor receptor KDR for the treatment of cancer. The compound has also been found to inhibit the p60c tyrosine kinase (L. Sun et al., J. Med. Chem. 43, 2000, p. 2655). In this context, it is interesting to note that compounds derived from a benzylidene malononitrile scaffold, termed tyrphostins, have been found to to be effective in the EAE model. Tyrphostins are known tyrosine kinase inhibitors. In particular, tyrphostin B42 (also termed AG490) has been found to inhibit IL-12 induced tyrosine phsophorylation and activation of JAK-2 kinase (cf. J. J. Bright et al., J. Immunol. 162, 1999, pp. 6255-6262). Furthermore, tyrphostin AG490 has been found to be an effective inhibitor of lymphocyte adhesion to inflamed vessels (cf. G. Constantin et al., J. Immunol. 162, 1999, pp. 1144-1149). It is suggested that the ability of AG490 to block the development of EAE may be ascribed, at least in part, to its ability to inhibit lymphocyte adhesion.

It is recognised that tyrosine kinases play an important role in the regulation of cell signalling by phosphorylating tyrosine residues of proteins and peptides, and that excessive activation of tyrosine kinases may lead to the development of various diseases of the immune system. In this regards, members of the src kinase family have been found to be of interest, in particular the p56lck kinase which is only expressed in T-cells and which is crucial for T-cell receptor mediated signal transduction, eventually leading to production of proinflammatory cytokines, including IL-2. It has been found that T-cells which lack the p56lck kinase cannot signal through the T-cells receptor (D. B. Straus and A. Weiss, Cell 70, 1992, p. 585). As other tyrosine kinases are seemingly unable to compensate for blocking p56lck activity (M. R. Myers et al., Curr. Pharm. Design 3, 1997, p. 473), this kinase may be an attractive target for therapeutic intervention.

It is well established that T-cells contribute to the development of several chronic inflammatory and autoimmune diseases. Initially in the disease process, naïve T-cells are activated by antigens and produce the proinflammatory cytokine interleukin-2 (IL-2) leading to clonal expansion and production of other inflammatory cytokines involved in the generation of the inflammatory or autoimmune response. Excessive T-cell activity is involved in allergies and immunoinflammatory diseases such as asthma, psoriasis, rheumatoid arthritis and multiple sclerosis. IL-2 has been found to have an important role in promoting the growth of T-cells in that it is a growth factor for both CD4+ and CD8+ T-cells as well as natural killer cells. Furthermore, IL-2 influences the differentiation of T helper cells into Th1 and Th2 cells and potentiates the production of cytokines by each cell type. IL-2 appears to be initially produced by activated CD4+ T-cells, inducing proliferation of CD8+ T-cells and production of proinflammatory cytokines such as IL-1, IL-6 and TNF-α. Thus, IL-2 is a key factor in the primary cellular immune response and, as such, it may be an attractive target for therapeutic intervention, such as in antiinflammatory or immunomodulatory therapy.

Proinflammatory cytokines produced by activated T-cells in the central nervous system are important factors in the demyelination process characteristic of multiple sclerosis (cf. B. Gran and A. Rostami, supra). Proinflammatory cytokines are believed to participate directly in myelin destruction and axonal damage (O'Connor et al., supra) and also to play a role in the upregulation of MHC class II molecules on astrocytes and microglia as well as adhesion molecules on the blood-brain barrier endothelium, facilitating the further influx of T-cells, B-cells and macrophages in the central nervous system (Hellings et al., supra). Such cytokines may also be attractive targets for therapeutic intervention.

The present inventors have indeed established a correlation between in vitro IL-2 inhibition and significant activity of compounds of formula I in the EAE model. Out of 50 compounds tested in vitro in an IL-2 assay (e.g. as described in example 7 below), 45% of those compounds that inhibited IL-2 with a pIC₅₀ (−log IC₅₀) value of 6 (i.e. 10⁻⁶ M) or more were also found to exhibit significant inhibitory activity in the EAE model, 50% of those compounds that inhibited IL-2 with a pIC₅₀ value of 7 (i.e. 10⁻⁷ M) or more were also found to exhibit significant inhibitory activity in the EAE model, and 61% of those compounds that inhibited IL-2 with a pIC₅₀ value of 8 (i.e. 10⁻⁸ M) or more were found to exhibit significant inhibitory activity against EAE. Conversely, only 38% of all 50 tested compounds, when not sorted by IL-2 inhibitory activity, showed any significant inhibitory activity in the EAE model. None of the compounds that did not significantly inhibit IL-2 in the assay showed any significant inhibitory activity in the EAE model. Thus, the in vitro IL-2 assay may be a useful tool when screening compounds that might exhibit activity in vivo in the EAE model.

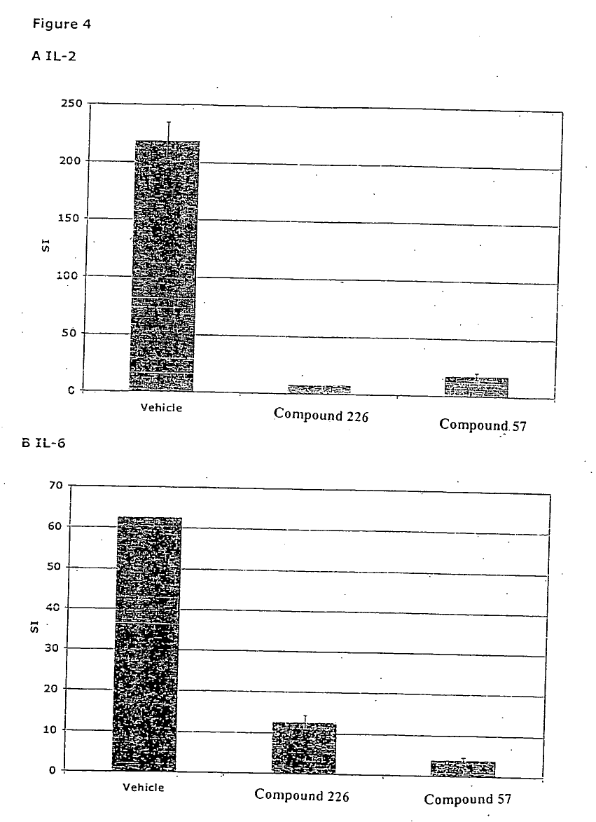

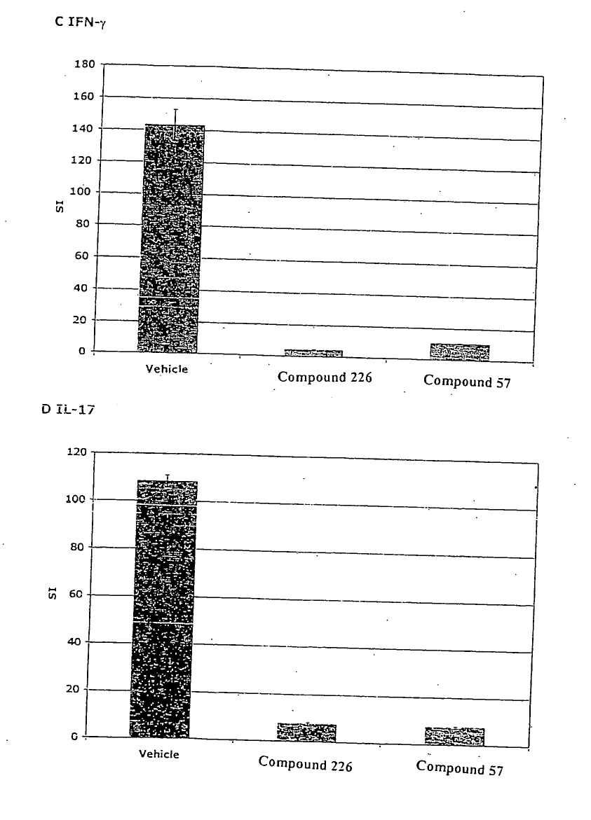

In a further embodiment, the invention therefore relates to the use of a compound of general formula I capable of inihibiting the production of proinflammatory cytokines, in particular IL-2, by T-cells or capable of blocking a cytokine receptor for the preparation of a medicament for the prevention, treatment or amelioration of multiple sclerosis, or to delay the onset of or reduce the relapse rate in multiple sclerosis. Examples of such compounds are 3-(3,5-dimethyl-1H-pyrrol-2-yl-methylene)-1,3-dihydro-indol-2-one and 3-(1H-indol-3-ylmethylene)-1,3-dihydro-indol-2-one) which, as discussed above, have been found to inhibit expression of IL-2, IL-6, INF-γ and IL-17 (cf. example 6 and FIG. 4A-4D). Other compounds capable of inhibiting IL-2 are shown in Table 6 below.

When spleen cell cDNA derived from EAE mice treated with 3-(3,5-dimethyl-1H-pyrrol-2-yl-methylene)-1,3-dihydro-indol-2-one and untreated EAE mice was tested in an oligonucleotide array of more than 5000 mouse genes (such as Atlas Plastic Mouse 5 K MicroArray, available from BD Biosciences, California, USA), a pattern emerged in cells from treated mice of downregulation of genes involved in the inflammatory response and upregulation of genes encoding adhesion molecules, suggesting that maturation and activation of inflammatory cells is retarded and consequently their migration into the CNS is reduced in the treated mice.

Pharmaceutical Compositions

For use in the present invention, the active ingredient may be formulated into a pharmaceutical composition together with a pharmaceutically acceptable vehicle and optionally one or more other therapeutic ingredients. The vehicle must be “pharmaceutically acceptable” in the sense of being compatible with the other ingredients of the formulation and not deleterious to the recipient thereof. The formulation may be in a form suitable for oral or parenteral (including subcutaneous, intramuscular, interperitoneal, intraarticular and intravenous) administration.

The formulations may conveniently be presented in dosage unit form and may be prepared by any of the methods well known in the art of pharmacy, e.g. as disclosed in Remington, The Science and Practise of Pharmacy, 20^{th} Ed., 2000. All methods include the step of bringing the active ingredient into association with the vehicle which constitutes one or more auxiliary constituents. In general, the formulations are prepared by uniformly and intimately bringing the active ingredient into association with a liquid vehicle or a finely divided solid vehicle or both, and then, if necessary, shaping the product into the desired formulation.

The term “dosage unit” is understood to mean a unitary, i.e. a single dose which is capable of being administered to a patient, and which may be readily handled and packed, remaining as a physically and chemically stable unit dose comprising either the active ingredient as such or a mixture of it with solid or liquid pharmaceutical vehicle materials.

Unlike IFN-β and glatiramer acetate which are peptidic in nature and only suitable for parenteral administration, such by injection, the compounds of formula I are small organic molecules and may therefore be administered orally. This represents a clear benefit for the patient as it permits self-medication and is less painful than for instance injections of IFN-β which often cause pain at the site of injection. Compounds of formula I, in particular compounds of formula II and III, have surprisingly exhibited an acceptable oral bioavailability and EAE inihibitory activity, cf. Table 7 below, an may therefore be suitable for oral administration.

Formulations suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid, such as ethanol or glycerol; or in the form of an oil-in-water emulsion or a water-in-oil emulsion. Such oils may be edible oils, such as e.g. cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose and polyvinylpyrrolidone. The active ingredient may also be administered in the form of a bolus, electuary or paste.

A tablet may be prepared by compressing or moulding the active ingredient optionally with one or more auxiliary constituents. Compressed tablets may be prepared by compressing, in a suitable machine, the active ingredient(s) in a free-flowing form such as a powder or granules, optionally mixed by a binder, such as e.g. lactose, glucose, starch, gelatine, acacia gum, tragacanth gum, sodium alginate, carboxymethylcellulose polyethylene glycol, waxes or the like; a lubricant such as e.g. sodium oleate, sodium stearate, magnesium steatrate, sodium benzoate, sodium acetate, sodium chloride or the like; a disintegrating agent such as e.g. starch, methyl cellulose, agar, bentonite, xanthan gum or the like or dispersing agent. Moulded tablets may be made by moulding, in a suitable machine, a mixture of the powdered active ingredient and suitable carrier moistened with an inert liquid diluent.

Formulations suitable for parenteral administration may conveniently comprise a sterile oily or aqueous preparation of the active ingredients, which is preferably isotonic with the blood of the recipient, e.g. an isotonic saline, isotonic glucose solution or buffer solution. Liposomal formulations may also be used to present the active ingredient for parenteral administration. The formulation may conveniently be sterilised by for instance filtration through a bacteria retaining filter, addition of sterilising agent to the formulation, irradiation of the formulation or heating of the formulation.

Alternatively, the formulation may be provided as a sterile, solid preparation, e.g. a freeze-dried powder, which is readily dissolved in a sterile media immediately prior to use.

In addition to the aforementioned ingredients, the formulations comprising a compound of formula I may include one or more additional ingredients such as diluents, buffers, flavouring agents, colourants, surface active agents, thickeners, preservatives, e.g. methyl hydroxybenzoate (including anti-oxidants), emulsifying agents and the like.

In addition to the formulations described above, compounds of formula I may also be formulated as a depot preparation. Such long-acting formulations may be administered by implantation (e.g. subcutaneously or intramuscularly) or by intramuscular injection. Thus, for example, the active ingredient may be formulated with suitable polymeric or hydrophobic materials (for example as an emulsion in a pharmaceutically acceptable oil), or an ion exchange resin.

In a still further aspect, the present invention relates to a method of preventing, treating or ameliorating multiple sclerosis, or delaying the onset of or reducing the relapse rate in multiple sclerosis, the method comprising administering, to a patient in need thereof, a pharmacologically effective amount of a compound of general formula I. The invention further relates to a method of preventing, treating or ameliorating multiple sclerosis, or delaying the onset of or reducing the relapse rate in multiple sclerosis, the method comprising administering, to a patient in need thereof, a pharmacologically effective amount of a compound capable of inhibiting the production of one or more proinflammatory cytokines, such as IL-2, by CD4+ Th1 cells, as discussed above.

For systemic treatment according to the present invention, daily doses of from 0.001-100 mg/kg body weight, preferably from 0.002-15 mg/kg body weight, for example 0.003-10 mg/kg of a compound of formula I or II are administered, typically corresponding to a daily dose for an adult human of from 0.2 to 750 mg of the active ingredient. Oral compositions are formulated, preferably as tablets, capsules, or drops, containing from 0.05-250 mg, preferably from 0.1-125 mg, of a compound of formula I per dosage unit.

The invention is further described in the following examples which are not in any way intended to limit the scope of the invention as claimed.

### EXAMPLES

The compounds of general formula I can be prepared in a number of ways well known to those skilled in the art of organic synthesis. The compounds of formula I can be synthesised using the methods outlined below, together with methods known in the art of synthetic organic chemistry, or variations thereof as appreciated by those skilled in the art. Preferred methods include, but are not limited to, those described below. The compounds of formula I may be prepared using the reactions and techniques described in this section. The reactions are performed in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognised by one skilled in the art. It is understood by one skilled in the art of organic synthesis that the functionalities present on various portions of the starting molecules in a reaction must be compatible with the reagents and reactions proposed. Not all compounds of formula I falling into a given class may be compatible with some of the reaction conditions required in some of the methods described. Such restrictions to the substituents which are compatible with the reaction conditions will be readily apparent to one skilled in the art and alternative methods can be used.

The compounds of formula I can be prepared by techniques and procedures readily available to one of ordinary skill in the art, for example by following the procedures as set forth in the following Schemes. These Schemes are not intended to limit the scope of the invention in any way. All substituents, unless otherwise indicated, are previously defined. The reagents and starting materials are readily available to one of ordinary skilled in the art.

General Method A

Some of the compounds of general formula II can be prepared according to the general method A described below. 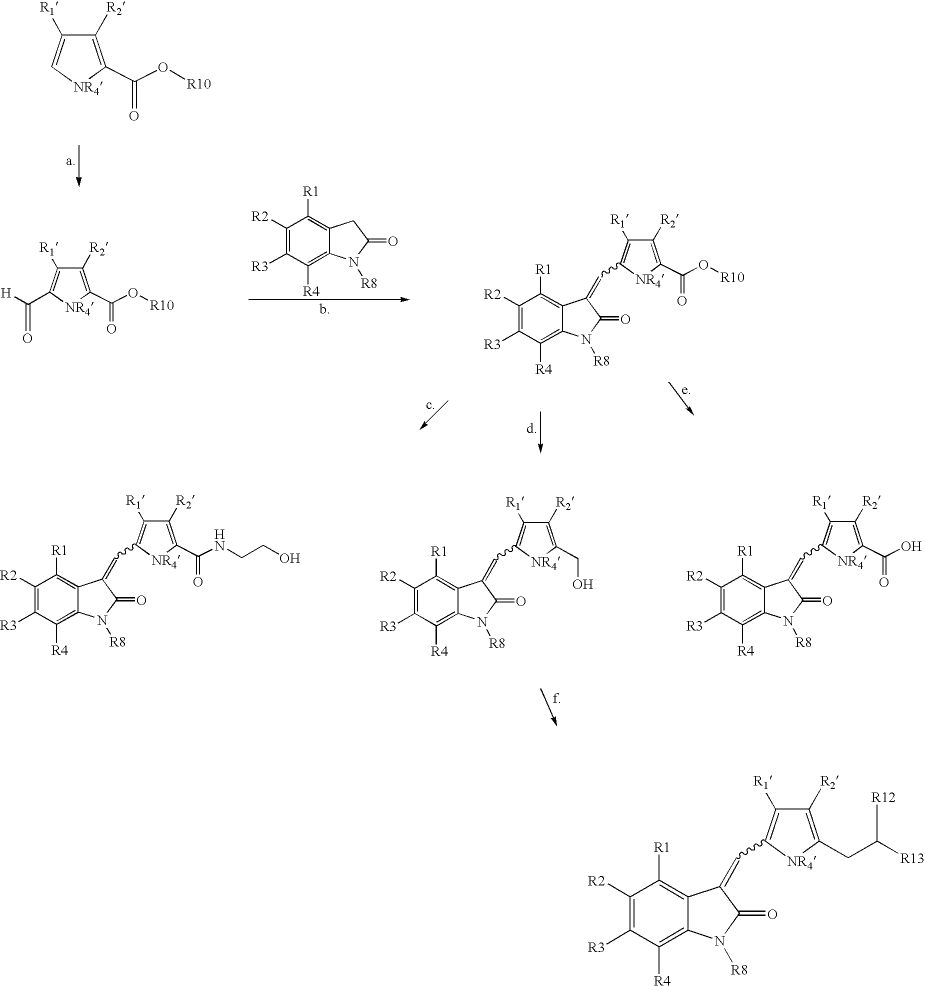
a. Oxidative agent such as (chloro-methylen)-dimethyl-ammonium-chloride in a solvent such as DCE or CH₂Cl₂ or trimethoxymethane in an appropriate solvent such as TFA.
b. Base such as piperidine, pyrrolidine or KOH in a solvent such as EtOH or toluene, under reflux.
c. Base such as K₂CO₃ and ethanolamine in a solvent such as acetonitrile.
d. Selective reductive agent such as DIBAL-H in a solvent such as toluene, at −78° C.
e. Standard hydrolysis conditions using first a base such as an aqueous solution of LiOH or NaOH followed by treatment with an acid such as an aqueous solution of HCl.
f. Sulfonyl-chloride such as methanesulfonyl chloride or 4-methyl-benzene sulfonylchloride and large excess of amine such as pyridine.
   General Method B

Some of the compounds of general formula I can be prepared according to the general method B described below. 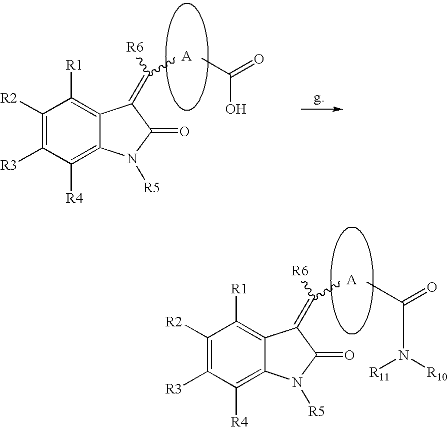
g. Formation of the amide using classical coupling agents. Preferred coupling agents include 1,1′-carbonyldiimidazole (CDI), diphenylphoshinic chloride (DPP-Cl), benzotriazol-yloxy-tripyrolidinophosphonium hexafluorophosphate (PyBOP), benzotriazol-1-yloxy-tris(dimethylamino)-phosphonium hexafluorophosphate (BOP), N,N′-dicyclohexylcarbodiimide (DCC), or 1-ethyl-3-(3-dimethylaminopropyl)-carbodiimide; hydrochloride (EDCI). Preferred bases include diisopropylethylamine, triethylamine, 4-methylmorpholine, or pyridine or a substituted pyridine, for example 4-dimethylaminopyridine or 2,6-dimethylpyridine. Preferred solvents are solvents such as diethylether, dichloromethane, tetrahydrofuran, 1-methyl-2-pyrrolidinone, dimethylsulfoxyde or dimethylformamide. The reactions are generally carried out in the presence of a base such as Et₃N or Bu₃N and in the presence of an activator such as HOBt (for example where HOBt is used to improve reactions rates, see Windridge, G. C.; Jorgensen, E. C. JACS 1971, 93, 6318), at a temperature between about −78° C. to about 60° C., and are normally complete within about 2 hours to about 5 days.
   General Method C

Some of the compounds of general formula I can be prepared according to the general method C described below. 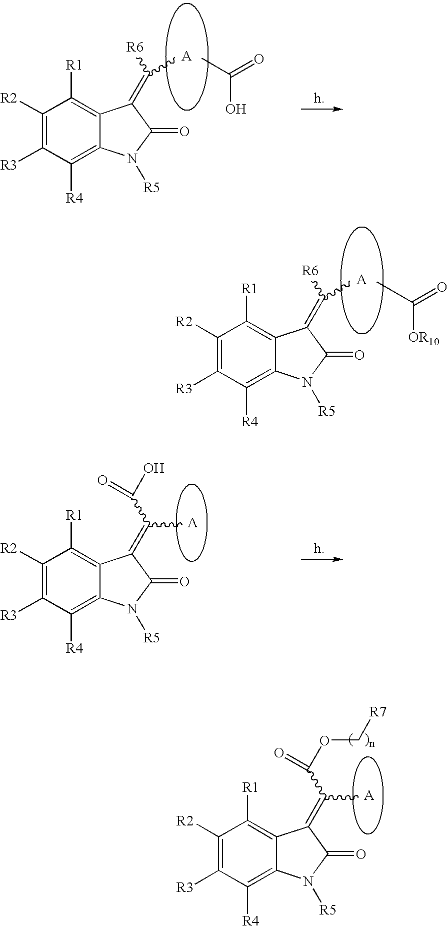
h. Esterification using classical esterification conditions (see March's Advanced Organic Chemistry Reactions, mechanisms, and Structure, 5^{th} edition, by M. B.

Smith and 1. March chapter 10-23 p. 484, chapter 10-26 p. 488 and chapter 10-28 p. 490) such as acid catalysed esterification (e.g. using sulphuric acid as a catalyst in MeOH), or such as base catalyzed esterification (e.g. using chlorosulfates as reagents under phase transfer conditions, see Synthetic Communications, 14 (9), 857-864, 1984) or such as using diazomethane in a suitable solvent such as Et₂O or EtOH, or using trimethylsilyldiazomethane in a suitable solvent such as toluene, or using an electrophilic reactant (as for example benzyl bromide or methyliodide) in the presence of a base such as K₂CO₃ or Cs₂CO₃ in a solvent such as DMF or acetonitrile.

General Method D

Some of the compounds of general formula II can be prepared according to the general method D described below. 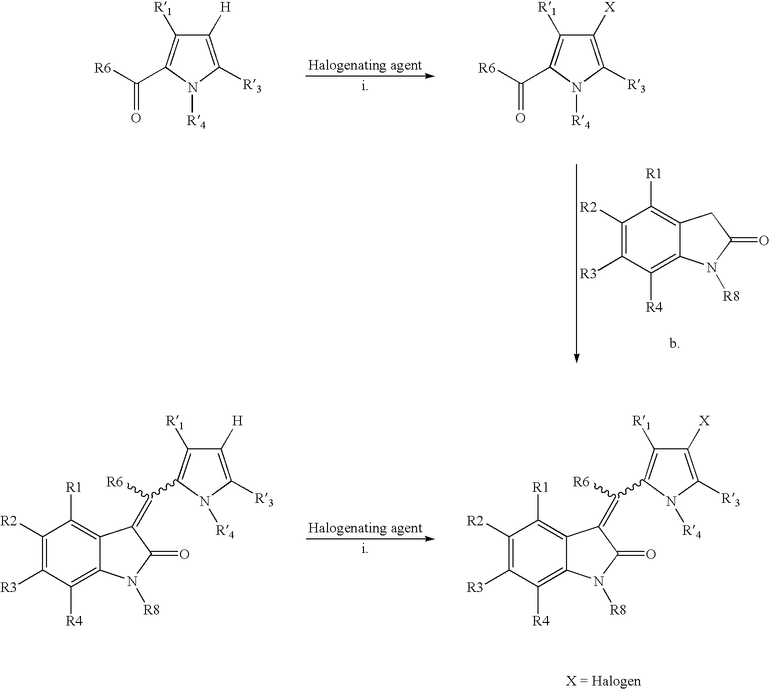
i. Halogenating agent such as NBS in the presence or absence of an initiator of radicals such as benzoylperoxide in an appropriate solvent such as CCl₄ or CH₂Cl₂ (introduction of Br) or SO₂Cl₂ in an appropriate solvent such as CH₂Cl₂ (introduction of Cl).
   General Method E

Some of the compounds of general formula IV can be prepared according to the general method E described below. 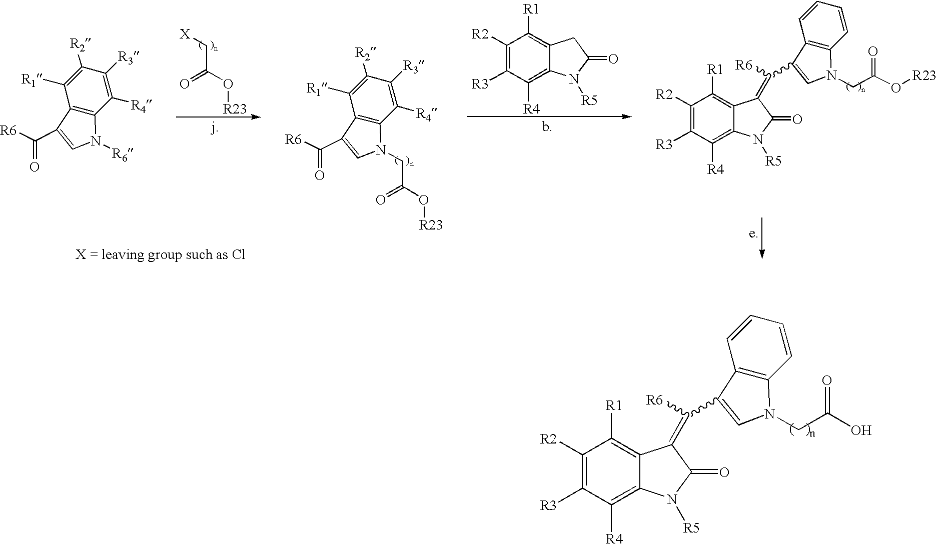
j. Base such as NaH, NAOH or KOH in the presence or absence of a activating salt such as NaI in an appropriate solvent such as DMF, DMSO, or CH₂Cl₂.
   General Method F

Some of the compounds of general formula III can be prepared according to the general method F described below.

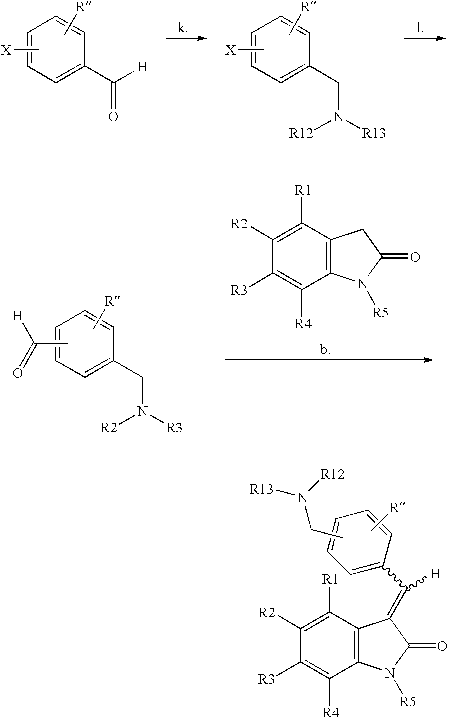

The alkyl- or dialkyl aminomethyl-benzaldehydes can be prepared in two steps:
k. Reductive amination using substituted bromo-benzaldehyde, alkyl- or dialkylamine and a reductive agent such as sodium triacetoxyborohydride.
l. Halogen-metal exchange, using for example n-BuLi in an appropriate solvent such as THF, at −78° C./−70° C., and quenching with for example N,N-dimethylformamide.
   General Method G

Some of the compounds of general formula III can be prepared according to the general method G described below. 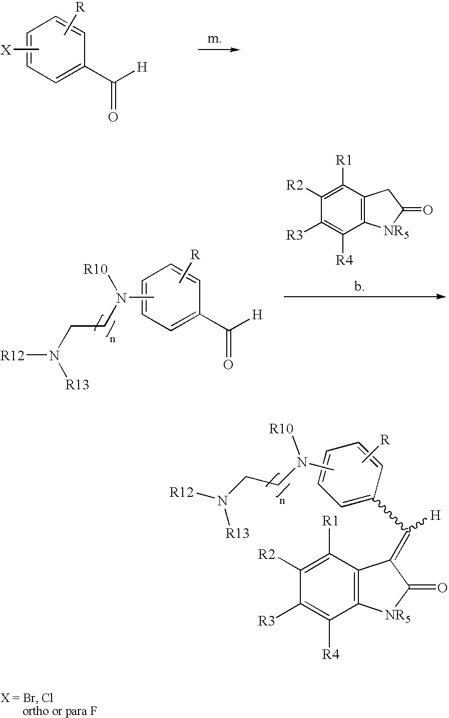
m. Aminoalkylamino-benzaldehydes can be prepared starting from bromo or chloro-benzaldehydes. As a first step, the aldehyde function will preferably be protected for example as a dialkyl acetal form, allowing palladium catalysed reaction in the presence of aminoalkylamine followed by a deprotection step in acidic media such as TFA or p-TsOH in an appropriate solvent such as CHCl₃ or acetone. Alternatively, the 2- or 4-aminoalkylamino-benzaldehydes can be prepared by nucleophilic aromatic substitution using 2- or 4-fluorobenzaldehydes, aminoalkylamines and a base such as K₂CO₃ in an appropriate solvent such as DMSO or DMF.
   General Method H

Some of the compounds of general formula I can be prepared according to the general method H described below. 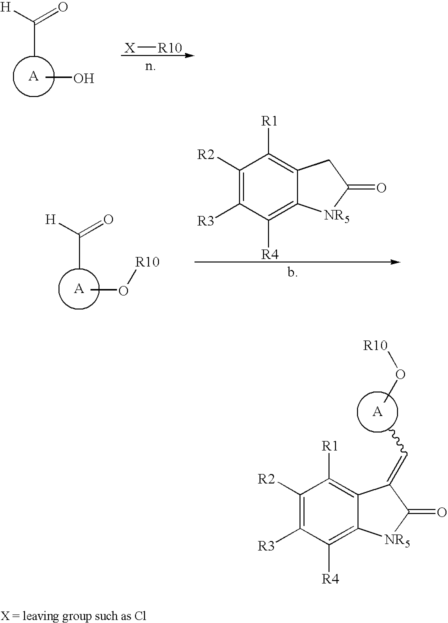
n. Base such as Cs₂CO₃ or K₂CO₃, in the presence or the absence of a salt such as NaI, in an appropriate solvent such as DMF or acetonitrile.
   General Method I

Some of the compounds of general formula I can be prepared according to the general method I described below. 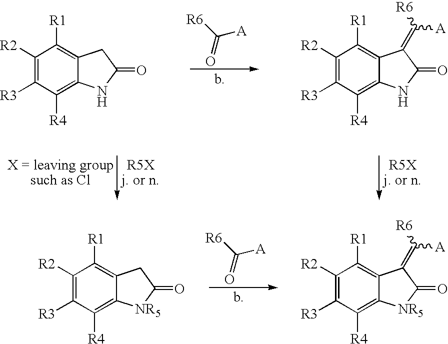
General Method J

Some of the compounds of general formula I can be prepared according to the general method J described below. 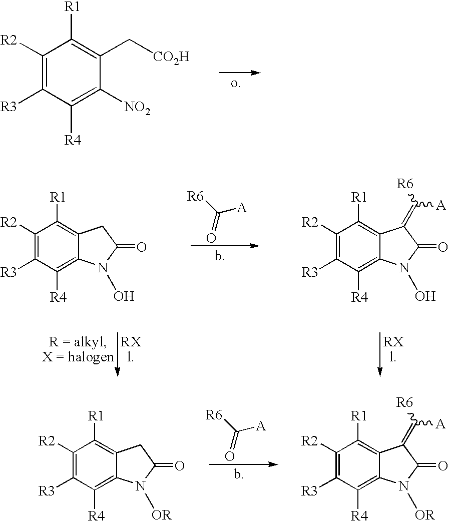
o. Reducing agent such as hydrogen in the presence of a catalyst such as Pt/C and DMSO in an appropriate solvent such as ethanol followed by treatment with an acid such as sulfuric acid in an appropriate solvent such as H₂O [Kende, A. S.;

Thurston, I., Synthetic Communication, 20 (14), 2133-2138 (1990)].

General Method K

Some of the compounds of general formula II can be prepared according to the general method K described below. 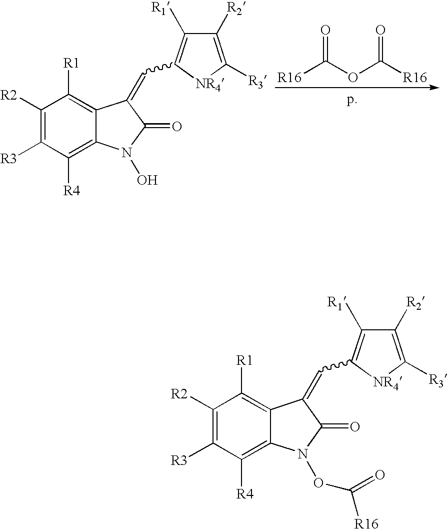
p. Base such as Et₃N, or basic catalyst such as pyridine or DMAP, or an acid or a lewis acid catalyst such as CoCl₂, in an appropriate solvent such as acetonitrile.
   General Method L

Some of the compounds of general formula I can be prepared according to the general method L described below. 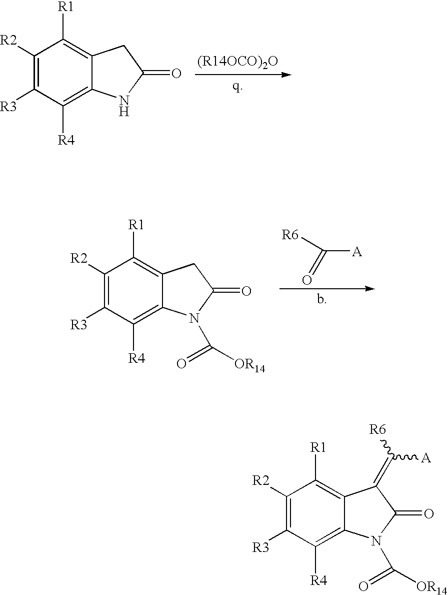
q. in the presence of a basic catalyst such as pyridine or DMAP or in the presence an acid or a lewis acid catalyst such as CoCl₂, in an appropriate solvent such as acetonitrile.

### General Procedures, Preparations and Examples

For ¹H nuclear magnetic resonance (NMR) spectra (300 MHz) and ¹³C NMR (75.6 MHz) chemical shift values (δ) (in ppm) are quoted for dimethyl-d₆ sulfoxide (DMSO-d₆) solutions relative to internal tetramethylsilane (δ=0) standard. The value of a multiplet, either defined (doublet (d), triplet (t), quartet (q)) or not (m) at the approximate mid point is given unless a range is quoted, (bs) indicates a broad singlet. The ES mass spectra were obtained on a VG Quattro II triple quadrapole mass spectrometer (Micromass, Manchester, UK) operating in either positive or negative electrospray mode with a cone voltage of 30V.

The compounds of the present invention can exist in two isomeric forms: the Z and the E isomeric forms. The NMR data characterize the isomer forms that are present in the solvent used to record the NMR spectrum and determine their molar ratio. For NMR solutions where both the E-isomer and the Z-isomer are present in equal or close to equal amounts, the chemical shifts of both forms are given. For NMR solutions where the equilibrium is shifted in favour of one form, the chemical shifts of the dominating form are given.

The organic solvents used were anhydrous unless otherwise specified. Flash chromatography was performed on silica gel.

The following abbreviations have been used throughout:
Ac Acetate or acetyl
Brine Saturated aqueous sodium chloride
Boc tert-Butoxycarbonyl
DCE Dichloro-ethane
DIBAL-H Diisobutyl aluminum hydride
DMAP 4-Dimethylaminopyridine
DMF N,N′-Dimethylformamide
DMSO Dimethylsulfoxide
EDCI 1-ethyl-3-(3-dimethylaminopropyl)-carbodiimide
EtOAc Ethyl acetate
eq. Equivalent
HOBt 1-hydroxybenzotriazole
M Molar (mol/L)
NBS N-Bromosuccinimide
NMR Nuclear magnetic resonance
PMB 4-Methoxybenzyl
PMBCl 4-Methoxybenzyl chloride
THF Tetrahydrofuran
TFA Trifluoroacetic acid
pTsOH Para-toluene sulfonic acid

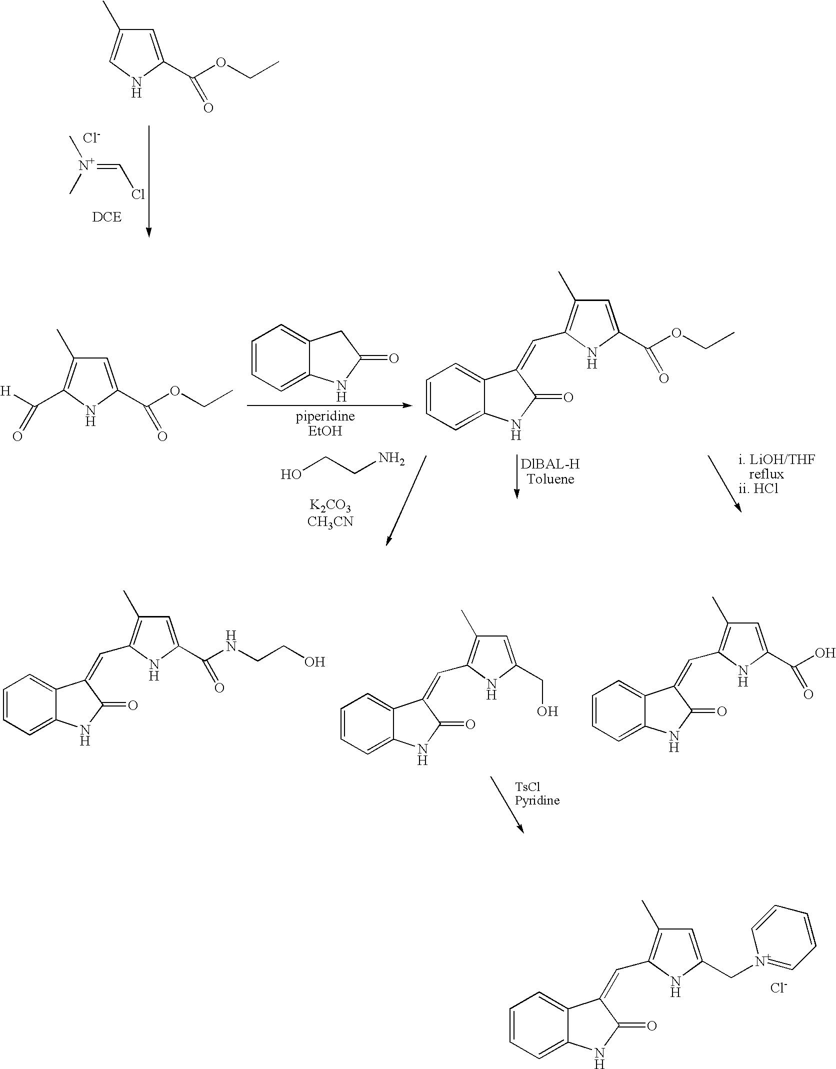

### Preparation 1

### 5-Formyl-4-methyl-1H-pyrrole-2-carboxylic acid ethyl ester

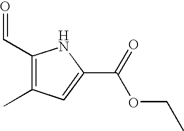

To a solution of (chloro-methylen)-dimethyl-ammonium-chloride (1.64 g, 12.8 mmoles) in dry DCE (6 mL) was added dropwise under argon atmosphere a solution of 4-methyl-1H-pyrrole-2-carboxylic acid ethyl ester (1.31 g, 8.6 mmoles) in DCE (5 mL). The reaction mixture was heated slightly to dissolve all solid particules and stirred at room temperature overnight. TLC indicated that there was no starting material left. The pH of the reaction mixture was adjusted to 10 by addition of NaOH (2 N). The crude mixture was extracted with CH₂Cl₂ (3×10 mL). The combined organic phases were washed once with H₂O, once with brine, dried over MgSO₄ and filtered. After removal of the solvent, 1.49 g of the title compound (99% yield) were obtained as a pale red/orange solid and used in the next step without further purification.

¹H NMR (DMSO-d₆) δ 12.6 (br, 1H), 9.80 (s, 1H), 6.70 (s, 1H), 4.27 (q, 2H), 2.30 (s, 3H), 1.30 (t, 3H).

### General Procedure 1

A solution of aldehyde (1 eq.), 1,3-dihydro-indol-2-one (1 eq.) and piperidine (catalytic amount) in EtOH is heated to reflux overnight. The crude mixture is allowed to come to room temperature, cooled in an ice bath. A precipitate is obtained which is filtered and washed with cold EtOH. If no precipitation occurs, the solvent is removed and the product is purified by flash chromatography.

### Preparation 2

### Compound 01, 4-methyl-5-(2-oxo-1,2-dihydro-indol-3-ylidenemethyl)-1H-pyrrole-2-carboxylic acid ethyl ester

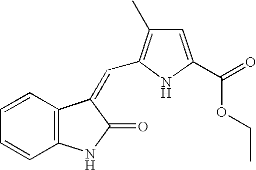

The general procedure 1 was followed using a mixture of 5-formyl-4-methyl-1H-pyrrole-2-carboxylic acid ethyl ester (0.93 g, 7 mmoles), 1,3-dihydro-indol-2-one (1.27 g, 7 mmoles) and piperidine (few drops) in EtOH (25 mL). After filtration, 1.98 g of the title compound as orange crystals were obtained (99% yield).

¹³C-NMR (DMSO-d₆, Z isomer) δ 169.0, 159.7, 139.4, 129.9, 128.8, 128.0, 124.5, 124.3, 122.5, 121.4, 120.7, 119.6, 116.4, 109.7, 60.3, 14.2, 11.0.

### Preparation 3

### Compound 02, 4-methyl-5-(2-oxo-1,2-dihydro-indol-3-ylidenemethyl)-1H-pyrrole-2-carboxylic acid (2-hydroxy-ethyl)-amide

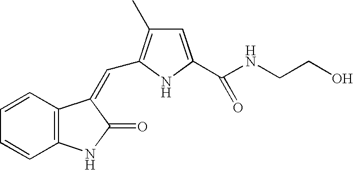

A mixture of 4-methyl-5-(2-oxo-1,2-dihydro-indol-3-ylidenemethyl)-1H-pyrrole-2-carboxylic acid ethyl ester (0.46 g, 1.57 mmoles) K₂CO₃ (0.35 g, 2.52 mmoles) and ethanolamine (28 mL, 466 mmoles) in acetonitrile (14 mL) was heated at 105° C. for 1 hour (the reaction was followed by TLC). The mixture was allowed to come to room temperature, poured into H₂O, extracted with EtOAc (3×20 mL). The combined organic phases were washed once with H₂O, once with brine and dried over MgSO₄. After filtration and removal of the solvent, 0.42 g of the title compound were obtained as an orange solid (85% yield).

¹H NMR (DMSO-d₆) δ 13.64 (s, 1H), 10.89 (br, 1H), 8.32 (t, 1H), 7.80 (d, 1H), 7.64 (s, 1H), 7.18 (t, 1H), 7.01 (t, 1H), 6.88 (d, 1H), 6.83 (d, 1H), 4.73 (t, 1H), 3.50 (m, 2H), 3.3 (m, 2H), 2.35 (s, 3H).

### Preparation 4

### Compound 03, 3-(5-hydroxymethyl-3-methyl-1H-pyrrol-2-ylmethylene)-1,3-dihydro-indol-2-one

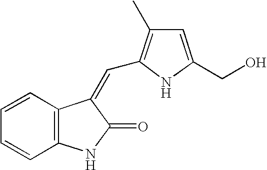

To a dry solution of 4-methyl-5-(2-oxo-1,2-dihydro-indol-3-ylidenemethyl)-1H-pyrrole-2-carboxylic acid ethyl ester (1 g, 3.38 mmoles) in dry toluene (20 mL) was added under argon and at −78° C. a solution 1 M of DIBAL-H in toluene (34 mL). The reaction was followed by TLC. After 1.5 hours, extra amounts (17 mL and 10 mL) of the solution of DIBAL-H (1 M in toluene) were added at −78° C. and the temperature was allowed to raise to −40° C.

MeOH (60 mL) was added at −75° C. and the mixture was allowed to come to room temperature. After addition of saturated aqueous NH₄Cl solution (40 mL), the crude mixture was extracted with EtOAc (3×60 mL). The combined organic phases were washed once with H₂O, once with brine and dried over MgSO₄. After filtration and removal of the solvent, 0.88 g of 3-(5-hydroxymethyl-3-methyl-1H-pyrrol-2-ylmethylene)-1,3-dihydro-indol-2-one. After crystallization from EtOAc and petroleum ether, 0.58 g of pure title compound were obtained (68% yield) as orange crystals.

¹³C-NMR (DMSO-d₆, Z isomer) δ 169.2, 140.0, 138.3, 130.7, 126.4, 125.9, 125.7, 123.3, 120.7, 118.1, 113.6, 110.2, 109.2, 56.6, 11.3.

### Preparation 5

### Compound 04, 1-[4-methyl-5-(2-oxo-1,2-dihydro-indol-3-ylidenemethyl)-1H-pyrrol-2-ylmethyl)-pyrridinum; chloride

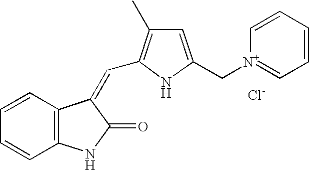

To a solution of 3-(5-hydroxymethyl-3-methyl-1H-pyrrol-2-ylmethylene)-1,3-dihydro-indol-2-one (50 mg, 0.2 mmole) in pyridine (0.5 mL) cooled in an ice bath was added dropwise 4-methyl-benzenesulfonyl chloride (41.7 mg, 0.22 mmole). The mixture was stirred at 0° C. for 2 hours. H₂O (3 mL) was added to the crude mixture and the aqueous phase was extracted with EtOAc (3×3 mL). A precipitate formed in the aqueous phase which was filtered to afford 35 mg of the title compound as an orange solid (50% yield).

¹³C-NMR (DMSO-d₆, Z isomer) δ 169.2, 146.1, 144.7, 138.9, 129.7, 129.1, 128.4, 128.4, 127.0, 124.9, 123.2, 121.1, 119.0, 117.1, 113.7, 109.6, 56.7, 11.1.

### General Procedure 2

A solution of the ester (1 eq.) in a 1:1 mixture of THF or MeOH and aqueous solution of LiOH is heated under reflux. The reaction is followed by TLC. When TLC indicates full conversion the pH of the solution is adjusted to acidic pH (6-7) by addition of HCl (1N). A precipitate forms which is filtered and dried furnishing the desired product.

### Preparation 6

### Compound 05, 4-methyl-5-(2-oxo-1,2-dihydro-indol-3-ylidenemethyl)-1H-pyrrole-2-carboxylic acid

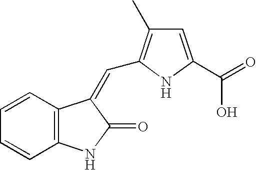

The general procedure 2 was followed using a solution of 4-methyl-5-(2-oxo-1,2-dihydro-indol-3-ylidenemethyl)-1H-pyrrole-2-carboxylic acid ethyl ester (2.8 g, 9.5 mmoles) in a mixture of THF (150 mL) and an aqueous solution of LiOH (1N). After heating at reflux for 4 hours, the pH of the reaction was adjusted to acidic pH by addition of an aqueous solution of HCl 1 N (about 150 mL). A bright orange precipitate formed which was filtered to afford the title compound as an orange solid (2.47 g, 97% yield).

¹³C-NMR (DMSO-d₆, Z isomer) δ 169.2, 161.3, 139.6, 129.7, 129.0, 128.0, 125.6, 124.8, 122.7, 121.5, 120.5, 119.6, 116.4, 109.8, 11.1.

### General Procedure 3

To a mixture of 4-methyl-5-(2-oxo-1,2-dihydro-indol-3-ylidenemethyl)-1H-pyrrole-2-carboxylic acid (1 eq.), EDCI (1.5 eq.), HOBt (1.6 eq.), Et₃N (2 eq. or 4 eq.) in DMF is added the corresponding amine as a free base or as a salt (2 eq.). The crude mixture is then stirred or shaked at room temperature overnight. After partial removal of the solvent, a saturated aqueous solution of Na₂CO₃ is added slowly. A precipitate forms. It is filtered and if necessary purified by flash chromatography.

### Preparation 7

### Compound 06, 4-methyl-5-(2-oxo-1,2-dihydro-indol-3-ylidenemethyl)-1H-pyrrole-2-carboxylic acid (2-diethylamino-ethyl)-amide

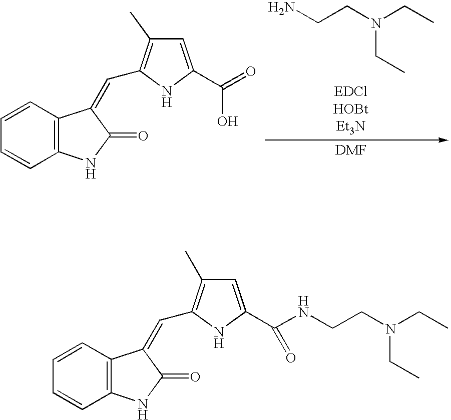

The title compound was obtained by following the general procedure 3 using 4-methyl-5-(2-oxo-1,2-dihydro-indol-3-ylidenemethyl)-1H-pyrrole-2-carboxylic acid (0.94 g, 3.5 mmoles), EDCI (1.01 g, 5.25 mmoles), HOBt (0.756 g, 5.6 mmoles), Et₃N (1 mL, 7 mmoles) in DMF (12 mL) and adding N,N-diethyl-ethane-1,2-diamine (1 mL, 7 mmoles). After filtration 0.45 g of the title compound were obtained as an orange solid (35% yield).

¹³C-NMR (DMSO-d₆, Z isomer) δ 169.0, 159.4, 139.2, 129.5, 129.1, 128.2, 127.4, 124.9, 122.8, 121.1, 119.2, 118.6, 112.1, 109.5, 51.4, 46.7, 37.0, 11.7, 11.3.

### Preparation 8

### Compound 07, 4-methyl-5-(2-oxo-1,2-dihydro-indol-3-ylidenemethyl)-1H-pyrrole-2-carboxylic acid (2-methoxy-ethyl)-amide

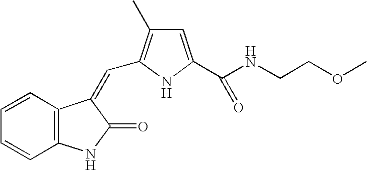

The title compound was obtained by following the general procedure 3 using 4-methyl-5-(2-oxo-1,2-dihydro-indol-3-ylidenemethyl)-1H-pyrrole-2-carboxylic acid (0.94 g, 3.5 mmoles), EDCI (1.01 g, 5.25 mmoles), HOBt (0.766 g, 5.7 mmoles), Et₃N (1 mL, 7 mmoles) in DMF (12 mL) and adding 2-methoxy-ethylamine (0.61 mL, 7 mmoles). The reaction was followed by LC/MS. After addition of H₂O (10 mL), a precipitate formed. After filtration, the solid was suspended in water and extracted with CH₂Cl₂ (3×20 mL). The organic phases were combined and concentrated in vacuo to obtain 0.68 g of the title compound as an orange solid (60% yield).

¹H NMR (DMSO-d₆) δ 13.65 (s, 1H), 10.91 (s, 1H), 8.40 (t, 1H), 7.80 (d, 1H), 7.65 (s, 1H), 7.17 (t, 1H), 7.00 (t, 1H), 6.89 (d, 1H), 6.84 (d, 1H), 3.43 (m, 4H), 3.27 (s, 3H), 2.35 (s, 3H).

### General Procedure 4

To a mixture of 4-methyl-5-(2-oxo-1,2-dihydro-indol-3-ylidenemethyl)-1H-pyrrole-2-carboxylic acid (1 eq.), EDCI (72 mg, 0.37 mmole, 1.5 eq.), HOBT (51 mg, 0.37 mmole, 1.5 eq.), Et₃N (0.07 mL or 0.14 mL, 0.5 or 1 mmole, 2 or 4 eq.) in DMF (0.8 mL) is added the corresponding amine as a free base or a salt (2 eq.). The crude mixture is shaked or stirred at room temperature overnight. After removal of DMF under high vacuum, the crude is purified by flash chromatography using MeOH/CH₂Cl₂ mixtures as eluent.

### Preparation 9

### Compound 08, 4-methyl-5-(2-oxo-1,2-dihydro-indol-3-ylidenemethyl)-1H-pyrrole-2-carboxylic acid [3-(1-formyl-piperidin-4-yl)-propyl]-amide

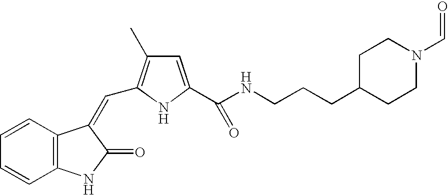

The general procedure 4 was followed using 4-methyl-5-(2-oxo-1,2-dihydro-indol-3-ylidenemethyl)-1H-pyrrole-2-carboxylic acid (67 mg, 0.25 mmole), N-formyl-4-(3-aminopropyl)-piperidin; hydrochloride (103 mg, 0.5 mmole) and Et₃N (0.14 mL, 1 mmole, 4 eq.). After purification by flash chromatography, 87 mg of the title compound were obtained as an orange solid (⁸⁴% yield).

¹H NMR (DMSO-d₆) δ 13.63 (s, 1H), 10.91 (s, 1H), 8.33 (t, 1H), 7.96 (s, 1H), 7.80 (d, 1H), 7.64 (s, 1H), 7.18 (t, 1H), 7.00 (t, 1H), 6.88 (d, 1H), 6.81 (d, 1H), 4.14 (m, 1H), 3.64 (m, 1H), 3.23 (q, 2H), 2.98 (m, 1H), 2.56 (m, 1H), 2.35 (s, 3H), 1.70 (m, 2H), 1.52 (m, 3H), 1.25 (m, 2H), 1.10-0.80 (m, 2H).

### Preparation 10

### Compound 09, 4-{[4-methyl-5-(2-oxo-1,2-dihydro-indol-3-ylidenemethyl)-1H-pyrrole-2-carbonyl]-amino}-butyric acid methyl ester

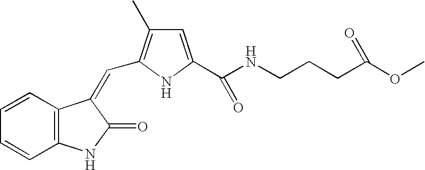

The general procedure 4 was followed using 4-methyl-5-(2-oxo-1,2-dihydro-indol-3-ylidenemethyl)-1H-pyrrole-2-carboxylic acid (67 mg, 0.25 mmole), 4-amino-butyric acid methyl ester; hydrochloride (77 mg, 0.5 mmole) and Et₃N (0.14 mL, 1 mmole, 4 eq.). After purification by flash chromatography, 60 mg of the title compound were obtained as an orange solid (65% yield).

¹H NMR (DMSO-d₆) δ 13.64 (s, 1H), 10.91 (s, 1H), 8.36 (t, 1H), 7.80 (d, 1H), 7.64 (s, 1H), 7.17 (t, 1H), 7.01 (t, 1H), 6.89 (d, 1H), 6.81 (d, 1H), 3.59 (s, 3H), 3.26 (q, 2H), 2.37 (t, 2H), 2.35 (s, 3H), 1.77 (m, 2H).

### Preparation 11

### Compound 10, 4-methyl-5-(2-oxo-1,2-dihydro-indol-3-ylidenemethyl)-1H-pyrrole-2-carboxylic acid (6-hydroxy-hexyl)-amide

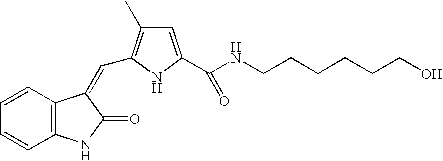

The general procedure 4 was followed using 4-methyl-5-(2-oxo-1,2-dihydro-indol-3-ylidenemethyl)-1H-pyrrole-2-carboxylic acid (67 mg, 0.25 mmole), 6-amino-hexan-1-ol (59 mg, 0.5 mmole) and Et₃N (0.07 mL, 0.5 mmole, 2 eq.). After purification by flash chromatography, 47 mg of the title compound were obtained as an orange solid (51% yield).

¹H NMR (DMSO-d₆) δ 13.63 (s, 1H), 10.91 (s, 1H), 8.31 (t, 1H), 7.80 (d, 1H), 7.64 (s, 1H), 7.18 (t, 1H), 7.00 (t, 1H), 6.88 (d, 1H), 6.81 (d, 1H), 4.32 (t, 1H), 3.38 (q, 2H), 3.23 (q, 2H), 2.35 (s, 3H), 1.6-1.2 (m, 8H).

### Preparation 12

### Compound 11, 4-methyl-5-(2-oxo-1,2-dihydro-indol-3-ylidenemethyl)-1H-pyrrole-2-carboxylic acid cyclohexylmethyl-amide

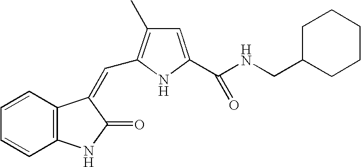

The general procedure 4 was followed using 4-methyl-5-(2-oxo-1,2-dihydro-indol-3-ylidenemethyl)-1H-pyrrole-2-carboxylic acid (67 mg, 0.25 mmole), aminomethyl-cyclohexane (57 mg, 0.5 mmole) and Et₃N (0.07 mL, 0.5 mmole, 2 eq.). After purification by flash chromatography, 24 mg of the title compound were obtained as an orange solid (26% yield).

¹H NMR (DMSO-d₆) δ 13.62 (s, 1H), 10.91 (s, 1H), 8.30 (t, 1H), 7.79 (d, 1H), 7.64 (s, 1H), 7.18 (t, 1H), 7.00 (t, 1H), 6.88 (d, 1H), 6.85 (d, 1H), 3.09 (t, 2H), 2.35 (s, 3H), 1.8-0.8 (m, 11H).

### Preparation 13

### Compound 12, 4-methyl-5-(2-oxo-1,2-dihydro-indol-3-ylidenemethyl)-1H-pyrrole-2-carboxylic acid (4-hydroxy-butyl)-amide

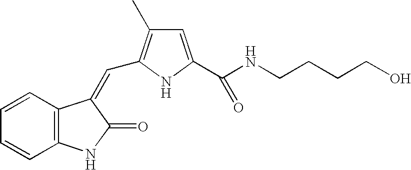

The general procedure 4 was followed using 4-methyl-5-(2-oxo-1,2-dihydro-indol-3-ylidenemethyl)-1H-pyrrole-2-carboxylic acid (67 mg, 0.25 mmole), 4-amino-1-butanol (45 mg, 0.5 mmole) and Et₃N (0.07 mL, 0.5 mmole, 2 eq.). After purification by flash chromatography, 33 mg of the title compound were obtained as an orange solid (³⁹% yield).

¹H NMR (DMSO-d₆) δ 13.63 (s, 1H), 10.91 (s, 1H), 8.32 (t, 1H), 7.80 (d, 1H), 7.64 (s, 1H), 7.18 (t, 1H), 7.00 (t, 1H), 6.88 (d, 1H), 6.81 (d, 1H), 4.40 (t, 1H), 3.42 (q, 2H), 3.24 (q, 2H), 2.35 (s, 3H), 1.6-1.35 (m, 4H).

### Preparation 14

### Compound 13, 6-{[4-methyl-5-(2-oxo-1,2-dihydro-indol-3-ylidenemethyl)-1H-pyrrole-2-carbonyl]-amino}-hexanoic acid ethyl ester

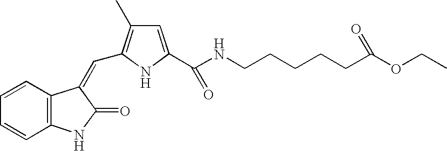

The general procedure 4 was followed using 4-methyl-5-(2-oxo-1,2-dihydro-indol-3-ylidenemethyl)-1H-pyrrole-2-carboxylic acid (67 mg, 0.25 mmole), 6-amino-hexanoic acid ethyl ester; hydrochloride (98 mg, 0.5 mmole) and Et₃N (0.14 mL, 1 mmole, 4 eq.). After purification by flash chromatography, 78 mg of the title compound were obtained as an orange solid (76% yield).

¹H NMR (DMSO-d₆) δ 13.63 (s, 1H), 10.91 (s, 1H), 8.31 (t, 1H), 7.80 (d, 1H), 7.64 (s, 1H), 7.18 (t, 1H), 7.00 (t, 1H), 6.88 (d, 1H), 6.81 (d, 1H), 4.04 (q, 2H), 3.23 (q, 2H), 2.35 (s, 3H), 2.29 (t, 2H), 1.53 (m, 4H), 1.31 (m, 2H), 1.17 (t, 3H).

### Preparation 15

### Compound 14, 4-methyl-5-(2-oxo-1,2-dihydro-indol-3-ylidenemethyl)-1H-pyrrole-2-carboxylic acid (tetrahydro-furan-2-ylmethyl)-amide

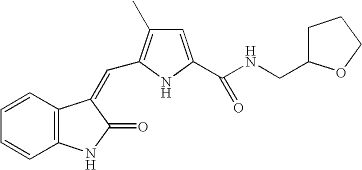

The general procedure 4 was followed using 4-methyl-5-(2-oxo-1,2-dihydro-indol-3-ylidenemethyl)-1H-pyrrole-2-carboxylic acid (67 mg, 0.25 mmole), tetrahydro-furfurylamine (51 mg, 0.5 mmole) and Et₃N (0.07 mL, 0.5 mmole, 2 eq.). After purification by flash chromatography, 64 mg of the title compound were obtained as an orange solid (73% yield).

¹H NMR (DMSO-d₆) δ 13.65 (s, 1H), 10.91 (s, 1H), 8.41 (t, 1H), 7.80 (d, 1H), 7.64 (s, 1H), 7.18 (t, 1H), 7.01 (t, 1H), 6.89 (m, 2H), 3.96 (m, 1H), 3.78 (m, 1H), 3.63 (m, 1H), 3.30 (t, 2H), 2.34 (s, 3H), 2.0-1.5 (m, 4H).

### Preparation 16

### Compound 15, 4-methyl-5-(2-oxo-1,2-dihydro-indol-3-ylidenemethyl)-1H-pyrrole-2-carboxylic acid [2-(1H-indol-3-yl)-ethyl]-amide

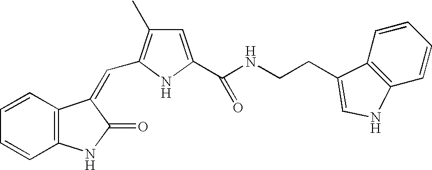

The general procedure 4 was followed using 4-methyl-5-(2-oxo-1,2-dihydro-indol-3-ylidenemethyl)-1H-pyrrole-2-carboxylic acid (67 mg, 0.25 mmole), 2-(1H-indol-3-yl)-ethylamine (80 mg, 0.5 mmole) and Et₃N (0.07 mL, 0.5 mmole, 2 eq.). After purification by flash chromatography, 34 mg of the title compound were obtained as a red solid (33% yield).

¹H NMR (DMSO-d₆) δ 13.67 (s, 1H), 10.92 (s, 1H), 10.80 (s, 1H), 8.49 (t, 1H), 7.80 (d, 1H), 7.65 (s, 1H), 7.59 (d, 1H), 7.34 (d, 1H), 7.18 (m, 2H), 7.10-6.95 (m, 3H), 6.89 (d, 1H), 6.81 (d, 1H), 3.54 (q, 2H), 2.94 (t, 2H), 2.35 (s, 3H).

### Preparation 17

### Compound 16, 4-methyl-5-(2-oxo-1,2-dihydro-indol-3-ylidenemethyl)-1H-pyrrole-2-carboxylic acid (3-phenyl-propyl)-amide

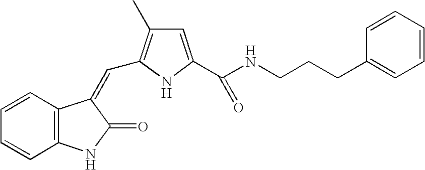

The general procedure 4 was followed using 4-methyl-5-(2-oxo-1,2-dihydro-indol-3-ylidenemethyl)-1H-pyrrole-2-carboxylic acid (67 mg, 0.25 mmole), 3-phenyl-propylamine (68 mg, 0.5 mmole) and Et₃N (0.07 mL, 0.5 mmole, 2 eq.). After purification by flash chromatography, 72 mg of the title compound were obtained as a yellow/orange solid (75% yield).

¹H NMR (DMSO-d₆) δ 13.64 (s, 1H), 10.91 (s, 1H), 8.36 (t, 1H), 7.80 (d, 1H), 7.65 (s, 1H), 7.33-7.11 (m, 6H), 7.00 (t, 1H), 6.89 (d, 1H), 6.83 (d, 1H), 3.27 (q, 2H), 2.63 (t, 2H), 2.35 (s, 3H), 1.82 (m, 2H).

### Preparation 18

### Compound 17, 4-methyl-5-(2-oxo-1,2-dihydro-indol-3-ylidenemethyl)-1H-pyrrole-2-carboxylic acid (4-phenyl-butyl)-amide

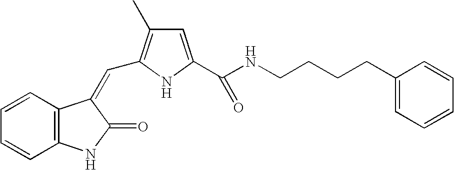

The general procedure 4 was followed using 4-methyl-5-(2-oxo-1,2-dihydro-indol-3-ylidenemethyl)-1H-pyrrole-2-carboxylic acid (67 mg, 0.25 mmole), 4-phenyl-butylamine (75 mg, 0.5 mmole) and Et₃N (0.07 mL, 0.5 mmole, 2 eq.). After purification by flash chromatography, 72 mg of the title compound were obtained as a yellow/orange solid (74% yield).

¹H NMR (DMSO-d₆) δ 13.63 (s, 1H), 10.90 (s, 1H), 8.32 (t, 1H), 7.79 (d, 1H), 7.64 (s, 1H), 7.32-7.10 (m, 6H), 7.01 (t, 1H), 6.89 (d, 1H), 6.80 (d, 1H), 3.27 (q, 2H), 2.61 (t, 2H), 2.34 (s, 3H), 1.68-1.44 (m, 4H).

### Preparation 19

### Compound 18, 4-methyl-5-(2-oxo-1,2-dihydro-indol-3-ylidenemethyl)-1H-pyrrole-2-carboxylic acid (5-hydroxy-pentyl)-amide

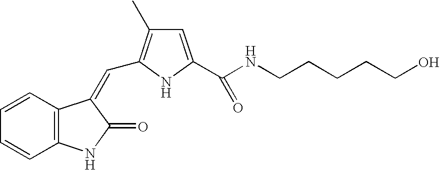

The general procedure 4 was followed using 4-methyl-5-(2-oxo-1,2-dihydro-indol-3-ylidenemethyl)-1H-pyrrole-2-carboxylic acid (67 mg, 0.25 mmole), 5-amino-1-pentanol (52 mg, 0.5 mmole) and Et₃N (0.07 mL, 0.5 mmole, 2 eq.). After purification by flash chromatography, 48 mg of the title compound were obtained as a yellow/orange solid (55% yield).

¹H NMR (DMSO-d₆) δ 13.63 (s, 1H), 10.90 (s, 1H), 8.31 (t, 1H), 7.80 (d, 1H), 7.64 (s, 1H), 7.18 (t, 1H), 7.00 (t, 1H), 6.89 (d, 1H), 6.82 (d, 1H), 4.34 (t, 1H), 3.39 (q, 2H), 3.23 (q, 2H), 2.35 (s, 3H), 1.6-1.25 (m, 6H).

### Preparation 20

### Compound 19, 4-{[4-methyl-5-(2-oxo-1,2-dihydro-indol-3-ylidenemethyl)-1H-pyrrole-2-carbonyl]-amino}-butyric acid ethyl ester

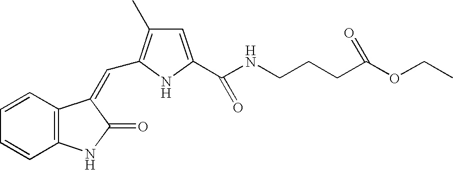

The general procedure 4 was followed using 4-methyl-5-(2-oxo-1,2-dihydro-indol-3-ylidenemethyl)-1H-pyrrole-2-carboxylic acid (67 mg, 0.25 mmole), 4-amino-butyric acid ethyl ester; hydrochloride (84 mg, 0.5 mmole) and Et₃N (0.14 mL, 1 mmole, 4 eq.). After purification by flash chromatography, 75 mg of the title compound were obtained as a yellow/orange solid (79% yield).

¹H NMR (DMSO-d₆) δ 13.64 (s, 1H), 10.91 (s, 1H), 8.36 (t, 1H), 7.80 (d, 1H), 7.64 (s, 1H), 7.17 (t, 1H), 7.01 (t, 1H), 6.88 (d, 1H), 6.82 (d, 1H), 4.05 (q, 2H), 3.26 (q, 2H), 2.35 (s, 3H), 2.35 (t, 2H), 1.77 (m, 2H), 1.18 (t, 3H).

### Preparation 21

### Compound 20, 4-methyl-5-(2-oxo-1,2-dihydro-indol-3-ylidenemethyl)-1H-pyrrole-2-carboxylic acid [1-(4-chloro-phenyl)-cyclopropylmethyl]-amide

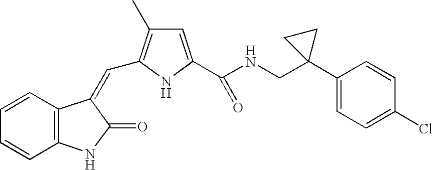

The general procedure 4 was followed using 4-methyl-5-(2-oxo-1,2-dihydro-indol-3-ylidenemethyl)-1H-pyrrole-2-carboxylic acid (67 mg, 0.25 mmole), C-[1-(4-chloro-phenyl)-cyclopropyl-methylamine (91 mg, 0.5 mmole) and Et₃N (0.07 mL, 0.5 mmole, 2 eq.). After purification by flash chromatography, 91 mg of the title compound were obtained as a yellow/orange solid (84% yield).

¹H NMR (DMSO-d₆) δ 13.60 (s, 1H), 10.91 (s, 1H), 8.28 (t, 1H), 7.79 (d, 1H), 7.63 (s, 1H), 7.32 (s, 4H), 7.17 (t, 1H), 7.00 (t, 1H), 6.88 (d, 1H), 6.85 (d, 1H), 3.52 (d, 2H), 2.33 (s, 3H), 0.99 (m, 2H), 0.77 (m, 2H).

### General Procedure 5

To a solution of 4-methyl-5-(2-oxo-1,2-dihydro-indol-3-ylidenemethyl)-1H-pyrrole-2-carboxylic acid (67 mg, 0.25 mmole, 1 eq.) in DMF (5 mL) is added K₂CO₃ in one portion followed by the corresponding bromide. The mixture is shaken or stirred at room temperature overnight. After addition of few drops of H₂O and cooling in an ice bath, a precipitate forms which is filtered and if necessary purified by flash chromatography.

### Preparation 22

### Compound 21, 4-methyl-5-(2-oxo-1,2-dihydro-indol-3-ylidenemethyl)-1H-pyrrole-2-carboxylic acid benzyl ester

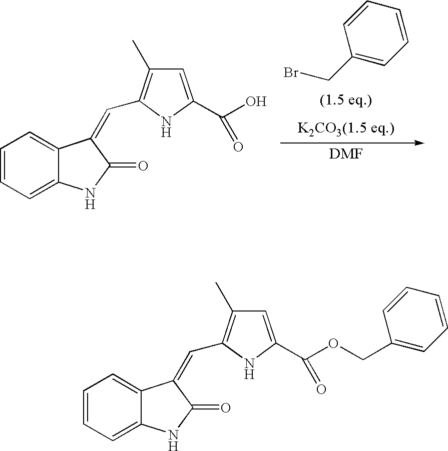

The general procedure 5 was followed using bromomethyl-benzene (45 μL, 0.37 mmole, 1.5 eq.) and K₂CO₃ (53 mg, 0.37 mmole, 1.5 eq.). After purification, 33 mg of the title compound were obtained as an orange solid (³⁷% yield).

¹H NMR (DMSO-d₆) δ 14.09 (s, 1H), 11.06 (s, 1H), 7.85 (d, 1H), 7.71 (s, 1H), 7.5-7.3 (m, 5H), 7.22 (t, 1H), 7.03 (t, 1H), 6.91 (d, 1H), 6.84 (d, 1H), 5.35 (s, 2H), 2.35 (s, 3H).

### General Procedure 6

To a suspension or solution of the starting pyrrole (1 eq.) in CCl₄ is added NBS (1.05 eq.) followed by benzoyl-peroxide (0.025 eq.) under argon. The mixture is then heated to reflux and the reaction is followed by LC/MS. The crude mixture is then allowed to come to room temperature and cooled in an ice bath. A precipitate forms which is filtered and purified by flash chromatography. If no precipitation occurs the solvent is removed under reduced pressure and the crude is purified by flash chromatography.

### Preparation 23

### 4-Bromo-3,5-dimethyl-1H-pyrrole-2-carbaldehyde

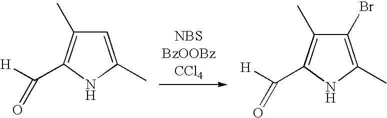

The general procedure 6 was followed using 3,5-dimethyl-1H-pyrrole-2-carbaldehyde (8 g, 65 mmoles), NBS (12.16 g, 68.3 mmoles) and benzoyl-peroxide (394 mg, 1.63 mmole) in CCl₄ (300 mL).

After purification 11.9 g of 4-bromo-3,5-dimethyl-1H-pyrrole-2-carbaldehyde were obtained as a dark solid (92% yield).

¹H NMR (DMSO-d₆) δ 12.06 (br, 1H), 9.50 (s, 1H), 2.22 (s, 3H), 2.19 (s, 3H).

### Preparation 24

### Compound 22, 3-(4-bromo-3,5-dimethyl-1H-pyrrol-2-ylmethylene)-1,3-dihydro-indol-2-one

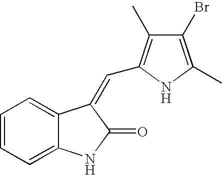

The general procedure 6 was followed using 3-(3,5-dimethyl-1H-pyrrol-2-ylmethylene)-1,3-dihydro-indol-2-one (2 g, 8.4 mmoles), NBS (1,57 g, 8.8 mmoles) and benzoyl-peroxide (51 mg, 0.21 mmole) in CCl₄ (80 mL).

After purification 0.7 g of the title compound were obtained as an orange/brown solid (26% yield).

¹H NMR (DMSO-d₆) δ 13.79 (s, 1H), 10.90 (s, 1H), 7.77 (s, 1H), 7.63 (s, 1H), 7.14 (t, 1H), 6.99 (t, 1H), 6.88 (d, 1H), 2.32 (s, 3H), 2.28 (s, 3H).

### Preparation 25

### Compound 23, 3-(4-chloro-3,5-dimethyl-1H-pyrrol-2-ylmethylene)-1,3-dihydro-indol-2-one

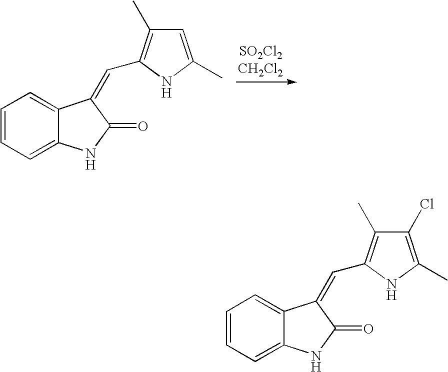

To a suspension of 3-(3,5-dimethyl-1H-pyrrol-2-ylmethylene)-1,3-dihydro-indol-2-one (2 g, 8.4 mmoles) in dry CH₂Cl₂ (30 mL) was added under argon at 0° C. sulfonyl chloride (0.84 mL, 8.4 mmoles). The dark mixture was stirred for 70 minutes at 0° C. and then allowed to come to room temperature for 15 minutes. After removal of the solvent and purification by flash chromatography, 770 mg of the title compound were obtained as an orange solid (34% yield).

¹H NMR (DMSO-d₆) δ 13.72 (s, 1H), 10.90 (s, 1H), 7.77 (d, 1H), 7.61 (s, 1H), 7.13 (t, 1H), 7.00 (t, 1H), 6.88 (d, 1H), 2.32 (s, 3H), 2.28 (s, 3H).

### General Procedure 7

To a suspension of NaH (60% dispersion in mineral oil, 1.5 eq.) in DMF is added a solution of aldehyde (1 eq.) in DMF. After stirring at room temperature for 30 minutes, the mixture is cooled down in an ice bath and the chloroester (1 eq.) is added dropwise. The crude mixture is then allowed to come to room temperature and left under stirring. The reaction is followed by TLC. The reaction mixture is poured into ice and extracted with Et₂O. The combined organic phases are washed once with brine and concentrated in vacuo together with silica gel. The residue is purified by chromatography.

### Preparation 26

### (3-Formyl-indol-1-yl)-acetic acid methyl ester

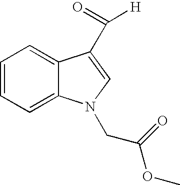

General procedure 7 was followed using NaH (0.49 g, 20.5 mmoles) in DMF (5 mL), 1H-indole-3-carbaldehyde (1.99 g, 13.7 mmoles) in DMF (15 mL), and chloro-acetic acid methyl ester (1.05 mL, 13.7 mmoles). Flash chromatography on silica gel using a gradient of EtOAc/Petroleum ether from 1:3 to 3:1 furnished the title compound (2.1 g, 71%).

¹H NMR (DMSO-d₆) δ 9.95 (s, 1H), 8.28 (s, 1H), 8.12 (m, 1H), 7.54 (m, 1H), 7.29 (m, 2H), 5.31 (s, 2H), 3.71 (s, 3H).

### Preparation 27

### Compounds 24 and 25, respectively [3-(2-oxo-1,2-dihydro-indol-3-ylidenemethyl)-indol-1-yl]-acetic acid methyl ester and [3-(2-oxo-1,2-dihydro-indol-3-ylidenemethyl)-indol-1-yl]-acetic acid ethyl ester

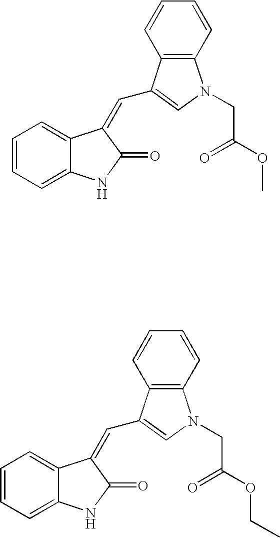

General procedure 1 was followed using 1,3-dihydro-indol-2-one (0.14 g; 1.84 mmoles), (3-formyl-indol-1-yl)-acetic acid methyl ester (1.87 g, 1.83 mmoles) and piperidine (10 drops). After filtration a mixture of [3-(2-oxo-1,2-dihydro-indol-3-ylidenemethyl)-indol-1-yl]-acetic acid methyl ester (45%) and [3-(2-oxo-1,2-dihydro-indol-3-ylidenemethyl)-indol-1-yl]-acetic acid ethyl ester (55%) was obtained as an orange solid (2.27 g, 80% yield).

¹H-NMR (DMSO-d₆, compound 24) δ 10.55 (s, 1H), 9.41 (s, 1H), 8.21 (m, 1H), 8.13 (s, 1H), 7.91 (d, 1H), 7.53 (m, 1H), 7.29 (m, 2H), 7.16 (t, 1H), 7.00 (t, 1H), 6.86 (d, 1H), 5.37 (s, 2H), 3.72 (s, 3H). ¹H-NMR (DMSO-d₆, compound 25) δ 10.55 (s, 1H), 9.41 (s, 1H), 8.21 (m, 1H), 8.13 (s, 1H), 7.91 (d, 1H), 7.53 (m, 1H), 7.29 (m, 2H), 7.16 (t, 1H), 7.00 (t, 1H), 6.86 (d, 1H), 5.35 (s, 2H), 4.19 (q, 2H), 1.23 (t, 3H).

### Preparation 28

### Compound 26, [3-(2-oxo-1,2-dihydro-indol-3-ylidenemethyl)-indol-1-yl]-acetic acid

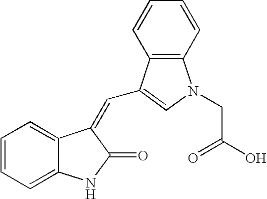

General procedure 2 was followed using a 45:55 mixture of [3-(2-oxo-1,2-dihydro-indol-3-ylidenemethyl)-indol-1-yl]-acetic acid methyl ester and [3-(2-oxo-1,2-dihydro-indol-3-ylidenemethyl)-indol-1-yl]-acetic acid ethyl ester (1.76 g, about 8 mmoles) in a 1:1 mixture of an aqueous solution of LiOH 1N (25 mL) and THF (25 mL). After heating to reflux for 6 hours, HCl (1N) was added and the title compound was obtained as an orange solid (1.37 g, 53% yield).

¹H-NMR (DMSO-d₆, Z isomer) δ 13.19 (br, 1H), 10.55 (s, 1H), 9.40 (s, 1H), 8.21 (m, 1H), 8.13 (s, 1H), 7.90 (d, 1H), 7.54 (m, 1H), 7.29 (m, 2H), 7.15 (t, 1H), 7.00 (t, 1H), 6.86 (d, 1H), 5.24 (s, 2H).

### Preparation 29

### 3-(3-Formyl-indol-1-yl)-propionic acid ethyl ester

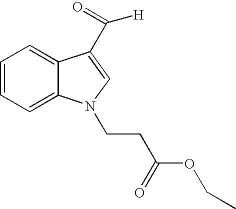

General procedure 7 was followed using NaH (0.41 g, 10.3 mmoles) in DMF (5 mL), 1H-indole-3-carbaldehyde (1.0 g, 6.9 mmoles) in DMF (15 mL), and 3-chloro-propionic acid ethyl ester (0.86 mL, 6.9 mmoles). Flash chromatography on silica gel using a gradient of EtOAc/Petroleum ether from 1:3 to 3:1 furnished the title compound (1.49 g, 88% yield).

¹H NMR (DMSO-d₆) δ 9.91 (s, 1H), 8.30 (s, 1H), 8.11 (d, 1H), 7.66 (d, 1H), 7.30 (m, 2H), 4.54 (t, 2H), 4.03 (q, 2H), 2.93 (t, 2H), 1.11 (t, 3H).

### Preparation 30

### Compound 27, 3-[3-(2-oxo-1,2-dihydro-indol-3-ylidenemethyl)-indol-1-yl]-propionic acid ethyl ester

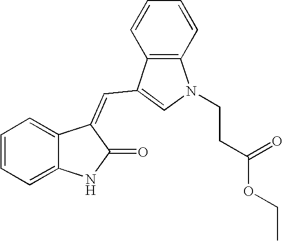

General procedure 1 was followed using 1,3-dihydro-indol-2-one (0.46 g, 3.43 mmoles), 3-(3-formyl-indol-1-yl)-propionic acid ethyl ester (0.84 g, 3.42 mmoles) and piperidine (10 drops). After filtration the title compound was obtained as a yellow solid (1.08 g, 88% yield).

¹³C-NMR (DMSO-d₆, Z isomer) δ 10.52 (s, 1H), 9.45 (s, 1H), 8.20 (m, 1H), 8.11 (s, 1H), 7.88 (d, 1H), 7.64 (m, 1H), 7.29 (m, 2H), 7.15 (t, 1H), 6.99 (t, 1H), 6.85 (d, 1H), 4.58 (t, 2H), 4.04 (q, 2H), 2.92 (t, 2H), 1.12 (t, 3H).

### Preparation 31

### Compound 28, 3-[3-(2-oxo-1,2-dihydro-indol-3-ylidenemethyl)-indol-1-yl]-propionic acid

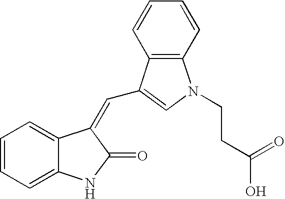

General procedure 2 was followed using 3-[3-(2-oxo-1,2-dihydro-indol-3-ylidenemethyl)-indol-1-yl]-propionic acid ethyl ester (0.62 g, 1.73 mmoles) in a 1:1 mixture of an aqueous solution of LiOH 0.2N (30 mL) and MeOH (30 mL). After heating to reflux for 4 hours, the pH was adjusted to acidic pH by addition of HCl (1N) and the title compound was obtained as orange crystals (0.57 g, 100% yield).

¹H-NMR (DMSO-d₆) δ 12.46 (br, 1H), 10.49 (s, 1H), 9.47 (s, 1H), 8.19 (m, 1H), 8.10 (s, 1H), 7.88 (d, 1H), 7.64 (m, 1H), 7.29 (m, 2H), 7.15 (t, 1H), 6.99 (t, 1H), 6.85 (d, 1H), 4.55 (t, 2H), 2.86 (t, 2H).

### Preparation 32

### Compound 29, 3-[1-(2-chloro-thiazol-5-ylmethyl)-1-indol-3-ylmethylene]-1,3-dihydro-indol-2-one

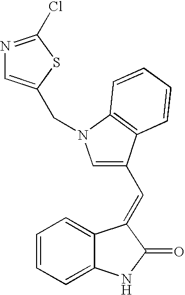

General procedure 1 was followed using commercially available 1-(2-chloro-thiazol-5-ylmethyl)-1H-indole-3-carbaldehyde (310 mg, 1.1 mmoles, from Bionet) and 1,3-dihydro-indol-2-one (149 g, 1.1 mmoles) as a yellow powder (127 mg, 29% yield).

¹H-NMR (DMSO-d₆, mixture of E and Z isomers, signals of the predominant isomer) δ 10.55 (s, 1H), 9.50 (s, 1H), 8.23 (dd, 1H), 8.12 (s, 1H), 7.90 (d, 1H), 7.86 (s, 1H), 7.75 (m, 1H), 7.30 (m, 2H), 7.16 (t, 1H), 7.00 (t, 1H), 6.86 (d, 1H), 5.87 (s, 2H).

### General Procedure 8

Under dry conditions is prepared a solution of KOH (4 eq., 2-3 N) in DMSO. After stirring at room temperature for 5 minutes is added the starting indole (1 eq.) under argon atmosphere and the mixture is stirred an additional 45 minutes.

The alkylating agent (1.1 eq.) is then added dropwise at 0° C. and the mixture is allowed to come to room temperature. The reaction is followed by TLC. H₂O is added and the aqueous phase is extracted with Et₂O (3×). The combined organic phases are washed once with H₂O, once with brine, dried over MgSO₄. Removal of solvent under vacuum affords the expected compound which can be used without further purification.

### Preparation 33

### 1-Methyl-1-H-indole-3-carbaldehyde

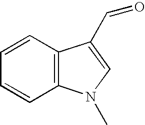

General procedure 8 was followed using KOH (15.46 g, 0.28 mole) in DMSO (100 mL), 1H-indole-3-carbaldehyde (10 g, 69 mmoles) and iodomethane (4.73 mL, 76 mmoles). The title compound was obtained as a yellow/brown solid (7.2 g, 66% yield).

¹H-NMR (DMSO-d₆) δ 9.90 (s, 1H), 8.26 (s, 1H), 8.11 (d, 1H), 7.58 (d, 1H), 7.30 (m, 2H), 3.90 (s, 3H).

### Preparation 34

### 1-Propyl-1-H-indole-3-carbaldehyde

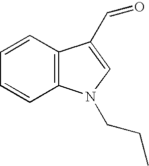

General procedure 8 was followed using KOH (1.58 g, 28 mmoles) in DMSO (15 mL), 1H-indole-3-carbaldehyde (1 g, 7.0 mmoles) and iodopropane (0.74 mL, 7.6 mmoles). The title compound was obtained as an orange oil (1.3 g, 100% yield).

¹³C NMR (DMSO-d₆) δ 184.4, 140.7, 137.0, 124.6, 123.4, 122.3, 120.9, 116.9, 111.0, 47.7, 22.6, 10.9.

### Preparation 35

### Compound 30, 3-(1-methyl-1H-indol-3-ylmethylene)-1,3-dihydro-indol-2-one

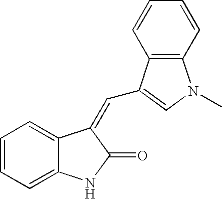

The title compound was obtained as a yellow powder following the general procedure 1 and using 1-methyl-1-H-indole-3-carbaldehyde (159 mg, 1 mmole) and 1,3-dihydro-indol-2-one (161 mg, 1.2 mmoles): 205 mg, 75% yield.

¹H-NMR (DMSO-d₆) δ 10.52 (s, 1H), 9.41 (s, 1H), 8.20 (m, 1H), 8.13 (s, 1H), 7.88 (d, 1H), 7.58 (m, 1H), 7.30 (m, 2H), 7.14 (t, 1H), 6.99 (t, 1H), 6.85 (d, 1H), 3.95 (s, 3H).

### Preparation 36

### Compound 31, 3-(1-propyl-1H-indol-3-ylmethylene)-1,3-dihydro-indol-2-one

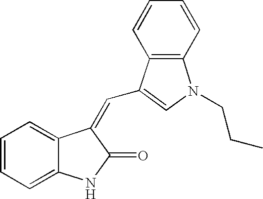

The title compound was obtained as orange crystals following the general procedure 1 using 1-propyl-1-H-indole-3-carbaldehyde (217 mg, 1.16 mmoles) and 1,3-dihydro-indol-2-one (154 mg, 1.16 mmoles): 330 mg, 94% yield.

³C-NMR (DMSO-d₆, Z isomer) δ 168.0, 139.1, 135.9, 135.8, 128.7, 126.7, 126.5, 125.6, 122.5, 120.9, 120.4, 119.0, 118.6, 110.7, 110.4, 108.9, 47.8, 22.9, 11.1.

### Preparation 37

### 5-Dimethylaminomethyl-2-methoxy-benzaldehyde

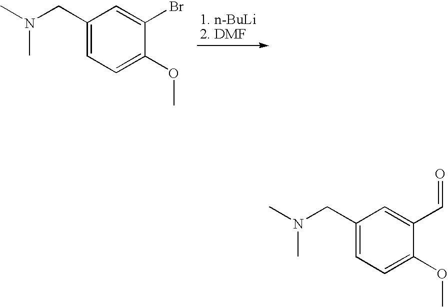

To a solution of (3-bromo-4-methoxy-benzyl)-dimethylamine (2.5 g, 10.2 mmoles) in dry THF (46 mL) at −78° C. was added n-BuLi (1.6 M, 7.0 mL, 11.3 mmoles) keeping the temperature below −70° C. The mixture was stirred for 60 minutes and dry DMF (1.6 mL, 20 mmoles) was added in one portion. The mixture was stirred for 2 h at −78° C., the cooling bath was removed and the mixture was allowed to warm to 0° C. Aqueous NaOH (2M, 20 mL) was added and the mixture was extracted with Et₂O (3×100 mL). The organic phase was dried over Na₂SO₄ and the solvent was removed under reduced pressure leaving a yellow oil. Distillation (100° C., 1 mbar) afforded the product as a colourless oil in 35% yield (0.7 g, 3.6 mmoles).

¹H-NMR (CDCl₃): 10.45 (1H, s), 7.80 (1H, d J=2.3 Hz), 7.54 (1H, dd J=8.8 Hz, 2.3 Hz), 6.96 (1H, d J=8.8 Hz), 3.93 (3H, s), 3.39 (2H, s), 2.22 (6H, s).

### Preparation 38

### Compound 32, 3-(5-dimethylaminomethyl-2-methoxy-benzylidene)-1,3-dihydro-indol-2-one

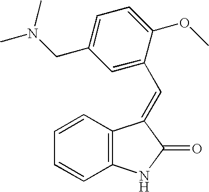

The title compound was prepared following the general procedure 1 using 1,3-dihydro-indol-2-one (0.36 g, 2.6 mmoles) and 5-dimethylaminomethyl-2-methoxy-benzaldehyde (0.5 g, 2.6 mmoles). Yield: 72% (0.58 g, 1.9 mmoles) as a yellow oil.

¹³C-NMR (DMSO-d₆, E isomer) δ 168.5, 156.7, 142.7, 132.0, 131.6, 130.5, 129.8, 127.2, 122.5, 122.3, 121.1, 120.8, 111.2, 109.9, 62.4, 55.6, 44.7

### Preparation 39

### 2-[(2-Dimethylamino-ethyl)-methyl-amino]-5-methoxy-benzaldehyde

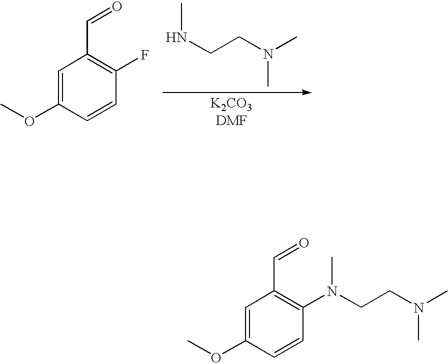

To a stirred solution of 2-fluoro-5-methoxy-benzaldehyde (10.6 mmoles) in dry DMF (15 mL) was added K₂CO₃ (1.8 g, 12.7 mmoles) and N,N,N′-trimethylethylendiamine (1.7 mL, 12.7 mmoles). After stirring at 100° C. overnight, the reaction mixture was cooled to room temperature. Water was added (50 mL) and the aqueous phase was extracted with ethyl acetate (3×50 mL). The combined organic phases were washed with water and dried (Na₂SO₄). Evaporation in vacuo and purification by chromatography gave the pure title product in 13% yield (0.33 g, 1.4 mmol) as a brown oil.

¹H-NMR (CDCl₃): 10.41 (1H, s), 7.31 (1H, d J=3.0 Hz), 7.19 (1H, d J=8.8 Hz), 7.10 (1H, dd J=8.8 Hz; 3.0 Hz), 3.82 (3H, s), 3.11 (2H, dd J≈6 Hz; 8 Hz), 2.82 (3H, s), 2.42 (2H, dd J≈6 Hz; 8 Hz), 2.31 (6H, s).

### Preparation 40

### Compound 33, 3-{2-[(2-dimethylamino-ethyl)-methyl-amino]-5-methoxy-benzylidene}-1,3-dihydro-indol-2-one

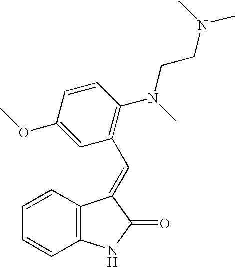

The title compound was prepared following the general procedure 1 using 1,3-dihydro-indol-2-one (0.12 g, 0.9 mmole) and 2-[(2-dimethylamino-ethyl)-methyl-amino]-5-methoxy-benzaldehyde (0.21 g, 0.9 mmole). Yield: 87% (0.28 g, 0.8 mmole) as yellow oil.

¹³C-NMR (CDCl₃, E isomer) δ 170.3, 154.3, 147.3, 141.3, 136.7, 129.6, 129.5, 126.1, 122.9, 122.1, 121.7, 120.3, 117.0, 114.2, 110.1, 57.4, 55.9, 55.8, 45.6, 42.5.

### Preparation 41

### Compound 34, 3-{4-[(2-dimethylamino-ethyl)-methyl-amino]-3′,5′-dimethyl-biphenyl-3-ylmethylene}-1,3-dihydro-indol-2-one

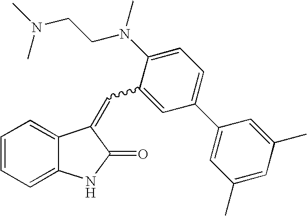

The title compound was prepared following the general procedure 1 using 1,3-dihydro-indol-2-one (0.085 g, 0.6 mmole) and 4-[(2-dimethylamino-ethyl)-methyl-amino]-3′,5′-dimethyl-biphenyl-3-carbaldehyde (0.2 g, 0.6 mmole, synthesis described in patent WO-03097576). Yield: 50% (0.14 g, 0.3 mmole) as yellow oil.

¹H-NMR (CDCl₃) δ 7.96 (d, 1H), 7.90 (s, 1H), 7.87 (br, 1H), 7.76 (d, 1H), 7.60 (dd, 1H), 7.3-7.1 (m, 4H), 7.0-6.8 (m, 3H), 3.18 (t, 2H), 2.88 (s, 3H), 2.60 (t, 2H), 2.35 (s, 6H), 2.19 (s, 6H).

### Preparation 42

### Compound 35, 3-(2-dimethylaminomethyl-benzylidene)-1,3-dihydro-indol-2-one

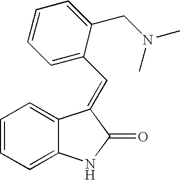

The title compound was prepared following the general procedure 1 using 1,3-dihydro-indol-2-one (0.77 g, 5.8 mmoles) and 2-dimethylaminomethyl-benzaldehyde (0.93 g, 5.8 mmoles, synthesis described in Journal of Organic Chemistry (1969), 34(8), 2482-4). Yield: 16% (0.26 g, 0.9 mmol) as yellow crystals.

¹³C-NMR (DMSO-d₆, E isomer) δ 168.3, 142.7, 138.0, 135.1, 134.3, 130.0, 129.7, 129.1, 128.5, 127.7, 127.1, 122.1, 121.2, 120.9, 109.9, 61.3, 44.9

### General Procedure 9

To a solution of 2-hydroxy-5-methoxy-benzaldehyde (1 eq.) in DMF is added Cs₂CO₃ (5 eq.), NaI (2.5 eq.) and the chloroalkyl-amine as a free base or as a salt (2.5 eq.). The suspension is stirred at 110° C. during 4 hours and the reaction is followed by LC/MS. The reaction mixture is then filtered, evaporated in high vacuo, and chromatographed to afford the expected product.

### Preparation 43

### 2-(2-Diethylamino-ethoxy)-5-methoxy-benzaldehyde

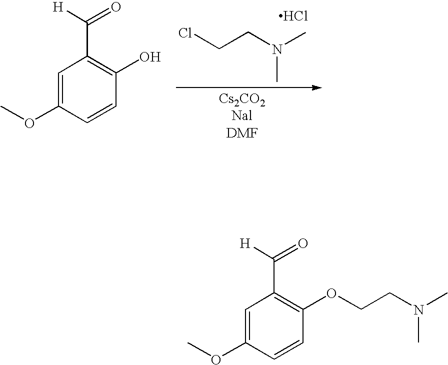

To a solution of 2-hydroxy-5-methoxy-benzaldehyde (2.5 g, 16.6 mmoles) in DMF (100 mL) were added Cs₂CO₃ (27 g, 82.9 mmoles), NaI (6.2 g, 41.4 mmoles) and (2-chloro-ethyl)-diethyl-amine; hydrochloride (7,1 g, 41.4 mmoles), following the general procedure 9. The title compound was obtained as a yellow oil (2.89 g, 69% yield).

¹³C-NMR (CDCl₃) δ 189.7, 156.2, 153.7, 125.2, 123.5, 114.6, 110.2, 68.3, 55.8, 51.8, 48.0, 12.0.

### Preparation 44

### 5-Methoxy-2-(2-morpholin-4-yl-ethoxy)-benzaldehyde

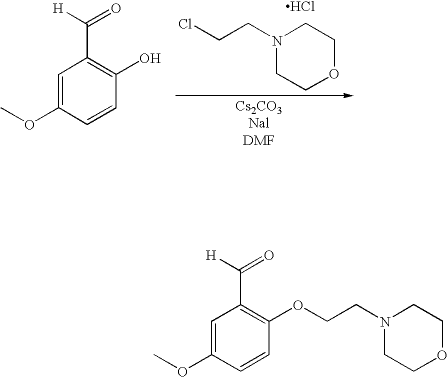

To a solution of 2-hydroxy-5-methoxy-benzaldehyde (2.5 g, 16.6 mmoles) in DMF (100 mL) were added Cs₂CO₃ (27 g, 82.9 mmoles), NaI (6.2 g, 41.5 mmoles) and 4-(2-chloro-ethyl)-morpholine; hydrochloride (7,7 g, 41.5 mmoles), following the general procedure 9. The title compound was obtained as a brown oil (3.3 g, 75% yield).

¹³C-NMR (DMSO-d₆) δ 189.0, 155.5, 153.2, 124.9, 123.0, 116.1, 109.8, 67.1, 66.1, 56.8, 55.4, 53.4.

### Preparation 45

### 5-Methoxy-2-(2-piperidin-1-yl-ethoxy)-benzaldehyde

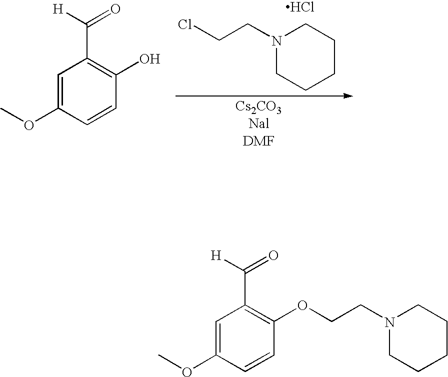

To a solution of 2-hydroxy-5-methoxy-benzaldehyde (2.5 g, 16.6 mmoles) in DMF (100 mL) were added Cs₂CO₃ (27 g, 82.9 mmoles), NaI (6.2 g, 41.5 mmoles) and 1-(2-chloro-ethyl)-piperidine; hydrochloride (7.8 g, 42.4 mmoles), following the general procedure 9. The title compound was obtained as a brown oil (2.26 g, 52% yield).

¹³C-NMR (DMSO-d₆) δ 189.0, 155.6, 153.2, 124.9, 123.0, 116.2, 109.7, 67.4, 57.2, 55.5, 54.2, 25.6, 23.8.

### Preparation 46

### Compound 36, 3-[2-(2-diethylamino-ethoxy)-5-methoxy-benzylidene]-1,3-dihydro-indol-2-one

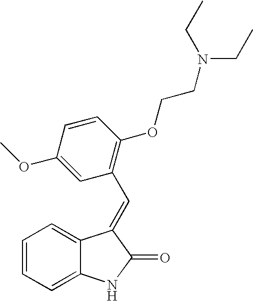

The title compound was obtained following the general procedure 1 using 1,3-dihydro-indol-2-one (0.8 g, 6 mmoles), 2-(2-diethylamino-ethoxy)-5-methoxy-benzaldehyde (1.53 g, 6 mmoles) and piperidine (0.35 mL) in EtOH (20 mL). After chromatography the title compound was obtained as a hygroscopic solid (1.54 g, 70% yield)

¹³C-NMR (DMSO-d₆, mixture of the E and Z isomers, signals of the predominant E isomer) δ 168.5, 152.5, 151.1, 142.7, 131.6, 129.8, 127.5, 123.7, 122.4, 121.1, 120.9, 116.8, 114.3, 113.7, 109.9, 67.6, 55.4, 51.0, 47.0, 11.9.

### Preparation 47

### Compound 37, 3-[2-(2-diethylamino-ethoxy)-5-methoxy-benzylidene]-1,3-dihydro-indol-2-one; hydrochloride

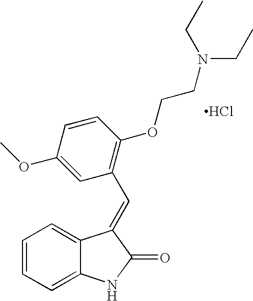

To a solution of 3-[2-(2-diethylamino-ethoxy)-5-methoxy-benzylidene]-1,3-dihydro-indol-2-one (1.5 g, 4 mmoles) in EtOAc (3 mL) was added a solution of HCl in dioxane (4N, 2 mL, 8.2 mmoles). After addition of Et₂O, a precipitate formed which was filtered and washed with Et₂O to afford the title compound (1.32 g, 80% yield).

¹³C-NMR (DMSO-d₆, mixture of the E and Z isomers, signals of the predominant E isomer) δ 168.3, 153.1, 149.9, 142.8, 131.0, 130.0, 128.0, 123.8, 122.4, 121.0, 120.8, 116.5, 114.5, 113.5, 110.1, 66.3, 63.3, 55.5, 47.2, 8.4.

### Preparation 48

### Compound 38, 3-[5-methoxy-2-(2-morpholin-4-yl-ethoxy)-benzylidene]-1,3-dihydro-indol-2-one

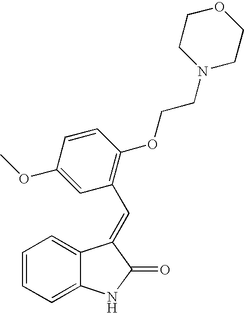

The title compound was obtained following the general procedure 1 using 1,3-dihydro-indol-2-one (0.74 g, 5.6 mmoles), 5-methoxy-2-(2-morpholin-4-yl-ethoxy)-benzaldehyde (1.50 g, 5.6 mmoles) and piperidine (0.35 mL) in EtOH (20 mL). After chromatography the title compound was obtained as an orange solid (2.04 g, 96% yield)

¹³C-NMR (DMSO-d₆, mixture of the E and Z isomers, signals of the predominant E isomer) δ 168.5, 152.7, 151.0, 142.7, 131.6, 129.8, 127.5, 124.0, 122.4, 121.0, 120.9, 116.8, 114.3, 114.2, 109.9, 66.8, 66.0, 56.8, 55.4, 53.5.

### Preparation 49

### Compound 39, 3-[5-methoxy-2-(2-piperidin-1-yl-ethoxy)-benzylidene]-1,3-dihydro-indol-2-one

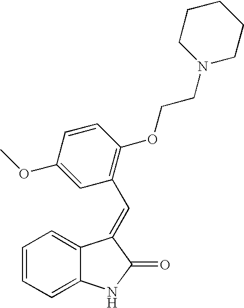

The title compound was obtained following the general procedure 1 using 1,3-dihydro-indol-2-one (0.76 g, 5.7 mmoles), 5-methoxy-2-(2-piperidin-1-yl-ethoxy)-benzaldehyde (1.52 g, 5.8 mmoles) and piperidine (0.35 mL) in EtOH (20 mL). After chromatography the title compound was obtained as an orange solid (2.07 g, 96% yield)

¹³C-NMR (DMSO-d₆, mixture of the E and Z isomers) δ 168.5, 167.0, 152.6, 152.2, 151.9, 151.1, 142.7, 131.6, 130.2, 129.8, 128.8, 127.5, 126.0, 123.9, 123.1, 122.4, 121.0, 120.9, 119.0, 118.1, 116.8, 116.2, 114.1, 113.6, 109.9, 109.3, 67.4, 67.0, 57.3, 57.1, 55.4, 55.3, 54.3, 25.6, 25.5, 23.8.

### Preparation 50

### Compound 40, 3-(1H-indol-3-ylmethylene)-2-oxo-2,3-dihydro-indole-1-carboxylic acid tert-butyl ester

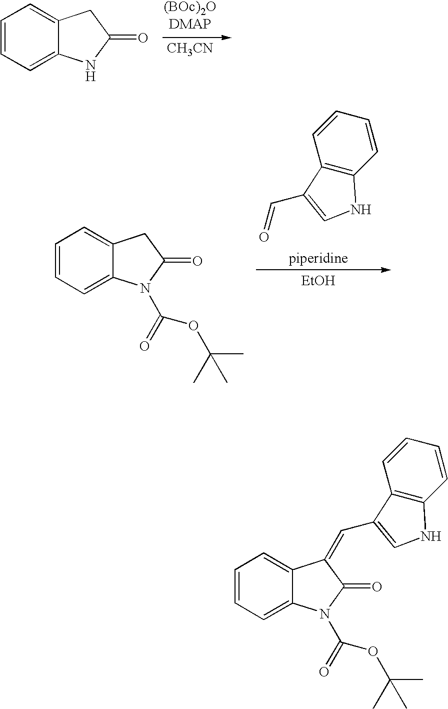

1—To a solution of 1,3-dihydro-indol-2-one (1 g, 7.57 mmoles) in acetonitrile (50 mL) were added DMAP (100 mg, 7.57 mmoles) and (Boc)₂O (1.82 g, 8.33 mmoles). The reaction was followed by TLC. The crude mixture was concentrated in vacuo and CH₂Cl₂ was added (50 mL). The organic phase was washed once with a 10% aqueous solution of KHSO₄ (50 mL), once with an aqueous saturated solution of NaHCO₃ (50 mL), twice with H₂O (2×50 mL) and was then concentrated in vacuo together with silica gel. The residue was purified by flash chromatography using a gradient of petroleum ether/EtOAc from 100:00 to 80:20, furnishing 2-oxo-2,3-dihydro-indole-1-carboxylic acid tert-butyl ester (619 mg, 35%).

2—The general procedure 1 was followed using 2-oxo-2,3-dihydro-indole-1-carboxylic acid tert-butyl ester (564 mg, 2.42 mmoles), 1H-indole-3-carbaldehyde (351 mg, 2.42 mmoles) and piperidine (3 drops). The pure title compound was obtained after filtration (482 mg, 55% yield).

¹H-NMR (DMSO-d₆, Z isomer) δ 12.17 (br, 1H), 9.36 (s, 1H), 8.31 (s, 1H), 8.24 (m, 1H), 8.06 (d, 1H), 7.75 (d, 1H), 7.56 (m, 1H), 7.34-7.16 (m, 4H), 1.63 (s, 9H).

### Preparation 51

### Compound 41, 3-(3,5-dimethyl-1H-pyrrol-2-ylmethylene)-1-(4-methoxy-benzyl)-1,3-dihydro-indol-2-one

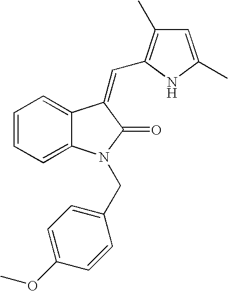

To a suspension of NaH (60% oil dispersion, 209 mg, 5.25 mmoles) in dry DMF (30 mL) was added under argon at 0° C. a solution of 3-(3,5-dimethyl-1H-pyrrol-2-ylmethylene)-1,3-dihydro-indol-2-one (1.2 g, 5 mmoles) in DMF (46 mL) followed by 1-chloromethyl-4-methoxy-benzene (2.1 mL, 15 mmoles). The reaction was left under stirring and allowed to come to room temperature. The reaction evolution was followed by TLC. After completion of the reaction a saturated aqueous solution of NaHCO₃ (25 mL) was added followed by H₂O (25 mL). The aqueous phase was extracted with EtOAc (3×50 mL). The combined organic phases were washed once with a saturated aqueous solution of NaHCO₃ (50 mL) and dried over MgSO₄. After filtration and removal of the solvent, a brown solid was obtained. crystallization from EtOH and H₂O afforded the pure title compound as a yellow solid (1.08 g, 60% yield).

¹³C-NMR (DMSO-d₆, Z isomer) δ 167.4, 158.4, 138.2, 136.3, 132.4, 128.8, 128.6, 126.6, 125.5, 124.8, 123.7, 121.4, 117.9, 113.9, 112.7, 111.1, 108.7, 54.9, 42.1, 13.5, 11.2.

### Preparation 52

### Compound 42, 3-(3,5-Dimethyl-1H-pyrrol-2-ylmethylene)-1-methyl-1,3-dihydro-indol-2-one

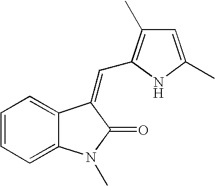

To a suspension of NaH (60% oil dispersion, 151 mg, 3.8 mmoles) in dry DMF (20 mL) was added under argon at 0° C. a solution of 3-(3,5-dimethyl-1H-pyrrol-2-ylmethylene)-1,3-dihydro-indol-2-one (741 mg, 3.1 mmoles) in DMF (28 mL) followed by iodomethane (0.24 mL, 3.8 mmoles). The reaction was stirred and allowed to come to room temperature. The reaction evolution was followed by LC/MS. H₂O (25 mL) was added and the mixture was stirred until a thick orange precipitate formed. The solid was filtered. Crystallization from MeOH and H₂O afforded the pure title compound as a yellow solid (504 mg, 63% yield).

¹³C-NMR (DMSO-d₆, Z isomer) δ 167.4, 139.2, 135.9, 131.9, 126.5, 125.6, 124.7, 123.3, 121.3, 117.7, 112.5, 111.4, 108.1, 25.9, 13.4, 11.2.

### Preparation 53

### Compound 43, acetic acid 3-(3,5-dimethyl-1H-pyrrol-2-ylmethylene)-2-oxo-2,3-dihydro-indol-1-ylmethyl ester

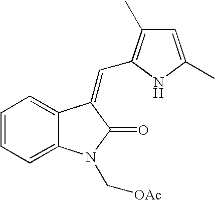

To a suspension of 3-(3,5-dimethyl-1H-pyrrol-2-ylmethylene)-1,3-dihydro-indol-2-one (2.02 g, 8.5 mmoles) and Cs₂CO₃ (11.05 g, 34 mmoles) in CH₃CN (200 mL) was added acetic acid chloromethyl ester (3.3 mL, 34 mmoles). The reaction mixture was heated at 40° C. and left under stirring overnight. The reaction evolution was followed by LC/MS. The mixture was then concentrated in vacuo together with silica gel. The residue was purified by chromatography using a gradient of petroleum ether/ethyl acetate from 85:15 to 50:50, furnishing the title compound (488 mg, 19% yield) as a deep orange solid.

¹H-NMR (DMSO-d₆, Z isomer) δ 13.01 (s, 1H), 7.81 (d, 1H), 7.65 (s, 1H), 7.26-7.05 (m, 3H), 6.07 (d, 1H), 5.89 (s, 2H), 2.35 (s, 3H), 2.33 (s, 3H), 2.04 (s, 3H).

### Preparation 54

### Compound 44, 1-acetyl-3-(2,5-dimethoxy-benzylidene)-1,3-dihydro-indol-2-one

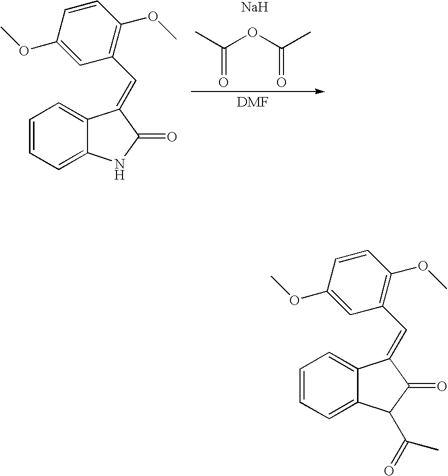

To a solution of 3-(2,5-dimethoxy-benzylidene)-1,3-dihydro-indol-2-one (422 mg, 1.5 mmoles) in DMF (10 mL) was added NaH (60% dispersion in mineral oil, 60 mg, 1.5 mmoles). The yellow solution was stirred at room temperature for 15 minutes and turned red. Acetic anhydride (284 μL, 3 mmoles) was added. The solution turned back to yellow and a precipitate formed. After 2 hours the solid was filtrated, washed with water and dried in vacuo to afford 449 mg of the title compound as a yellow solid (92% yield).

¹H-NMR (DMSO-d₆, mixture of the E and Z isomers, signals of the predominant isomer) δ 8.21 (s, 1H), 7.80 (s, 1H), 7.59 (d, 1H), 7.39 (t, 1H), 7.23 (d, 1H), 7.19-7.05 (m, 3H), 3.81 (s, 3H), 3.73 (s, 3H), 2.66 (s, 3H).

### Preparation 55

### Compound 45, 3-(3,5-dimethyl-1H-pyrrol-2-ylmethylene)-1-hydroxy-1,3-dihydro-indol-2-one

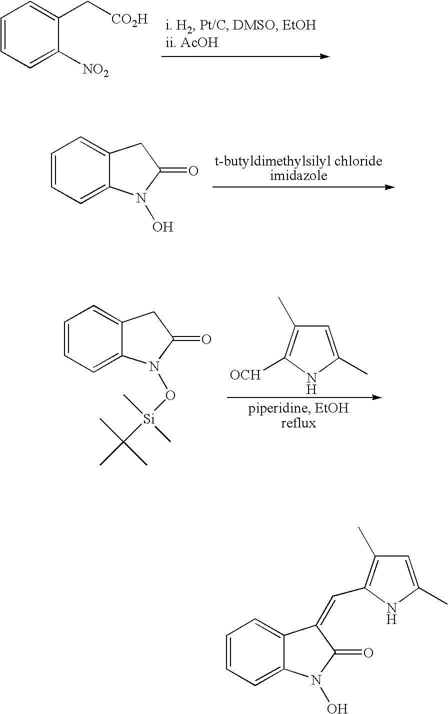

1) 2-Nitrophenylacetic acid (7.10 g, 39.2 mmoles) was hydrogenated in the presence of DMSO (5.8 mL) and 5% Pt/C (0.58 g) in EtOH (350 mL) at room temperature under 1 atmosphere for 24 hours. Then the catalyst was filtered off and washed with EtOH. The filtrate was concentrated in vacuo. The residue was redissolved in AcOH. The obtained solution was stirred at room temperature for 40 hours and concentrated in vacuo. The residue was taken up in toluene. The obtained mixture was concentrated in vacuo to remove the residual acetic acid, giving crude 1-hydroxy-1,3-dihydro-indol-2-one.

2) Crude 1-hydroxy-1,3-dihydro-indol-2-one was taken up in CH₂Cl₂ (50 mL). To the obtained mixture were added imidazole (6.00 g, 88.1 mmoles) and tert-butyldimethylsilyl chloride (5.90 g, 39.2 mmoles) at room temperature. After being stirred at room temperature for 2 hours, the reaction solution was washed twice with H₂O, dried over MgSO₄, and concentrated in vacuo. The residue was purified by chromatography, providing 1.90 g (18% yield based on 2-nitrophenylacetic acid) of 1-(tert-butyidimethylsilyloxy)-1,3-dihydroindol-2-one as a reddish solid.

¹³C-NMR (DMSO-d₆) δ 169.7, 143.2, 127.7, 124.5, 122.3, 121.4, 107.0, 33.2, 25.6, 17.9, −4.8.

3) A mixture of 1-(tert-butyidimethylsilyloxy)-1,3-dihydroindol-2-one (0.80 g, 3.0 mmoles), 3,5-dimethyl-1H-pyrrole-2-carbaldehyde (0.80 g, 6.5 mmol), and piperidine (0.80 mL) in EtOH (20 mL) was heated to reflux for 5 h. The mixture was then concentrated in vacuo together with silica gel. The residue was purified by chromatography first with petroleum ether/ethyl acetate 1:1 and then with ethyl acetate/MeOH 10:1 as eluents, furnishing the title compound (0.61 g, 79% yield based on 1-(tert-butyidimethylsilyloxy)-1,3-dihydroindol-2-one) as a red solid.

¹³C-NMR (DMSO-d₆, Z isomer) δ 163.1, 137.4, 136.2, 132.3, 126.3, 125.7, 123.8, 121.4, 121.0, 117.7, 112.6, 109.0, 106.5, 13.4, 11.2.

### Preparation 56

### Compound 46, 3-(4-bromo-3,5-dimethyl-1H-pyrrol-2-ylmethylene)-1-hydroxy-1,3-dihydro-indol-2-one

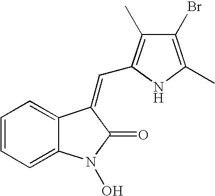

The general procedure 1 was followed using 4-bromo-3,5-dimethyl-1H-pyrrole-2-carbaldehyde (0.63 g, 3.1 mmoles), 1-hydroxy-1,3-dihydro-indol-2-one (0.46 g, 3.1 mmoles) and piperidine (10 drops) in EtOH (5 mL). After filtration, 0.80 g of the title compound were obtained (77% yield).

¹H-NMR (DMSO-d₆, Z isomer) δ 13.65 (s, 1H), 10.95 (br, 1H), 7.84 (d, 1H), 7.69 (s, 1H), 7.23 (t, 1H), 7.06 (t, 1H), 7.00 (d, 1H), 2.34 (s, 3H), 2.28 (s, 3H).

### Preparation 57

### Compound 47, 1-hydroxy-3-(1H-indol-3-ylmethylene)-1,3-dihydro-indol-2-one

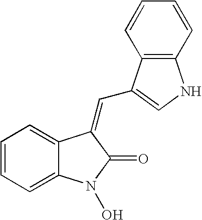

In a sealed glass vessel, a mixture of 1H-indole-3-carbaldehyde (1.95 g, 13.4 mmoles), 1-hydroxy-1,3-dihydro-indol-2-one (1.00 g, 6.7 mmoles), and piperidine (1.14 g, 13.4 mmoles) in EtOH (40 mL) was stirred at 100° C. for 8 hours. The reaction mixture was then concentrated in vacuo. The residue was purified by flash chromatography (silica gel, CH₂Cl₂/AcOH 40:1), providing the title compound (0.90 g, 49%) as a yellow solid.

¹³C-NMR (DMSO-d₆, Z isomer) δ 162.2, 138.5, 135.8, 133.7, 128.2, 128.1, 126.7, 122.5, 121.1, 121.0, 120.8, 118.4, 118.3, 115.5, 112.2, 111.1, 106.2.

### Preparation 58

### Compound 48, 3-(2,5-dimethoxy-benzylidene)-1-hydroxy-1,3-dihydro-indol-2-one

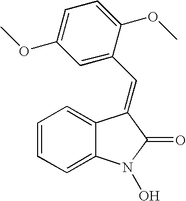

A mixture of 2,5-dimethoxybenzaldehyde (2.23 g, 13.4 mmoles), 1-hydroxy-1,3-dihydro-indol-2-one (1.00 g, 6.7 mmoles), and piperidine (1.14 g, 13.4 mmoles) in EtOH (40 mL) was treated as described in preparation 57. Flash chromatography (silica gel, using a gradient of eluents CH₂Cl₂/AcOH from 100:0 to 40:1) furnished the title compound (1.14 g, 57% yield) as a yellow solid.

¹³C-NMR (DMSO-d₆, E isomer) δ 162.9, 152.5, 151.8, 142.1, 132.3, 130.0, 124.6, 123.0, 122.0, 121.8, 117.3, 117.2, 114.4, 112.7, 107.3, 55.9, 55.5.

### Preparation 59

### Compound 49, 3-(3,5-dimethyl-1H-pyrrol-2-ylmethylene)-1-methoxy-1,3-dihydro-indol-2-one

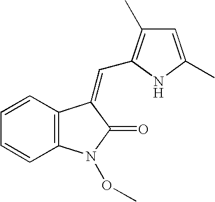

The title compound was prepared following the general procedure 1 using 1-methoxy-oxindole (0.49 g, 3 mmoles, prepared as described in Journal of Enzyme Inhibition and Medicinal Chemistry 2003, 18(3), 243-252) and 3,5-dimethyl-1H-pyrrole-2-carbaldehyde (0.37 g, 3 mmoles). Yield: 61% (0.49 g, 1.8 mmoles) as orange crystals.

³C-NMR (DMSO-d₆, Z isomer) δ 162.8, 137.1, 134.6, 133.3, 126.5, 125.7, 124.6, 122.0, 121.2, 118.1, 113.0, 108.0, 106.5, 63.6, 13.4, 11.3.

### Preparation 60

### Compound 50, 3-(2,5-dimethoxy-benzylidene)-1-methoxy-1,3-dihydro-indol-2-one

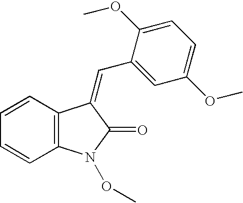

The title compound was prepared following the general procedure 1 using 1-methoxy-oxindole (0.49 g, 3 mmoles) and 2,5-dimethoxybenzaldehyde (0.5 g, 3 mmoles). Yield: 63% (0.59 g, 1.9 mmoles) as yellow crystals.

¹³C-NMR (DMSO-d₆, mixture of the Z and E isomers, signals of the predominant form) δ 162.4, 152.5, 151.9, 139.4, 133.2, 130.1, 123.9, 122.7, 122.5, 122.4, 117.5, 117.4, 114.3, 112.7, 107.4, 63.5, 55.9, 55.5.

### Preparation 61

### Compound 51, acetic acid 3-(3,5-dimethyl-1H-pyrrol-2-ylmethylene)-2-oxo-2,3-dihydro-indol-1-yl ester

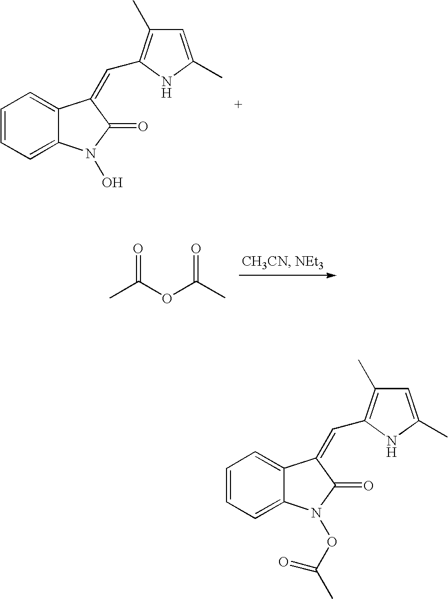

To a suspension of 3-(3,5-dimethyl-1H-pyrrol-2-ylmethylene)-1-hydroxy-1,3-dihydro-indol-2-one (416 mg, 1.64 mmoles) in acetonitrile (12 mL) were added acetic anhydride (2.5 mL) and triethylamine (2.5 mL). The suspension turned to a solution, which was stirred for 1 hour at room temperature. Concentration in vacuo and purification by flash chromatography (using a gradient of petroleum ether/EtOAc from 100:0 to 85:15) afforded 348 mg of the title compound (71% yield).

¹H NMR (d₆-DMSO, Z isomer) δ 12.77 (s, 1H), 7.87 (d, 1H), 7.72 (s, 1H), 7.20 (t, 1H), 7.12 (t, 1H), 7.04 (d, 1H), 6.08 (d, 1H), 2.45 (s, 3H), 2.34 (s, 6H).

### Preparation 62

### 2-[3-(2-Oxo-2,3-dihydro-indol-1-yloxy)-propyl]-isoindole-1,3-dione

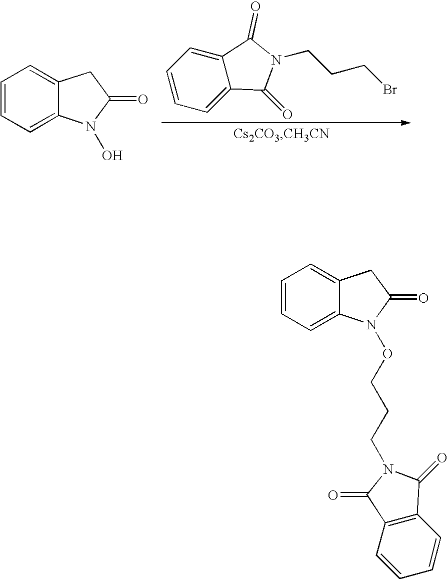

To a solution of 1-hydroxy-1,3-dihydro-indol-2-one (1 g, 6.7 mmoles) and 2-(3-bromo-propyl)-isoindole-1,3-dione (2 g, 7.4 mmoles) in CH₃CN (12 mL) was added Cs₂CO₃ (4.36 g, 13.4 mmoles). The reaction mixture was stirred under argon at 30° C. The reaction evolution was followed by LC/MS. The mixture was then concentrated in vacuo together with silica gel. The residue was purified by chromatography to afford the pure title compound (1.25 g, 56% yield).

¹³C-NMR (DMSO-d₆) δ 169.4, 167.9, 141.5, 134.2, 131.7, 127.7, 124.7, 122.9, 122.4, 121.1, 107.0, 73.5, 34.5, 33.4, 27.1.

### Preparation 63

### Compound 52, 2-{3-[3-(3,5-dimethyl-1H-pyrrol-2-ylmethylene)-2-oxo-2,3-dihydro-indol-1-yloxy]-propyl}-isoindole-1,3-dione

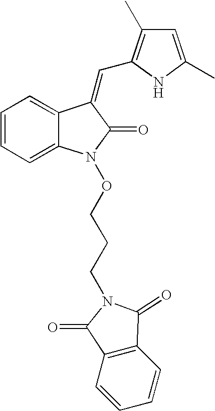

The title compound was obtained following the general procedure 1 using 2-[3-(2-oxo-2,3-dihydro-indol-1-yloxy)-propyl]-isoindole-1,3-dione (0.63 g, 1.9 mmoles) and 3,5-dimethyl-1H-pyrrole-2-carbaldehyde (0.23 g, 1.9 mmoles, synthesis described in J. Chem. Soc. Perkin trans I, 1988, p. 1195 (448 mg, 54%).

¹³C-NMR (DMSO-d₆, Z isomer) δ 12.94 (s, 1H), 7.91-7.78 (m, 5H), 7.63 (s, 1H), 7.21 (t, 1H), 7.12 (d, 1H), 7.07 (t, 1H), 6.05 (s, 1H), 4.28 (t, 2H), 3.83 (t, 2H), 2.35 (s, 3H), 2.31 (s, 3H), 2.12 (m, 2H).

### Preparation 64

### Compound 53, 3-(phenyl-4-tolyl-methylene)-1,3-dihydro-indol-2-one

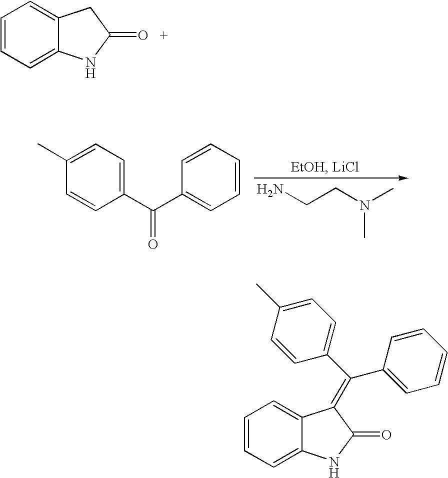

1,3-Dihydro-indol-2-one (100 mg, 0.76 mmole), phenyl-p-tolyl-methanone (164 mg, 0.83 mmole) and LiCl (cat.) were suspended in dry ethanol (1 mL). 2-Dimethylamino ethylamine (0.42 mL, 3.8 mmoles) was added. The flask was flushed with argon, sealed and exposed to microwave irradiations (125 W, 120° C.) for 1 hour. After concentration in vacuo and purification by flash chromatography (using a gradient of petroleum ether/EtOAc from 1:0 to 2:3) the title compound was obtained as a red-orange oil (43 mg, 18% yield).

¹³C NMR (CDCl₃, mixture of E and Z isomers) δ 168.3, 168.3, 155.4, 141.6, 140.5, 140.3, 140.1, 139.6, 139.5, 138.4, 136.8, 130.6, 130.4, 129.6, 129.5, 129.2, 128.9, 128.6, 128.5, 127.8, 124.4, 124.4, 124.1, 124.0, 123.3, 121.2, 109.4, 109.3, 21.5, 21.5.

### Preparation 65

### Compound 54, 3-[bis-(4-methoxy-phenyl)-methylene]-1,3-dihydro-indol-2-one

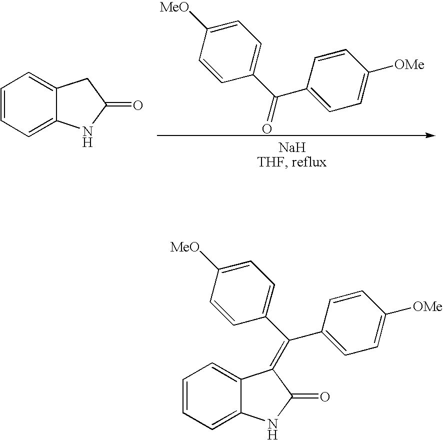

NaH (50% dispersion in oil, 50 mg, 1 mmole) was added in one portion to a solution of 1,3-dihydro-indol-2-one (130 mg, 1 mmole) in dry THF (5 mL). When the resulting gas evolution ceased bis-(4-methoxy-phenyl)-methanone (290 mg, 1.2 mmoles) was added in one portion. The reaction mixture was heated to reflux for about 3 days. Extra portions of NaH (in total 75 mg, 1.5 mmoles) were added over the next 2 days. H₂O was then added (10 mL) and the pH of the crude was adjusted to 6-7 by addition of cold acetic acid. Crystals formed which were filtered and recrystallized from MeOH to afford 200 mg of the pure title compound (55% yield)

¹H NMR (DMSO-d₆) δ 10.37 (s, 1H), 7.18 (m, 4H), 7.04 (m, 3H), 6.88 (d, 2H), 6.75 (d, 1H), 6.59 (t, 1H), 6.27 (d, 1H), 3.83 (s, 3H), 3.79 (s, 3H).

### Preparation 66

### Compound 55, 3-[1-(2,5-dimethoxy-phenyl)-ethylidene]-1,3-dihydro-indol-2-one

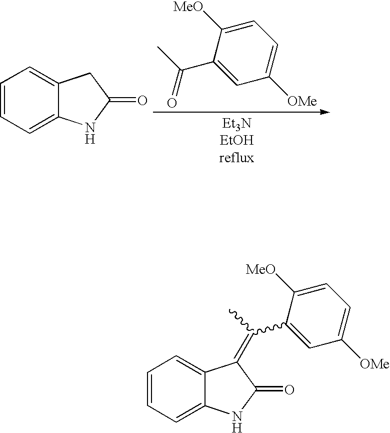

To a solution of 1,3-dihydro-indol-2-one (1.0 g, 7.5 mmoles) and 1-(2,5-dimethoxy-phenyl)-ethanone (2.43 g, 13.5 mmoles) in EtOH (5 mL) was added diethyl-amine (1 mL). After 5 days at reflux the mixture was allowed to come to room temperature, diluted with MeOH/EtOH and filtered. The filtrate was concentrated in vacuo and purified by flash chromatography using a mixture of petroleum ether and EtOAc as eluent. The title compound was obtained as a yellow powder (300 mg, 13% yield).

¹³C-NMR (DMSO-d₆, Z isomer) δ 168.7, 153.7, 150.7, 148.7, 140.6, 131.6, 128.1, 124.0, 122.8, 122.0, 120.5, 114.1, 113.3, 113.1, 109.1, 56.0, 55.5, 21.0.

### Preparation 67

### Compound 56, (1-Methyl-1H-indol-3-yl)-(2-oxo-1,2-dihydro-indol-3-ylidene)-acetic acid

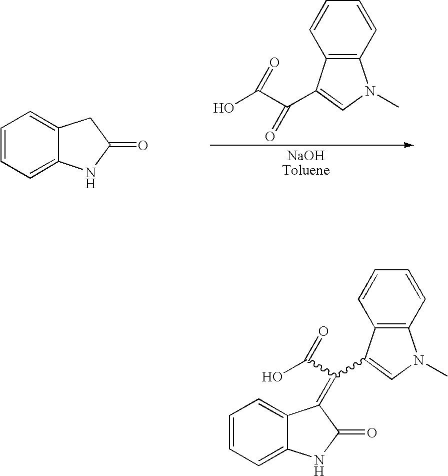

In a flask equipped with a Dean Stark apparatus was heated to reflux a solution of sodium hydroxide (1.6 g, 40 mmoles), 1,3-dihydro-indol-2-one (2.62 g, 19.7 mmoles) and (1-methyl-1H-indol-3-yl)-oxo-acetic acid (4.0 g, 19.7 mmoles) in toluene (70 mL) overnight. After removal of the solvent in vacuo and addition of water (50 mL), the solution was acidified by addition of an aqueous solution of HCl (1.5 N) at 0° C. A precipitate formed which was filtered and purified by flash chromatography to afford the pure title compound (1.41 g, 22% yield).

¹³C-NMR (DMSO-d₆) δ 169.6, 167.5, 142.2, 137.1, 137.0, 132.4, 129.0, 125.0, 123.0, 122.4, 121.9, 120.7, 120.6, 120.5, 111.0, 109.5, 108.2, 33.0.

### Preparation 68

The following compounds were obtained following general procedure 1 and corresponding starting materials:

### Compound 57, 3-(1H-indol-3-ylmethylene)-1,3-dihydro-indol-2-one

¹³C-NMR (DMSO-d₆ mixture of E and Z isomers) δ 169.6, 168.0, 141.6, 139.1, 136.3, 135.8, 133.4, 129.7, 128.1, 127.6, 127.1, 126.7, 125.6, 122.6, 122.4, 122.2, 122.1, 121.3, 120.8, 120.7, 120.4, 119.0, 118.8, 118.6, 118.3, 112.4, 112.2, 111.2, 110.4, 109.4, 108.9.

### Compound 58, 3-(2-phenyl-1H-indol-3-ylmethylene)-1,3-dihydro-indol-2-one

¹H-NMR (DMSO-d₆) δ 12.28 (s, 1H), 10.56 (s, 1H), 7.70 (m, 3H), 7.57 (m, 3H), 7.48 (t, 1H), 7.28 (m, 1H), 7.20-7.07 (m, 3H), 6.90 (d, 1H), 6.75 (m, 2H).

### Compound 59, 3-(1-methyl-2-phenyl-1H-indol-3-ylmethylene)-1,3-dihydro-indol-2-one

¹³C-NMR (DMSO-d₆, E isomer) δ 169.2, 144.0, 141.9, 137.7, 130.8, 129.7, 129.1, 128.6, 128.4, 128.0, 124.4, 124.1, 124.1, 122.6, 122.0, 121.6, 120.6, 120.3, 111.2, 109.2, 31.5.

### Compound 60, 3-[2-(4-chloro-phenyl)-1H-indol-3-ylmethylene]-1,3-dihydro-indol-2-one

¹H-NMR (DMSO-d₆) δ 12.32 (s, 1H), 10.56 (s, 1H), 7.69 (d, 2H), 7.64 (d, 2H), 7.57 (d, 1H), 7.28 (m, 1H), 7.19-7.07 (m, 3H), 6.88 (d, 1H), 6.72 (m, 2H).

### Compound 61, 3-(2-naphthalen-2-yl-1H-indol-3-ylmethylene)-1,3-dihydro-indol-2-one

¹³C-NMR (DMSO-d₆, E isomer) δ 169.2, 142.0, 140.6, 136.8, 132.7, 132.5, 128.8, 128.5, 128.5, 128.2, 128.1, 127.6, 126.9, 125.9, 125.5, 125.0, 124.4, 122.8, 122.1, 121.4, 120.4, 120.2, 112.3, 109.2, 108.4.

### Compound 62, 5-chloro-3-(1H-indol-3-ylmethylene)-1,3-dihydro-indol-2-one

¹H-NMR (DMSO-d₆, Z isomer) δ 12.09 (br, 1H), 10.65 (s, 1H), 9.48 (s, 1H), 8.30 (m, 2H), 8.08 (d, 1H), 7.53 (m, 1H), 7.25 (m, 2H), 7.15 (dd, 1H), 6.84 (d, 1H).

### Compound 63, 3-(5-methyl-1H-indol-3-ylmethylene)-1,3-dihydro-indol-2-one

¹H-NMR (DMSO-d₆, mixture of E and Z isomers) δ 11.90 (s, 1H), 10.49 (s, 0.6H), 10.46 (s, 0.4H), 9.42 (d, 0.6H), 8.20 (s, 0.4H), 8.13 (s, 0.6H), 7.98 (s, 0.6H), 7.89 (s, 0.6H), 7.87 (s, 0.4H), 7.80 (d, 0.4H), 7.48 (s, 0.4H), 7.42 (t, 1H), 7.24-6.80 (m, 4H), 2.48 (s, 1.8H), 2.43 (s, 1.2H).

### Compound 64, 5,7-difluoro-3-(1H-indol-3-ylmethylene)-1,3-dihydro-indol-2-one

¹H-NMR (DMSO-d₆, Z isomer) δ 12.18 (br, 1H), 11.02 (s, 1H), 9.52 (s, 1H), 8.32 (s, 1H), 8.26 (m, 1H), 7.80 (dd, 1H), 7.55 (m, 1H), 7.27 (m, 2H), 7.05 (t, 1H).

### Compound 65, 5-bromo-3-(1H-indol-3-ylmethylene)-1,3-dihydro-indol-2-one

¹H-NMR (DMSO-d₆, Z isomer) δ 12.08 (br, 1H), 10.65 (s, 1H), 9.48 (s, 1H), 8.30 (s, 1H), 8.28 (m, 1H), 8.20 (d, 1H), 7.53 (m, 1H), 7.31-7.20 (m, 3H), 6.80 (d, 1H).

### Compound 66, 6-fluoro-3-(1H-indol-3-ylmethylene)-1,3-dihydro-indol-2-one

¹H-NMR (DMSO-d₆, E isomer) δ 11.99 (br, 1H), 10.62 (br, 1H), 8.22 (s, 1H), 7.86 (s, 1H), 7.73 (dd, 1H), 7.64 (d, 1H), 7.55 (d, 1H), 7.30-7.15 (m, 2H), 6.80-6.65 (m, 2H).

### Compound 67, 6-bromo-3-(1H-indol-3-ylmethylene)-1,3-dihydro-indol-2-one

¹H-NMR (DMSO-d₆, mixture of E and Z isomers, signals of the predominant isomer) δ 12.08 (br, 1H), 10.66 (br, 1H), 9.46 (s, 1H), 8.23 (s, 1H), 8.20 (m, 1H), 7.87 (d, 1H), 7.53 (m, 1H), 7.25 (m, 2H), 7.17 (dd, 1H), 6.99 (d, 1H).

### Compound 68, 5-hydroxy-3-(1H-indol-3-ylmethylene)-1,3-dihydro-indol-2-one

¹H-NMR (DMSO-d₆, mixture of E and Z isomers) δ 11.99 (br, 1H), 10.20 (s, 0.5H), 10.16 (s, 0.5H), 9.44 (s, 0.5H), 8.92 (br, 1H), 8.14 (s, 1H), 8.00 (s, 0.5H), 7.82 (s, 0.5H), 7.68 (d, 0.5H), 7.52 (m, 1H), 7.34-7.15 (m, 3H), 6.72-6.54 (m, 2H).

### Compound 69, 3-(4,5,6,7-tetrafluoro-1H-indol-3-ylmethylene)-1,3-dihydro-indol-2-one

¹H-NMR (DMSO-d₆, E isomer) δ 13.00 (br, 1H), 10.63 (s, 1H), 9.46 (s, 1H), 7.95 (s, 1H), 7.56 (d, 1H), 7.19 (t, 1H), 7.00 (t, 1H), 6.86 (d, 1H).

### Compound 70, 3-(6-fluoro-1H-indol-3-ylmethylene)-1,3-dihydro-indol-2-one

¹H-NMR (DMSO-d₆, Z isomer) δ 12.03 (s, 1H), 10.54 (s, 1H), 9.43 (s, 1H), 8.20 (dd, 1H), 8.13 (s, 1H), 7.90 (d, 1H), 7.33 (dd, 1H), 7.2-7.05 (m, 2H), 6.99 (t, 1H), 6.85 (d, 1H).

### Compound 71, 3-[2-(4-chloro-phenyl)-5-nitro-1H-indol-3-ylmethylene)-1,3-dihydro-indol-2-one

¹H-NMR (DMSO-d₆, mixture of E and Z isomers, signals of the predominant isomer) δ 12.95 (br, 1H), 10.67 (s, 1H), 8.16 (dd, 1H), 8.08 (d, 1H), 7.80-7.60 (m, 6H), 7.17 (m, 1H), 6.91 (d, 1H), 6.72 (m, 2H).

### Compound 72, 7-bromo-3-(1H-indol-3-ylmethylene)-1,3-dihydro-indol-2-one

¹H-NMR (DMSO-d₆, mixture of E and Z isomers, signals of the predominant isomer) δ 12.13 (br, 1H), 10.81 (s, 1H), 9.50 (s, 1H), 8.24 (s, 1H), 8.21 (m, 1H), 7.93 (d, 1H), 7.55 (d, 1H), 7.32 (d, 1H), 7.26 (m, 2H), 6.96 (t, 1H).

### Compound 73, 3-(6-methyl-1H-indol-3-ylmethylene)-1,3-dihydro-indol-2-one

¹H-NMR (DMSO-d₆, E isomer) δ 11.87 (br, 1H), 10.50 (s, 1H), 9.38 (s, 1H), 8.12 (s, 1H), 8.05 (d, 1H), 7.87 (d, 1H), 7.31 (s, 1H), 7.13 (t, 1H), 7.07 (d, 1H), 6.98 (t, 1H), 6.84 (d, 1H), 2.45 (s, 3H).

### Compound 74, 3-(7-methyl-1H-indol-3-ylmethylene)-1,3-dihydro-indol-2-one

¹H-NMR (DMSO-d₆, E isomer) δ 11.99 (br, 1H), 10.50 (s, 1H), 9.46 (s, 1H), 8.13 (s, 1H), 7.99 (d, 1H), 7.87 (d, 1H), 7.2-7.0 (m, 4H), 6.86 (d, 1H), 2.54 (s, 3H).

### Compound 75, 3-(2-methyl-1H-indol-3-ylmethylene)-1,3-dihydro-indol-2-one

¹³C-NMR (DMSO-d₆, E isomer) δ 169.2, 141.8, 140.5, 135.8, 128.3, 127.7, 125.7, 123.9, 122.8, 122.3, 121.4, 120.4, 120.3, 119.8, 111.4, 109.1, 108.2, 12.6.

### Compound 76, 3-(5-fluoro-1-methyl-1H-indol-3-ylmethylene)-1,3-dihydro-indol-2-one

¹H-NMR (DMSO-d₆, Z isomer) δ 10.55 (s, 1H), 9.43 (s, 1H), 8.10 (dd, 1H), 8.09 (s, 1H), 7.92 (d, 1H), 7.60 (dd, 1H), 7.2-7.1 (m, 2H), 6.99 (t, 1H), 6.85 (d, 1H), 3.95 (s, 3H).

### Compound 77, 3-(5-fluoro-1H-indol-3-ylmethylene)-1,3-dihydro-indol-2-one

¹H-NMR (DMSO-d₆, Z isomer) δ 12.07 (s, 1H), 10.53 (s, 1H), 9.48 (s, 1H), 8.12 (s, 1H), 8.07 (dd, 1H), 7.92 (d, 1H), 7.53 (dd, 1H), 7.2-6.95 (m, 3H), 6.84 (d, 1H).

### Compound 78, 3-(5-methoxy-1H-indol-3-ylmethylene)-1,3-dihydro-indol-2-one

¹³C-NMR (DMSO-d₆, mixture of E and Z isomers) δ 169.7, 168.1, 155.0, 154.7, 141.6, 139.0, 134.0, 131.2, 130.8, 130.2, 129.1, 128.1, 127.9, 127.6, 126.5, 125.9, 122.3, 120.8, 120.4, 118.7, 118.4, 113.3, 112.9, 112.4, 111.4, 110.5, 109.5, 108.9, 100.8, 55.6.

### Compound 79, 3-(5-benzyloxy-1H-indol-3-ylmethylene)-1,3-dihydro-indol-2-one

¹³C-NMR (DMSO-d₆ mixture of E and Z isomers) δ 169.6, 168.1, 154.0, 153.8, 141.6, 139.0, 137.5, 134.0, 131.4, 130.9, 130.2, 129.1, 128.3, 128.0, 127.7, 127.6, 127.5, 127.4, 127.3, 126.5, 125.8, 122.3, 122.1, 120.7, 120.3, 118.6, 118.4, 113.4, 113.2, 112.9, 112.7, 111.3, 110.4, 109.4, 108.8, 102.3, 70.0, 69.8.

### Compound 80, 3-(6-methoxy-1H-indol-3-ylmethylene)-1,3-dihydro-indol-2-one

¹³C-NMR (DMSO-d₆, mixture of E and Z isomers) δ 169.7, 168.1, 156.5, 141.7, 139.1, 137.2, 136.7, 132.6, 128.8, 128.2, 127.9, 127.3, 126.7, 125.8, 122.3, 121.3, 120.9, 120.5, 119.7, 119.1, 119.0, 118.6, 111.4, 110.8, 110.6, 109.5, 109.0, 95.4, 55.3.

### Compound 81, 3-(5-methoxy-1-methyl-1H-indol-3-ylmethylene)-1,3-dihydro-indol-2-one

¹³C-NMR (DMSO-d₆, mixture of E and Z isomers) δ 169.7, 168.0, 155.3, 155.0, 141.5, 139.0, 137.2, 133.6, 131.9, 131.6, 129.6, 128.4, 128.0, 127.2, 126.8, 126.5, 125.8, 122.3, 120.8, 120.3, 118.6, 118.2, 112.8, 112.2, 111.6, 111.4, 110.3, 109.3, 108.8, 101.0, 55.7, 55.4, 33.5, 33.3.

### Compound 82, 3-(6-methoxy-1-methyl-1H-indol-3-ylmethylene)-1,3-dihydro-indol-2-one

¹³C-NMR (DMSO-d₆, mixture of E and Z isomers) δ 169.6, 167.9, 156.7, 141.6, 139.1, 137.8, 137.4, 136.0, 132.3, 128.1, 127.0, 126.6, 125.7, 122.6, 122.3, 122.2, 121.8, 120.8, 120.4, 119.6, 119.2, 118.9, 118.6, 110.7, 110.5, 109.7, 109.4, 108.9, 94.2, 55.5, 33.3, 33.1.

### Compound 83, 3-(4-benzyloxy-1H-indol-3-ylmethylene)-1,3-dihydro-indol-2-one

¹³C-NMR (DMSO-d₆, mixture of E and Z isomers) δ 167.7, 153.8, 138.8, 137.6, 137.2, 137.0, 132.9, 131.7, 130.0, 128.8, 128.6, 128.3, 128.2, 127.3, 126.7, 126.3, 125.8, 123.5, 123.3, 121.0, 120.7, 120.3, 118.3, 117.2, 116.4, 112.0, 108.9, 105.9, 102.9, 69.9, 69.1.

### Compound 84, 3-(1H-indol-3-ylmethylene)-2-oxo-2,3-dihydro-1H-indol-6-carbonitrile

¹H-NMR (DMSO-d₆, mixture of E and Z isomers, signals of the predominant isomer) δ 12.26 (br, 1H), 10.84 (br, 1H), 9.56 (s, 1H), 8.41 (s, 1H), 8.24 (m, 1H), 8.11 (d, 1H), 7.55 (m, 1H), 7.45 (d, 1H), 7.27 (m, 2H), 7.18 (s, 1H).

### Compound 85, 3-(1H-indol-3-ylmethylene)-2-oxo-2,3-dihydro-1H-indol-7-carbonitrile

¹H-NMR (DMSO-d₆, mixture of E and Z isomers, signals of the predominant isomer) δ 11.76 (br, 2H), 9.50 (s, 1H), 8.34 (s, 1H), 8.22 (m, 2H), 7.6-7.45 (m, 2H), 7.27 (m, 2H), 7.14 (t, 1H).

### Compound 86, 3-(1H-indol-3-ylmethylene)-2-oxo-2,3-dihydro-1H-indol-5-carbonitrile

¹H-NMR (DMSO-d₆, Z isomer) δ 12.15 (br, 1H), 11.02 (br, 1H), 9.49 (s, 1H), 8.46 (s, 1H), 8.41 (s, 1H), 8.27 (m, 1H), 7.55 (m, 2H), 7.27 (m, 2H), 7.00 (d, 1H).

### Compound 87, 7-fluoro-3-(1H-indol-3-ylmethylene)-1,3-dihydro-indol-2-one

¹H-NMR (DMSO-d₆, mixture of E and Z isomers, signals of the predominant isomer) δ 12.08 (br, 1H), 10.98 (br, 1H), 9.49 (s, 1H), 8.24 (s, 1H), 8.20 (m, 1H), 7.76 (d, 1H), 7.54 (m, 1H), 7.25 (m, 2H), 7.00 (m, 2H).

### Compound 88, 3-(1H-indol-3-ylmethylene)-6-trifluoromethyl-1,3-dihydro-indol-2-one

¹H-NMR (DMSO-d₆, mixture of E and Z isomers, signals of the predominant isomer) δ 12.15 (br, 1H), 10.78 (br, 1H), 9.54 (s, 1H), 8.38 (s, 1H), 8.24 (m, 1H), 8.12 (d, 1H), 7.55 (m, 1H), 7.34 (d, 1H), 7.27 (m, 2H), 7.08 (s, 1H).

### Compound 89, 3-(1H-indol-3-ylmethylene)-6-methanesulfonyl-1,3-dihydro-indol-2-one

¹H-NMR (DMSO-d₆) δ 10.85 (br, 1H), 9.57 (s, 1H), 8.40 (s, 1H), 8.22 (br, 1H), 8.13 (br, 1H), 7.55 (br, 2H), 7.26 (br, 3H), 3.20 (s, 3H).

### Compound 90, 3-(1H-indol-3-ylmethylene)-5-trifluoromethyl-1,3-dihydro-indol-2-one

¹H-NMR (DMSO-d₆, Z isomer) δ 12.14 (br, 1H), 10.89 (br, 1H), 9.51 (s, 1H), 8.44 (s, 1H), 8.37 (s, 1H), 8.32 (m, 1H), 7.54 (m, 1H), 7.47 (d, 1H), 7.26 (m, 2H), 7.00 (d, 1H).

### Compound 91, 3-(1H-indol-3-ylmethylene)-5,6-dimethoxy-1,3-dihydro-indol-2-one

¹H-NMR (DMSO-d₆, Z isomer) δ 11.87 (br, 1H), 10.24 (s, 1H), 9.36 (s, 1H), 8.20 (m, 1H), 7.99 (s, 1H), 7.58 (s, 1H), 7.50 (m, 1H), 7.22 (m, 2H), 6.49 (s, 1H), 3.83 (s, 3H), 3.77 (s, 3H)

### Compound 92, 4,5-difluoro-3-(1H-indol-3-ylmethylene)-1,3-dihydro-indol-2-one

¹H-NMR (DMSO-d₆, Z isomer) δ 12.19 (br, 1H), 10.76 (br, 1H), 9.48 (s, 1H), 8.18 (d, 1H), 7.84 (m, 1H), 7.56 (m, 1H), 7.27 (m, 2H), 7.16 (m, 1H), 6.65 (dd, 1H)

### Compound 92 A, 3-(1H-indol-3-ylmethylene)-5-methoxy-1,3-dihydro-indol-2-one

¹H-NMR (DMSO-d₆, Z isomer) δ 11.99 (br, 1H), 10.28 (br, 1H), 9.45 (s, 1H), 8.22 (m, 1H), 8.15 (s, 1H), 7.57 (s, 1H), 7.51 (m, 1H), 7.24 (m, 2H), 6.73 (m, 2H), 3.81 (s, 3H)

### Compound 93, 6-chloro-3-(1H-indol-3-ylmethylene)-1,3-dihydro-indol-2-one

¹H-NMR (DMSO-d₆, Z isomer) δ 12.06 (br, 1H), 10.65 (br, 1H), 9.44 (s, 1H), 8.21 (s, 1H), 8.18 (m, 1H), 7.91 (d, 1H), 7.53 (m, 1H), 7.25 (m, 2H), 7.03 (dd, 1H), 6.86 (d, 1H)

### Compound 94, 3-[1-Methyl-2-(4-methyl-piperazin-1-yl)-1H-indol-3-ylmethylene]-1,3-dihydro-indol-2-one

¹³C-NMR (DMSO-d₆, mixture of E and Z isomers) δ 169.3, 166.7, 151.2, 149.8, 141.6, 139.6, 135.0, 134.7, 128.0, 127.5, 126.8, 125.6, 124.2, 123.9, 122.2, 122.2, 121.1, 120.7, 120.3, 120.2, 120.1, 119.9, 118.5, 110.1, 109.2, 109.0, 108.7, 100.5, 99.5, 55.1, 51.0, 50.3, 45.8, 29.6.

### Compound 95, 3-(4-hydroxy-3,5-dimethyl-benzylidene)-1,3-dihydro-indol-2-one

¹³C-NMR (DMSO-d₆, mixture of E and Z isomers) δ 169.0, 167.3, 156.3, 155.3, 142.4, 139.9, 137.6, 136.8, 133.4, 130.2, 129.2, 127.6, 125.6, 125.5, 125.0, 124.5, 124.4, 123.6, 122.7, 121.8, 121.4, 120.9, 120.7, 118.8, 109.8, 108.9, 16.6, 16.4.

### Compound 96, 3-(3,5-di-tert-butyl-4-hydroxy-benzylidene)-1,3-dihydro-indol-2-one

¹³C-NMR (DMSO-d₆) δ 169.0, 156.0, 142.5, 138.8, 137.8, 137.5, 130.2, 129.3, 126.9, 125.4, 124.4, 121.9, 121.3, 120.6, 109.9, 34.6, 30.1.

### Compound 97, 3-(4-bromo-benzylidene)-1,3-dihydro-indol-2-one

¹H-NMR (DMSO-d₆) δ 10.62 (br, 1H), 7.72 (d, 2H), 7.66 (d, 2H), 7.56 (s, 1H), 7.48 (d, 1H), 7.24 (t, 1H), 6.86 (m, 2H).

### Compound 98, 3-(2-methyl-benzylidene)-1,3-dihydro-indol-2-one

¹H-NMR (DMSO-d₆) δ 10.60 (br, 1H), 7.68 (s, 1H), 7.54 (d, 1H), 7.40-7.05 (m, 5H), 6.86 (d, 1H), 6.77 (t, 1H), 2.30 (s, 3H).

### Compound 99, 3-(2,4-dimethyl-benzylidene)-1,3-dihydro-indol-2-one

¹H-NMR (DMSO-d₆) δ 10.57 (br, 1H), 7.64 (s, 1H), 7.47 (d, 1H), 7.30-7.05 (m, 4H), 6.85 (d, 1H), 6.78 (t, 1H), 2.34 (s, 3H), 2.27 (s, 3H).

### Compound 100, 3-(2,6-dichloro-benzylidene)-1,3-dihydro-indol-2-one

¹H-NMR (DMSO-d₆) δ 10.7 (br, 1H), 7.65 (d, 2H), 7.53 (dd, 1H), 7.42 (s, 1H), 7.23 (t, 1H), 6.80 (t, 1H), 6.66 (d, 1H), 6.48 (d, 1H).

### Compound 101, 3-(3,4-dichloro-benzylidene)-1,3-dihydro-indol-2-one

¹H-NMR (DMSO-d₆) δ 10.64 (br, 1H), 7.95 (d, 1H), 7.78 (d, 1H), 7.70 (dd, 1H), 7.56 (s, 1H), 7.41 (d, 1H), 7.25 (t, 1H), 6.86 (m, 2H).

### Compound 102, 3-(4-chloro-benzylidene)-1,3-dihydro-indol-2-one

¹H-NMR (DMSO-d₆) δ 10.62 (br, 1H), 7.73 (d, 2H), 7.58 (m, 3H), 7.48 (d, 1H), 7.23 (t, 1H), 6.86 (m, 2H).

### Compound 103, 3-(2,4-dimethyl-benzylidene)-1,3-dihydro-indol-2-one

¹³C-NMR (DMSO-d₆, E isomer) δ 168.3, 142.6, 139.1, 136.8, 134.7, 131.0, 130.7, 129.8, 128.3, 127.7, 126.4, 122.2, 121.1, 121.0, 109.9, 20.8, 19.3.

### Compound 104, 3-(2,5-dimethyl-benzylidene)-1,3-dihydro-indol-2-one

¹³C-NMR (DMSO-d₆, E isomer) δ 168.3, 142.8, 134.8, 134.8, 133.6, 133.5, 130.3, 129.9, 128.5, 128.3, 122.2, 121.1, 121.1, 110.0, 20.4, 18.9.

### Compound 105, 3-(2,6-dimethoxy-benzylidene)-1,3-dihydro-indol-2-one

¹H-NMR (DMSO-d₆) δ 10.47 (br, 1H), 7.43 (m, 2H), 7.16 (t, 1H), 6.80 (m, 5H), 3.75 (s, 6H).

### Compound 106, 3-benzylidene-1,3-dihydro-indol-2-one

¹H-NMR (DMSO-d₆) δ 10.60 (br, 1H), 7.70 (d, 2H), 7.64 (s, 1H), 7.50 (m, 4H), 7.23 (t, 1H), 6.85 (m, 2H).

### Compound 107, 3-(4-methoxy-benzylidene)-1,3-dihydro-indol-2-one

¹H-NMR (DMSO-d₆) δ 10.54 (br, 1H), 7.71 (d, 2H), 7.65 (d, 1H), 7.58 (s, 1H), 7.22 (t, 1H), 7.09 (d, 2H), 6.87 (m, 2H), 3.85 (s, 3H).

### Compound 108, 3-(2,4-dimethoxy-benzylidene)-1,3-dihydro-indol-2-one

¹³C-NMR (DMSO-d₆, E isomer) δ 168.8, 162.6, 159.3, 142.4, 131.6, 130.5, 129.3, 125.2, 121.9, 121.3, 120.9, 115.3, 109.8, 105.3, 98.4, 55.7, 55.5.

### Compound 109, 3-(3,5-dimethoxy-benzylidene)-1,3-dihydro-indol-2-one

¹H-NMR (DMSO-d₆) δ 10.59 (br, 1H), 7.59 (d, 1H), 7.56 (s, 1H), 7.23 (t, 1H), 6.90-6.83 (m, 4H), 6.61 (m, 1H), 3.79 (s, 6H).

### Compound 110, 3-(2,5-dimethoxy-benzylidene)-1,3-dihydro-indol-2-one

¹³C-NMR (DMSO-d₆, E isomer) δ 168.6, 152.5, 151.8, 142.8, 131.4, 129.9, 127.5, 123.3, 122.4, 121.0, 121.0, 116.8, 114.4, 112.6, 110.0, 55.9, 55.5.

### Compound 111, 3-(3,4-dimethyl-benzylidene)-1,3-dihydro-indol-2-one

¹H-NMR (DMSO-d₆) δ 10.56 (br, 1H), 7.59 (d, 1H), 7.57 (s, 1H), 7.46 (m, 2H), 7.29 (d, 1H), 7.20 (t, 1H), 6.85 (m, 2H), 2.30 (s, 3H), 2.29 (s, 3H).

### Compound 112, 3-naphtalen-2-ylmethylene-1,3-dihydro-indol-2-one

¹H-NMR (DMSO-d₆) δ 10.63 (br, 1H), 8.28 (s, 1H), 8.06 (d, 1H), 8.03-7.97 (m, 2H), 7.82 (m, 2H), 7.64-7.52 (m, 3H), 7.24 (t, 1H), 6.90 (d, 1H), 6.85 (t, 1H).

### Compound 113, 3-naphtalen-1-ylmethylene-1,3-dihydro-indol-2-one

¹H-NMR (DMSO-d₆) δ 10.67 (br, 1H), 8.07 (m, 3H), 7.93 (m, 1H), 7.83 (d, 1H), 7.66 (d, 1H), 7.61 (m, 2H), 7.18 (t, 1H), 6.89 (m, 2H), 6.68 (t, 1H).

### Compound 114, 3-(2,3-dimethoxy-benzylidene)-1,3-dihydro-indol-2-one

¹H-NMR (DMSO-d₆) δ 10.60 (br, 1H), 7.64 (s, 1H), 7.36 (d, 1H), 7.26-7.17 (m, 4H), 6.82 (m, 2H), 3.87 (s, 3H), 3.75 (s, 3H).

### Compound 115, 3-(3-nitro-benzylidene)-1,3-dihydro-indol-2-one

¹H-NMR (DMSO-d₆) δ 10.71 (br, 1H), 9.39 (s, 1H), 8.64 (d, 1H), 8.27 (dd, 1H), 7.97 (s, 1H), 7.76 (m, 2H), 7.26 (t, 1H), 7.03 (t, 1H), 6.85 (d, 1H).

### Compound 116, 3-(2-fluoro-benzylidene)-1,3-dihydro-indol-2-one

¹H-NMR (DMSO-d₆) δ 10.65 (br, 1H), 7.76 (t, 1H), 7.54 (m, 2H), 7.37 (m, 2H), 7.24 (m, 2H), 6.86 (m, 2H).

### Compound 117, 3-(3-methyl-benzylidene)-1,3-dihydro-indol-2-one

¹³C-NMR (CDCl₃) δ 169.9, 138.4, 137.9, 134.8, 130.5, 129.9, 129.8, 128.6, 126.4, 123.1, 121.9, 110.0, 21.4.

### Compound 118, 3-(3-fluoro-benzylidene)-1,3-dihydro-indol-2-one

¹³C-NMR (CDCl₃, E isomer) δ 170.3, 162.8, 142.0, 137.0, 135.6, 130.4, 130.3, 128.7, 125.1, 123.2, 122.0, 121.3, 116.5, 115.9, 110.5.

### Compound 119, 3-(4-fluoro-benzylidene)-1,3-dihydro-indol-2-one

¹H-NMR (DMSO-d₆) δ 10.46 (br, 1H), 7.77 (d, 1H), 7.63 (d, 2H), 7.51 (s, 1H), 7.19 (t, 1H), 7.03 (d, 2H), 6.87 (m, 2H).

### Compound 120, 3-anthracen-9-ylmethylene-1,3-dihydro-indol-2-one

¹H-NMR (DMSO-d₆) δ 10.74 (br, 1H), 8.78 (s, 1H), 8.35 (s, 1H), 8.21 (d, 2H), 7.98 (d, 2H), 7.62-7.48 (m, 4H), 7.07 (t, 1H), 6.84 (d, 1H), 6.39 (t, 1H), 5.65 (d, 1H).

### Compound 121, 3-(5-bromo-2-hydroxy-benzylidene)-1,3-dihydro-indol-2-one

¹H-NMR (DMSO-d₆) δ 10.58 (s, 1H), 7.72 (d, 1H), 7.56 (s, 1H), 7.46 (dd, 1H), 7.38 (d, 1H), 7.23 (t, 1H), 6.94 (d, 1H), 6.88 (m, 2H).

### Compound 122, 3-(2-hydroxy-benzylidene)-1,3-dihydro-indol-2-one

¹H-NMR (DMSO-d₆) δ 10.53 (s, 1H), 10.16 (br, 1H), 7.69 (s, 1H), 7.62 (d, 1H), 7.50 (d, 1H), 7.38-7.12 (m, 2H), 7.00-6.78 (m, 4H).

### Compound 123, 5-chloro-3-(4-isopropyl-benzylidene)-1,3-dihydro-indol-2-one

¹³C-NMR (CDCl₃, mixture of the E and Z isomers) δ 170.2, 167.8, 152.7, 151.8, 139.9, 139.6, 139.3, 137.9, 132.6, 131.8, 131.2, 129.7, 129.3, 128.2, 127.3, 127.1, 127.0, 126.6, 125.8, 124.4, 123.3, 123.0, 119.4, 111.0, 110.5, 34.3, 34.2, 23.8, 23.7.

### Compound 124, 5-chloro-3-(4-dimethylamino-benzylidene)-1,3-dihydro-indol-2-one

¹H-NMR (DMSO-d₆) δ 10.58 (br, 1H), 7.72 (d, 1H), 7.64 (d, 2H), 7.60 (s, 1H), 7.23 (dd, 1H), 6.86 (m, 3H), 3.05 (s, 6H).

### Compound 125, 5-chloro-3-(3,4-dichloro-benzylidene)-1,3-dihydro-indol-2-one

¹H-NMR (DMSO-d₆) δ 10.84 (br, 1H), 8.83 (s, 1H), 8.23 (d, 1H), 7.92 (s, 1H), 7.78 (m, 1H), 7.33 (s, 1H), 7.28 (d, 1H), 6.85 (d, 1H).

### Compound 126, 5-chloro-3-(2,5-dimethoxy-benzylidene)-1,3-dihydro-indol-2-one

¹H-NMR (DMSO-d₆) δ 10.73 (br, 1H), 7.69 (s, 1H), 7.39 (d, 1H), 7.28 (dd, 1H), 7.23 (d, 1H), 7.15 (m, 2H), 6.88 (d, 1H), 3.81 (s, 3H), 3.75 (s, 3H)

### Compound 127, 5-chloro-3-(2-chloro-benzylidene)-1,3-dihydro-indol-2-one

¹H-NMR (DMSO-d₆) δ 10.82 (br, 1H), 7.77 (m, 1H), 7,68 (m, 2H), 7.54 (m, 2H), 7.30 (dd, 1H), 7.01 (d, 1H), 6.90 (d, 1H).

### Compound 128, 5-chloro-3-(2,3-dimethoxy-benzylidene)-1,3-dihydro-indol-2-one

¹H-NMR (DMSO-d₆) δ 10.75 (br, 1H), 7.71 (s, 1H), 7.28-7.22 (m, 5H), 6.88 (d, 1H), 3.88 (s, 3H), 3.75 (s, 3H).

### Compound 129, 5-chloro-3-(2,6-dichloro-benzylidene)-1,3-dihydro-indol-2-one

¹H-NMR (DMSO-d₆) δ 10.55 (br, 1H), 7.70 (d, 2H), 7.57 (m, 2H), 7.31 (dd, 1H), 6.91 (d, 1H), 6.39 (d, 1H).

### Compound 130, 5-chloro-3-(2-hydroxy-benzylidene)-1,3-dihydro-indol-2-one

¹H-NMR (DMSO-d₆) δ 7.78 (s, 1H), 7.59 (dd, 1H), 7.42 (d, 1H), 7.33 (t, 1H), 7.25 (dd, 1H), 6.99 (d, 1H), 6.94 (t, 1H), 6.88 (d, 1H), 5.3 (br, 2H).

### Compound 131, 5-chloro-3-(4-methoxy-benzylidene)-1,3-dihydro-indol-2-one

¹H-NMR (DMSO-d₆) δ 10.70 (br, 1H), 7.73 (s, 1H), 7.68 (d, 2H), 7.57 (d, 1H), 7.28 (dd, 1H), 7.12 (d, 2H), 6.89 (d, 1H), 3.86 (s, 3H).

### Compound 132, 5-chloro-3-(4-chloro-benzylidene)-1,3-dihydro-indol-2-one

¹H-NMR (DMSO-d₆) δ 10.80 (br, 1H), 8.42 (d, 1H), 7.94 (s, 0.5H), 7.85 (s, 0.5H), 7.74 (d, 1H), 7.68 (s, 0.5H), 7.62 (, 1H), 7.55 (d, 1H), 7.38 (d, 0.5H), 7.25 (m, 1H), 6.89 (d, 0.5H), 6.83 (d, 0.5H).

### Compound 133, 5-chloro-3-naphtalen-1-ylmethylene-1,3-dihydro-indol-2-one

¹H-NMR (DMSO-d₆) δ 10.82 (br, 1H), 8.18 (s, 1H), 8.10 (m, 2H), 7.91 (d, 1H), 7.84 (d, 1H), 7.73-7.57 (m, 3H), 7.24 (dd, 1H), 6.89 (d, 1H), 6.77 (d, 1H).

### Compound 134, 5-chloro-3-(4-bromo-benzylidene)-1,3-dihydro-indol-2-one

¹H-NMR (DMSO-d₆, mixture of the E and Z isomers) δ 10.75 (br, 1H), 8.33 (d, 1H), 7.92 (s, 0.5H), 7.85 (s, 0.5H), 7.76 (d, 1H), 7.67 (m, 2.5H), 7.38 (d, 0.5H), 7.29 (m, 1H), 6.90 (d, 0.5H), 6.83 (d, 0.5H).

### Compound 135, 5-chloro-3-(4-methyl-benzylidene)-1,3-dihydro-indol-2-one

¹H-NMR (DMSO-d₆, mixture of the E and Z isomers) δ 10.72 (br, 1H), 8.33 (d, 1H), 7.90 (s, 0.5H), 7.85 (s, 0.5H), 7.69 (s, 0.5H), 7.61 (d, 1H), 7.50 (d, 0.5H), 7.37 (d, 1H), 7.28 (m, 2H), 6.89 (d, 0.5H), 6.82 (d, 0.5H), 2.40 (s, 1.5H), 2.38 (s, 1.5H).

### Compound 136, 3-anthracen-9-ylmethylene-5-chloro-1,3-dihydro-indol-2-one

¹H-NMR (DMSO-d₆) δ 10.89 (br, 1H), 8.82 (s, 1H), 8.47 (s, 1H), 8.23 (d, 2H), 7.97 (d, 2H), 7.62-7.54 (m, 4H), 7.13 (dd, 1H), 6.86 (d, 1H), 5.52 (d, 1H).

### Compound 137, 5-chloro-3-naphtalen-2-ylmethylene-1,3-dihydro-indol-2-one

¹H-NMR (DMSO-d₆, mixture of the E and Z isomers) δ 10.78 (br, 1H), 8.86 (s, 0.5H), 8.60 (d, 0.5H), 8.29 (s, 0.5H), 8.18-7.74 (m, 5H), 7.63 (m, 2H), 7.48 (d, 0.5H), 7.28 (m, 1H), 6.92 (d, 0.5H), 6.86 (d, 0.5H).

### Compound 138, 5-chloro-3-(2,4-dimethoxy-benzylidene)-1,3-dihydro-indol-2-one

¹H-NMR (DMSO-d₆) δ 10.65 (br, 1H), 7.69 (s, 1H), 7.65 (d, 1H), 7.43 (d, 1H), 7.25 (dd, 1H), 6.87 (d, 1H), 6.73 (m, 2H), 3.87 (s, 6H).

### Compound 139, 5-chloro-3-(2,4-dichloro-benzylidene)-1,3-dihydro-indol-2-one

¹H-NMR (DMSO-d₆) δ 10.84 (br, 1H), 7.88 (m, 1H), 7.81 (d, 1H), 7.63 (dd, 1H), 7.59 (d, 1H), 7.30 (t, 1H), 7.05 (d, 1H), 6.91 (d, 1H).

### Compound 140, 5-chloro-3-(3,4-dimethoxy-benzylidene)-1,3-dihydro-indol-2-one

¹H-NMR (DMSO-d₆) δ 10.66 (br, 1H), 8.68 (d, 1H), 7.89 (s, 1H), 7.80 (d, 1H), 7.36 (m, 1H), 7.20 (dd, 1H), 7.09 (d, 1H), 6.83 (d, 1H), 3.86 (s, 3H), 3.84 (s, 3H).

### Compound 141, 5-Chloro-3-(3,5-dimethoxy-benzylidene)-1,3-dihydro-indol-2-one

¹H-NMR (DMSO-d₆, mixture of the E and Z isomers) δ 10.74 (br, 1H), 7.89 (s, 0.5H), 7.83 (d, 0.5H), 7.75 (d, 1H), 7.64 (s, 0.5H), 7.56 (s, 0.5H), 7.29 (dd, 0.5H), 7.25 (dd, 0.5H), 6.86 (m, 2H), 6.64 (t, 1H), 3.80 (s, 6H).

### Compound 142, 5-chloro-3-(3,5-di-tert-butyl-4-hydroxy-benzylidene)-1,3-dihydro-indol-2-one

¹H-NMR (DMSO-d₆) δ 10.64 (s, 1H), 8.44 (s, 1H), 7.68 (s, 1H), 7.6 (br, 1H), 7.54 (s, 2H), 7.26 (dd, 1H), 6.88 (d, 1H), 1.43 (s, 18H).

### Compound 143, 5-chloro-3-(3,4,5-trimethoxy-benzylidene)-1,3-dihydro-indol-2-one

¹H-NMR (DMSO-d₆, mixture of the E and Z isomers) δ 10.71 (br, 1H), 8.04 (s, 1H), 7.91 (s, 0.5H), 7.80 (d, 0.5H), 7.73 (d, 0.5H), 7.65 (s, 0.5H), 7.29 (dd, 0.5H), 7.26 (dd, 0.5H), 7.10 (s, 1H), 6.89 (d, 0.5H), 6.85 (d, 0.5H), 3.85 (s, 3H), 3.83 (s, 3H), 3.76 (s, 3H)

### Compound 144, 3-benzylidene-5-chloro-1,3-dihydro-indol-2-one

¹H-NMR (DMSO-d₆, mixture of the E and Z isomers) 10.75 (br, 1H), 8.38 (m, 0.5H), 7.95 (s, 0.5H), 7.86 (d, 0.5H), 7.71 (m, 2H), 7.57-7.47 (m, 3H), 7.40 (d, 0.5H), 7.29 (dd, 0.5H), 7.25 (dd, 0.5H), 6.89 (d, 0.5H), 6.83 (d, 0.5H).

### Compound 145, 5-chloro-3-(3-hydroxy-benzylidene)-1,3-dihydro-indol-2-one

¹H-NMR (DMSO-d₆) δ 10.71 (br, 1H), 9.77 (br, 1H), 7.64 (s, 1H), 7.49 (d, 1H), 7.35 (t, 1H), 7.27 (t, 1H), 7.07 (m, 2H), 6.89 (m, 2H).

### Compound 146, 5-chloro-3-(2-methoxy-benzylidene)-1,3-dihydro-indol-2-one

¹H-NMR (DMSO-d₆) δ 10.72 (br, 1H), 7.73 (s, 1H), 7.65 (dd, 1H), 7.53 (t, 1H), 7.28 (s, 1H), 7.27 (dd, 1H), 7.20 (dd, 1H), 7.11 (t, 1H), 6.88 (d, 1H), 3.86 (s, 3H).

### Compound 147, 5-chloro-3-(2-methyl-benzylidene)-1,3-dihydro-indol-2-one

¹H-NMR (DMSO-d₆) δ 10.75 (br, 1H), 7.78 (s, 1H), 7.53 (d, 1H), 7.37 (m, 3H), 7.26 (dd, 1H), 6.98 (d, 1H), 6.88 (d, 1H), 2.30 (s, 3H).

### Compound 148, 3-(3,5-dibromo-4-hydroxy-benzylidene)-1,3-dihydro-indol-2-one

¹³C-NMR (DMSO-d₆, mixture of the E and Z isomers) δ 168.3, 167.2, 152.5, 151.8, 143.0, 140.5, 136.0, 133.8, 133.1, 133.0, 130.2, 128.8, 128.6, 128.5, 127.5, 125.9, 124.8, 121.9, 121.1, 121.0, 120.6, 119.5, 111.7, 111.0, 110.2, 109.3.

### Compound 149, 3-(3,4-difluoro-benzylidene)-1,3-dihydro-indol-2-one

¹³C-NMR (DMSO-d₆, mixture of the E and Z isomers) δ 168.3, 167.1, 150.3, 149.8, 149.4, 148.7, 143.1, 140.9, 134.2, 133.2, 132.0, 131.6, 130.5, 130.0, 129.3, 128.6, 127.5, 126.4, 124.5, 122.4, 121.2, 121.2, 120.4, 120.0, 119.9, 118.4, 118.0, 117.2, 110.2, 109.5.

### Compound 150, 3-(2-hydroxy-naphtalen-1-ylmethylene)-1,3-dihydro-indol-2-one

¹³C-NMR (DMSO-d₆, E isomer) δ 168.4, 153.7, 142.4, 131.3, 130.9, 129.6, 129.4, 129.2, 128.5, 127.6, 127.0, 124.0, 123.6, 123.1, 121.7, 120.7, 118.1, 113.7, 109.4.

### Compound 151, 3-(4-methyl-benzylidene)-1,3-dihydro-indol-2-one

¹H-NMR (DMSO-d₆, E isomer) δ 10.57 (br, 1H), 7.60 (m, 4H), 7.34 (d, 2H), 7.23 (t, 1H), 6.86 (m, 2H), 2.39 (s, 3H).

### Compound 152, 3-(3,4-dimethoxy-benzylidene)-1,3-dihydro-indol-2-one

¹H-NMR (DMSO-d₆, E isomer) δ 10.54 (br, 1H), 8.68 (d, 1H), 7.84 (dd, 1H), 7.75 (s, 1H), 7.67 (d, 1H), 7.18 (t, 1H), 7.07 (d, 1H), 6.98 (t, 1H), 6.83 (d, 1H), 3.85 (s, 3H), 3.84 (s, 3H).

### Compound 153, 3-(3-methoxy-benzylidene)-1,3-dihydro-indol-2-one

¹³C-NMR (DMSO-d₆, mixture of the E and Z isomers) δ 168.5, 167.0, 159.2, 158.8, 142.9, 140.7, 136.6, 135.7, 135.5, 135.2, 130.1, 129.8, 129.1, 128.9, 127.7, 126.9, 124.8, 122.5, 121.4, 121.0, 120.8, 119.7, 116.5, 116.4, 115.5, 114.2, 110.1, 109.3, 55.1.

### Compound 154, 3-(2-chloro-benzylidene)-1,3-dihydro-indol-2-one

¹H-NMR (DMSO-d₆, E isomer) δ 10.67 (br, 1H), 7.77 (m, 1H), 7.66 (m, 1H), 7.59 (s, 1H), 7.51 (m, 2H), 7.24 (t, 1H), 7.14 (d, 1H), 6.88 (d, 1H), 6.81 (t, 1H).

### Compound 155, 3-(3-chloro-benzylidene)-1,3-dihydro-indol-2-one

¹H-NMR (DMSO-d₆, E isomer) δ 10.65 (br, 1H), 7.74 (s, 1H), 7.68 (m, 1H), 7.59 (s, 1H), 7.55 (m, 2H), 7.41 (d, 1H), 7.25 (t, 1H), 6.87 (m, 2H).

### Compound 156, 3-(2,4-dichloro-benzylidene)-1,3-dihydro-indol-2-one

¹H-NMR (DMSO-d₆, E isomer) δ 10.69 (br, 1H), 7.83 (d, 1H), 7.79 (d, 1H), 7.58 (dd, 1H), 7.51 (s, 1H), 7.25 (t, 1H), 7.14 (d, 1H), 6.88 (d, 1H), 6.83 (t, 1H).

### Compound 157, 3-(3,4-dichloro-benzylidene)-1,3-dihydro-indol-2-one

¹H-NMR (DMSO-d₆, E isomer) δ 10.66 (br, 1H), 7.95 (d, 1H), 7.79 (d, 1H), 7.71 (dd, 1H), 7.56 (s, 1H), 7.42 (d, 1H), 7.26 (t, 1H), 6.87 (m, 2H).

### Compound 158, 3-(3-bromo-benzylidene)-1,3-dihydro-indol-2-one

¹H-NMR (DMSO-d₆, E isomer) δ 10.63 (br, 1H), 7.87 (s, 1H), 7.69 (m, 2H), 7.59 (s, 1H), 7.49 (t, 1H), 7.40 (d, 1H), 7.25 (t, 1H), 6.87 (m, 2H).

### Compound 159, 3-(4-hydroxy-benzylidene)-1,3-dihydro-indol-2-one

¹H-NMR (DMSO-d₆, E isomer) δ 10.51 (br, 1H), 10.13 (br, 1H), 7.70 (d, 1H), 7.62 (d, 2H), 7.54 (s, 1H), 7.21 (t, 1H), 6.89 (m, 4H).

### Compound 160, 3-(3-hydroxy-benzylidene)-1,3-dihydro-indol-2-one

¹H-NMR (DMSO-d₆, E isomer) δ 10.58 (br, 1H), 9.72 (br, 1H), 7.59 (d, 1H), 7.55 (s, 1H), 7.33 (t, 1H), 7.23 (t, 1H), 7.10 (m, 2H), 6.87 (m, 3H).

### Compound 161, 3-(2,4-dihydroxy-benzylidene)-1,3-dihydro-indol-2-one

¹H-NMR (DMSO-d₆, E isomer) δ 10.43 (br, 1H), 10.08 (br, 2H), 7.69 (s, 1H), 7.63 (d, 1H), 7.55 (d, 1H), 7.18 (t, 1H), 6.86 (m, 2H), 6.44 (d, 1H), 6.38 (dd, 1H).

### Compound 162, 5-chloro-3-(2,5-dimethoxy-benzylidene)-1,3-dihydro-indol-2-one

¹H-NMR (DMSO-d₆, E isomer) δ 10.71 (br, 1H), 7.69 (s, 1H), 7.39 (d, 1H), 7.28 (dd, 1H), 7.24 (d, 1H), 7.11 (m, 2H), 6.89 (d, 1H), 3.81 (s, 3H), 3.75 (s, 3H).

### Compound 163, 3-(3,4,5-trimethoxy-benzylidene)-1,3-dihydro-indol-2-one

¹H-NMR (DMSO-d₆, E isomer) δ 10.57 (br, 1H), 8.02 (s, 2H), 7.76 (s, 1H), 7.68 (d, 1H), 7.21 (t, 1H), 7.00 (t, 1H), 6.85 (d, 1H), 3.85 (s, 6H), 3.76 (s, 3H).

### Compound 164, 3-(3,5-dimethoxy-benzylidene)-1,3-dihydro-indol-2-one

¹H-NMR (DMSO-d₆, E isomer) δ 10.59 (br, 1H), 7.59 (d, 1H), 7.56 (s, 1H), 7.23 (t, 1H), 6.93-6.81 (m, 4H), 6.61 (t, 1H), 3.79 (s, 6H).

### Compound 165, 3-(2,3-dimethoxy-benzylidene)-1,3-dihydro-indol-2-one

¹H-NMR (DMSO-d₆, E isomer) δ 10.60 (br, 1H), 7.64 (s, 1H), 7.37 (d, 1H), 7.27-7.15 (m, 4H), 6.85 (m, 2H), 3.87 (s, 3H), 3.75 (s, 3H).

### Compound 166, 3-(2-methoxy-naphtalen-1-ylmethylene)-1,3-dihydro-indol-2-one

¹H-NMR (DMSO-d₆, E isomer) δ 10.62 (br, 1H), 8.11 (d, 1H), 7.98 (m, 1H), 7.83 (s, 1H), 7.61 (m, 2H), 7.43 (m, 2H), 7.12 (t, 1H), 6.84 (d, 1H), 6.59 (t, 1H), 6.20 (d, 1H), 3.93 (s, 3H)

### Compound 167, 3-(2-methoxy-benzylidene)-1,3-dihydro-indol-2-one

¹³C-NMR (DMSO-d₆, E isomer) δ 168.5, 157.6, 142.7, 131.6, 131.6, 129.8, 129.4, 127.3, 122.9, 122.2, 121.1, 121.0, 120.2, 111.5, 110.0, 55.6.

### Compound 168, 3-(4-hydroxy-3-methoxy-benzylidene)-1,3-dihydro-indol-2-one

¹H-NMR (DMSO-d₆, E isomer) δ 10.50 (br, 1H), 8.69 (d, 1H), 7.74 (m, 1H), 7.69 (s, 1H), 7.65 (d, 1H), 7.16 (t, 1H), 7.0-6.8 (m, 3H), 3.86 (s, 3H).

### Compound 169, 3-(3-hydroxy-4-methoxy-benzylidene)-1,3-dihydro-indol-2-one

¹H-NMR (DMSO-d₆, E isomer) δ 10.52 (br, 1H), 9.37 (br, 1H), 7.73 (d, 1H), 7.50 (s, 1H), 7.20 (m, 3H), 7.06 (d, 1H), 6.88 (m, 2H), 3.85 (s, 3H).

### Compound 170, 5-bromo-3-(2,5-dimethoxy-benzylidene)-1,3-dihydro-indol-2-one

¹H-NMR (DMSO-d₆, E isomer) δ 10.72 (br, 1H), 7.68 (s, 1H), 7.54 (d, 1H), 7.40 (dd, 1H), 7.24 (d, 1H), 7.11 (m, 2H), 6.84 (d, 1H), 3.81 (s, 3H), 3.76 (s, 3H).

### Compound 171, 6-bromo-3-(2,5-dimethoxy-benzylidene)-1,3-dihydro-indol-2-one

¹H-NMR (DMSO-d₆, E isomer) δ 10.71 (br, 1H), 7.67 (s, 1H), 7.36 (d, 1H), 7.20 (d, 1H), 7.08 (m, 3H), 7.01 (d, 1H), 3.80 (s, 3H), 3.73 (s, 3H).

### Compound 172, 7-bromo-3-(2,5-dimethoxy-benzylidene)-1,3-dihydro-indol-2-one

¹H-NMR (DMSO-d₆, mixture of the E and Z isomers, signals of the predominant E isomer) δ 10.85 (br, 1H), 7.69 (s, 1H), 7.43 (m, 2H), 7.21 (d, 1H), 7.09 (m, 2H), 6.84 (t, 1H), 3.81 (s, 3H), 3.73 (s, 3H).

### Compound 173, 3-(2,5-dimethoxy-benzylidene)-6-fluoro-1,3-dihydro-indol-2-one

¹H-NMR (DMSO-d₆, E isomer) δ 10.76 (br, 1H), 7.59 (s, 1H), 7.43 (m, 1H), 7.20 (d, 1H), 7.09 (m, 2H), 6.70 (m, 2H), 3.80 (s, 3H), 3.74 (s, 3H).

### Compound 174, 3-(2,5-dimethoxy-benzylidene)-5-trifluoromethyl-1,3-dihydro-indol-2-one

¹³C-NMR (DMSO-d₆, E isomer) δ 168.4, 152.6, 151.9, 145.8, 133.6, 127.0, 126.1, 122.5, 121.4, 121.3, 118.8, 118.4, 113.8, 113.0, 110.2, 55.8, 55.5.

### Compound 175, 5-amino-3-(2,5-dimethoxy-benzylidene)-1,3-dihydro-indol-2-one

¹H-NMR (DMSO-d₆, E isomer) δ 10.08 (s, 1H), 7.51 (s, 1H), 7.22 (d, 1H), 7.06 (m, 2H), 6.87 (d, 1H), 6.56 (d, 1H), 6.47 (dd, 1H), 4.66 (br, 2H), 3.81 (s, 3H), 3.74 (s, 3H).

### Compound 176, 6-chloro-5-(2-chloro-acetyl)-3-(2,5-dimethoxy-benzylidene)-1,3-dihydro-indol-2-one

¹H-NMR (DMSO-d₆, mixture of the E and Z isomers) δ 11.10 (br, 0.5H), 10.81 (br, 0.5H), 7.86 (s, 0.5H), 7.73 (d, 1H), 7.25-6.88 (m, 3.5H), 5.00 (s, 1H), 4.88 (s, 1H), 3.81 (s, 3H), 3.75 (s, 3H).

### Compound 177, 3-(2,5-dimethoxy-benzylidene)-5-hydroxy-1,3-dihydro-indol-2-one

¹³C-NMR (DMSO-d₆, E isomer) δ 168.5, 152.5, 151.7, 135.1, 131.0, 128.1, 123.3, 121.6, 116.9, 116.4, 114.0, 112.6, 110.3, 109.9, 55.9, 55.4

### Compound 178, 3-(2,5-dimethoxy-benzylidene)-2-oxo-2,3-dihydro-1H-indole-5-carboxylic acid methyl ester

¹H-NMR (DMSO-d₆, E isomer) δ 11.01 (br, 1H), 8.21 (s, 1H), 7.88 (d, 1H), 7.73 (s, 1H), 7.31 (d, 1H), 7.12 (m, 2H), 6.98 (d, 1H), 3.82 (s, 3H), 3.77 (s, 3H), 3.77 (s, 3H).

### Compound 179, 3-(9-ethyl-9H-carbazol-3-ylmethylene)-1,3-dihydro-indol-2-one

¹³C-NMR (DMSO-d₆, E isomer) δ 169.1, 142.6, 140.3, 140.1, 137.8, 129.5, 127.5, 126.4, 125.0, 124.9, 122.7, 122.4, 122.1, 121.6, 121.5, 121.0, 120.7, 119.6, 110.0, 109.6, 109.4, 37.3, 13.8.

### Compound 180, 3-(2-hydroxy-3-methoxy-benzylidene)-1,3-dihydro-indol-2-one

¹H-NMR (DMSO-d₆, E isomer) δ 10.57 (s, 1H), 9.41 (s, 1H), 7.70 (s, 1H), 7.51 (d, 1H), 7.21 (m, 2H), 7.08 (d, 1H), 6.88 (m, 3H), 3.86 (s, 3H).

### Compound 181, 3-(2,5-dimethoxy-benzylidene)-4,5-difluoro-1,3-dihydro-indol-2-one

¹³C-NMR (DMSO-d₆, E isomer) δ 166.1, 152.5, 152.0, 143.6, 137.6, 135.9, 122.1, 118.8, 116.7, 116.5, 111.8, 105.0, 56.2, 55.3.

### Compound 182, 3-(3,5-dichloro-2-hydroxy-benzylidene)-1,3-dihydro-indol-2-one

¹³C-NMR (DMSO-d₆, E isomer) δ 168.2, 150.8, 142.9, 130.3, 130.0, 129.8, 128.8, 127.6, 125.5, 122.8, 122.6, 122.4, 121.1, 120.6, 110.1.

### Compound 183, 3-(2,5-diethoxy-benzylidene)-1,3-dihydro-indol-2-one

¹H-NMR (DMSO-d₆, E isomer) δ 10.56 (br, 1H), 7.62 (s, 1H), 7.45 (d, 1H), 7.21 (m, 2H), 7.1-6.95 (m, 2H), 6.86 (m, 2H), 4.06 (q, 2H), 3.98 (q, 2H), 1.31(t, 3H), 1.27 (t, 3H).

### Compound 184, 3-(2,5-dihydroxy-benzylidene)-1,3-dihydro-indol-2-one

¹³C-NMR (DMSO-d₆, mixture of the E and Z isomers, signals of the predominant E isomer) δ 168.9, 149.4, 142.6, 132.7, 129.6, 126.1, 122.6, 121.5, 121.3, 121.0, 119.0, 116.9, 114.8, 109.9.

### Compound 185, 3-(2,4,5-trimethoxy-benzylidene)-1,3-dihydro-indol-2-one

¹H-NMR (DMSO-d₆, E isomer) δ 10.52 (br, 1H), 7.66 (s, 1H), 7.61 (d, 1H), 7.28 (s, 1H), 7.20 (t, 1H), 6.88 (m, 2H), 6.82 (s, 1H), 3.90 (s, 3H), 3.88 (s, 3H), 3.71 (s, 3H).

### Compound 186, 3-(9-methyl-9H-carbazol-3-ylmethylene)-1,3-dihydro-indol-2-one

¹H-NMR (DMSO-d₆, E isomer) δ 10.58 (s, 1H), 8.57 (s, 1H), 8.21 (d, 1H), 7.91 (dd, 1H), 7.86 (s, 1H), 7.77 (m, 2H), 7.67 (d, 1H), 7.53 (t, 1H), 7.27 (t, 1H), 7.23 (t, 1H), 6.89 (m, 2H), 3.95 (s, 3H).

### Compound 187, 3-(2-hydroxy-5-trifluoromethoxy-benzylidene)-1,3-dihydro-indol-2-one

¹³C-NMR (DMSO-d₆, E isomer) δ 168.4, 155.4, 142.9, 139.9, 130.4, 130.1, 127.8, 124.2, 122.3, 122.2, 122.0, 120.9, 120.8, 117.0, 110.1.

### Compound 188, 3-(1H-indol-5-ylmethylene)-1,3-dihydro-indol-2-one

¹H-NMR (DMSO-d₆, mixture of the E and Z isomers, signals of the predominant isomer) δ 11.44 (br, 1H), 10.56 (br, 1H), 7.98 (s, 1H), 7.79 (m, 2H), 7.49 (m, 3H), 7.21 (t, 1H), 6.88 (d, 2H), 6.56 (d, 1H).

### Compound 189, 3-(1H-indol-4-ylmethylene)-1,3-dihydro-indol-2-one

¹H-NMR (DMSO-d₆) δ 11.44 (br, 1H), 10.62 (br, 1H), 7.94 (s, 1H), 7.56 (d, 1H), 7.49 (m, 2H), 7.38 (d, 1H), 7.25 (d, 1H), 7.20 (t, 1H), 6.88 (d, 1H), 6.79 (t, 1H), 6.42 (s, 1H).

### Compound 190, 3-(1H-indol-7-ylmethylene)-1,3-dihydro-indol-2-one

¹H-NMR (DMSO-d₆, E isomer) δ 11.45 (br, 1H), 10.62 (br, 1H), 7.98 (s, 1H), 7.70 (d, 1H), 7.53 (d, 1H), 7.39 (t, 1H), 7.31 (d, 1H), 7.18 (m, 2H), 6.88 (d, 1H), 6.78 (t, 1H), 6.56 (m, 1H).

### Compound 191, 3-(1,4-dimethyl-9H-carbazol-2-ylmethylene)-1,3-dihydro-indol-2-one

¹H-NMR (DMSO-d₆, E isomer) δ 11.52 (br, 1H), 10.58 (br, 1H), 8.21 (d, 1H), 7.96 (s, 1H), 7.60 (d, 1H), 7.46 (m, 2H), 7.31-7.10 (m, 3H), 6.87 (d, 1H), 6.77 (t, 1H), 2.80 (s, 3H), 2.57 (s, 3H).

### Compound 192, 3-(2-benzyloxy-4,5-dimethoxy-benzylidene)-1,3-dihydro-indol-2-one

¹H-NMR (DMSO-d₆, mixture of the E and Z isomers) δ 10.52 (br, 1H), 8.94 (s, 0.5H), 8.00 (s, 0.5H), 7.69 (s, 0.5H), 7.62 (d, 0.5H), 7.54 (m, 1H), 7.48-7.31 (m, 4H), 7.28 (s, 0.5H), 7.18 (m, 1H), 7.0-6.8 (m, 3.5H), 5.28 (s, 1H), 5.23 (s, 1H), 3.86 (s, 3H), 3.77 (s, 1.5H), 3.71 (s, 1.5H).

### Compound 193, 3-(2,5-dichloro-benzylidene)-1,3-dihydro-indol-2-one

¹³C-NMR (DMSO-d₆, mixture of the E and Z isomers, signals of the predominant E isomer) δ 167.9, 143.4, 135.0, 132.0, 131.5, 131.5, 131.0, 130.8, 130.6, 129.9, 129.7, 122.6, 121.5, 120.4, 110.5.

### Compound 194, 3-(2,5-dimethoxy-benzylidene)-2-oxo-2,3-dihydro-1H-indole-7-carbonitrile

¹H-NMR (DMSO-d₆, mixture of the E and Z isomers, signals of the predominant E isomer) δ 11.53 (br, 1H), 7.75 (s, 1H), 7.67 (d, 1H), 7.59 (d, 1H), 7.20 (d, 1H), 7.15-7.06 (m, 2H), 7.03 (t, 1H), 3.80 (s, 3H), 3.73 (s, 3H).

### Compound 195, 3-(2,5-dimethoxy-benzylidene)-6-methanesulfonyl-1,3-dihydro-indol-2-one

¹H-NMR (DMSO-d₆, mixture of the E and Z isomers, signals of the predominant E isomer) δ 10.92 (br, 1H), 7.82 (s, 1H), 7.65 (d, 1H), 7.45 (d, 1H), 7.32 (s, 1H), 7.24 (s, 1H), 7.18-7.03 (m, 2H), 3.82 (s, 3H), 3.75 (s, 3H), 3.20 (s, 3H).

### Compound 196, 3-(2,5-dimethoxy-benzylidene)-2-oxo-2,3-dihydro-1H-indole-5-carbonitrile

¹H-NMR (DMSO-d₆, mixture of the E and Z isomers, signals of the predominant E isomer) δ 11.09 (br, 1H), 7.76 (s, 1H), 7.70 (dd, 1H), 7.66 (m, 1H), 7.25 (d, 1H), 7.15-7.08 (m, 2H), 7.03 (d, 1H), 3.81 (s, 3H), 3.76 (s, 3H).

### Compound 197, 3-(2,5-dimethoxy-benzylidene)-6-trifluoromethyl-1,3-dihydro-indol-2-one

¹H-NMR (DMSO-d₆, mixture of the E and Z isomers, signals of the predominant E isomer) δ 10.85 (br, 1H), 7.79 (s, 1H), 7.62 (d, 1H), 7.26 (m, 2H), 7.16-7.03 (m, 3H), 3.82 (s, 3H), 3.74 (s, 3H).

### Compound 198, 3-(2,5-dimethoxy-benzylidene)-7-fluoro-1,3-dihydro-indol-2-one

¹H-NMR (DMSO-d₆, mixture of the E and Z isomers, signals of the predominant E isomer) δ 11.08 (br, 1H), 7.70 (s, 1H), 7.29 (d, 1H), 7.22 (d, 1H), 7.15 (m, 1H), 7.10 (m, 2H), 6.89 (m, 1H), 3.81 (s, 3H), 3.74 (s, 3H).

### Compound 199, 3-(2,5-dimethoxy-benzylidene)-2-oxo-2,3-dihydro-1H-indole-6-carbonitrile

¹H-NMR (DMSO-d₆, mixture of the E and Z isomers, signals of the predominant E isomer) δ 10.90 (br, 1H), 7.81 (s, 1H), 7.56 (d, 1H), 7.35 (dd, 1H), 7.21 (m, 2H), 7.12 (m, 2H), 3.81 (s, 3H), 3.74 (s, 3H).

### Compound 200, 6-chloro-3-(2,5-dimethoxy-benzylidene)-1,3-dihydro-indol-2-one

¹H-NMR (DMSO-d₆, mixture of the E and Z isomers, signals of the predominant E isomer) δ 10.72 (br, 1H), 7.64 (s, 1H), 7.42 (d, 1H), 7.20 (d, 1H), 7.11-7.05 (m, 2H), 6.94 (dd, 1H), 6.88 (d, 1H), 3.80 (s, 3H), 3.73 (s, 3H).

### Compound 201, 3-(2,5-dibromo-benzylidene)-1,3-dihydro-indol-2-one

¹H-NMR (DMSO-d₆, E isomer) δ 10.70 (br, 1H), 7.89 (d, 1H), 7.76 (d, 1H), 7.62 (dd, 1H), 7.43 (s, 1H), 7.25 (t, 1H), 6.99 (d, 1H), 6.84 (t, 1H), 6.83 (d, 1H).

### Compound 202, 3-(5-bromo-2-ethoxy-benzylidene)-1,3-dihydro-indol-2-one

¹H-NMR (DMSO-d₆, E isomer) δ 10.60 (br, 1H), 7.77 (d, 1H), 7.62 (dd, 1H), 7.53 (s, 1H), 7.29 (d, 1H), 7.23 (t, 1H), 7.13 (d, 1H), 6.86 (m, 2H), 4.12 (q, 2H), 1.28 (t, 3H).

### Compound 203, 3-(5-bromo-2-methoxy-benzylidene)-1,3-dihydro-indol-2-one

¹H-NMR (DMSO-d₆, E isomer) δ 10.60 (br, 1H), 7.77 (d, 1H), 7.65 (dd, 1H), 7.52 (s, 1H), 7.28 (d, 1H), 7.23 (t, 1H), 7.15 (d, 1H), 6.86 (m, 2H), 3.85 (s, 3H).

### Compound 204, 3-(2-fluoro-5-methoxy-benzylidene)-1,3-dihydro-indol-2-one

¹H-NMR (CDCl₃, E isomer) δ 8.07 (br, 1H), 7.77 (s, 1H), 7.49 (d, 1H), 7.24-7.14 (m, 2H), 7.11 (t, 1H), 6.98-6.86 (m, 3H), 3.80 (s, 3H).

### Compound 205, 3-(2,5-difluoro-benzylidene)-1,3-dihydro-indol-2-one

¹H-NMR (CDCl₃, mixture of the E and Z isomers, signals of the predominant E isomer) δ 7.90 (br, 1H), 7.70 (s, 1H), 7.40 (m, 1H), 7.30-7.01 (m, 4H), 6.89 (m, 2H).

### Compound 206, 3-(2-chloro-5-nitro-benzylidene)-1,3-dihydro-indol-2-one

¹H-NMR (DMSO-d₆, E isomer) δ 10.64 (s, 1H), 8.97 (d, 1H), 8.24 (dd, 1H), 7.84 (d, 1H), 7.82 (s, 1H), 7.76 (d, 1H), 7.29 (t, 1H), 7.02 (t, 1H), 6.85 (d, 1H).

### Compound 207, 3-(2,5-bis-trifluoromethyl-benzylidene)-1,3-dihydro-indol-2-one

¹H-NMR (CDCl₃, E isomer) δ 7.97 (m, 3H), 7.88 (m, 1H), 7.83 (d, 1H), 7.24 (t, 1H), 6.88 (m, 2H), 6.80 (t, 1H).

### Compound 208, 3-(2,4-dichloro-benzylidene)-1,3-dihydro-indol-2-one

¹H-NMR (DMSO-d₆, Z isomer) δ 10.61 (br, 1H), 8.15 (d, 1H), 7.72 (m, 3H), 7.46 (dd, 1H), 7.26 (t, 1H), 7.00 (t, 1H), 6.82 (d, 1H).

### Compound 209, 3-(2-hydroxy-5-methoxy-benzylidene)-1,3-dihydro-indol-2-one

¹³C-NMR (DMSO-d₆) δ 159.8, 155.5, 147.7, 146.6, 142.3, 130.6, 129.1, 120.1, 119.8, 118.8, 116.9, 115.6, 115.1, 110.6, 55.8.

### Compound 210, 3-(1H-indol-6-ylmethylene)-1,3-dihydro-indol-2-one

¹³C-NMR (DMSO-d₆, mixture of the E and Z isomers) δ 169.0, 167.4, 142.5, 139.9, 139.3, 138.3, 135.5, 129.8, 129.4, 129.1, 128.9, 128.1, 127.8, 127.1, 126.8, 125.6, 124.9, 124.4, 123.3, 121.8, 121.4, 120.9, 120.8, 120.7, 120.1, 119.3, 119.0, 115.9, 113.2, 109.9, 109.0, 101.7.

### Compound 211, 3-(2,5-dimethoxy-benzylidene)-5-fluoro-1,3-dihydro-indol-2-one

¹³C-NMR (DMSO-d₆, E isomer) δ 168.4, 157.1, 152.6, 151.8, 139.1, 133.0, 127.3, 122.8, 122.0, 117.2, 116.1, 114.3, 112.8, 110.7, 109.3, 55.9, 55.5.

### Compound 212, 3-[4-(quinolin-2-ylmethoxy)-benzylidene]-1,3-dihydro-indol-2-one

¹³C-NMR (DMSO-d₆) δ 168.8, 159.3, 157.1, 146.9, 142.6, 137.1, 135.7, 134.3, 131.4, 129.8, 129.7, 128.5, 127.9, 127.2, 127.1, 126.6, 125.8, 122.0, 121.0, 121.0, 119.6, 115.1, 114.5, 109.9, 70.9.

### Compound 213, 3-[4-(naphthalen-2-ylmethoxy)-benzylidene]-1,3-dihydro-indol-2-one

¹H-NMR (DMSO-d₆) δ 8.49 (d, 1H), 8.08-7.86 (m, 4H), 7.78-7.48 (m, 6H), 7.29-7.10 (m, 3H), 6.85 (m, 2H), 5.38 (s, 2H).

### Compound 214, 3-[3,5-dichloro-2-(quinolin-2-ylmethoxy)-benzylidene]-1,3-dihydro-indol-2-one

¹H-NMR (DMSO-d₆, mixture of the E and Z isomers) δ 10.51 (br, 1H), 8.49 (d, 0.5H), 8.35 (d, 0.5H), 8.29 (d, 0.5H), 7.98-7.82 (m, 2.5H), 7.80-7.50 (m, 4.5H), 7.46 (s, 0.5H), 7.35 (d, 0.5H), 7.20 (t, 1H), 7.09 (d, 0.5H), 6.89-6.72 (m, 2H), 5.29 (s, 1H), 5.23 (s, 1H).

### Compound 215, 2-[4-(2-oxo-1,2-dihydro-indol-3-ylidenemethyl)-phenoxy]-propionic acid

¹H-NMR (DMSO-d₆) δ 13.11 (br, 1H), 10.54 (s, 1H), 7.70 (d, 2H), 7.64 (d, 1H), 7.57 (s, 1H), 7.22 (t, 1H), 7.02 (d, 2H), 6.87 (m, 2H), 4.96 (q, 1H), 1.54 (d, 3H).

### Compound 216, 2-benzyl-3-butylamino-5-(2-oxo-1,2-dihydro-indol-3-ylidenemethyl)-benzenesulfonamide

¹³C-NMR (DMSO-d₆, mixture of the E and Z isomers) δ 168.6, 167.1, 147.9, 147.5, 144.0, 143.4, 143.0, 140.6, 138.7, 138.6, 136.5, 135.4, 133.1, 132.5, 130.1, 128.9, 128.2, 128.0, 127.6, 126.9, 125.8, 124.9, 123.5, 122.9, 122.6, 121.2, 121.0, 120.7, 119.8, 119.4, 115.6, 114.9, 113.5, 110.1, 109.2, 42.7, 42.5, 32.6, 32.4, 30.1, 19.2, 19.1, 13.5, 13.5.

### Compound 217, 2-benzyl-3-benzylamino-5-(2-oxo-1,2-dihydro-indol-3-ylidenemethyl)-benzenesulfonamide

ES+(MSQ14914):[M+H]⁺32 496; ES−(MSQ14914):[M−H]⁻=494.

### Compound 218, 3-[(furan-2-ylmethyl)-amino]-5-(2-oxo-1,2-dihydro-indol-3-ylidenemethyl)-2-phenoxy-benzenesulfonamide

¹H-NMR (DMSO-d₆, mixture of the E and Z isomers) δ 10.63 (br, 1H), 8.62 (d, 0.5H), 8.01 (d, 0.5H), 7.82 (s, 0.5H), 7.77 (d, 0.5H), 7.59 (d, 0.5H), 7.55 (s, 0.5H), 7.51 (m, 1.5H), 7.38-7.12 (m, 5.5H), 7.07-6.96 (m, 1.5H), 6.95-6.80 (m, 3.5H), 6.32 (m, 1H), 6.20 (d, 0.5H), 6.10 (d, 0.5H), 5.78 (t, 0.5H), 5.56 (t, 0.5H), 4.36 (t, 2H).

### Compound 219, 3-methylamino-5-(2-oxo-1,2-dihydro-indol-3-ylidenemethyl)-2-phenoxy-benzenesulfonamide

¹H-NMR (DMSO-d₆) δ 10.59 (br, 1H), 8.49 (d, 1H), 7.97 (d, 1H), 7.85 (s, 1H), 7.78 (d, 1H), 7.35-7.10 (m, 5H), 7.00 (m, 2H), 6.93-6.78 (m, 3H), 5.22 (q, 1H), 2.74 (d, 3H).

### Compound 220, 2-benzyl-3-ethoxy-5-(2-oxo-1,2-dihydro-indol-3-ylidenemethyl)-benzenesulfonamide

¹H-NMR (DMSO-d₆, E isomer) δ 10.60 (br, 1H), 8.77 (s, 1H), 8.23 (s, 1H), 7.85 (s, 1H), 7.77 (d, 1H), 7.54 (br, 2H), 7.06 (m, 6H), 7.00 (t, 1H), 6.85 (d, 1H), 4.35 (s, 2H), 4.01 (q, 2H), 1.17 (t, 3H).

### Compound 221, [2-(2-oxo-1,2-dihydro-indol-3-ylidenemethyl)-phenoxy]-acetic acid

¹H-NMR (DMSO-d₆, E isomer) δ 13.04 (br, 1H), 10.58 (br, 1H), 7.74 (s, 1H), 7.71 (d, 1H), 7.45 (m, 2H), 7.22 (t, 1H), 7.09 (m, 2H), 6.84 (m, 2H), 4.82 (s, 2H).

### Compound 222, 3-[4-(6-methyl-pyridin-2-ylmethoxy)-benzylidene]-1,3-dihydro-indol-2-one

¹H-NMR (DMSO-d₆, E isomer) δ 10.55 (br, 1H), 7.74 (m, 3H), 7.65 (d, 1H), 7.58 (s, 1H), 7.34 (d, 1H), 7.22 (m, 2H), 7.18 (d, 2H), 6.87 (m, 2H), 5.22 (s, 2H), 2.50 (s, 3H).

### Compound 223, 4-[4-(5-chloro-2-oxo-1,2-dihydro-indol-3-ylidenemethyl)-phenyl]-piperazine-1-carbaldehyde

¹H-NMR (DMSO-d₆) δ 10.63 (br, 1H), 8.11 (s, 1H), 7.68-7.60 (m, 4H), 7.25 (dd, 1H), 7.13 (d, 2H), 6.88 (d, 1H), 3.51 (m, 4H), 3.45-3.34 (m, 4H).

### Compound 224, 5-chloro-3-(4-isopropyl-benzylidene)-1,3-dihydro-indol-2-one

¹³C-NMR (CDCl₃, mixture of the E and Z isomers) δ 170.2, 167.8, 152.7, 151.8, 139.9, 139.6, 139.3, 137.9, 132.6, 131.8, 131.2, 129.7, 129.3, 128.2, 127.3, 127.1, 127.0, 126.6, 125.8, 124.4, 123.3, 123.0, 119.4, 111.0, 110.5, 34.3, 34.2, 23.8, 23.7.

### Compound 225, 4-[4-(2-oxo-1,2-dihydro-indol-3-ylidenemethyl)-phenyl]-piperazine-1-carbaldehyde

¹³C-NMR (DMSO-d₆, E isomer) δ 169.2, 161.0, 151.5, 142.4, 136.6, 131.6, 129.2, 124.1, 124.0, 121.9, 121.5, 120.9, 114.7, 109.9, 48.0, 46.8, 44.3. ¹³C-NMR (DMSO-d₆, Z isomer) δ 167.5, 161.0, 151.7, 139.9, 137.3, 134.4, 127.6, 125.8, 124.5, 122.2, 120.7, 118.8, 113.9, 109.0, 47.7, 46.5, 44.3.

### Compound 226, 3-(3,5-dimethyl-1H-pyrrol-2-ylmethylene)-1,3-dihydro-indol-2-one

### Compound 227, 2,4-dimethyl-5-(2-oxo-1,2-dihydro-indol-3-ylidenemethyl)-1H-pyrrole-3-carboxylic acid (2-diethylamino-ethyl)-amide

### Compound 228, 5-(5-fluoro-2-oxo-1,2-dihydro-indol-3-ylidenemethyl)-2,4-dimethyl-1H-pyrrole-3-carboxylic acid (2-diethylamino-ethyl)-amide

### Compound 229, (3,5-dimethyl-1H-pyrrol-2-yl)-(2-oxo-1,2-dihydro-indol-3-ylidene)-acetic acid

### Compound 230, 3-[2,4-dimethyl-5-(2-oxo-1,2-dihydro-indol-3-ylidenemethyl)-1H-pyrrol-3-yl]-propionic acid

### Compound 231, 3-(3,5-dimethyl-1H-pyrrol-2-ylmethylene)-4-iodo-1,3-dihydro-indol-2-one

### Compound 232, 3-(3,5-dimethyl-1H-pyrrol-2-ylmethylene)-5-methoxy-1,3-dihydro-indol-2-one

### Compound 233, 5-chloro-3-(3-methoxy-1H-pyrrol-2-ylmethylene)-1,3-dihydro-indol-2-one

### Compound 234, 3-(3-methoxy-1H-pyrrol-2-ylmethylene)-1,3-dihydro-indol-2-one

### Compound 235, 3-[5-(4-chloro-2-oxo-1,2-dihydro-indol-3-ylidenemethyl)-2,4-dimethyl-1H-pyrrol-3-yl]-propionic acid

### Compound 236, 4-chloro-3-(3,5-dimethyl-1H-pyrrol-2-ylmethylene)-1,3-dihydro-indol-2-one

### Compound 237, 4-chloro-3-(3-methoxy-1H-pyrrol-2-ylmethylene)-1,3-dihydro-indol-2-one

### Compound 238, 3-(3,5-dimethyl-1H-pyrrol-2-ylmethylene)-2-oxo-2,3-dihydro-1H-indole-4-carboxylic acid

### Compound 239, 3-(3,5-dimethyl-1H-pyrrol-2-ylmethylene)-5-pyridin-3-yl-1,3-dihydro-indol-2-one

### Compound 240, 3-(3,5-dimethyl-1H-pyrrol-2-ylmethylene)-5-pyridin-3-yl-1,3-dihydro-indol-2-one; methanesulfonic acid

### Compound 241, 5-pyridin-3-yl-3-(1H-pyrrol-2-ylmethylene)-1,3-dihydro-indol-2-one

### Compound 242, 5-pyridin-3-yl-3-(1H-pyrrol-2-ylmethylene)-1,3-dihydro-indol-2-one; methanesulfonic acid

### Compound 243, 3-(3,5-dimethyl-1H-pyrrol-2-ylmethylene)-5-hydroxy-1,3-dihydro-indol-2-one

### Compound 244, 3-(3,5-dimethyl-1H-pyrrol-2-ylmethylene)-5-fluoro-1,3-dihydro-indol-2-one

### Compound 245, 3-(1-methyl-1H-indol-2-ylmethylene)-1,3-dihydro-indol-2-one

### Compound 246, 2,4-dimethyl-5-(2-oxo-1,2-dihydro-indol-3-ylidenemethyl)-1H-pyrrole-3-carboxylic acid ethyl ester

### Compound 247, 3-(5-phenyl-furan-2-ylmethylene)-1,3-dihydro-indol-2-one

### Compound 248, 3-(5-bromo-thiophen-2-ylmethylene)-1,3-dihydro-indol-2-one

### Compound 249, 3-(4-bromo-thiophen-2-ylmethylene)-1,3-dihydro-indol-2-one

### Compound 250, 3-(5-bromo-thiophen-2-ylmethylene)-1,3-dihydro-indol-2-one

### Compound 251, 3-(5-nitro-thiophen-2-ylmethylene)-1,3-dihydro-indol-2-one

### Compound 252, 3-furan-3-ylmethylene-1,3-dihydro-indol-2-one

### Compound 253, 3-(2,4-dimethoxy-pyrimidin-5-ylmethylene)-1,3-dihydro-indol-2-one

### Compound 254, 3-(4-phenyl-thiophen-2-ylmethylene)-1,3-dihydro-indol-2-one

### Compound 255, 3-(4-bromo-furan-2-ylmethylene)-1,3-dihydro-indol-2-one

### Compound 256, 3-(4,5-dimethyl-thiophen-2-ylmethylene)-1,3-dihydro-indol-2-one

### Compound 257, 3-(4,5-dimethyl-furan-2-ylmethylene)-1,3-dihydro-indol-2-one

### Compound 258, 3-[5-methoxy-2-(2-morpholin-4-yl-ethoxy)-benzylidene]-1,3-dihydro-indol-2-one; hydrochloride

### Compound 259, 3-[5-methoxy-2-(2-piperidin-1-yl-ethoxy)-benzylidene]-1,3-dihydro-indol-2-one; hydrochloride

### Compound 260, 3-(2,5-dimethoxy-benzylidene)-5,7-difluoro-1,3-dihydro-indol-2-one

¹H-NMR (DMSO-d₆, E isomer) δ 11.14 (br, 1H), 7.75 (s, 1H), 7.25 (t, 1H), 7.21 (d, 1H), 7.12 (m, 2H), 7.00 (dd, 1H), 3.81 (s, 3H), 3.74 (s, 3H).

### Compound 261, 3-[4-(1-quinolin-4-yl-ethoxy)-benzylidene]-1,3-dihydro-indol-2-one

¹H-NMR (DMSO-d₆) δ 10.52 (s, 1H), 8.88 (d, 1H), 8.41 (d, 1H), 8.09 (d, 1H), 7.83 (t, 1H), 7.72 (t, 1H), 7.67-7.53 (m, 4H), 7.50 (s, 1H), 7.20 (t, 1H), 7.07 (d, 2H), 6.83 (m, 2H), 6.44 (q, 1H), 1.75 (d, 3H).

### Compound 262, 3-[4-(pyridin-4-ylmethoxy)-benzylidene]-1,3-dihydro-indol-2-one

¹H-NMR (DMSO-d₆) δ 10.55 (br, 1H), 8.61 (d, 2H), 7.72 (d, 2H), 7.63 (d, 1H), 7.58 (s, 1H), 7.47 (d, 2H), 7.23 (t, 1H), 7.18 (d, 2H), 6.87 (m, 2H), 5.29 (s, 2H).

### Preparation 69

### Compound 263, 4-methyl-5-(2-oxo-1,2-dihydro-indol-3-ylidenemethyl)-1H-pyrrole-2-carboxylic acid pyridin-4-ylmethyl ester

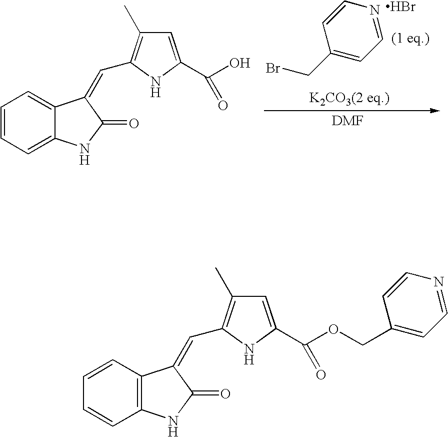

The title compound was obtained as a brown solid following the general procedure 5 using 4-bromomethyl-pyridine; hydrobromide (63 mg, 0.25 mmole, 1 eq.) and K₂CO3 (70 mg, 0.5 mmole, 2 eq.): 45 mg, 50% yield.

¹H NMR (DMSO-d₆, Z isomer) δ 14.25 (s, 1H), 12-10 (bs, 1H), 8.60 (m, 2H), 7.85 (d, 1H), 7.72 (s, 1H), 7.47 (m, 2H), 7.21 (bt, 1H), 7.03 (bt, 1H), 6.95-6.88 (m, 2H), 5.40 (s, 2H), 2.37 (s, 3H).

### Preparation 70

### Compound 264, (3,5-dimethyl-1H-pyrrol-2-yl)-(2-oxo-1,2-dihydro-indol-3-ylidene)-acetic acid benzyl ester

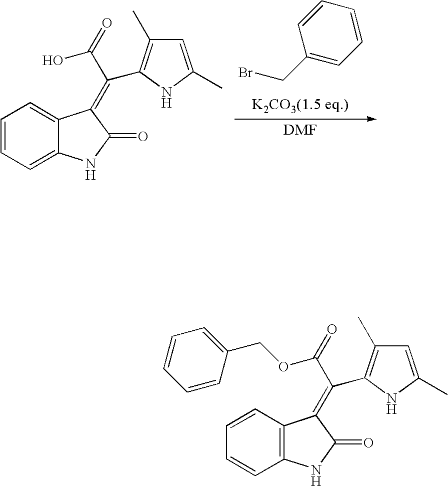

The title compound was obtained as an orange/red solid following the general procedure 5 using bromomethyl-benzene (30 μL, 0.25 mmole, 1 eq.) and K₂CO₃ (53 mg, 0.37 mmole, 1.5 eq.): 56 mg, 61% yield.

¹³C NMR (DMSO-d₆) δ 169.4, 166.9, 138.4, 136.2, 133.9, 132.8, 130.5, 129.3, 128.5, 128.4, 126.6, 122.6, 122.4, 121.1, 120.0, 115.4, 109.9, 109.6, 68.2, 13.3, 12.9.

### Preparation 71

### Compound 265, 5-amino-3-(2,5-dimethoxy-benzylidene)-1,3-dihydro-indol-2-one; methanesulfonic acid

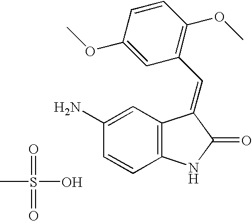

To a hot dark red solution of 5-amino-3-(2,5-dimethoxy-benzylidene)-1,3-dihydro-indol-2-one (0.70 g, 2.4 mmoles) in THF (35 mL) was added methanesulfonic acid (0.15 mL, 2.4 mmoles) and the formation of a yellow precipitate was observed. The precipitate was filtered and dried to afford the title compound as a yellow/orange solid (0.83 g, 90% yield).

¹³C-NMR (DMSO-d₆, mixture of the E and Z isomers, signals of the predominant E isomer) δ 168.4, 152.7, 151.8, 142.3, 133.3, 126.6, 124.8, 124.6, 122.8, 121.9, 117.9, 117.2, 113.7, 112.9, 110.6, 55.9, 55.5, 39.6.

### Example 1

Materials and Methods

Compounds: Compound 226=3-(3,5-dimethyl-1H-pyrrol-2-yl-methylene)-1,3-dihydro-indol-2-one; Compound A=3-[2,4-dimethyl-5-(oxo-1,2-dihydroindol-3-ylidenemethyl)-1H-pyrrol-3-yl]-propionic acid; Compound B=a semisynthetic analogue of fumagillol, CAS RN 129298-91-5.

Peptide The following peptide from myelin proteolipid protein was used; PLP₁₃₉₋₁₅₃ H-HCLGKWLGHPDKFVG-OH. The peptide was synthesized by Fmoc chemistry (Schafer-N, Copenhagen, Denmark). Purity (>95%) was verified by reversed-phase HPLC and integrity by mass spectrometry.

Mice Female SJL/J (H-2^{s}) inbred mice purchased from Charles River.

Immunization The SJL/J mice (about 8 weeks old) were immunized on day 0 with the PLP₁₃₉₋₁₅₃ peptide (dissolved in sterile NaCl) emulsified 1:1 (vol/vol) in Complete Freund's Adjuvant (5 mg Mycobacterium tuberculosis/ml) (SSI, Copenhagen, Denmark). Intradermal injections corresponding to 100 μg peptide and 125 μg Mycobacterium tuberculosis were given at the base of the tail in a total volume of 50 μl. The mice were additionally given an i.v. injection with 100 ng pertussis toxin (Sigma) dissolved in sterile NaCl on day 0 and day 2, injection volume was 100 μl.

Compound treatment Groups of 10 mice were dosed daily with compound 226 (from 4 mg/kg to 50 mg/kg), compound A (from 50 mg/kg to 200 mg/kg) or compound B (5 mg/kg to 10 mg/kg) in suspension vehicle (4 g Tween-80, 2 g Carboxy-methyl cellulose 7H4XF, 8 g NaCl, 1 liter H₂O), starting on day 0 unless otherwise specified. Control groups were given either suspension vehicle or dexamethasone (Dexadreson Vet, Intervet, Holland). Suspension vehicle was given from day 0 i.p. whereas dexamethasone (0.5 or 1 mg/kg) was given p.o. from day 1.

Clinical evaluation Mice were weighed and assessed clinically daily from day 5 p.i. according to the following criteria: 0, no disease; 1, tail paralysis; 2, clumsy gait/poor righting ability and limb weakness; 3, moderate or total hind limb paralysis; 4, moribund state or dead.

Statistics

Area-under-curve (AUC) of the disease score was calculated for all mice. The medians of AUC of all groups were compared using Kruskal-Wallis test. When P<0.05 in the Kruskal-Wallis test, the Mann-Whitney test was used to compare drug treated groups with the suspension vehicle treated control group (P<0.05). AUC were calculated from day 0 to the termination of the individual experiment (day 21, day 28 or day 35).

Results

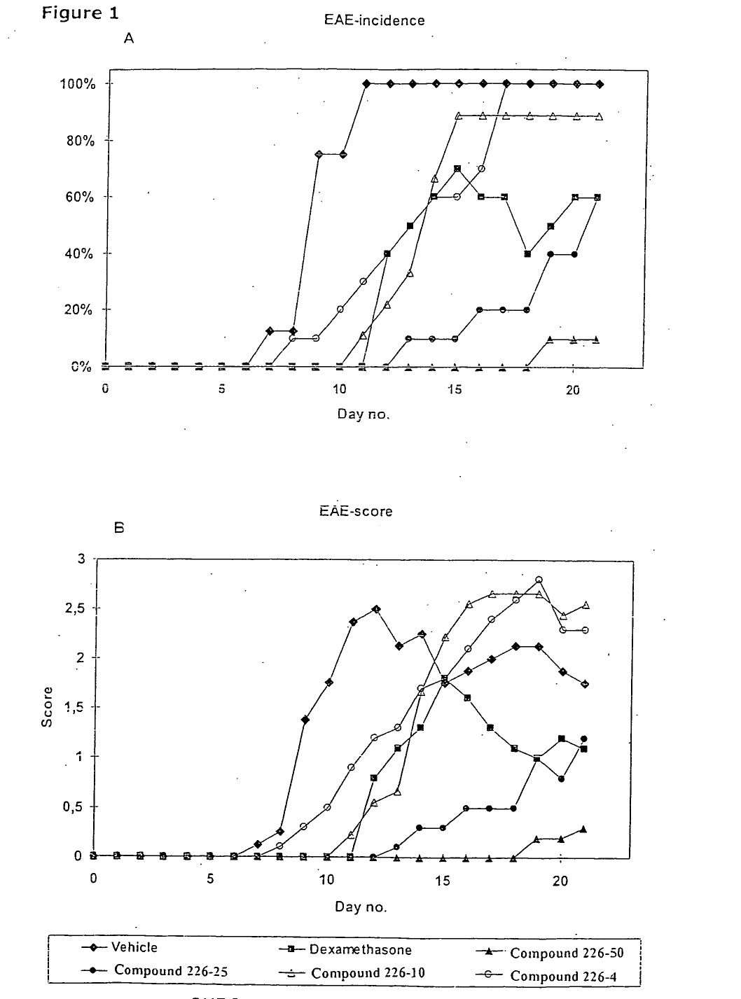

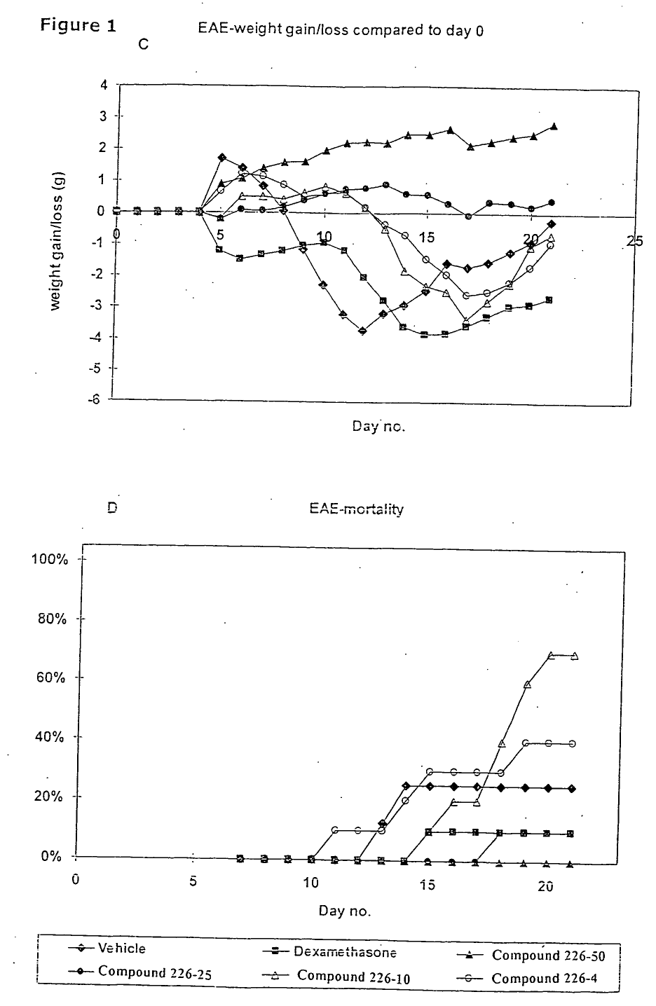

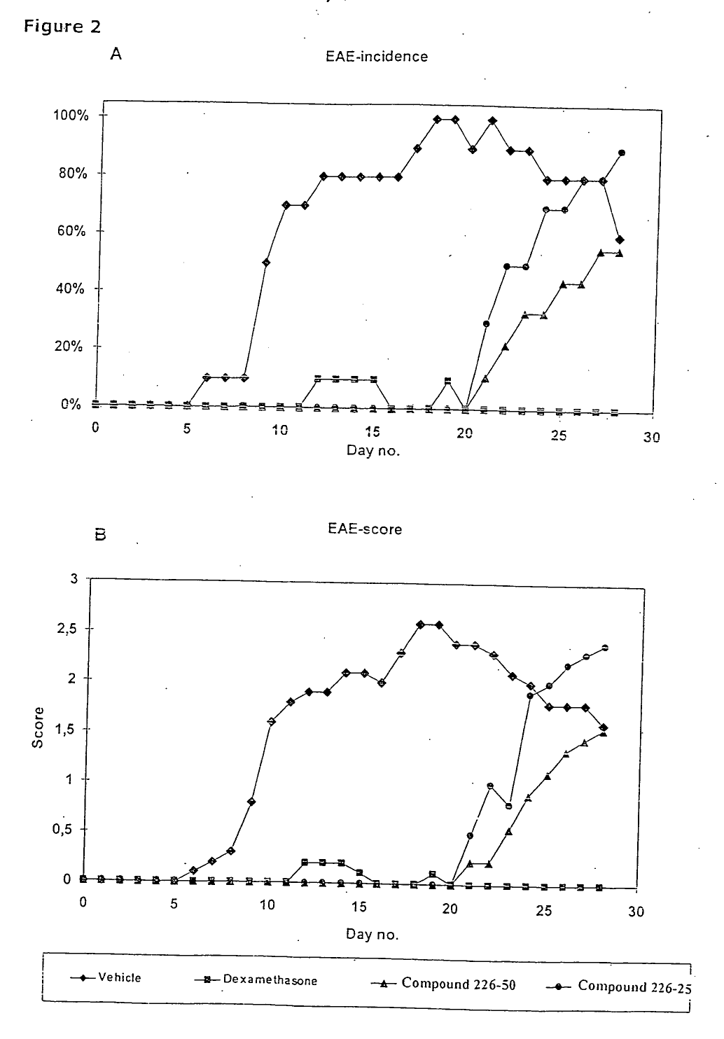

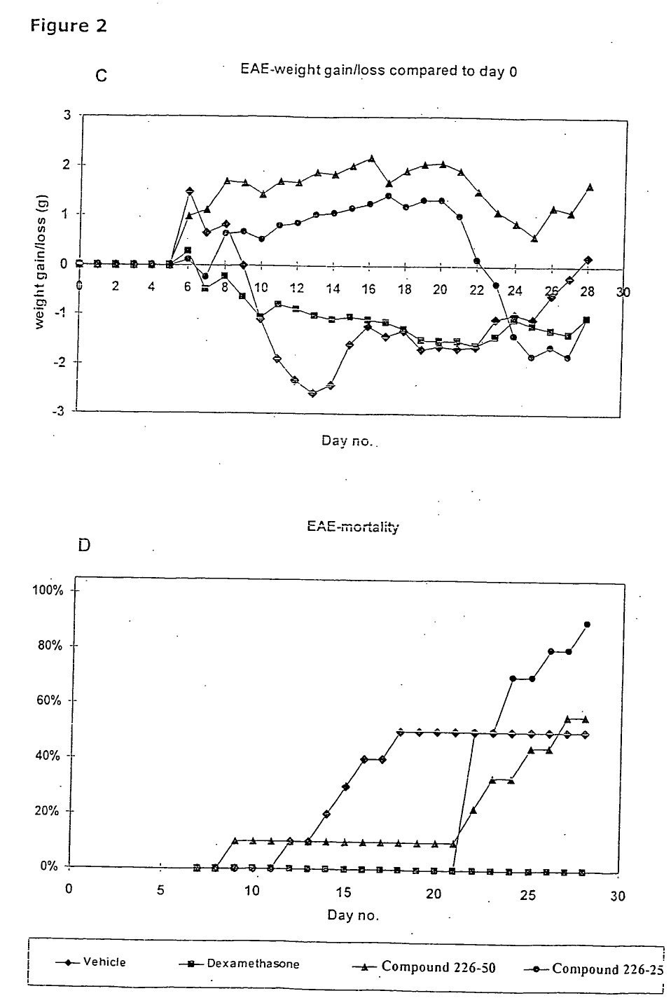

In four separate experiments, compound 226 (50 mg/kg) was shown to significantly inhibit EAE induced in SJL/J mice with a peptide from myelin proteolipid protein when compared with the suspension vehicle group (table 1, FIGS. 1 and 2). Compound 226 is a known KDR inhibitor and has also been described to have an anti-angiogenic effect. In order to evaluate whether the inhibition of EAE could be ascribed to inhibition of KDR or inhibition of angiogenesis, a known KDR inhibitor and angiogenese inhibitor were also tested, compound A and compound B, respectively (table 1). There is no significant inhibition of EAE when mice are dosed with compound A or compound B suggesting that a mechanism other than KDR and angiogenesis inhibition or possibly a combination of KDR inhibition and another mechanism of action may account for the effect of compound 226. A dose-response course is observed when compound 226 is given in different doses (table 1, FIG. 1) with a significant inhibition of EAE by dosing compound 226 as 50 mg/kg as well as 25 mg/kg.

From the data obtained in experiment 3 (table 1, FIG. 2) and 4 (table 1) which were prolonged with one and two weeks respectively, it appears that treatment with compound 226 delays development of EAE rather than prevents the disease.

TABLE 1
Inhibition of EAE with compound 226.

| | | | | inhibition | |
|---|---|---|---|---|---|
| Exp. | Compound | AUC# (mean) | SD | (%) | Significance |
| 1* | Vehicle | 17.2 | 16.1 | | |
| | Dexamethasone from day 0 | 4.3 | 7.4 | 75 | p = 0.022 |
| | (0.5 mg/kg) | | | | |
| | 226 (50 mg/kg i.p.) | 1.8 | 5.5 | 90 | p < 0.01 |
| | A (50 mg/kg i.p.) | 23.4 | 12.5 | −36 | NS |
| 2** | Vehicle | 25.4 | 9.0 | | |
| | Dexamethasone from day 1 | 11.8 | 8.1 | 54 | p < 0.01 |
| | (1 mg/kg) | | | | |
| | 226 (50 mg/kg i.p.) | 0.6 | 1.7 | 98 | p < 0.01 |
| | 226 (25 mg/kg i.p.) | 4.6 | 7.7 | 82 | p < 0.01 |
| | 226 (10 mg/kg i.p.) | 19.6 | 8.7 | 23 | NS |
| | 226 (4 mg/kg i.p.) | 21.2 | 14.5 | 17 | NS |
| | B (10 mg/kg i.p.) | 19.3 | 12.7 | 24 | NS |
| | B (5 mg/kg i.p.) | 19.0 | 13.6 | 25 | NS |
| 3*** | Vehicle | 39.7 | 17.9 | | |
| | Dexamethasone from day 1 | 0.8 | 2.2 | 98 | p < 0.01 |
| | (1 mg/kg) | | | | |
| | 226 (50 mg/kg i.p.) | 6.6 | 7.8 | 83 | p < 0.01 |
| | 226 (25 mg/kg i.p.) | 11.9 | 7.8 | 70 | p < 0.01 |
| 4**** | Vehicle | 67.2 | 37.0 | | |
| | 226 (50 mg/kg i.p.) | 21.9 | 19.7 | 65 | p = 0.011 |
| | A (200 mg/kg p.o.) | 55.5 | 36.5 | 11 | NS |

Area-under-curve of the disease score for four individually experiments. In each experiment a suspension vehicle group was included. AUC of the disease score were calculated for all groups and the Mann-Whitney test was used to compare the drug treated groups with the suspension vehicle group.
*This experiment was terminated on day 21.
**This experiment was terminated on day 21.
***This experiment was terminated on day 28.
****This experiment was terminated on day 35.
#AUC for the experiments are calculated from day 0 to termination of the individual experiment.

### Example 2

Inhibition of IL-2 Production in Spleen Cells of Mice Dosed with Compound 226

Materials and Methods

Peptide The following peptide from myelin proteolipid protein was used; PLP₁₃₉₋₁₅₃ H-HCLGKWLGHPDKFVG-OH. The peptide was synthesized by Fmoc chemistry (Schafer-N, Copenhagen, Denmark). Purity (>95%) was verified by reversed-phase HPLC and integrity by mass spectrometry.

Mice Female SJL/J (H-2^{s}) inbred mice purchased from Charles River.

Immunization The SJL/J mice (about 8 weeks old) were immunized on day 0 with the PLP₁₃₉₋₁₅₃ peptide (dissolved in sterile NaCl) emulsified 1:1 (vol/vol) in Complete Freund's Adjuvant (5 mg Mycobacterium tuberculosis/ml) (SSI, Copenhagen, Denmark). Intradermal injections corresponding to 100 μg peptide and 125 μg Mycobacterium tuberculosis were given at the base of the tail in a total volume of 50 μl. The mice were additionally given an i.v. injection with 100 ng pertussis toxin (Sigma) dissolved in sterile NaCl on day 0 and day 2, injection volume was 100 μl.

Compound treatment A group of 6 mice were dosed i.p. daily for 10 days with compound 226 (50 mg/kg), in suspension vehicle (4 g Tween®-80, 2 g carboxymethylcellulose 7H4XF, 8 g NaCl, 1 liter H₂O), starting on day 0. A control group was given suspension vehicle daily.

Clinical evaluation Mice were weighed and assessed clinically daily from day 5 p.i. according to the following criteria: 0, no disease; 1, tail paralysis; 2, clumsy gait/poor righting ability and limb weakness; 3, moderate or total hind limb paralysis; 4, moribund state or dead.

In vitro restimulation Ten days after immunization individual spleens were collected and single-cell suspensions were prepared in RPMI (Bio Whittaker) supplemented with 1% mouse serum, 1% penicillin/streptomycin (Gibco) and 1% L-glutamine (Gibco). The erythrocytes were lysed by 0.⁸³% NH₄Cl treatment. Subsequently the cells were washed and plated out (5×10⁵ cells/well) on 96-well flat-bottomed microtiter plates (Nunclon, NUNC, Denmark). Different concentrations of the PLP₁₃₉₋₁₅₃ peptide were added to the wells. Control wells did not contain the PLP₁₃₉₋₁₅₃ peptide. Cells from each mouse were plated with or without 10⁻⁷M of compound 226 on the microtiter plate. The plates were incubated at 37° C. for 3 days. Afterwards 100 μl of the supernatant from each well was transferred to a corresponding 96-well flat-bottomed microtiter plate (Maxisorp, NUNC, Denmark) coated with 50 μl/well of an anti-IL-2 antibody (2 μg/ml) (PharMingen, Becton Dickinson) in coating buffer (sodium bicarbonate, 0.1 mol/L, pH 9.5). The Maxisorp plates were blocked with blocking buffer (10% FCS in PBS) and washed with (0.05% Tween®-20 in PBS) before addition of the supernatant. After incubation overnight at 4° C. the plates were washed and 100 μl of biotinylated anti-IL-2 antibody (1 μg/ml) (PharMingen, Becton Dickinson), diluted in blocking buffer, was added to each well. After 45 min. incubation at room temperature, the plates were washed and incubated for 2 hrs. at room temperature on a plate-shaker with 100 μl/well of Eu³⁺-labeled streptavidin (Wallac, Perkin Elmer) diluted 1/1000 in assay buffer (Wallac, Perkin Elmer). After washing, the plates were incubated for 10 min. on a plate-shaker with 180 μl enhancement solution (Wallac, Perkin Elmer) which releases europium from streptavidin and forms a highly fluorescent micellar solution. Finally, fluorescence was measured in a time-resolved fluorometer VICTOR (Wallac, Perkin Elmer).

Conclusion

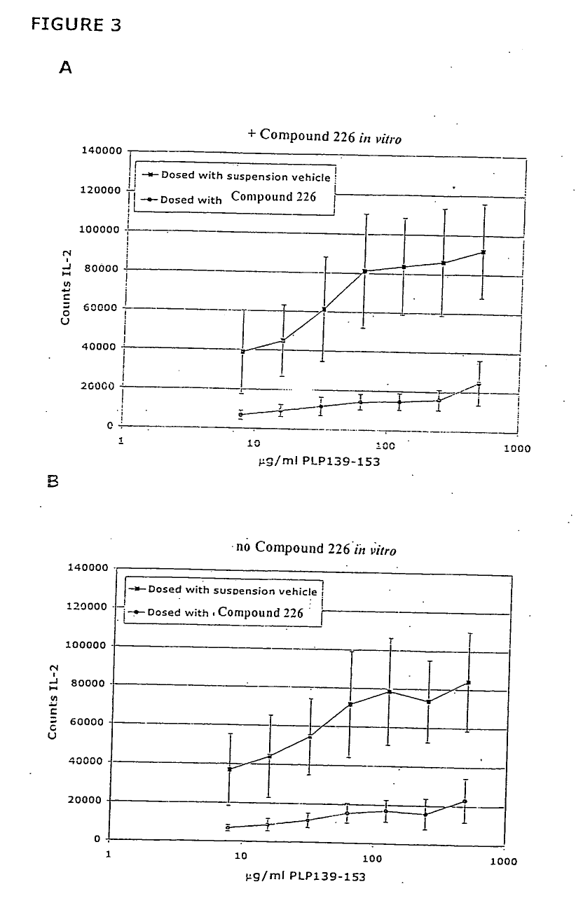

Two separate in vitro restimulation experiments (one example is given) show that the IL-2 production is markedly inhibited in vivo after dosing with compound 226 (50 mg/kg) for 10 days (FIG. 3A). The inhibition of IL-2 is antigen dependent as there were no IL-2 production in wells without PLP₁₃₉₋₁₅₃ (data not shown). Normal mice (not immunized) were also included in the experiment and did not produce any IL-2 after incubation with or without PLP₁₃₉₋₁₅₃ (data not shown). Furthermore, the results show that incubation for 3 days in vitro with 10⁻⁷ M of compound 226 did not inhibit the IL-2 production from cells originating from the suspension vehicle treated mice (FIGS. 3A and 3B) indicating that in vivo dosing is necessary and sufficient to obtain the observed IL-2 inhibition.

### Example 3

Inhibition of EAE in a Second Model for EAE

Materials and Methods

Compounds: Compound 226=3-(3,5-dimethyl-1H-pyrrol-2-yl-methylene)-1,3-dihydro-indol-2-one

Peptide The following peptide from myelin oligodendrocyte glycoprotein was used; MOG₃₅₋₅₅ H-MEVGWYRSPFSRVVHLYRNGK-OH. The peptide was synthesized by Fmoc chemistry (Schafer-N, Copenhagen, Denmark). Purity (>95%) was verified by reversed-phase HPLC and integrity by mass spectrometry.

Mice Female C57BL/6JBom (H-2^{b}) inbred mice purchased from Taconic M&B, Denmark.

Immunization The C57BL/6JBom mice (about 8 weeks old) were immunized at day 0 with the MOG₃₅₋₅₅ peptide (dissolved in sterile NaCL) emulsified 1:1 (vol/vol) in Complete Freund's Adjuvant (5 mg Mycobacterium tuberculosis/ml) (Statens Serum Institut, Copenhagen, Denmark). Intradermal injections corresponding to 200 μg peptide and 250 μg Mycobacterium tuberculosis were given at the base of the tail in a total volume of 50 μl. The mice were additionally given an i.v. injection with 100 ng Pertussis Toxin (Sigma) dissolved in sterile NaCl at day 0 and day 2, injection volume was 100 μl.

Compound treatment Groups of 10 mice were dosed daily with compound 226 (50 mg/kg i.p.) in suspension vehicle (4 g Tween-80, 2 g carboxy-methyl cellulose 7H4XF, 8 g NaCl, 1 liter H₂O) starting at day 0. Control groups were given either suspension vehicle or dexamethasone (Dexadreson Vet, Intervet, Holland). Suspension vehicle was given from day 0 i.p. whereas dexamethasone (1 mg/kg) was given p.o. from day 1.

Clinical evaluation Mice were weighed and assessed clinically daily from day 5 post immunization (p.i.) according to the following criteria: 0, no disease; 1, tail paralysis; 2, clumsy gait/poor righting ability and limb weakness; 3, moderate or total hind limb paralysis; 4, moribund state or dead.

Statistics

Area under curve (AUC) of the disease score was calculated for all mice. The medians of AUC of all groups were compared using Kruskal-Wallis test. When p<0.05 in the Kruskal-Wallis test, the Mann-Whitney test was used to compare drug treated groups with the suspension vehicle treated control group (p<0.05). AUC were calculated from day 0 to the termination of the experiment (day 21).

Results

The results presented in table 2 demonstrate that compound 226 has a significant effect on disease development in C57BL/6 mice immunized with a peptide from myelin oligodendrocyte glycoprotein, indicating that the effect of compound 226 on EAE is independent of the mouse strain used in the experiment and the immunization protocol used for the induction of EAE.

TABLE 2
Inhibition of EAE with compound 226 in a second EAE model

| | AUC# | | Inhibition | |
|---|---|---|---|---|
| Compound | (mean) | SD | (%) | Significance |
| Vehicle | 15.40 | 8.4 | | |
| Dexamethasone (1 mg/kg p.o.) | 0.00 | 0.0 | 100 | p < 0.01 |
| Compound 226 (50 mg/kg i.p.) | 2.65 | 6.1 | 83 | p < 0.01 |

#Area-under-curve of the disease score. AUC of the disease score were calculated for all groups and the Mann-Whitney test was used to compare the treated groups with the suspension vehicle group.

### Example 4

Inhibition of EAE with Analogues of Compound 226

Materials and Methods

Compounds: Compound 57=3-(1H-indol-3-ylmethylene)-1,3-dihydro-indol-2-one) and Compound 110=3-(2,5-dimethoxybenzylidene)-1,3-dihydro-indol-2-one.

Peptide The following peptide from myelin proteolipid protein was used; PLP₁₃₉₋₁₅₃ H-HCLGKWLGHPDKFVG-OH. The peptide was synthesized by Fmoc chemistry (Schafer-N, Copenhagen, Denmark). Purity (>95%) was verified by reversed-phase HPLC and integrity by mass spectrometry.

Mice Female SJL/J (H-2^{s}) inbred mice purchased from Charles River or Taconic M&B (Denmark).

Immunization The SJL/J mice (about 8 weeks old) were immunized at day 0 with the PLP₁₃₉₋₁₅₃ peptide (dissolved in sterile NaCL) emulsified 1:1 (vol/vol) in Complete Freund's Adjuvant (5 mg Mycobacterium tuberculosis/ml) (Statens Serum Institut, Copenhagen, Denmark). Intradermal injections corresponding to 100 μg peptide and 125 μg Mycobacterium tuberculosis were given at the base of the tail in a total volume of 50 μl. The mice were additionally given an i.v. injection with 100 ng Pertussis Toxin (Sigma) dissolved in sterile NaCl at day 0 and day 2, injection volume was 100 μl.

Compound treatment Groups of 10 mice were dosed daily with compound 57 (50 mg/kg) or compound 110 (50 mg/kg) in suspension vehicle (4 g Tween-80, 2 g Carboxy-methyl cellulose 7H4XF, 8 g NaCl, 1 liter H₂O), starting at day 0. Control groups were given either suspension vehicle or dexamethasone (Dexadreson Vet, Intervet, Holland). Suspension vehicle was given from day 0 i.p. whereas dexamethasone (1 mg/kg) was given p.o. from day 1.

Clinical evaluation Mice were weighed and assessed clinically daily from day 5 p.i. according to the following criteria: 0, no disease; 1, tail paralysis; 2, clumsy gait/poor righting ability and limb weakness; 3, moderate or total hind limb paralysis; 4, moribund state or dead.

Statistics

Area under curve (AUC) of the disease score was calculated for all mice. The medians of AUC of all groups were compared using Kruskal-Wallis test. When p<0.05 in the Kruskal-Wallis test, the Mann-Whitney test was used to compare drug treated groups with the suspension vehicle treated control group (p<0.05). AUC were calculated from day 0 to the termination of the experiment (day 21).

Results

Analogues of the potent compound 226 were tested in the EAE model in order to further evaluate the in vivo effect of this group of compounds. The results are shown in table 3 from which it appears that the two analogues, compound 57 and compound 110, both show a clear and significant inhibition of the disease. Furthermore, in another experiment compound 110 showed 90% inhibition of EAE when dosed with 50 mg/kg and a clear dose-response course was observed when compound 110 was given in different doses (50 mg/kg, 25 mg/kg, 10 mg/kg, 4 mg/kg), data not shown. There was significant inhibition of EAE by dosing compound 110 as 50 mg/kg, 25 mg/kg and 10 mg/kg with inhibition of disease ranging from 42% with the lowest dose to 90% with the highest dose.

TABLE 3
Inhibition of EAE with compounds 57 and 110

| | AUC# | | Inhibition | |
|---|---|---|---|---|
| Compound | (mean) | SD | (%) | Significance |
| Vehicle | 28.6 | 9.5 | | |
| Dexamethasone (1 mg/kg | 13.15 | 10.7 | 54 | p < 0.01 |
| p.o.) | | | | |
| Compound 57 (50 mg/kg i.p.) | 4.70 | 6.5 | 84 | p < 0.01 |
| Vehicle | 42.75 | 5.3 | | |
| Dexamethasone (1 mg/kg | 8.85 | 8.6 | 79 | p < 0.01 |
| p.o.) | | | | |
| Compound 110 (50 mg/kg | 12.15 | 11.8 | 72 | p < 0.01 |
| i.p.) | | | | |

#Area-under-curve of the disease score. A suspension vehicle group was included. AUC of the disease score were calculated for all groups and the Mann-Withney test was used to compare the treated groups with the suspension vehicle group.

### Example 5

Inhibition of EAE with Further Oxindole Compounds

Materials and Methods

Compounds: The compounds shown in Table 4 below dosed in an amount of 50 mg/kg.

The immunization and treatment of the mice was otherwise carried out as described in example 4.

Results

A series of oxindole compounds were tested in the EAE model to further evaluate the in vivo effect of this group of compounds. The results are shown in table 4. The inhibitory effects shown in Table 4 are all significant (p<0.05). The oxindole compounds inhibit EAE with varying effects ranging from 34% to 96% inhibition.

TABLE 4
Inhibition of EAE with further oxindole compounds

| Compound | Structure | *Inhibition (%) |
|---|---|---|
| 104 | 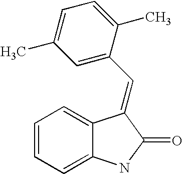 | 71 |
| 31 | 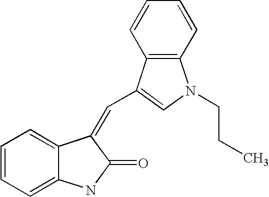 | 60 |
| 51 | 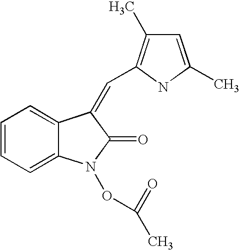 | 48 |
| 01 | 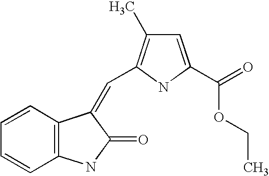 | 44 |
| 03 | 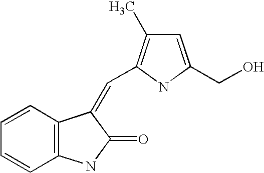 | 57 |
| 75 | 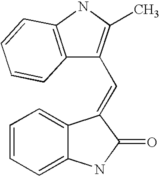 | 63 |
| 26 | 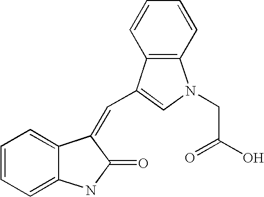 | 34 |
| 153 | 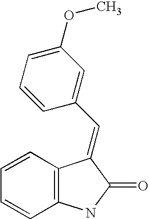 | 38 |
| 171 | 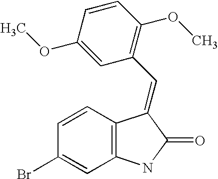 | 40 |
| 02 | 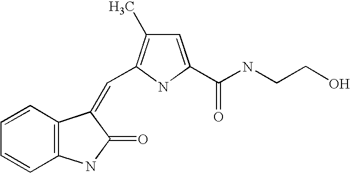 | 41 |
| 229 | 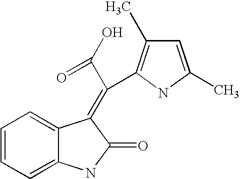 | 59 |
| 23 | 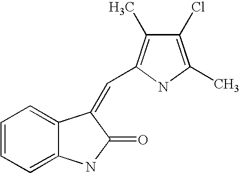 | 96 |
| 22 | 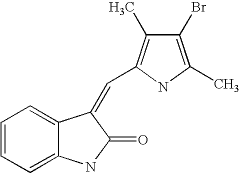 | 78 |
| 193 | 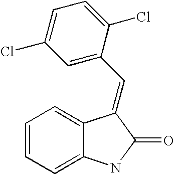 | 37 |
| 45 | 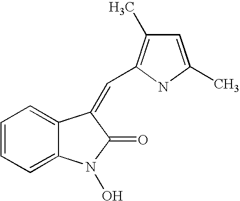 | 67 |
| 227 | 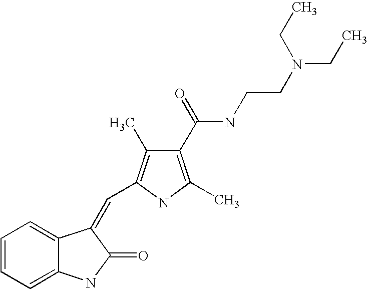 | 49 |
| 228 | 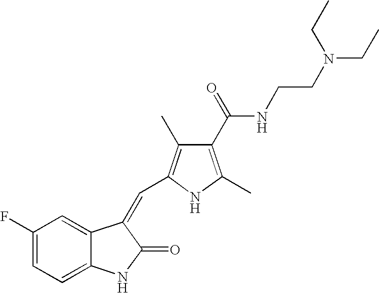 | 43 |

*Area-under-curve of the disease score. A suspension vehicle group was included in all experiments. AUC of the disease score were calculated for all groups and the Mann-Withney test was used to compare the treated groups with the suspension vehicle group.

### Example 6

Inhibition of Pro-inflammatory Cytokines

Materials and Methods

Peptide The following peptide from myelin proteolipid protein was used; PLP₁₃₉₋₁₅₃ H-HCLGKWLGHPDKFVG-OH. The peptide was synthesized by Fmoc chemistry (Schafer-N, Copenhagen, Denmark). Purity (>95%) was verified by reversed-phase HPLC and integrity by mass spectrometry.

Mice Female SJL/J (H-2^{s}) inbred mice purchased from Charles River.

Immunization The SJL/J mice (about 8 weeks old) were immunized at day 0 with the PLP₁₃₉₋₁₅₃ peptide (dissolved in sterile NaCl) emulsified 1:1 (vol/vol) in Complete Freund's Adjuvant (5 mg Mycobacterium tuberculosis/ml) (Statens Serum Institut, Copenhagen, Denmark). Intradermal injections corresponding to 100 μg peptide and 125 μg Mycobacterium tuberculosis were given at the base of the tail in a total volume of 50 μl. The mice were additionally given an i.v. injection with 100 ng Pertussis Toxin (Sigma) dissolved in sterile NaCl at day 0 and day 2, injection volume was 100 μl.

Compound treatment Groups of 4 mice were dosed i.p. daily for 10 days with compound 226 (50 mg/kg) or compound 57 (50 mg/kg) in suspension vehicle (4 g Tween-80, 2 g Carboxy-methyl cellulose 7H4XF, 8 g NaCl, 1 liter H₂O), starting at day 0. A control group were given suspension vehicle daily.

Clinical evaluation Mice were weighed and assessed clinically daily from day 5 p.i. according to the following criteria: 0, no disease; 1, tail paralysis; 2, clumsy gait/poor righting ability and limb weakness; 3, moderate or total hind limb paralysis; 4, moribund state or dead.

In vitro restimulation Ten days after immunization individual spleens were collected and single-cell suspensions were prepared in RPMI (Bio Whittaker) supplemented with 1% mouse serum, 1% penicillin/streptomycin (Gibco) and 1% L-glutamine (Gibco). The erythrocytes were lysed by 0.83% NH₄CL treatment. Subsequently the cells were washed and plated out (5×10⁵ cells/well) on 96-well flat-bottomed microtiter plates (Nunclon, NUNC, Denmark). Different concentrations of the PLP₁₃₉₋₁₅₃ peptide were added to the wells. Control wells were without the PLP₁₃₉₋₁₅₃ peptide. The plates were incubated at 37° C. for 3 days. Afterwards the supernatants from each mouse were pooled within the respective group and frozen for later analysis. For analysis the supernatants were transferred to a 96-well flat-bottomed microtiter plate (Maxisorp, NUNC, Denmark) coated with 50 μl/well of an anti-cytokine antibody (table 5) in coating buffer (natriumbicarbonate, 0.1 mol/L, pH 9.5). The Maxisorp plates were blocked with blocking buffer (10% FCS in PBS) and washed with (0.05% Tween-20 in PBS) before addition of the supernatant. After incubation O/N at 4° C. the plates were washed and 100 μl of biotinylated anti-cytokine antibody (table 5) diluted in blocking buffer was added to each well. After 45 min. incubation at room temperature, the plates were washed and incubated for 2 hr. at room temperature on a plate-shaker with 100 μl/well of EU³⁺-labeled streptavidin (Wallac, Perkin Elmer) diluted 1/1000 in assay buffer (Wallac, Perkin Elmer). After washing, the plates were incubated for 10 min. on a plate-shaker with 180 μl enhancement solution (Wallac, Perkin Elmer) which releases europium from streptavidin and forms a highly fluorescent micellar solution. Finally, fluorescence was measured in a time-resolved fluorometer VICTOR (Wallac, Perkin Elmer).

TABLE 5
dilution of used anti-cytokine antibodies

| | | | Working |
|---|---|---|---|
| Cytokine | Company | Format | concentration |
| IL-2 | BD Biosciences | Purified Ab | 2 μg/ml |
| | | Biotinylated Ab | 1 μg/ml |
| IL-6 | BD Biosciences | Purified Ab | 2 μg/ml |
| | | Biotinylated Ab | 1 μg/ml |
| IL-17 | BD Biosciences | Purified Ab | 10 μg/ml |
| | | Biotinylated Ab | 5 μg/ml |
| IFN-γ | BD Biosciences | Purified Ab | 10 μg/ml |
| | | Biotinylated Ab | 5 μg/ml |

Results

It appears from FIG. 4A-4D that there is a pronounced difference in the level of antigen-specific cytokine production of spleen cells in vivo after treatment of immunized mice with either compound 226 or compound 57 compared to the vehicle treated group. The level of the cytokines IL-2, IL-6, IFN-γ and IL-17 are clearly reduced when mice have been dosed with either of these two compounds which also inhibits the disease cause very efficiently. The reduction in one or more of these pro-inflammatory cytokines can be involved in the disease inhibiting process.

### Example 7

Inhibition of IL-2 by Oxindole Compounds

Peripheral blood mononuclear cells (PBMCs) were isolated from human blood. The lymphocytes, a fraction of the PBMCs, were activated to secrete IL-2 using the polyclonal mitogen phytohemagglutinin. The test compounds were added in concentrations from 10⁻⁵ to 10⁻¹⁰, and the cells (10⁶/ml) were incubated in microtiter wells at 37° C. for approximately 46 hours.

After 46 hours the cells were centrifuged down for 25 minutes at 1000×g and the supernatants were transferred to transferred to microtiter wells precoated with a monoclonal antibody against human IL-2. The IL-2 concentration in the supernatants was determined by a sandwich ELISA. Microtiter plates were coated (1 μg/ml) with a monoclonal antibody (R&D, UK) against human IL-2, washed 4 times, blocked with 1% casein buffer for 2 hours and washed 2 times. 100 μl sample was added to each well and incubated overnight. All samples were tested in triplicate. 100 μl IL-2 standards (R&D, UK) at a concentration range of 10000-0 pg/ml were tested in triplicate. After incubation the plates were washed and incubated with biotinylated polyclonal secondary antibody against human IL-2 (R&D, UK) for 45 minutes and thereafter washed 4 times. 100 μl enzyme conjugate, horseradish peroxidase conjugated streptavidin diluted 1:4000 (Zymed, USA), was added to all wells and incubated for 30 minutes. 100 μl OPD substrate (KEM EN TEC-DK) was added and the enzyme/substrate reaction stopped after 10 minutes at room temperature with 50 μl 1 M H₂SO₄. The colour development (optical density—OD) was determined at 492 nm on an ELISA reader and the background OD at 620 nm subtracted).

The results are shown in Table 6 below, including the tested oxindole compounds that may subsequently be subjected to testing in the EAE model as described above in examples 1, 3, 4 or 5. The results are expressed as a percentage of the control (PHA+DMSO) and the potency of the test compound was expressed as the concentration resulting in 50% inhibition of the response of the control stimulated cells (pIC₅₀).

TABLE 6
Inhibition of IL-2 production

| | IL-2 |
|---|---|
| Structure | pIC₅₀ |
| 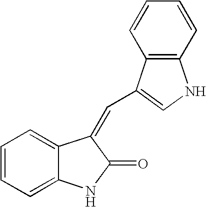 | 9 |
| 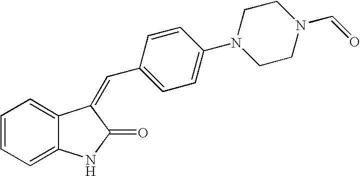 | 6.5 |
| 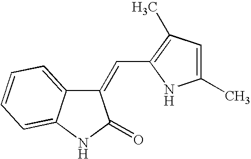 | 9.7 |
| 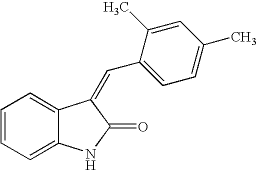 | 6.2 |
| 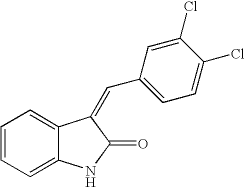 | 7.7 |
| 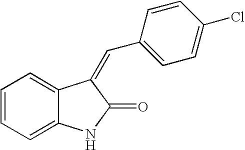 | 7.1 |
| 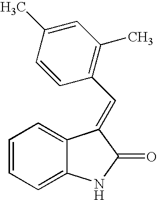 | 9 |
| 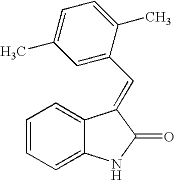 | 7 |
| 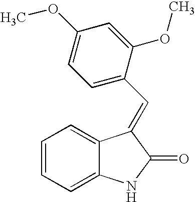 | 8.7 |
| 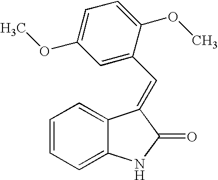 | 8.4 |
| 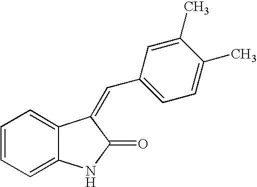 | 7.3 |
| 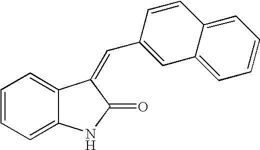 | 6.2 |
| 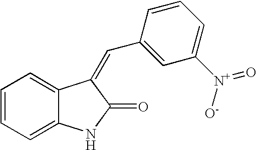 | 7.7 |
| 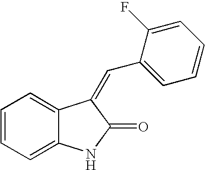 | 7.2 |
| 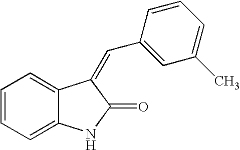 | 6.2 |
| 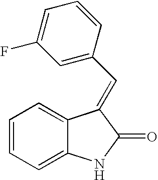 | 6.7 |
| 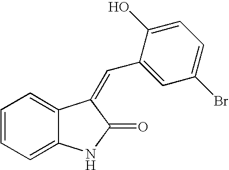 | 7.7 |
| 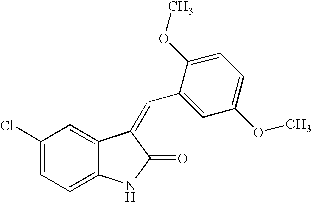 | 7.7 |
| 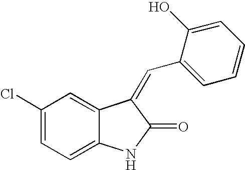 | 7 |
| 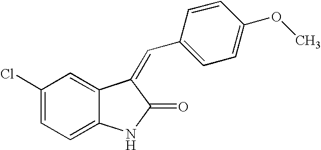 | 8 |
| 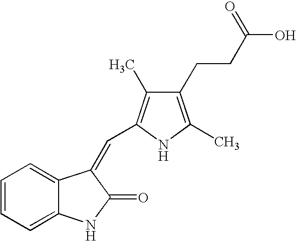 | 7.3 |
| 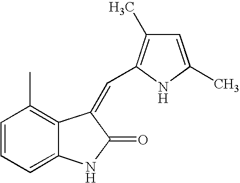 | 7.4 |
| 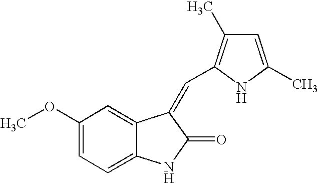 | 7.2 |
| 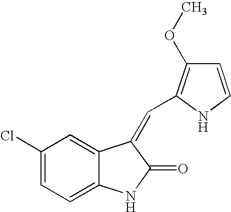 | 7.3 |
| 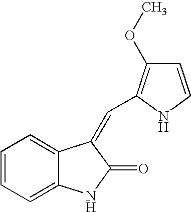 | 7.7 |
| 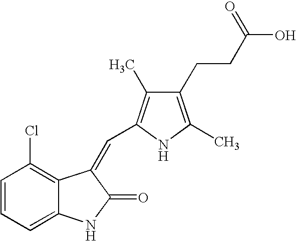 | 7.3 |
| 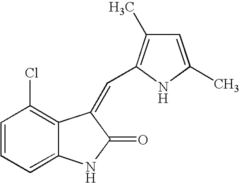 | 9 |
| 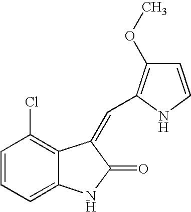 | 10 |
| 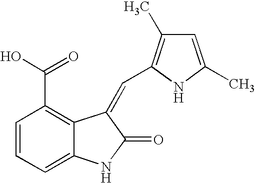 | 7 |
| 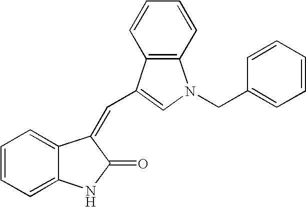 | 8.4 |
| 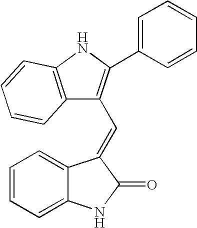 | 7 |
| 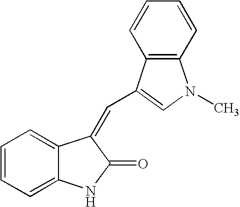 | 8.7 |
| 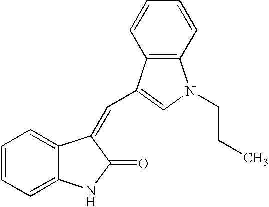 | 9.7 |
| 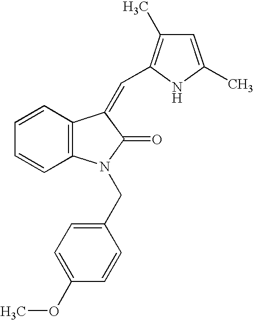 | 7.5 |
| 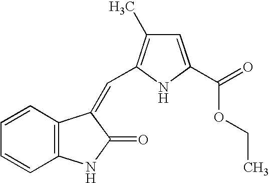 | 8 |
| 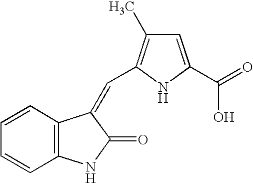 | 6.7 |
| 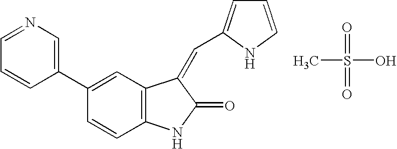 | 7 |
| 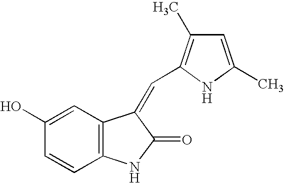 | 6.1 |
| 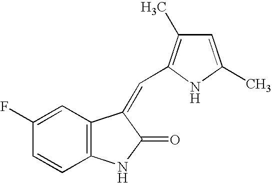 | 7 |
| 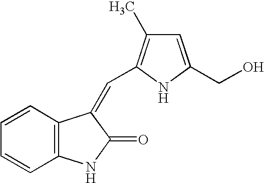 | 7.4 |
| 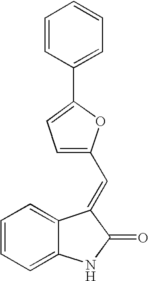 | 8.4 |
| 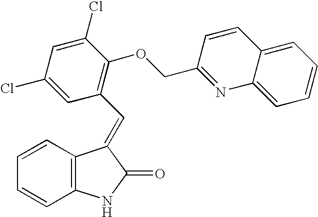 | 7 |
| 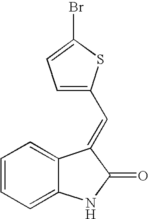 | 7.7 |
| 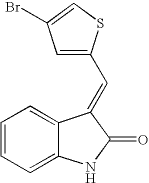 | 8.2 |
| 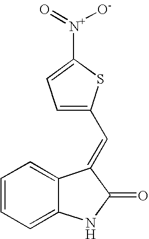 | 7.5 |
| 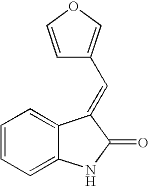 | 6.2 |
| 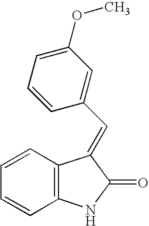 | 8.2 |
| 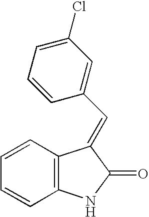 | 8 |
| 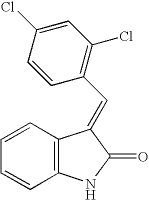 | 9 |
| 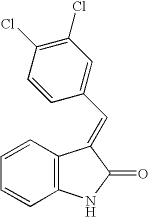 | 9 |
| 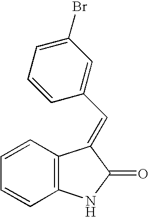 | 8 |
| 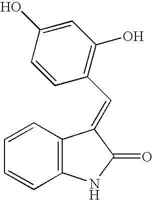 | 6.5 |
| 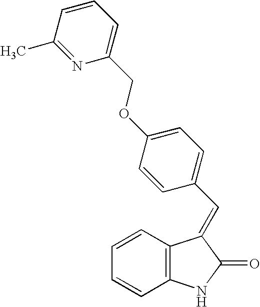 | 6.4 |
| 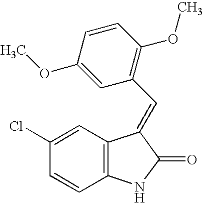 | 8.2 |
| 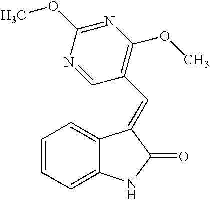 | 8.2 |
| 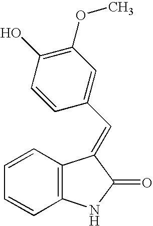 | 7 |
| 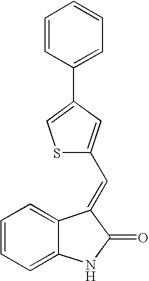 | 7.5 |
| 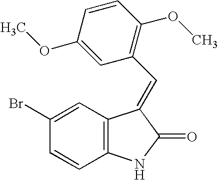 | 7 |
| 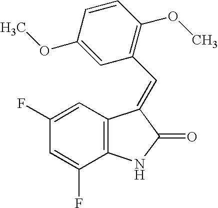 | 8 |
| 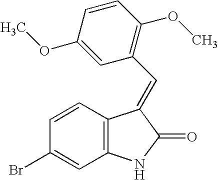 | 9 |
| 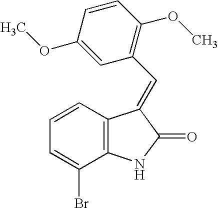 | 8 |
| 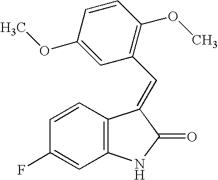 | 8 |
| 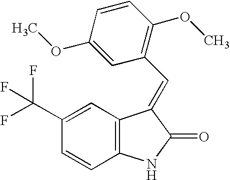 | 6.2 |
| 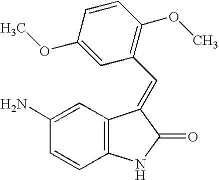 | 6.5 |
| 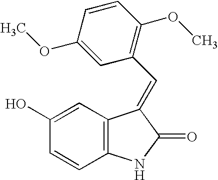 | 6.7 |
| 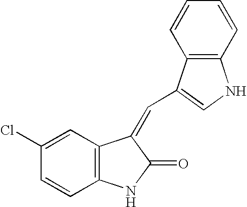 | 8 |
| 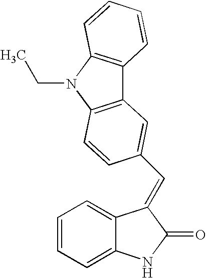 | 6.7 |
| 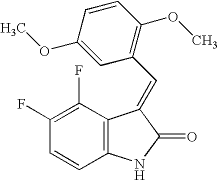 | 7.7 |
| 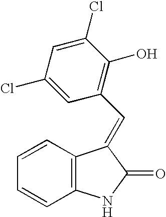 | 6.4 |
| 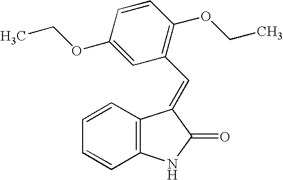 | 6.2 |
| 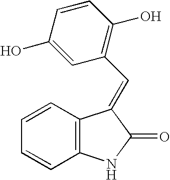 | 7 |
| 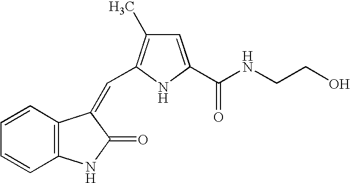 | 7.5 |
| 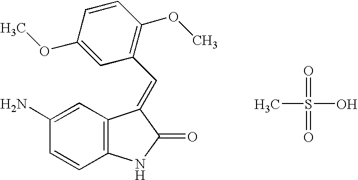 | 7.2 |
| 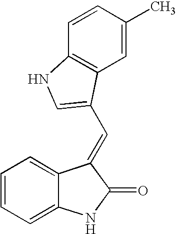 | 7.7 |
| 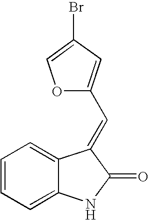 | 7.5 |
| 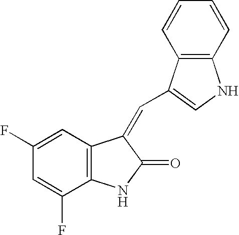 | 8.7 |
| 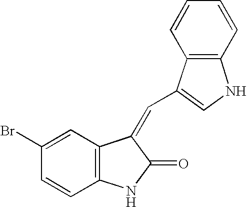 | 8.7 |
| 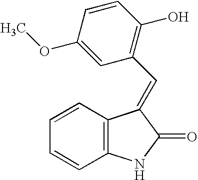 | 6.5 |
| 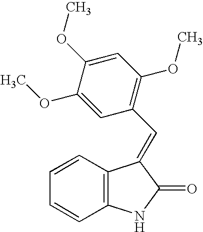 | 8.7 |
| 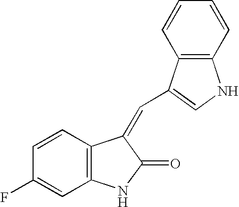 | 8.5 |
| 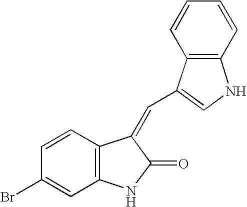 | 8.5 |
| 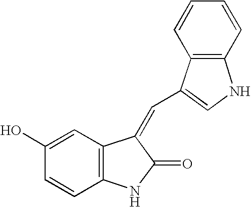 | 7.1 |
| 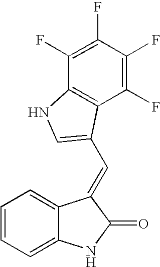 | 8.7 |
| 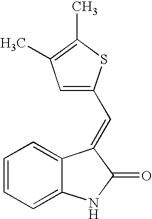 | 7 |
| 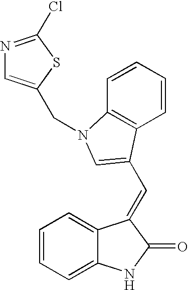 | 8 |
| 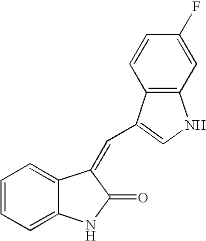 | 9 |
| 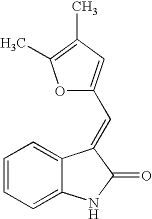 | 8.4 |
| 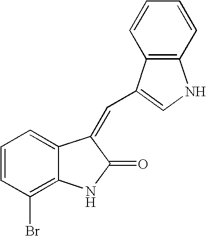 | 9.7 |
| 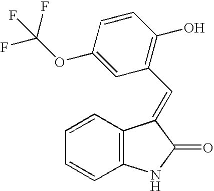 | 7.2 |
| 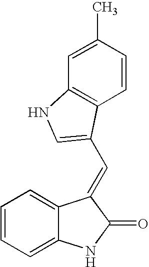 | 10 |
| 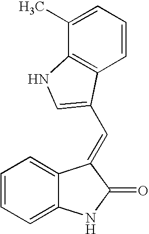 | 8.4 |
| 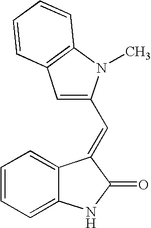 | 7 |
| 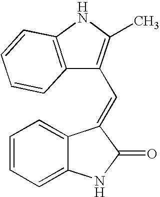 | 6.1 |
| 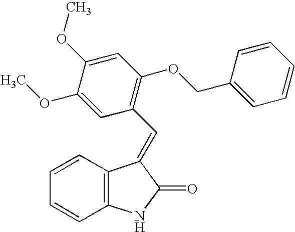 | 6.1 |
| 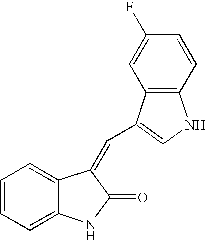 | 7 |
| 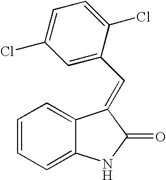 | 8.5 |
| 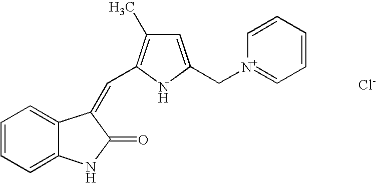 | 8.2 |
| 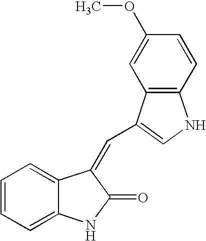 | 8 |
| 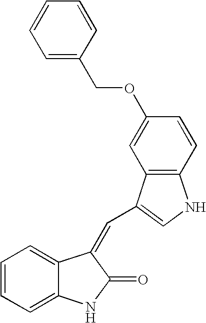 | 8 |
| 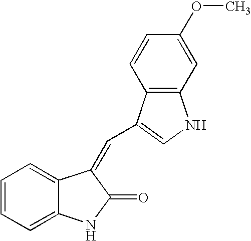 | 9.7 |
| 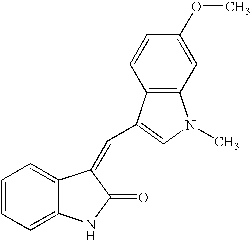 | 8.2 |
| 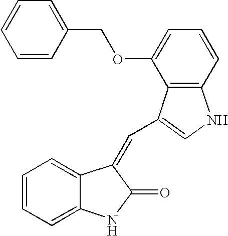 | 7 |
| 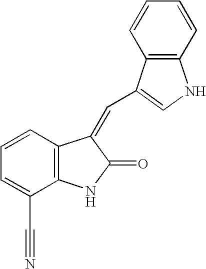 | 9 |
| 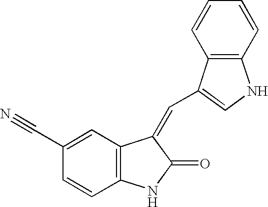 | 9 |
| 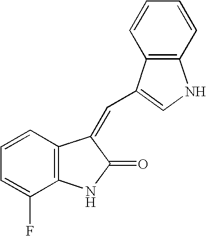 | 9.5 |
| 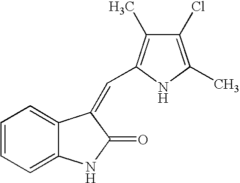 | 9 |
| 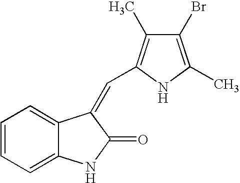 | 7.1 |
| 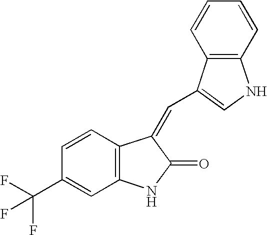 | 7.7 |
| 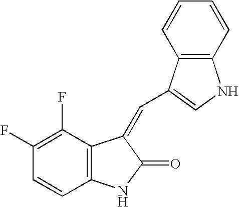 | 8.4 |
| 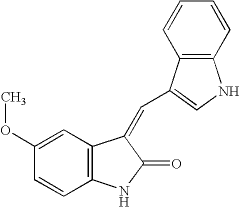 | 7.5 |
| 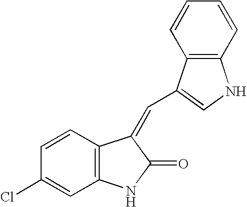 | 7.2 |
| 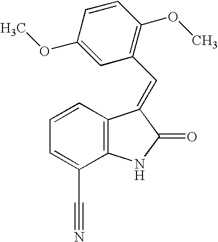 | 7.5 |
| 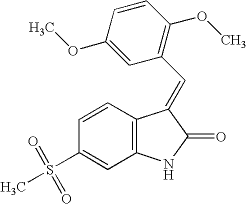 | 6.1 |
| 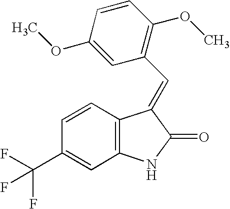 | 8 |
| 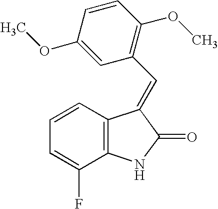 | 6.3 |
| 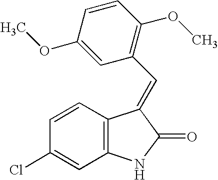 | 7.5 |
| 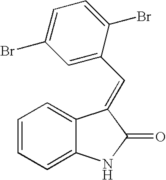 | 7.4 |
| 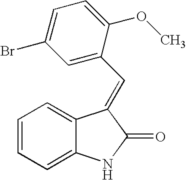 | 8 |
| 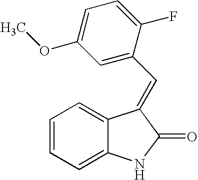 | 7.2 |
| 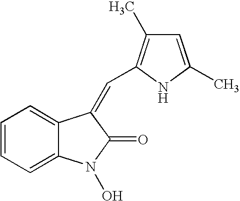 | 8 |
| 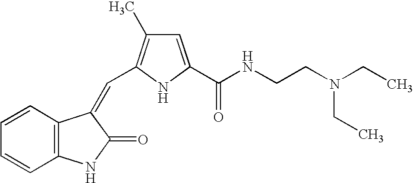 | 7.4 |
| 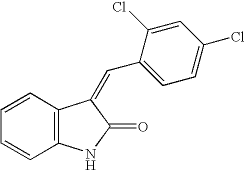 | 6.7 |
| 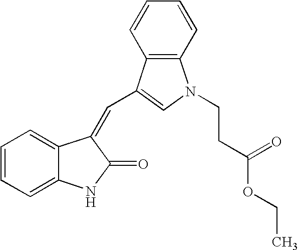 | 6.7 |
| 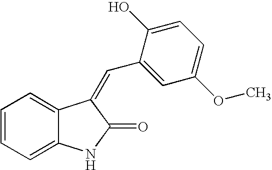 | 6.7 |
| 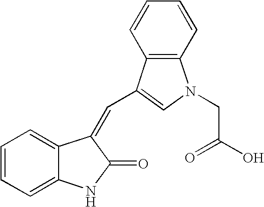 | 7.4 |
| 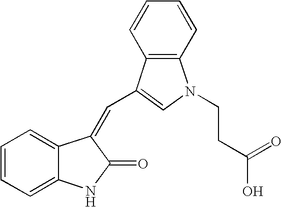 | 6.7 |
| 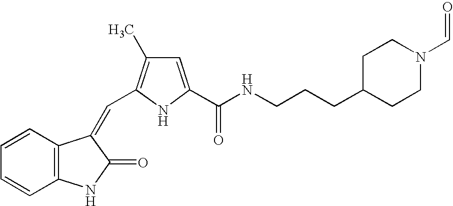 | 7.3 |
| 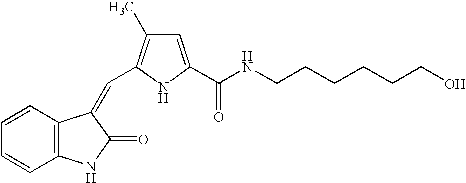 | 7 |
| 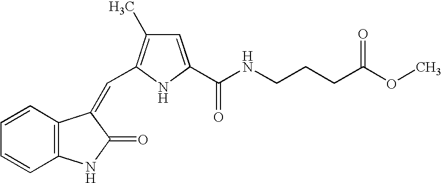 | 7.3 |
| 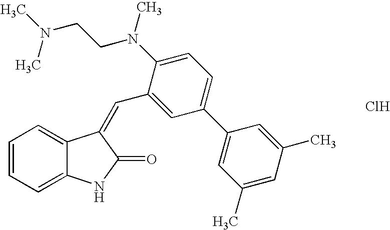 | 7.2 |
| 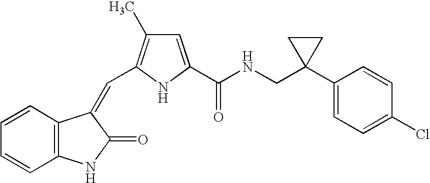 | 7.2 |
| 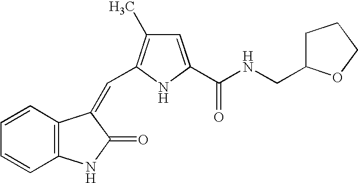 | 7.7 |
| 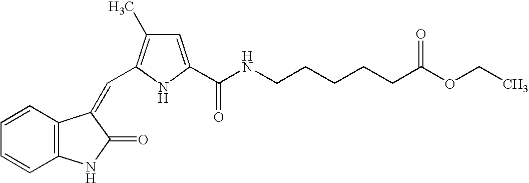 | 7.1 |
| 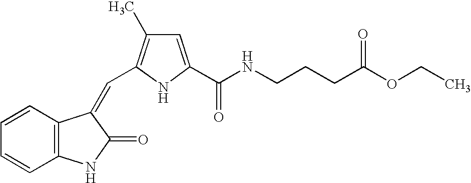 | 7.4 |
| 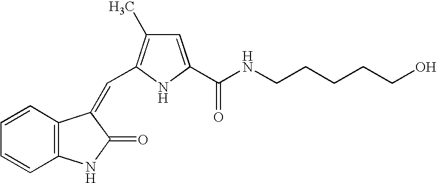 | 7.2 |
| 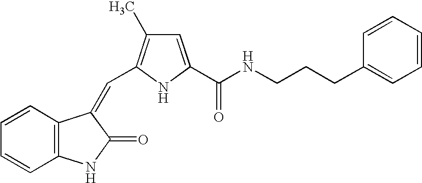 | 7.3 |
| 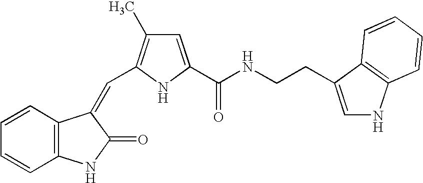 | 7.4 |
| 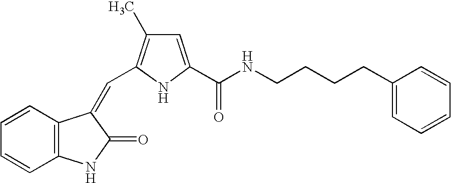 | 7 |
| 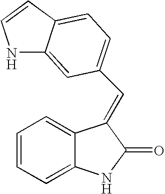 | 8.2 |
| 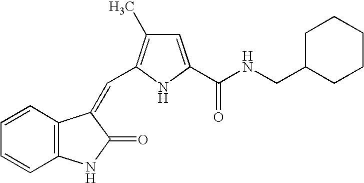 | 7.5 |
| 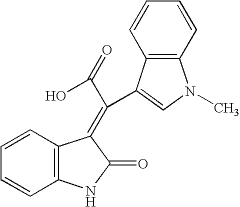 | 6.1 |
| 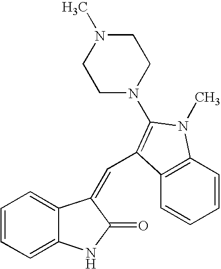 | 7.1 |
| 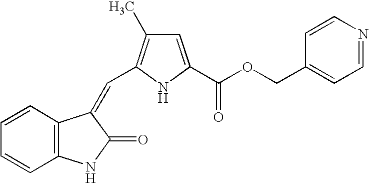 | 7.5 |
| 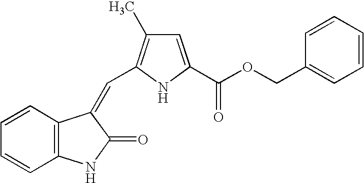 | 7.7 |
| 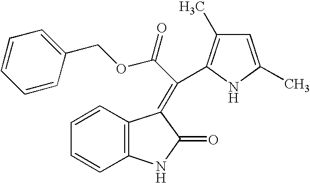 | 7.7 |
| 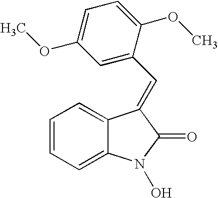 | 8 |
| 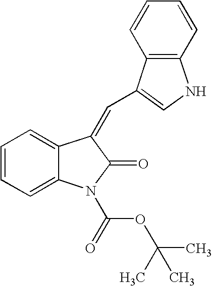 | 9 |
| 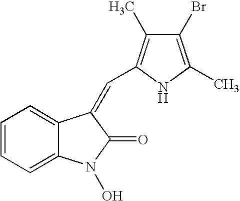 | 9 |
| 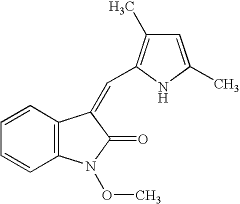 | 8 |
| 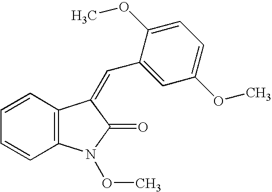 | 7.7 |

### Example 8

Differential Up- and Downregulation of Genes in EAE Mice Treated with Compound A Compared to Untreated Controls

The mouse spleen is one of the organs where autoimmune cells accumulate, differentiate and maturate prior to entering the CNS via the bloodstream. The process of activation and maturation of inflammatory cells, primarily T-cells, involves activation of antigen presenting cells, e.g. macrophages, which present the antigen to a T-cell receptor and thereby activate a cascade of adhesion molecules, proteases, cytokines and chemokines (O. Neuhaus et al., Trends Pharmacol. Sci. 24, 2003, pp. 131-138). Oligonucleotide DNA arrays have been used to obtain an image of the differences in gene expression in inflammatory cells in the spleen with or without treatment with Compound 226.

RNA Extraction, Probe Synthesis and Hybridization

SJL mice immunized with an encephalitogenic epitope of the proteolipid protein (PLP₁₃₉₋₁₅₃) were dosed daily with compound 226 from day 0 as described in example 1. Control mice were given suspension vehicle as described in example 1. On day 7 after treatment, the mice (both treated and control) were sacrificed and their spleens were excised, submerged into the RNA storage solution RNA-later (Sigma-Aldrich, St. Louis, USA) and stored at −80° C. Total RNA was extracted from the mouse spleens using the TRIZOL extraction procedure according to the manufacturer's protocol followed by DNAse treatment (Invitrogen, Carlsbad, Calif., USA). The RNA quality was visualized by agarose gel electrophoresis. Ten μg total RNA was converted into purified ³³P-labelled cDNA probe according to the Atlas Pure Total RNA labelling protocol (BD Biosciences, Palo Alto, Calif., USA).

Labelled probes (10×10⁶ CPM) were denatured at 95° C. for 2 minutes and cooled at 4° C. for 2 minutes prior to hybridization to oligonucleotide arrays comprising about 5.000 mouse genes in duplicate dots (Atlas Plastic Mouse 5K MicroArray, BD Biosciences, Palo Alto, Calif., USA). Arrays were hybridized for 16 h at 60° C. with rotation according to the manufacturer's protocol. After hybridization and appropriate washing steps, the arrays were imaged using phospho imager technology together with a STORM 860 scanner (Molecular Dynamics, Sunnyvale, Calif., USA). Subsequently, the images were analysed using the Atlas Image 2.7 software (BD Biosciences, Palo Alto, Calif., USA).

Results

Approximately 1.5% of the genes (75 genes) located on the Atlas MicroArray were differentially expressed when arrays from the spleen cDNA of treated and control mice were compared. Several immune response genes were found to be upregulated (class II major histocompatilibility complex [NM_{—}010379, NM_{—}010388]), immunoglobulin-associated beta [NM_{—}008339], transforming growth factor beta-3 [NM_{—}009368], Fc receptor (IgE, high affinity I, gamma polypeptide [NM_{—}010185]) and beta-2 microglobulin [NM_{—}009735]) in the treated group. Genes encoding adhesion molecules (lymphocyte antigens 6 locus D [NM_{—}010742], lymphocyte antigens 6 locus E [NM_{—}008529] and lymphocyte antigens 84 [NM_{—}010743]) were found to be upregulated in the treated group. Genes encoding the interleukin 1 receptor [M20658] and the interleukin 10 receptor alpha [L12120] together with genes encoding tumor necrosis factor related proteins (TNF receptor [M59378] and TNF receptor-associated factor 1 [NM_{—}009421]) were downregulated in the treated group. Upregulation of genes encoding adhesion molecules as well as downregulation of genes involved in the inflammatory response (interleukin and TNF related genes) suggests that maturation and activation of inflammatory cells in the spleen is reduced as is their migration into the bloodstream and further into the CNS. This is in agreement with the observation that treatment with Compound 226 delays the onset of EAE.

Genes related to anti-apoptosis (Bcl-2 like protein [U51279] and elastase [NM_{—}015779]) were found to be downregulated in the treated group allowing apoptosis of inflammatory cells to take place.

Genes having a proposed role in re-myelination were found to be differentially expressed (complement factor [NM_{—}009777], myelin transcription factor [AF004294], fibroblast growth factor 15 [AF007268]) in treated and control mice, indicating a role of compound 226 in the regression of symptoms of EAE and consequent remyelination of axons.

The figures in square brackets above refer to the GenBank Accession No. of the respective genes.

### Example 9

Activity of Oxindole Compounds on Oral Administration

Materials and Methods

Compounds: The compounds shown in Table 7 below.

All compounds were dosed p.o. at 50 mg/kg. In other respects, immunization and treatment of the mice was carried out as described in example 4.

Results

A series of oxindole compounds were administered orally in the EAE model. The results are shown in Table 7 below. The inhibitory effects shown in the table are significant (p<0.05). The oxindole compounds inhibit EAE with varying effects ranging from 32% to 67% inhibition.

Conclusion

Several oxindole analogues have the potential to inhibit EAE disease when given orally.

TABLE 7
Inhibition of EAE with p.o. dosed oxindole compounds

| | | *Inhibition |
|---|---|---|
| Compound | Structure | (%) |
| 57 | 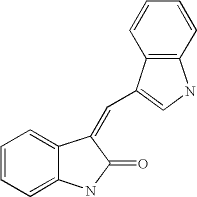 | 32 |
| 110 | 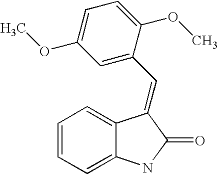 | 47 |
| 110 | 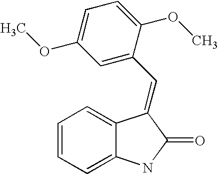 | 67** |
| 263 | 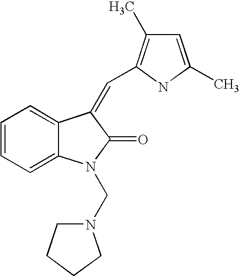 | 41 |
| 264 | 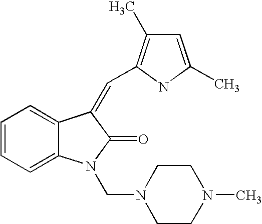 | 56 |
| 265 | 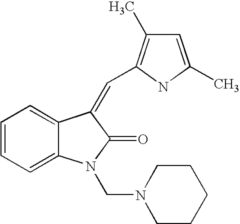 | 63 |
| 32 | 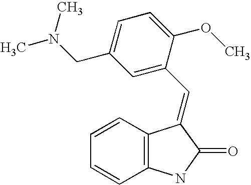 | 40 |
| 211 | 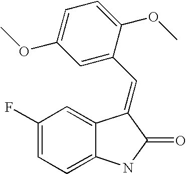 | 37** |

*Area-under-curve of the disease score. A suspension vehicle group was included in all experiments. AUC of the disease score were calculated for all groups and the Mann-Withney test was used to compare the treated groups with the suspension vehicle group.
**In these experiments compounds 110 and 211 were dosed in a suspension vehicle adjusted to pH 4 by addition of aqueous HCl.

1. Use of a compound of general formula I 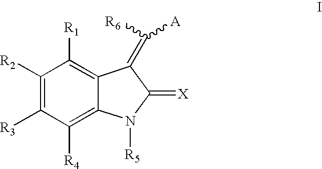
wherein
R₁, R₂, R₃ and R₄ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂;
X is O or S;
R₅ is hydrogen, hydroxy, C₁₋₆ alkyl, C₂₋₆ alkenyl, C₂₋₆ alkynyl, cycloalkyl, heterocyclyl, aryl, heteroaryl, C₁₋₆ alkoxy, carbonyl, carboxy, amido, thioamido, guanyl, guanidinyl, ureidyl, sulfonyl, trihalomethanesulfonyl, —C(O)OR₁₄, —C(O)R₁₄, wherein R₁₄ is hydrogen, C₁₋₆ alkyl, C₂₋₆ alkenyl, C₂₋₆ alkynyl, cycloalkyl or aryl;
R₆ is hydrogen, C₁₋₆ alkyl, cycloalkyl, aryl, heteroaryl, heterocyclyl, halogen, —OR₇, —C(O)R₇, —C(O)OR₇, —NR₇R₈, S(O)₂NR₇R₈, wherein R₇ and R₈ are independently hydrogen, C₁₋₆ alkyl, aryl or heterocyclyl, said C₁₋₆ alkyl or heterocyclyl being optionally substituted by heterocyclyl, —OR₇, —C(O)R₇ or C(O)OR₇, the zigzag line indicating that the group denoted R₆ is present as the E- or Z-isomer;
A is phenyl or a monocyclic or bicyclic heteroaryl ring, optionally substituted at one or more positions with hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂ or S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; the zigzag line indicating that the group denoted A is present as the E- or Z-isomer;
or pharmaceutically acceptable salts thereof, for the preparation of a medicament for the prevention, treatment or amelioration of multiple sclerosis, or to delay of the onset of or reduce the relapse rate in multiple sclerosis.

2. The use according to claim 1 wherein, in the compound of formula I,
X is O or S;
R₁, R₂, R₃ and R₄ are the same or different and independently selected from the group consisting of hydrogen, C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, S(O)R₁₈, S(O)₂R₁₈, S(O)₂NR₁₈R₁₉, S(O)₃R₁₈, SR₁₈, NO₂, NR₁₈R₁₉, OR₁₈, CN, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆ alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆ alkyl or aryl, and n is 0-3;
A is phenyl or a monoclyclic or bicyclic heteroaryl ring selected from the group consisting of pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,3,4-thiatriazole, 1,2,3,5-thiatriazole, tetrazole and indole, optionally substituted at one or more positions with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OH, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈, R₁₉ and n are as indicated above;
R₅ is hydrogen or C₁₋₆ alkyl; and
R₆ is hydrogen.

3. The use of claim 1 wherein, in the compound of formula I, R₅ is hydrogen.

4. The use of claim 1 wherein, in the compound of formula I, X is oxygen.

5. The use of claim 1 wherein, in the compound of formula I, R₁, R₂, R₃ and R₄ are the same or different and independently selected from hydrogen and C₁₋₆ alkyl.

6. The use of claim 1 wherein, in the compound of formula I, R₆ is hydrogen or COOH.

7. The use of any of claims 1-6 wherein, in the compound of formula I, A is pyrrole, phenyl or indole, said pyrrole, phenyl or indole being optionally substituted at one or more positions with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OH, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈, R₁₉ and n are as indicated in claim 2.

8. The use of claim 7 wherein, in the compound of formula I, A is pyrrole substituted at position 3 and 5 with C₁₋₆ alkyl, or at position 3 with C₁₋₆ alkyl and at position 5 with CH₂OH, COOH or a sugar residue, or at position 3 and 5 with C₁₋₆ alkyl and at position 4 with halogen, or at position 5 with C(O)O—C₁₋₆ alkyl, and at position 3 with C₁₋₆ alkyl.

9. The use of claim 7 wherein, in the compound of formula I, A is phenyl substituted at position 2 and 5 with C₁₋₆ alkyl, C₁₋₆ alkoxy, halogen, C₁₋₆ alkyl-NR₂₆R₂₇, NH—C₁₋₆ alkyl-NR₂₆R₂₇ or O—C₁₋₆ alkyl-NR₂₆R₂₇, wherein R₂₆ and R₂₇ are independently hydrogen or C₁₋₆ alkyl or, together with the nitrogen atom to which they are attached, form a heteroaryl or heterocyclic ring.

10. The use of claim 7 wherein, in the compound of formula I, A is indole.

11. The use of claim 7 wherein the compound is 3-(3,5-dimethyl-1H-pyrrol-2-yl-methylene-1,3-dihydro-indol-2-one.

12. The use of claim 7 wherein the compound is 3-(2,5-dimethoxy-benzylidene)-1,3-dihydroindol-2-one.

13. The use of claim 7 wherein the compound is 3-(1H-indol-3-ylmethylene)-1,3-dihydroindol-2-one.

14. The use of claim 1, wherein the compound is a compound of formula II 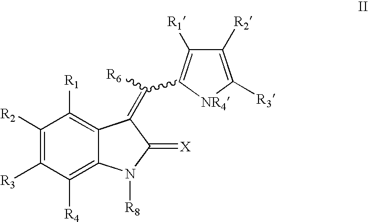
wherein R₁, R₂, R₃, R₄, R₆ and X are as indicated in claim 1,
R₈ and R₄′ are independently hydrogen, hydroxy, C₁₋₆alkyl, C₂₋₆alkenyl, C₂₋₆alkynyl, cycloalkyl, heterocyclyl, aryl, heteroaryl, C₁₋₆alkoxy, carbonyl, carboxy, amido, thioamido, guanyl, guanidinyl, ureidyl, sulfonyl, trihalomethanesulfonyl, —PO(OR)(OR′), wherein R and R′ are independently selected from hydrogen or C₁₋₆ alkyl, —OR₁₀, —C(O)R₁₀, —C(O)OR₁₀, OC(O)R₁₀, —OC(O)OR₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁, —S(O)OR₁₀ and CH₂-aryl-OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —OC(O)OR₁₂, —NR₁₂R₁₃, —C(O)NR₁₂R₁₃, —OC(O)NR₁₀R₁₁, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂, —S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; —C(R₂₄R₂₅)—OR₁₆ or —OC(O)R₁₆, wherein R₁₆ is hydrogen, C₁₋₆ alkyl, aralkyl, acyl or —PO(OR)(OR′), —C(R₂₄R₂₅)—NR₂₆R₂₇, wherein R₂₄ is hydrogen, C₁₋₆ alkyl or aryl, R₂₅ is hydrogen, and R₂₆ and R₂₇ are independently hydrogen or C₁₋₆ alkyl or, together with the nitrogen atom to which they are attached, form a heteroaryl or heteroaryl ring optionally substituted with hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂ or —S(O)NH₂; —NR₂₀R₂₁, —O(CH₂)ₘNR₂₀R₂₁, —N(CH₂)ₘNR₂₀R₂1, —O(CH₂)ₘC(O)R₂₂, —N(CH₂)ₘC(O)R₂₂, wherein m is 0, 1, 2 or 3, R₂₀ and R₂₁ are the same or different and independently selected from the group consisting of hydrogen, C₁₋₆ alkyl, cycloalkyl, aryl, carbonyl, acetyl, trihalomethylcarbonyl, carboxy, sulfonyl or trihalomethanesulfonyl, or R₂₀ and R₂₁ together with the nitrogen atom to which they are attached form a heterocyclic or heteroaryl ring, and R₂₂ is hydroxy, C₁₋₆ alkoxy, aryloxy, amino, hydroxylamino, carboxy or —NR₂₀R₂₁, wherein R₂₀ and R₂₁ are as indicated above; and
R₁′, R₂′ and R₃′ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, OC(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂.

15. The use of claim 14 wherein, in the compound of formula II, R₁, R₂, R₃, R₄, R₆ and X are as indicated in claim 2, and R₁′, R₂′ and R₃′ are the same or different and independently selected from the group consisting of with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆ alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆ alkyl or aryl, and n is 0-3.

16. The use of claim 15 wherein, in the compound of formula II, R₁, R₂, R₃ and R₄ are the same or different and independently selected from hydrogen, halogen and C₁₋₆ alkyl, or R₂ is hydroxy or heteroaryl, such as pyridyl, or a group C(O)R₂₀, wherein R₂₀ is heteroaryl, such as pyridyl or thienyl, and R₁, R₃ and R₄ are hydrogen.

17. The use of claim 15 wherein, in the compound of formula II, R₁′, R₂′ and R₃′ are the same or different and independently selected from hydrogen, halogen, C₁₋₆ alkyl, C₁₋₆ alkoxy, CH₂OH, C(O)OR₁₈ or C(O)NR₁₈R₁₉, wherein R₁₈ and R₁₉ are as defined in claim 15.

18. The use of claim 14 or 15 wherein, in the compound of formula II, R₁′ and R₃′ are both C₁₋₆ alkyl, in particular methyl, and R₂′ is hydrogen, or wherein R₁′ is C₁₋₆ alkyl and R₃′ is C₁₋₆ alkoxy, CH₂OH, C(O)OR₁₈ or C(O)NR₁₈R₁₉, wherein R₁₈ and R₁₉ are as defined in claim 15, or wherein R₁′ and R₃′ are both C₁₋₆ alkyl, in particular methyl, and R₂′ is halogen, in particular chloro or bromo, or wherein R₁′ is C₁₋₆ alkyl and R₃′ is C(O)O—C₁₋₆ alkyl, or wherein R₁′ is C₁₋₆ alkyl and R₃′ is C(O)NH—C₁₋₆alkyl substituted with hydroxy.

19. The use of claim 14 wherein, in the compound of formula II, R₈ and R₄′ are independently hydrogen, hydroxy, —PO(OR)(OR′), —OR₁₀, —C(O)OR₁₀, —C(O)NR₁₀R₁₁, —C(O)R₁₄, —C(R₂₄R₂₅)OR₁₆, —OC(O)R₁₆ or —C(R₂₄R₂₅)NR₂₆R₂₇, wherein R, R′, R₁₀, R₁₁, R₁₄, R₁₆, R₂₄, R₂₅, R₂₆, R₂₇ are as defined in claim 14.

20. The use of claim 14 wherein the compound is selected from the group consisting of
3-(3,5-Dimethyl-1H-pyrrol-2-ylmethylene)-1,3-dihydro-indol-2-one (Compound 226)
4-methyl-5-(2-oxo-1,2-dihydro-indol-3-ylidenemethyl)-1H-pyrrole-2-carboxylic acid ethyl ester (Compound 01)
4-methyl-5-(2-oxo-1,2-dihydro-indol-3-ylidenemethyl)-1H-pyrrole-2-carboxylic acid (2-hydroxy-ethyl)-amide (Compound 02)
3-(5-hydroxymethyl-3-methyl-1H-pyrrol-2-ylmethylene)-1,3-dihydro-indol-2-one (Compound 03)
1-[4-methyl-5-(2-oxo-1,2-dihydro-indol-3-ylidenemethyl)-1H-pyrrol-2-ylmethyl)-pyrridinum; chloride (Compound 04)
4-methyl-5-(2-oxo-1,2-dihydro-indol-3-ylidenemethyl)-1H-pyrrole-2-carboxylic acid (Compound 05)
4-methyl-5-(2-oxo-1,2-dihydro-indol-3-ylidenemethyl)-1H-pyrrole-2-carboxylic acid (2-diethylamino-ethyl)-amide (Compound 06)
4-methyl-5-(2-oxo-1,2-dihydro-indol-3-ylidenemethyl)-1H-pyrrole-2-carboxylic acid (2-methoxy-ethyl)-amide (Compound 07)
4-methyl-5-(2-oxo-1,2-dihydro-indol-3-ylidenemethyl)-1H-pyrrole-2-carboxylic acid [3-(1-formyl-piperidin-4-yl)-propyl]-amide (Compound 08)
4-{[4-methyl-5-(2-oxo-1,2-dihydro-indol-3-ylidenemethyl)-5H-pyrrole-2-carbony-4]-amino}-butyric acid methyl ester (Compound 09)
4-methyl-5-(2-oxo-1,2-dihydro-indol-3-ylidenemethyl)-1H-pyrrole-2-carboxylic acid (6-hydroxy-hexyl)-amide (Compound 10)
4-methyl-5-(2-oxo-1,2-dihydro-indol-3-ylidenemethyl)-1H-pyrrole-2-carboxylic acid cyclohexylmethyl-amide (Compound 11)
4-methyl-5-(2-oxo-1,2-dihydro-indol-3-ylidenemethyl)-H-pyrrole-2-carboxylic acid (4-hydroxy-butyl)-amide (Compound 12)
6-{[4-methyl-5-(2-oxo-1,2-dihydro-indol-3-ylidenemethyl)-1H-pyrrole-2-carbonyl]-amino}-hexanoic acid ethyl ester (Compound 13)
4-methyl-5-(2-oxo-1,2-dihydro-indol-3-ylidenemethyl)-1H-pyrrole-2-carboxylic acid (tetrahydro-furan-2-ylmethyl)-amide (Compound 14)
4-methyl-5-(2-oxo-1,2-dihydro-indol-3-ylidenemethyl)-1H-pyrrole-2-carboxylic acid [2-(1H-indol-3-yl)-ethyl]-amide (Compound 15)
4-methyl-5-(2-oxo-1,2-dihydro-indol-3-ylidenemethyl)-1H-pyrrole-2-carboxylic acid (3-phenyl-propyl)-amide (Compound 16)
4-methyl-5-(2-oxo-1,2-dihydro-indol-3-ylidenemethyl)-1H-pyrrole-2-carboxylic acid (4-phenyl-butyl)-amide (Compound 17)
4-methyl-5-(2-oxo-1,2-dihydro-indol-3-ylidenemethyl)-1H-pyrrole-2-carboxylic acid (5-hydroxy-pentyl)-amide (Compound 18)
4-{[4-methyl-5-(2-oxo-1,2-dihydro-indol-3-ylidenemethyl)-1H-pyrrole-2-carbonyl]-amino}-butyric acid ethyl ester (Compound 19)
4-methyl-5-(2-oxo-1,2-dihydro-indol-3-ylidenemethyl)-1H-pyrrole-2-carboxylic acid [1-(4-chloro-phenyl)-cyclopropylmethyl]-amide (Compound 20)
4-methyl-5-(2-oxo-1,2-dihydro-indol-3-ylidenemethyl)-1H-pyrrole-2-carboxylic acid benzyl ester (Compound 21)
3-(4-bromo-3,5-dimethyl-1H-pyrrol-2-ylmethylene)-1,3-dihydro-indol-2-one (Compound 22)
3-(4-chloro-3,5-dimethyl-1H-pyrrol-2-ylmethylene)-1,3-dihydro-indol-2-one (Compound 23)
3-(3,5-dimethyl-1H-pyrrol-2-ylmethylene)-1-(4-methoxy-benzyl)-1,3-dihydro-indol-2-one (Compound 41)
3-(3,5-Dimethyl-1H-pyrrol-2-ylmethylene)-1-methyl-1,3-dihydro-indol-2-one (Compound 42)
acetic acid 3-(3,5-dimethyl-1H-pyrrol-2-ylmethylene)-2-oxo-2,3-dihydro-indol-1-ylmethyl ester (Compound 43)
3-(3,5-dimethyl-1H-pyrrol-2-ylmethylene)-1-hydroxy-1,3-dihydro-indol-2-one (Compound 45)
3-(4-bromo-3,5-dimethyl-1H-pyrrol-2-ylmethylene)-1-hydroxy-1,3-dihydro-indol-2-one (Compound 46)
3-(3,5-dimethyl-1H-pyrrol-2-ylmethylene)-1-methoxy-1,3-dihydro-indol-2-one (Compound 49)
acetic acid 3-(3,5-dimethyl-1H-pyrrol-2-ylmethylene)-2-oxo-2,3-dihydro-indol-1-yl ester (Compound 51)
2-{3-[3-(3,5-dimethyl-1H-pyrrol-2-ylmethylene)-2-oxo-2,3-dihydro-indol-1-yloxy]-propyl}-isoindole-1,3-dione (Compound 52)
2,4-Dimethyl-5-(2-oxo-1,2-dihydro-indol-3-ylidenemethyl)-1H-pyrrole-3-carboxylic acid (2-diethylamino-ethyl)-amide (Compound 227)
5-(5-Fluoro-2-oxo-1,2-dihydro-indol-3-ylidenemethyl)-2,4-dimethyl-1H-pyrrole-3-carboxylic acid (2-diethylamino-ethyl)-amide (Compound 228)
(3,5-dimethyl-1H-pyrrol-2-yl)-(2-oxo-1,2-dihydro-indol-3-ylidene)-acetic acid (Compound 229)
3-[2,4-Dimethyl-5-(2-oxo-1,2-dihydro-indol-3-ylidenemethyl)-1H-pyrrol-3-yl]-propionic acid (Compound 230)
3-(3,5-dimethyl-1H-pyrrol-2-ylmethylene)-4-iodo-1,3-dihydro-indol-2-one (Compound 231)
3-(3,5-dimethyl-1H-pyrrol-2-ylmethylene)-5-methoxy-1,3-dihydro-indol-2-one (Compound 232)
5-chloro-3-(3-methoxy-1H-pyrrol-2-ylmethylene)-1,3-dihydro-indol-2-one (Compound 233)
3-(3-methoxy-1H-pyrrol-2-ylmethylene)-1,3-dihydro-indol-2-one (Compound 234)
3-[5-(4-chloro-2-oxo-1,2-dihydro-indol-3-ylidenemethyl)-2,4-dimethyl-1H-pyrrol-3-yl]-propionic acid (Compound 235)
4-chloro-3-(3,5-dimethyl-1H-pyrrol-2-ylmethylene)-1,3-dihydro-indol-2-one (Compound 236)
4-chloro-3-(3-methoxy-1H-pyrrol-2-ylmethylene)-1,3-dihydro-indol-2-one (Compound 237)
3-(3,5-dimethyl-1H-pyrrol-2-ylmethylene)-2-oxo-2,3-dihydro-1H-indole-4-carboxylic acid (Compound 238)
3-(3,5-dimethyl-1H-pyrrol-2-ylmethylene)-5-pyridin-3-yl-1,3-dihydro-indol-2-one (Compound 239)
3-(3,5-dimethyl-1H-pyrrol-2-ylmethylene)-5-pyridin-3-yl-1,3-dihydro-indol-2-one; methanesulfonic acid (Compound 240)
5-pyridin-3-yl-3-(1H-pyrrol-2-ylmethylene)-1,3-dihydro-indol-2-one (Compound 241)
5-pyridin-3-yl-3-(1H-pyrrol-2-ylmethylene)-1,3-dihydro-indol-2-one; methanesulfonic acid (Compound 242)
3-(3,5-dimethyl-1H-pyrrol-2-ylmethylene)-5-hydroxy-1,3-dihydro-indol-2-one (Compound 243)
3-(3,5-dimethyl-1H-pyrrol-2-ylmethylene)-5-fluoro-1,3-dihydro-indol-2-one (Compound 244)
3-(1-methyl-1H-indol-2-ylmethylene)-1,3-dihydro-indol-2-one (Compound 245)
2,4-dimethyl-5-(2-oxo-1,2-dihydro-indol-3-ylidenemethyl)-1H-pyrrole-3-carboxylic acid ethyl ester (Compound 246)
4-methyl-5-(2-oxo-1,2-dihydro-indol-3-ylidenemethyl)-1H-pyrrole-2-carboxylic acid pyridin-4-ylmethyl ester (Compound 263)
(3,5-dimethyl-1H-pyrrol-2-yl)-(2-oxo-1,2-dihydro-indol-3-ylidene)-acetic acid benzyl ester (Compound 264)
3-(3,5-dimethyl-1H-pyrrol-2-ylmethylene)-1-pyrrolidin-1-ylmethyl-1,3-dihydro-indol-2-one (Compound 266)
3-(3,5-dimethyl-1H-pyrrol-2-ylmethylene)-1-(4-methyl-piperazin-1-ylmethyl)-1,3-dihydro-indol-2-one (Compound 267) and
3-(3,5-dimethyl-1H-pyrrol-2-ylmethylene)-1-piperidin-1-ylmethyl-1,3-dihydro-indol-2-one (Compound 268)

21. The use of claim 1, wherein the compound is a compound of formula III 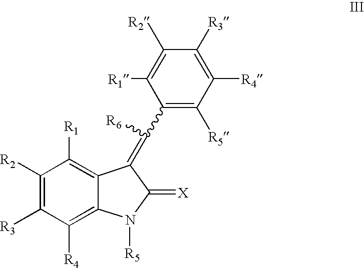
wherein R₁, R₂, R₃, R₄, R₅, R₆ and X are as indicated in claim 1, and
R₁″, R₂″, R₃″, R₄″ and R₅″ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, halogen, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂.

22. The use of claim 21 wherein, in the compound of formula III, R₂, R₃, R₄, R₅, R₆ and X are as indicated in claim 2, and R₁″, R₂″, R₃″, R₄″ and R₅″ are the same or different and independently selected from the group consisting of with C₁₋₁₀alkyl, C₁₋₁₀alkoxy, aryl, heteroaryl, aryloxy, C₁₋₁₀alkylaryl, C₁₋₁₀alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆alkyl or aryl, and n is 0-3.

23. The use of claim 22 wherein, in the compound of formula III, R₂″ and R₅″ are the same or different and independently are C₁₋₆alkyl, in particular methyl, or C₁₋₆alkoxy, in particular methoxy, or halogen, in particular chloro or bromo.

24. The use of claim 21 wherein, in the compound of formula III, R₅ is hydrogen, hydroxy, C(O)R₁₄ or C(O)OR₁₄, wherein R₁₄ is as defined in claim 1.

25. The use of claim 21, wherein the compound is selected from the group consisting of
3-(2,5-dimethoxy-benzylidene)-1,3-dihydro-indol-2-one (Compound 110)
3-(5-dimethylaminomethyl-2-methoxy-benzylidene)-1,3-dihydro-indol-2-one (Compound 32)
3-{2-[(2-dimethylamino-ethyl)-methyl-amino]-5-methoxy-benzylidene}-1,3-dihydro-indol-2-one (Compound 33)
3-{4-[(2-dimethylamino-ethyl)-methyl-amino]-3′,5′-dimethyl-biphenyl-3-ylmethylene}-1,3-dihydro-indol-2-one (Compound 34)
3-(2-dimethylaminomethyl-benzylidene)-1,3-dihydro-indol-2-one (Compound 35)
3-[2-(2-diethylamino-ethoxy)-5-methoxy-benzylidene]-1,3-dihydro-indol-2-one (Compound 36)
3-[2-(2-diethylamino-ethoxy)-5-methoxy-benzylidene]-1,3-dihydro-indol-2-one; hydrochloride (Compound 37)
3-[5-methoxy-2-(2-morpholin-4-yl-ethoxy)-benzylidene]-1,3-dihydro-indol-2-one (Compound 38)
3-[5-methoxy-2-(2-piperidin-1-yl-ethoxy)-benzylidene]-1,3-dihydro-indol-2-one (Compound 39)
1-acetyl-3-(2,5-dimethoxy-benzylidene)-1,3-dihydro-indol-2-one (Compound 44)
3-(2,5-dimethoxy-benzylidene)-1-hydroxy-1,3-dihydro-indol-2-one (Compound 48)
3-(2,5-dimethoxy-benzylidene)-1-methoxy-1,3-dihydro-indol-2-one (Compound 50)
3-(phenyl-4-tolyl-methylene)-1,3-dihydro-indol-2-one (Compound 53)
3-[bis-(4-methoxy-phenyl)-methylene]-1,3-dihydro-indol-2-one (Compound 54)
3-[1-(2,5-dimethoxy-phenyl)-ethylidene]-1,3-dihydro-indol-2-one (Compound 55)
3-(4-hydroxy-3,5-dimethyl-benzylidene)-1,3-dihydro-indol-2-one (Compound 95)
3-(3,5-di-tert-butyl-4-hydroxy-benzylidene)-1,3-dihydro-indol-2-one (Compound 96)
3-(4-bromo-benzylidene)-1,3-dihydro-indol-2-one (Compound 97)
3-(2-methyl-benzylidene)-1,3-dihydro-indol-2-one (Compound 98)
3-(2,4-dimethyl-benzylidene)-1,3-dihydro-indol-2-one (Compound 99)
3-(2,6-dichloro-benzylidene)-1,3-dihydro-indol-2-one (Compound 100)
3-(3,4-dichloro-benzylidene)-1,3-dihydro-indol-2-one (Compound 101)
3-(4-chloro-benzylidene)-1,3-dihydro-indol-2-one (Compound 102)
3-(2,4-dimethyl-benzylidene)-1,3-dihydro-indol-2-one (Compound 103)
3-(2,5-dimethyl-benzylidene)-1,3-dihydro-indol-2-one (Compound 104)
3-(2,6-dimethoxy-benzylidene)-1,3-dihydro-indol-2-one (Compound 105)
3-benzylidene-1,3-dihydro-indol-2-one (Compound 106)
3-(4-methoxy-benzylidene)-1,3-dihydro-indol-2-one (Compound 107)
3-(2,4-dimethoxy-benzylidene)-1,3-dihydro-indol-2-one (Compound 108)
3-(3,5-dimethoxy-benzylidene)-1,3-dihydro-indol-2-one (Compound 109)
3-(3,4-dimethyl-benzylidene)-1,3-dihydro-indol-2-one (Compound 111)
3-naphtalen-2-ylmethylene-1,3-dihydro-indol-2-one (Compound 112)
3-naphtalen-1-ylmethylene-1,3-dihydro-indol-2-one (Compound 113)
3-(2,3-dimethoxy-benzylidene)-1,3-dihydro-indol-2-one (Compound 114)
3-(3-nitro-benzylidene)-1,3-dihydro-indol-2-one (Compound 115)
3-(2-fluoro-benzylidene)-1,3-dihydro-indol-2-one (Compound 116)
3-(3-methyl-benzylidene)-1,3-dihydro-indol-2-one (Compound 117)
3-(3-fluoro-benzylidene)-1,3-dihydro-indol-2-one (Compound 118)
3-(4-fluoro-benzylidene)-1,3-dihydro-indol-2-one (Compound 119)
3-anthracen-9-ylmethylene-1,3-dihydro-indol-2-one (Compound 120)
3-(5-bromo-2-hydroxy-benzylidene)-1,3-dihydro-indol-2-one (Compound 121)
3-(2-hydroxy-benzylidene)-1,3-dihydro-indol-2-one (Compound 122)
5-chloro-3-(4-isopropyl-benzylidene)-1,3-dihydro-indol-2-one (Compound 123)
5-chloro-3-(4-dimethylamino-benzylidene)-1,3-dihydro-indol-2-one (Compound 124)
5-chloro-3-(3,4-dichloro-benzylidene)-1,3-dihydro-indol-2-one (Compound 125)
5-chloro-3-(2,5-dimethoxy-benzylidene)-1,3-dihydro-indol-2-one (Compound 126)
5-Chloro-3-(2-chloro-benzylidene)-1,3-dihydro-indol-2-one (Compound 127)
5-chloro-3-(2,3-dimethoxy-benzylidene)-1,3-dihydro-indol-2-one (Compound 128)
5-Chloro-3-(2,6-dichloro-benzylidene)-1,3-dihydro-indol-2-one (Compound 129)
5-Chloro-3-(2-hydroxy-benzylidene)-1,3-dihydro-indol-2-one (Compound 130)
5-chloro-3-(4-methoxy-benzylidene)-1,3-dihydro-indol-2-one (Compound 131)
5-chloro-3-(4-chloro-benzylidene)-1,3-dihydro-indol-2-one (Compound 132)
5-chloro-3-naphtalen-1-ylmethylene-1,3-dihydro-indol-2-one (Compound 133)
5-chloro-3-(4-bromo-benzylidene)-1,3-dihydro-indol-2-one (Compound 134)
5-chloro-3-(4-methyl-benzylidene)-1,3-dihydro-indol-2-one (Compound 135)
3-anthracen-9-ylmethylene-5-chloro-1,3-dihydro-indol-2-one (Compound 136)
5-chloro-3-naphtalen-2-ylmethylene-1,3-dihydro-indol-2-one (Compound 137)
5-chloro-3-(2,4-dimethoxy-benzylidene)-1,3-dihydro-indol-2-one (Compound 138)
5-chloro-3-(2,4-dichloro-benzylidene)-1,3-dihydro-indol-2-one (Compound 139)
5-chloro-3-(3,4-dimethoxy-benzylidene)-1,3-dihydro-indol-2-one (Compound 140)
5-Chloro-3-(3,5-dimethoxy-benzylidene)-1,3-dihydro-indol-2-one (Compound 141)
5-chloro-3-(3,5-di-tert-butyl-4-hydroxy-benzylidene)-1,3-dihydro-indol-2-one (Compound 142)
5-chloro-3-(3,4,5-trimethoxy-benzylidene)-1,3-dihydro-indol-2-one (Compound 143)
3-benzylidene-5-Chloro-1,3-dihydro-indol-2-one (Compound 144)
5-chloro-3-(3-hydroxy-benzylidene)-1,3-dihydro-indol-2-one (Compound 145)
5-chloro-3-(2-methoxy-benzylidene)-1,3-dihydro-indol-2-one (Compound 146)
5-chloro-3-(2-methyl-benzylidene)-1,3-dihydro-indol-2-one (Compound 147)
3-(3,5-dibromo-4-hydroxy-benzylidene)-1,3-dihydro-indol-2-one (Compound 148)
3-(3,4-difluoro-benzylidene)-1,3-dihydro-indol-2-one (Compound 149)
3-(2-hydroxy-naphtalen-1-ylmethylene)-1,3-dihydro-indol-2-one (Compound 150)
3-(4-methyl-benzylidene)-1,3-dihydro-indol-2-one (Compound 151)
3-(3,4-dimethoxy-benzylidene)-1,3-dihydro-indol-2-one (Compound 152)
3-(3-methoxy-benzylidene)-1,3-dihydro-indol-2-one (Compound 153)
3-(2-chloro-benzylidene)-1,3-dihydro-indol-2-one (Compound 154)
3-(3-chloro-benzylidene)-1,3-dihydro-indol-2-one (Compound 155)
3-(2,4-dichloro-benzylidene)-1,3-dihydro-indol-2-one (Compound 156)
3-(3,4-dichloro-benzylidene)-1,3-dihydro-indol-2-one (Compound 157)
3-(3-bromo-benzylidene)-1,3-dihydro-indol-2-one (Compound 158)
3-(4-hydroxy-benzylidene)-1,3-dihydro-indol-2-one (Compound 159)
3-(3-hydroxy-benzylidene)-1,3-dihydro-indol-2-one (Compound 160)
3-(2,4-dihydroxy-benzylidene)-1,3-dihydro-indol-2-one (Compound 161)
5-chloro-3-(2,5-dimethoxy-benzylidene)-1,3-dihydro-indol-2-one (Compound 162)
3-(3,4,5-trimethoxy-benzylidene)-1,3-dihydro-indol-2-one (Compound 163)
3-(3,5-dimethoxy-benzylidene)-1,3-dihydro-indol-2-one (Compound 164)
3-(2,3-dimethoxy-benzylidene)-1,3-dihydro-indol-2-one (Compound 165)
3-(2-methoxy-naphtalen-1-ylmethylene)-1,3-dihydro-indol-2-one (Compound 166)
3-(2-methoxy-benzylidene)-1,3-dihydro-indol-2-one (Compound 167)
3-(4-hydroxy-3-methoxy-benzylidene)-1,3-dihydro-indol-2-one (Compound 168)
3-(3-hydroxy-4-methoxy-benzylidene)-1,3-dihydro-indol-2-one (Compound 169)
5-bromo-3-(2,5-dimethoxy-benzylidene)-1,3-dihydro-indol-2-one (Compound 170)
6-bromo-3-(2,5-dimethoxy-benzylidene)-1,3-dihydro-indol-2-one (Compound 171)
7-bromo-3-(2,5-dimethoxy-benzylidene)-1,3-dihydro-indol-2-one (Compound 172)
3-(2,5-dimethoxy-benzylidene)-6-fluoro-1,3-dihydro-indol-2-one (Compound 173)
3-(2,5-dimethoxy-benzylidene)-5-trifluoromethyl-1,3-dihydro-indol-2-one (Compound 174)
5-amino-3-(2,5-dimethoxy-benzylidene)-1,3-dihydro-indol-2-one (Compound 175)
6-chloro-5-(2-chloro-acetyl)-3-(2,5-dimethoxy-benzylidene)-1,3-dihydro-indol-2-one (Compound 176)
3-(2,5-dimethoxy-benzylidene)-5-hydroxy-1,3-dihydro-indol-2-one (Compound 177)
3-(2,5-dimethoxy-benzylidene)-2-oxo-2,3-dihydro-1H-indole-5-carboxylic acid methyl ester (Compound 178)
3-(9-ethyl-9H-carbazol-3-ylmethylene)-1,3-dihydro-indol-2-one (Compound 179)
3-(2-hydroxy-3-methoxy-benzylidene)-1,3-dihydro-indol-2-one (Compound 180)
3-(2,5-dimethoxy-benzylidene)-4,5-difluoro-1,3-dihydro-indol-2-one (Compound 181)
3-(3,5-dichloro-2-hydroxy-benzylidene)-1,3-dihydro-indol-2-one (Compound 182)
3-(2,5-diethoxy-benzylidene)-1,3-dihydro-indol-2-one (Compound 183)
3-(2,5-dihydroxy-benzylidene)-1,3-dihydro-indol-2-one (Compound 184)
3-(2,4,5-trimethoxy-benzylidene)-1,3-dihydro-indol-2-one (Compound 185)
3-(9-methyl-9H-carbazol-3-ylmethylene)-1,3-dihydro-indol-2-one (Compound 186)
3-(2-hydroxy-5-trifluoromethoxy-benzylidene)-1,3-dihydro-indol-2-one (Compound 187)
3-(1H-indol-5-ylmethylene)-1,3-dihydro-indol-2-one (Compound 188)
3-(1H-indol-4-ylmethylene)-1,3-dihydro-indol-2-one (Compound 189)
3-(1H-indol-7-ylmethylene)-1,3-dihydro-indol-2-one (Compound 190)
3-(1,4-dimethyl-9H-carbazol-2-ylmethylene)-1,3-dihydro-indol-2-one (Compound 191)
3-(2-benzyloxy-4,5-dimethoxy-benzylidene)-1,3-dihydro-indol-2-one (Compound 192)
3-(2,5-dichloro-benzylidene)-1,3-dihydro-indol-2-one (Compound 193)
3-(2,5-dimethoxy-benzylidene)-2-oxo-2,3-dihydro-1H-indole-7-carbonitrile (Compound 194)
3-(2,5-dimethoxy-benzylidene)-6-methanesulfonyl-1,3-dihydro-indol-2-one (Compound 195)
3-(2,5-dimethoxy-benzylidene)-2-oxo-2,3-dihydro-1H-indole-5-carbonitrile (Compound 196)
3-(2,5-dimethoxy-benzylidene)-6-trifluoromethyl-1,3-dihydro-indol-2-one (Compound 197)
3-(2,5-dimethoxy-benzylidene)-7-fluoro-1,3-dihydro-indol-2-one (Compound 198)
3-(2,5-dimethoxy-benzylidene)-2-oxo-2,3-dihydro-1H-indole-6-carbonitrile (Compound 199)
6-chloro-3-(2,5-dimethoxy-benzylidene)-1,3-dihydro-indol-2-one (Compound 200)
3-(2,5-dibromo-benzylidene)-1,3-dihydro-indol-2-one (Compound 201)
3-(5-bromo-2-ethoxy-benzylidene)-1,3-dihydro-indol-2-one (Compound 202)
3-(5-bromo-2-methoxy-benzylidene)-1,3-dihydro-indol-2-one (Compound 203)
3-(2-fluoro-5-methoxy-benzylidene)-1,3-dihydro-indol-2-one (Compound 204)
3-(2,5-difluoro-benzylidene)-1,3-dihydro-indol-2-one (Compound 205)
3-(2-chloro-5-nitro-benzylidene)-1,3-dihydro-indol-2-one (Compound 206)
3-(2,5-bis-trifluoromethyl-benzylidene)-1,3-dihydro-indol-2-one (Compound 207)
3-(2,4-dichloro-benzylidene)-1,3-dihydro-indol-2-one (Compound 208)
3-(2-hydroxy-5-methoxy-benzylidene)-1,3-dihydro-indol-2-one (Compound 209)
3-(1H-indol-6-ylmethylene)-1,3-dihydro-indol-2-one (Compound 210)
3-(2,5-dimethoxy-benzylidene)-5-fluoro-1,3-dihydro-indol-2-one (Compound 211)
3-[4-(quinolin-2-ylmethoxy)-benzylidene]-1,3-dihydro-indol-2-one (Compound 212)
3-[4-(naphthalen-2-ylmethoxy)-benzylidene]-1,3-dihydro-indol-2-one (Compound 213)
3-[3,5-dichloro-2-(quinolin-2-ylmethoxy)-benzylidene]-1,3-dihydro-indol-2-one (Compound 214)
2-[4-(2-oxo-1,2-dihydro-indol-3-ylidenemethyl)-phenoxy]-propionic acid (Compound 215)
2-benzyl-3-butylamino-5-(2-oxo-1,2-dihydro-indol-3-ylidenemethyl)-benzenesulfonamide (Compound 216)
2-benzyl-3-benzylamino-5-(2-oxo-1,2-dihydro-indol-3-ylidenemethyl)-benzenesulfonamide (Compound 217)
3-[(furan-2-ylmethyl)-amino]-5-(2-oxo-1,2-dihydro-indol-3-ylidenemethyl)-2-phenoxy-benzenesulfonamide (Compound 218)
3-methylamino-5-(2-oxo-1,2-dihydro-indol-3-ylidenemethyl)-2-phenoxy-benzenesulfonamide (Compound 219)
2-benzyl-3-ethoxy-5-(2-oxo-1,2-dihydro-indol-3-ylidenemethyl)-benzenesulfonamide (Compound 220)
[2-(2-oxo-1,2-dihydro-indol-3-ylidenemethyl)-phenoxy]-acetic acid (Compound 221)
3-[4-(6-methyl-pyridin-2-ylmethoxy)-benzylidene]-1,3-dihydro-indol-2-one (Compound 222)
4-[4-(5-chloro-2-oxo-1,2-dihydro-indol-3-ylidenemethyl)-phenyl]-piperazine-1-carbaldehyde (Compound 223)
5-chloro-3-(4-isopropyl-benzylidene)-1,3-dihydro-indol-2-one (Compound 224)
4-[4-(2-oxo-1,2-dihydro-indol-3-ylidenemethyl)-phenyl]-piperazine-1-carbaldehyde (Compound 225)
3-[5-methoxy-2-(2-morpholin-4-yl-ethoxy)-benzylidene]-1,3-dihydro-indol-2-one; hydrochloride (Compound 258)
3-[5-methoxy-2-(2-piperidin-1-yl-ethoxy)-benzylidene]-1,3-dihydro-indol-2-one; hydrochloride (Compound 259)
3-(2,5-dimethoxy-benzylidene)-5,7-difluoro-1,3-dihydro-indol-2-one (Compound 260)
3-[4-(1-quinolin-4-yl-ethoxy)-benzylidene]-1,3-dihydro-indol-2-one (Compound 261)
3-[4-(pyridin-4-ylmethoxy)-benzylidene]-1,3-dihydro-indol-2-one (Compound 262) and
5-amino-3-(2,5-dimethoxy-benzylidene)-1,3-dihydro-indol-2-one; methanesulfonic acid (Compound 265)

26. The use of claim 1 wherein the compound is a compound of general formula IV 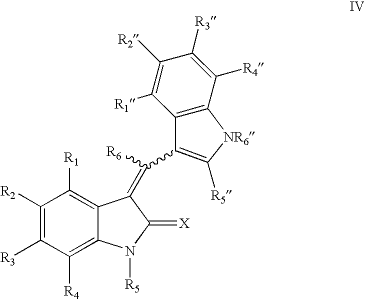
wherein R₁, R₂, R₃, R₄, R₅, R₆ and X are as indicated in claim 1,
R₁″, R₂″, R₃″, R₄″ and R₅″ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄alkyl, C₁₋₄alkoxy, nitro, cyano, amino, oxo, halogen, trihalomethyl, C₁₋₄alkylthio, C₁₋₄alkylamino, C₁₋₄alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; and
R₆″ is hydrogen, heterocyclyl, heteroaryl, —C(O)R₂₃, —S(O)₂R₂₃, —C(O)OR₂₃ or C₁₋₆alkyl optionally substituted with heterocyclyl, heteroaryl or —C(O)OR₂₃, wherein R₂₃ is hydrogen, C₁₋₆alkyl, aryl, heteroaryl or heterocyclyl.

27. The use of claim 26 wherein, in the compound of formula IV, R₂, R₃, R₄, R₅, R₆ and X are as indicated in claim 2, and R₁″, R₂″, R₃″, R₄″ and R₅″ are the same or different and independently selected from the group consisting of with C₁₋₁₀alkyl, C₁₋₁₀alkoxy, aryl, heteroaryl, aryloxy, C₁₋₁₀alkylaryl, C₁₋₁₀alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆alkyl or aryl, and n is 0-3; and R₆″ is hydrogen, C₁₋₆ alkyl, heteroaryl, heteroaryl-C₁₋₆ alkyl, C(O)R₁₈, C(O)OR₁₈ or S(O)₂R₁₈, wherein R₁₈ is as indicated above.

28. The use of claim 26 wherein, in the compound of formula IV, R₅″ is hydrogen or C₁₋₆ alkyl.

29. The use of claim 26 wherein, in the compound of formula IV, R₆″ is hydrogen or C₁₋₆ alkyl.

30. The use of claim 26 wherein, in the compound of formula IV, R₅ is hydrogen, hydroxy, C(O)R₁₄ or C(O)OR₁₄, wherein R₁₄ is as defined in claim 1.

31. The use of claim 26 wherein the compound is selected from the group consisting of
3-(1H-indol-3-ylmethylene)-1,3-dihydro-indol-2-one (Compound 57)
[3-(2-oxo-1,2-dihydro-indol-3-ylidenemethyl)-indol-1-yl]-acetic acid methyl ester (Compound 24)
[3-(2-oxo-1,2-dihydro-indol-3-ylidenemethyl)-indol-1-yl]-acetic acid ethyl ester (Compound 25)
[3-(2-oxo-1,2-dihydro-indol-3-ylidenemethyl)-indol-1-yl]-acetic acid (Compound 26)
3-[3-(2-oxo-1,2-dihydro-indol-3-ylidenemethyl)-indol-1-yl]-propionic acid ethyl ester (Compound 27)
3-[3-(2-oxo-1,2-dihydro-indol-3-ylidenemethyl)-indol-1-yl]-propionic acid (Compound 28)
3-[1-(2-chloro-thiazol-5-ylmethyl)-1H-indol-3-ylmethylene]-1,3-dihydro-indol-2-one (Compound 29)
3-(1-methyl-1H-indol-3-ylmethylene)-1,3-dihydro-indol-2-one (Compound 30)
3-(1-propyl-1H-indol-3-ylmethylene)-1,3-dihydro-indol-2-one (Compound 31)
3-(1H-indol-3-ylmethylene)-2-oxo-2,3-dihydro-indole-1-carboxylic acid tert-butyl ester (Compound 40)
1-hydroxy-3-(1H-indol-3-ylmethylene)-1,3-dihydro-indol-2-one (Compound 47)
(1-Methyl-1H-indol-3-yl)-(2-oxo-1,2-dihydro-indol-3-ylidene)-acetic acid (Compound 56)
3-(2-phenyl-1H-indol-3-ylmethylene)-1,3-dihydro-indol-2-one (Compound 58)
3-(1-methyl-2-phenyl-1H-indol-3-ylmethylene)-1,3-dihydro-indol-2-one (Compound 59)
3-[2-(4-chloro-phenyl)-1H-indol-3-ylmethylene]-1,3-dihydro-indol-2-one (Compound 60)
3-(2-naphthalen-2-yl-1H-indol-3-ylmethylene)-1,3-dihydro-indol-2-one (Compound 61)
5-chloro-3-(1H-indol-3-ylmethylene)-1,3-dihydro-indol-2-one (Compound 62)
3-(5-methyl-1H-indol-3-ylmethylene)-1,3-dihydro-indol-2-one (Compound 63)
5,7-difluoro-3-(1H-indol-3-ylmethylene)-1,3-dihydro-indol-2-one (Compound 64)
5-bromo-3-(1H-indol-3-ylmethylene)-1,3-dihydro-indol-2-one (Compound 65)
6-fluoro-3-(1H-indol-3-ylmethylene)-1,3-dihydro-indol-2-one (Compound 66)
6-bromo-3-(1H-indol-3-ylmethylene)-1,3-dihydro-indol-2-one (Compound 67)
5-hydroxy-3-(1H-indol-3-ylmethylene)-1,3-dihydro-indol-2-one (Compound 68)
3-(4,5,6,7-tetrafluoro-1H-indol-3-ylmethylene)-1,3-dihydro-indol-2-one (Compound 69)
3-(6-fluoro-1H-indol-3-ylmethylene)-1,3-dihydro-indol-2-one (Compound 70)
3-[2-(4-chloro-phenyl)-5-nitro-1H-indol-3-ylmethylene)-1,3-dihydro-indol-2-one (Compound 71)
7-bromo-3-(1H-indol-3-ylmethylene)-1,3-dihydro-indol-2-one (Compound 72)
3-(6-methyl-1H-indol-3-ylmethylene)-1,3-dihydro-indol-2-one (Compound 73)
3-(7-methyl-1H-indol-3-ylmethylene)-1,3-dihydro-indol-2-one (Compound 74)
3-(2-methyl-1H-indol-3-ylmethylene)-1,3-dihydro-indol-2-one (Compound 75)
3-(5-fluoro-1-methyl-1H-indol-3-ylmethylene)-1,3-dihydro-indol-2-one (Compound 76)
3-(5-fluoro-1H-indol-3-ylmethylene)-1,3-dihydro-indol-2-one (Compound 77)
3-(5-methoxy-1H-indol-3-ylmethylene)-1,3-dihydro-indol-2-one (Compound 78)
3-(5-benzyloxy-1H-indol-3-ylmethylene)-1,3-dihydro-indol-2-one (Compound 79)
3-(6-methoxy-1H-indol-3-ylmethylene)-1,3-dihydro-indol-2-one (Compound 80)
3-(5-methoxy-1-methyl-1H-indol-3-ylmethylene)-1,3-dihydro-indol-2-one (Compound 81)
3-(6-methoxy-1-methyl-1H-indol-3-ylmethylene)-1,3-dihydro-indol-2-one (Compound 82)
3-(4-benzyloxy-1H-indol-3-ylmethylene)-1,3-dihydro-indol-2-one (Compound 83)
3-(1H-indol-3-ylmethylene)-2-oxo-2,3-dihydro-1H-indol-6-carbonitrile (Compound 84)
3-(1H-indol-3-ylmethylene)-2-oxo-2,3-dihydro-1H-indol-7-carbonitrile (Compound 85)
3-(1H-indol-3-ylmethylene)-2-oxo-2,3-dihydro-1H-indol-5-carbonitrile (Compound 86)
7-fluoro-3-(1H-indol-3-ylmethylene)-1,3-dihydro-indol-2-one (Compound 87)
3-(1H-indol-3-ylmethylene)-6-trifluoromethyl-1,3-dihydro-indol-2-one (Compound 88)
3-(1H-indol-3-ylmethylene)-6-methanesulfonyl-1,3-dihydro-indol-2-one (Compound 89)
3-(1H-indol-3-ylmethylene)-5-trifluoromethyl-1,3-dihydro-indol-2-one (Compound 90)
3-(1H-indol-3-ylmethylene)-5,6-dimethoxy-1,3-dihydro-indol-2-one (Compound 91)
4,5-difluoro-3-(1H-indol-3-ylmethylene)-1,3-dihydro-indol-2-one (Compound 92)
3-(1H-indol-3-ylmethylene)-5-methoxy-1,3-dihydro-indol-2-one (Compound 92A)
6-chloro-3-(1H-indol-3-ylmethylene)-1,3-dihydro-indol-2-one (Compound 93) and
3-[l-Methyl-2-(4-methyl-piperazin-1-yl)-1H-indol-3-ylmethylene]-1,3-dihydro-indol-2-one (Compound 94)

32. A method of preventing, treating or ameliorating multiple sclerosis, or delaying the onset of or reducing the relapse rate in multiple sclerosis, the method comprising administering, to a patient in need thereof, a pharmacologically effective amount of a compound of general formula I 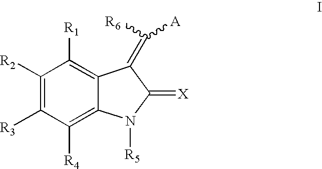
wherein
R₁, R₂, R₃ and R₄ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂;
X is O or S;
R₅ is hydrogen, hydroxy, C₁₋₆alkyl, C₂₋₆alkenyl, C₂₋₆alkynyl, cycloalkyl, heterocyclyl, aryl, heteroaryl, C₁₋₆alkoxy, carbonyl, carboxy, amido, thioamido, guanyl, guanidinyl, ureidyl, sulfonyl, trihalomethanesulfonyl, —C(O)OR₁₄, —C(O)R₁₄, wherein R₁₄ is hydrogen, C₁₋₆alkyl, C₂₋₆alkenyl, C₂₋₆alkynyl, cycloalkyl or aryl;
R₆ is hydrogen, C₁₋₆alkyl, cycloalkyl, aryl, heteroaryl, heterocyclyl, halogen, —OR₇, —C(O)R₇, —C(O)OR₇, —NR₇R₈, S(O)₂NR₇R₈, wherein R₇ and R₈ are independently hydrogen, C₁₋₆alkyl, aryl or heterocyclyl, said C₁₋₆alkyl or heterocyclyl being optionally substituted by heterocyclyl, —OR₇, —C(O)R₇ or C(O)OR₇, the zigzag line indicating that the group denoted R₆ is present as the E- or Z-isomer;
A is phenyl or a monocyclic or bicyclic heteroaryl ring, optionally substituted at one or more positions with hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; the zigzag line indicating that the group denoted A is present as the E- or Z-isomer;
or pharmaceutically acceptable salts thereof.

33. The method of claim 32, wherein, in the compound of formula I,
X is O or S;
R₁, R₂, R₃ and R₄ are the same or different and independently selected from the group consisting of hydrogen, C₁₋₁₀alkyl, C₁₋₁₀alkoxy, aryl, heteroaryl, aryloxy, C₁₋₁₀alkylaryl, C₁₋₁₀alkylaryloxy, halogen, trihalomethyl, S(O)R₁₈, S(O)₂R₁₈, S(O)₂NR₁₈R₁₉, S(O)₃R₁₈, SR₁₈, NO₂, NR₁₈R₁₉, OH, CN, C(O)R₁₈, C(O)OR₁₈, OC(O)R₁₈, NHC(O)R₁₈, (CH₂)—C(O)₂R₁₈ and C(O)NR₁₈R₁₉, wherein R₁₈ is hydrogen, C₁₋₆alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆alkyl or aryl, and n is 0-3;
A is phenyl or a monoclyclic or bicyclic heteroaryl ring selected from the group consisting of pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,3,4-thiatriazole, 1,2,3,5-thiatriazole, tetrazole and indole, optionally substituted at one or more positions with C₁₋₁₀alkyl, C₁₋₁₀alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈, R₁₉ and n are as indicated above;
R₅ is hydrogen or C₁₋₆alkyl; and
R₆ is hydrogen.

34. The method of claim 33 wherein, in the compound of formula I, R₅ is hydrogen.

35. The method of claim 33 wherein, in the compound of formula I, X is oxygen.

36. The method of claim 33 wherein, in the compound of formula I, R₆ is hydrogen or COOH.

37. The method of claim 33 wherein, in the compound of formula I, R₁, R₂, R₃ and R₄ are the same or different and independently selected from hydrogen and C₁₋₆alkyl.

38. The method of claim 33 wherein, in the compound of formula I, A is pyrrole, phenyl or indole, said pyrrole, phenyl or indole being optionally substituted at one or more positions with C₁₋₁₀alkyl, C₁₋₁₀alkoxy, aryl, heteroaryl, aryloxy, C₁₋₁₀alkylaryl, C₁₋₁₀alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈, R₁₉ and n are as indicated in claim 33.

39. The method of claim 38 wherein, in the compound of formula I, A is pyrrole substituted at position 3 and 5 with C₁₋₆alkyl, or at position 3 with C₁₋₆alkyl and at position 5 with CH₂OH, COOH or a sugar residue, or or at position 3 and 5 with C₁₋₆alkyl and at position 4 with halogen, or at position 5 with C(O)O—C₁₋₆alkyl, and at position 3 with C₁₋₆alkyl.

40. The method of claim 38 wherein, in the compound of formula I, A is phenyl substituted at position 2 and 5 with C₁₋₆ alkyl, C₁₋₆alkoxy, halogen, C₁₋₆ alkyl-NR₂₆R₂₇, NH—C₁₋₆ alkyl-NR₂₆R₂₇ or O—C₁₋₆ alkyl-NR₂₆R₂₇, wherein R₂₆ and R₂₇ are independently hydrogen or C₁₋₆ alkyl or, together with the nitrogen atom to which they are attached, form a heteroaryl or heterocyclic ring.

41. The method of claim 38 wherein, in the compound of formula I, A is indole.

42. The the method of claim 38 wherein the compound is 3-(3,5-dimethyl-1H-pyrrol-2-yl-methylene)-1,3-dihydro-indol-2-one.

43. The method of claim 38 wherein the compound is 3-(2,5-dimethoxy-benzylidene)-1,3-dihydroindol-2-one.

44. The method of claim 38 wherein the compound is 3-(1H-indol-3-ylmethylene)-1,3-dihydroindol-2-one.

45. The method of claim 32 wherein the compound is a compound of general formula II 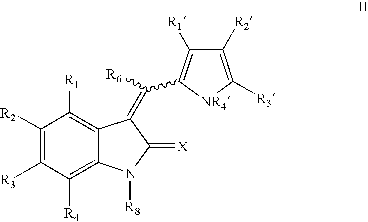
wherein R₁, R₂, R₃, R₄, R₅, R₆ and X are as indicated in claim 1,
R₈ and R₄′ are independently hydrogen, hydroxy, C₁₋₆alkyl, C₂₋₆alkenyl, C₂₋₆alkynyl, cycloalkyl, heterocyclyl, aryl, heteroaryl, C₁₋₆alkoxy, carbonyl, carboxy, amido, thioamido, guanyl, guanidinyl, ureidyl, sulfonyl, trihalomethanesulfonyl, —PO(OR)(OR′), wherein R and R′ are independently selected from hydrogen or C₁₋₆ alkyl, , —OR₁₀, —C(O)R₁₀, —C(O)OR₁₀, OC(O)R₁₀, OC(O)OR₁₀, —NR₁₀R₁₁, —C(O)NR₁₀R₁₁, —OC(O)NR₁₀R₁₁, —OC(O)NR₁₀R₁₁, —NHC(O)R₁₀, —SR₁₀, —S(O)R₁₀, —S(O)₂R₁₀, —S(O)₂NR₁₀R₁₁, —S(O)OR₁₀ and CH₂-aryl-OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, OC(O)OR₁₂, —NR₁₂R₁₃, —C(O)NR₁₂R₁₃, —OC(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂, —S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; —C(R₂₄R₂₅)—OR₁₆ or —OC(O)R₁₆, wherein R₁₆ is hydrogen, C₁₋₆ alkyl, aralkyl, acyl or —PO(OR)(OR′), —C(R₂₄R₂₅)—NR₂₆R₂₇, wherein R₂₄ is hydrogen, C₁₋₆ alkyl or aryl, R₂₅ is hydrogen, and R₂₆ and R₂₇ are independently hydrogen or C₁₋₆ alkyl or, together with the nitrogen atom to which they are attached, form a heteroaryl or heteroaryl ring optionally substituted with hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂ or —S(O)NH₂; —NR₂₀R₂₁, —O(CH₂)ₘNR₂₀R₂₁, —N(CH₂)ₘNR₂₀R₂₁, —O(CH₂)ₘC(O)R₂₂, —N(CH₂)ₘC(O)R₂₂, wherein m is 0, 1, 2 or 3, R₂₀ and R₂₁ are the same or different and independently selected from the group consisting of hydrogen, C₁₋₆ alkyl, cycloalkyl, aryl, carbonyl, acetyl, trihalomethylcarbonyl, carboxy, sulfonyl or trihalomethanesulfonyl, or R₂₀ and R₂₁ together with the nitrogen atom to which they are attached form a heterocyclic or heteroaryl ring, and R₂₂ is hydroxy, C₁₋₆ alkoxy, aryloxy, amino, hydroxylamino, carboxy or —NR₂₀R₂₁, wherein R₂₀ and R₂₁ are as indicated above; and
R₁′, R₂′ and R₃′ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₁₆-alkyl, C₂₋₁₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄alkyl, C₁₋₄alkoxy, nitro, cyano, amino, oxo, halogen, trihalomethyl, C₁₋₄alkylthio, C₁₋₄alkylamino, C₁₋₄alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄alkylamino, C₁₋₄alkoxycarbonyl, carboxy, —CONH₂ or —S(O)NH₂.

46. The method of claim 45 wherein, in the compound of formula II, R₁, R₂, R₃, R₄, R₅, R₆ and X are as indicated in claim 33, and R₁′, R₂′ and R₃′ are the same or different and independently selected from the group consisting of with C₁₋₁₀alkyl, C₁₋₁₀alkoxy, aryl, heteroaryl, aryloxy, C₁₋₁₀alkylaryl, C₁₋₁₀alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆alkyl or aryl, and n is 0-3.

47. The method of claim 46 wherein, in the compound of formula II, R₁, R₂, R₃ and R₄ are the same or different and independently selected from hydrogen, halogen and C₁₋₆alkyl, or R₂ is hydroxy or heteroaryl, such as pyridyl, or a group C(O)R₂₀, wherein R₂₀ is heteroaryl, such as pyridyl or thienyl, and R₁, R₃ and R₄ are hydrogen.

48. The method of claim 46 wherein, in the compound of formula II, R₁′, R₂′ and R₃′ are the same or different and independently selected from hydrogen, halogen, C₁₋₆alkyl, C₁₋₆alkoxy, CH₂OH, C(O)OR₁₈ or C(O)NR₁₈R₁₉, wherein R₁₈ and R₁₉ are as defined in claim 46.

49. The method of claim 45 or 46 wherein, in the compound of formula II, R₁′ and R₃′ are both C₁₋₆alkyl, in particular methyl, and R₂′ is hydrogen, or wherein R₁′ is C₁₋₆alkyl and R₃′ is C₁₋₆alkoxy, CH₂OH, C(O)OR₁₈ or C(O)NR₁₈R₁₉, wherein R₁₈ and R₁₉ are as defined in claim 46, or wherein R₁′ and R₃′ are both C₁₋₆ alkyl, in particular methyl, and R₂′ is halogen, in particular chloro or bromo, or wherein R₁′ is C₁₋₆alkyl and R₃′ is C(O)O—C₁₋₆alkyl, or wherein R₁′ is C₁₋₆alkyl and R₃′ is C(O)NH—C₁₋₆alkyl substituted with hydroxy.

50. The method of claim 45 wherein, in the compound of formula II, R₈ and R₄′ are independently hydrogen, hydroxy, —PO(OR)(OR′), —OR₁₀, —C(O)OR₁₀, —C(O)NR₁₀R₁₁, —C(O)R₁₄, —C(R₂₄R₂₅)OR₁₆, —OC(O)R₁₆ or —C(R₂₄R₂₅)NR₂₆R₂₇, wherein R, R′, R₁₀, R₁₁, R₁₄, R₁₆, R₂₄, R₂₅, R₂₆, R₂₇ are as defined in claim 45.

51. The method of claim 45 wherein the compound is selected from the group consisting of
3-(3,5-Dimethyl-1H-pyrrol-2-ylmethylene)-1,3-dihydro-indol-2-one (Compound 226)
4-methyl-5-(2-oxo-1,2-dihydro-indol-3-ylidenemethyl)-1H-pyrrole-2-carboxylic acid ethyl ester (Compound 01)
4-methyl-5-(2-oxo-1,2-dihydro-indol-3-ylidenemethyl)-1H-pyrrole-2-carboxylic acid (2-hydroxy-ethyl)-amide (Compound 02)
3-(5-hydroxymethyl-3-methyl-1H-pyrrol-2-ylmethylene)-1,3-dihydro-indol-2-one (Compound 03)
1-[4-methyl-5-(2-oxo-1,2-dihydro-indol-3-ylidenemethyl)-1H-pyrrol-2-ylmethyl)-pyrridinum; chloride (Compound 04)
4-methyl-5-(2-oxo-1,2-dihydro-indol-3-ylidenemethyl)-1H-pyrrole-2-carboxylic acid (Compound 05)
4-methyl-5-(2-oxo-1,2-dihydro-indol-3-ylidenemethyl)-1H-pyrrole-2-carboxylic acid (2-diethylamino-ethyl)-amide (Compound 06)
4-methyl-5-(2-oxo-1,2-dihydro-indol-3-ylidenemethyl)-1H-pyrrole-2-carboxylic acid (2-methoxy-ethyl)-amide (Compound 07)
4-methyl-5-(2-oxo-1,2-dihydro-indol-3-ylidenemethyl)-1H-pyrrole-2-carboxylic acid [3-(1-formyl-piperidin-4-yl)-propyl]-amide (Compound 08)
4-{[4-methyl-5-(2-oxo-1,2-dihydro-indol-3-ylidenemethyl)-1H-pyrrole-2-carbonyl]-amino}-butyric acid methyl ester (Compound 09)
4-methyl-5-(2-oxo-1,2-dihydro-indol-3-ylidenemethyl)-1H-pyrrole-2-carboxylic acid (6-hydroxy-hexyl)-amide (Compound 10)
4-methyl-5-(2-oxo-1,2-dihydro-indol-3-ylidenemethyl)-1H-pyrrole-2-carboxylic acid cyclohexylmethyl-amide (Compound 11)
4-methyl-5-(2-oxo-1,2-dihydro-indol-3-ylidenemethyl)-1H-pyrrole-2-carboxylic acid (4-hydroxy-butyl)-amide (Compound 12)
6-{[4-methyl-5-(2-oxo-1,2-dihydro-indol-3-ylidenemethyl)-1H-pyrrole-2-carbonyl]-amino}-hexanoic acid ethyl ester (Compound 13)
4-methyl-5-(2-oxo-1,2-dihydro-indol-3-ylidenemethyl)-1H-pyrrole-2-carboxylic acid (tetrahydro-furan-2-ylmethyl)-amide (Compound 14)
4-methyl-5-(2-oxo-1,2-dihydro-indol-3-ylidenemethyl)-1H-pyrrole-2-carboxylic acid [2-(1H-indol-3-yl)-ethyl]-amide (Compound 15)
4-methyl-5-(2-oxo-1,2-dihydro-indol-3-ylidenemethyl)-1H-pyrrole-2-carboxylic acid (3-phenyl-propyl)-amide (Compound 16)
4-methyl-5-(2-oxo-1,2-dihydro-indol-3-ylidenemethyl)-1H-pyrrole-2-carboxylic acid (4-phenyl-butyl)-amide (Compound 17)
4-methyl-5-(2-oxo-1,2-dihydro-indol-3-ylidenemethyl)-1H-pyrrole-2-carboxylic acid (5-hydroxy-pentyl)-amide (Compound 18)
4-{[4-methyl-5-(2-oxo-1,2-dihydro-indol-3-ylidenemethyl)-1H-pyrrole-2-carbonyl]-amino}-butyric acid ethyl ester (Compound 19)
4-methyl-5-(2-oxo-1,2-dihydro-indol-3-ylidenemethyl)-1H-pyrrole-2-carboxylic acid [1-(4-chloro-phenyl)-cyclopropylmethyl]-amide (Compound 20)
4-methyl-5-(2-oxo-1,2-dihydro-indol-3-ylidenemethyl)-1H-pyrrole-2-carboxylic acid benzyl ester (Compound 21)
3-(4-bromo-3,5-dimethyl-1H-pyrrol-2-ylmethylene)-1,3-dihydro-indol-2-one (Compound 22)
3-(4-chloro-3,5-dimethyl-1H-pyrrol-2-ylmethylene)-1,3-dihydro-indol-2-one (Compound 23)
3-(3,5-dimethyl-1H-pyrrol-2-ylmethylene)-1-(4-methoxy-benzyl)-1,3-dihydro-indol-2-one (Compound 41)
3-(3,5-Dimethyl-1H-pyrrol-2-ylmethylene)-1-methyl-1,3-dihydro-indol-2-one (Compound 42)
acetic acid 3-(3,5-dimethyl-1H-pyrrol-2-ylmethylene)-2-oxo-2,3-dihydro-indol-1-ylmethyl ester (Compound 43)
3-(3,5-dimethyl-1H-pyrrol-2-ylmethylene)-1-hydroxy-1,3-dihydro-indol-2-one (Compound 45)
3-(4-bromo-3,5-dimethyl-1H-pyrrol-2-ylmethylene)-1-hydroxy-1,3-dihydro-indol-2-one (Compound 46)
3-(3,5-dimethyl-1H-pyrrol-2-ylmethylene)-1-methoxy-1,3-dihydro-indol-2-one (Compound 49)
acetic acid 3-(3,5-dimethyl-1H-pyrrol-2-ylmethylene)-2-oxo-2,3-dihydro-indol-1-yl ester (Compound 51)
2-{3-[3-(3,5-dimethyl-1H-pyrrol-2-ylmethylene)-2-oxo-2,3-dihydro-indol-1-yloxy]-propyl}-isoindole-1,3-dione (Compound 52)
2,4-Dimethyl-5-(2-oxo-1,2-dihydro-indol-3-ylidenemethyl)-1H-pyrrole-3-carboxylic acid (2-diethylamino-ethyl)-amide (Compound 227)
5-(5-Fluoro-2-oxo-1,2-dihydro-indol-3-ylidenemethyl)-2,4-dimethyl-1H-pyrrole-3-carboxylic acid (2-diethylamino-ethyl)-amide (Compound 228)
(3,5-dimethyl-1H-pyrrol-2-yl)-(2-oxo-1,2-dihydro-indol-3-ylidene)-acetic acid (Compound 229)
3-[2,4-Dimethyl-5-(2-oxo-1,2-dihydro-indol-3-ylidenemethyl)-1H-pyrrol-3-yl]-propionic acid (Compound 230)
3-(3,5-dimethyl-1H-pyrrol-2-ylmethylene)-4-iodo-1,3-dihydro-indol-2-one (Compound 231)
3-(3,5-dimethyl-1H-pyrrol-2-ylmethylene)-5-methoxy-1,3-dihydro-indol-2-one (Compound 232)
5-chloro-3-(3-methoxy-1H-pyrrol-2-ylmethylene)-1,3-dihydro-indol-2-one (Compound 233)
3-(3-methoxy-1H-pyrrol-2-ylmethylene)-1,3-dihydro-indol-2-one (Compound 234)
3-[5-(4-chloro-2-oxo-1,2-dihydro-indol-3-ylidenemethyl)-2,4-dimethyl-1H-pyrrol-3-yl]-propionic acid (Compound 235)
4-chloro-3-(3,5-dimethyl-1H-pyrrol-2-ylmethylene)-1,3-dihydro-indol-2-one (Compound 236)
4-chloro-3-(3-methoxy-1H-pyrrol-2-ylmethylene)-1,3-dihydro-indol-2-one (Compound 237)
3-(3,5-dimethyl-1H-pyrrol-2-ylmethylene)-2-oxo-2,3-dihydro-1H-indole-4-carboxylic acid (Compound 238)
3-(3,5-dimethyl-1H-pyrrol-2-ylmethylene)-5-pyridin-3-yl-1,3-dihydro-indol-2-one (Compound 239)
3-(3,5-dimethyl-1H-pyrrol-2-ylmethylene)-5-pyridin-3-yl-1,3-dihydro-indol-2-one; methanesulfonic acid (Compound 240)
5-pyridin-3-yl-3-(1H-pyrrol-2-ylmethylene)-1,3-dihydro-indol-2-one (Compound 241)
5-pyridin-3-yl-3-(1H-pyrrol-2-ylmethylene)-1,3-dihydro-indol-2-one; methanesulfonic acid (Compound 242)
3-(3,5-dimethyl-1H-pyrrol-2-ylmethylene)-5-hydroxy-1,3-dihydro-indol-2-one (Compound 243)
3-(3,5-dimethyl-1H-pyrrol-2-ylmethylene)-5-fluoro-1,3-dihydro-indol-2-one (Compound 244)
3-(1-methyl-1H-indol-2-ylmethylene)-1,3-dihydro-indol-2-one (Compound 245)
2,4-dimethyl-5-(2-oxo-1,2-dihydro-indol-3-ylidenemethyl)-1H-pyrrole-3-carboxylic acid ethyl ester (Compound 246)
4-methyl-5-(2-oxo-1,2-dihydro-indol-3-ylidenemethyl)-1H-pyrrole-2-carboxylic acid pyridin-4-ylmethyl ester (Compound 263)
(3,5-dimethyl-1H-pyrrol-2-yl)-(2-oxo-1,2-dihydro-indol-3-ylidene)-acetic acid benzyl ester (Compound 264)
3-(3,5-dimethyl-1H-pyrrol-2-ylmethylene)-1-pyrrolidin-1-ylmethyl-1,3-dihydro-indol-2-one (Compound 266)
3-(3,5-dimethyl-1H-pyrrol-2-ylmethylene)-1-(4-methyl-piperazin-1-ylmethyl)-1,3-dihydro-indol-2-one (Compound 267) and
3-(3,5-dimethyl-1H-pyrrol-2-ylmethylene)-1-piperidin-1-ylmethyl-1,3-dihydro-indol-2-one (Compound 268)

52. The method of claim 32, wherein the compound is a compound of formula III 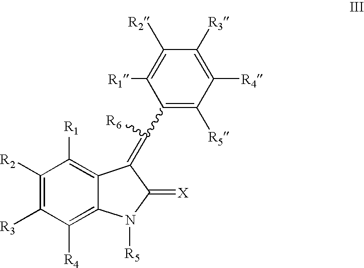
wherein R₁, R₂, R₃, R₄, R₅, R₆ and X are as indicated in claim 1, and
R₁″, R₂″, R₃″, R₄″ and R₅″ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein OR₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, halogen, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄ alkoxycarbonyl, carboxy, —CONH₂, —S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂.

53. The method of claim 52 wherein, in the compound of formula III, R₂, R₃, R₄, R₅, R₆ and X are as indicated in claim 31, and R₁″, R₂″, R₃″, R₄″ and R₅″ are the same or different and independently selected from the group consisting of with C₁₋₁₀ alkyl, C₁₋₁₀ alkoxy, aryl, heteroaryl, aryloxy, C₁₋₁₀ alkylaryl, C₁₋₁₀ alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆ alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆ alkyl or aryl, and n is 0-3.

54. The method of claim 53 wherein, in the compound of formula III, R₂″ and R₅″ are the same or different and independently are C₁₋₆ alkyl, in particular methyl, or C₁₋₆ alkoxy, in particular methoxy, or halogen, in particular chloro or bromo.

55. The method of claim 52 wherein, in the compound of formula III, R₅ is hydrogen, hydroxy, C(O)R₁₄ or C(O)OR₁₄, wherein R₁₄ is as defined in claim 32.

56. The method of claim 52, wherein the compound is selected from the group consisting of
3-(2,5-dimethoxy-benzylidene)-1,3-dihydro-indol-2-one (Compound 110)
3-(5-dimethylaminomethyl-2-methoxy-benzylidene)-1,3-dihydro-indol-2-one (Compound 32)
3-{2-[(2-dimethylamino-ethyl)-methyl-amino]-5-methoxy-benzylidene}-1,3-dihydro-indol-2-one (Compound 33)
3-{4-[(2-dimethylamino-ethyl)-methyl-amino]-3′,5′-dimethyl-biphenyl-3-ylmethylene}-1,3-dihydro-indol-2-one (Compound 34)
3-(2-dimethylaminomethyl-benzylidene)-1,3-dihydro-indol-2-one (Compound 35)
3-[2-(2-diethylamino-ethoxy)-5-methoxy-benzylidene]-1,3-dihydro-indol-2-one (Compound 36)
3-[2-(2-diethylamino-ethoxy)-5-methoxy-benzylidene]-1,3-dihydro-indol-2-one; hydrochloride (Compound 37)
3-[5-methoxy-2-(2-morpholin-4-yl-ethoxy)-benzylidene]-1,3-dihydro-indol-2-one (Compound 38)
3-[5-methoxy-2-(2-piperidin-1-yl-ethoxy)-benzylidene]-1,3-dihydro-indol-2-one (Compound 39)
1-acetyl-3-(2,5-dimethoxy-benzylidene)-1,3-dihydro-indol-2-one (Compound 44)
3-(2,5-dimethoxy-benzylidene)-1-hydroxy-1,3-dihydro-indol-2-one (Compound 48)
3-(2,5-dimethoxy-benzylidene)-1-methoxy-1,3-dihydro-indol-2-one (Compound 50)
3-(phenyl-4-tolyl-methylene)-1,3-dihydro-indol-2-one (Compound 53)
3-[bis-(4-methoxy-phenyl)-methylene]-1,3-dihydro-indol-2-one (Compound 54)
3-[1-(2,5-dimethoxy-phenyl)-ethylidene]-1,3-dihydro-indol-2-one (Compound 55)
3-(4-hydroxy-3,5-dimethyl-benzylidene)-1,3-dihydro-indol-2-one (Compound 95)
3-(3,5-di-tert-butyl-4-hydroxy-benzylidene)-1,3-dihydro-indol-2-one (Compound 96)
3-(4-bromo-benzylidene)-1,3-dihydro-indol-2-one (Compound 97)
3-(2-methyl-benzylidene)-1,3-dihydro-indol-2-one (Compound 98)
3-(2,4-dimethyl-benzylidene)-1,3-dihydro-indol-2-one (Compound 99)
3-(2,6-dichloro-benzylidene)-1,3-dihydro-indol-2-one (Compound 100)
3-(3,4-dichloro-benzylidene)-1,3-dihydro-indol-2-one (Compound 101)
3-(4-chloro-benzylidene)-1,3-dihydro-indol-2-one (Compound 102)
3-(2,4-dimethyl-benzylidene)-1,3-dihydro-indol-2-one (Compound 103)
3-(2,5-dimethyl-benzylidene)-1,3-dihydro-indol-2-one (Compound 104)
3-(2,6-dimethoxy-benzylidene)-1,3-dihydro-indol-2-one (Compound 105)
3-benzylidene-1,3-dihydro-indol-2-one (Compound 106)
3-(4-methoxy-benzylidene)-1,3-dihydro-indol-2-one (Compound 107)
3-(2,4-dimethoxy-benzylidene)-1,3-dihydro-indol-2-one (Compound 108)
3-(3,5-dimethoxy-benzylidene)-1,3-dihydro-indol-2-one (Compound 109)
3-(3,4-dimethyl-benzylidene)-1,3-dihydro-indol-2-one (Compound 111)
3-naphtalen-2-ylmethylene-1,3-dihydro-indol-2-one (Compound 112)
3-naphtalen-1-ylmethylene-1,3-dihydro-indol-2-one (Compound 113)
3-(2,3-dimethoxy-benzylidene)-1,3-dihydro-indol-2-one (Compound 114)
3-(3-nitro-benzylidene)-1,3-dihydro-indol-2-one (Compound 115)
3-(2-fluoro-benzylidene)-1,3-dihydro-indol-2-one (Compound 116)
3-(3-methyl-benzylidene)-1,3-dihydro-indol-2-one (Compound 117)
3-(3-fluoro-benzylidene)-1,3-dihydro-indol-2-one (Compound 118)
3-(4-fluoro-benzylidene)-1,3-dihydro-indol-2-one (Compound 119)
3-anthracen-9-ylmethylene-1,3-dihydro-indol-2-one (Compound 120)
3-(5-bromo-2-hydroxy-benzylidene)-1,3-dihydro-indol-2-one (Compound 121)
3-(2-hydroxy-benzylidene)-1,3-dihydro-indol-2-one (Compound 122)
5-chloro-3-(4-isopropyl-benzylidene)-1,3-dihydro-indol-2-one (Compound 123)
5-chloro-3-(4-dimethylamino-benzylidene)-1,3-dihydro-indol-2-one (Compound 124)
5-chloro-3-(3,4-dichloro-benzylidene)-1,3-dihydro-indol-2-one (Compound 125)
5-chloro-3-(2,5-dimethoxy-benzylidene)-1,3-dihydro-indol-2-one (Compound 126)
5-Chloro-3-(2-chloro-benzylidene)-1,3-dihydro-indol-2-one (Compound 127)
5-chloro-3-(2,3-dimethoxy-benzylidene)-1,3-dihydro-indol-2-one (Compound 128)
5-Chloro-3-(2,6-dichloro-benzylidene)-1,3-dihydro-indol-2-one (Compound 129)
5-Chloro-3-(2-hydroxy-benzylidene)-1,3-dihydro-indol-2-one (Compound 130)
5-chloro-3-(4-methoxy-benzylidene)-1,3-dihydro-indol-2-one (Compound 131)
5-chloro-3-(4-chloro-benzylidene)-1,3-dihydro-indol-2-one (Compound 132)
5-chloro-3-naphtalen-1-ylmethylene-1,3-dihydro-indol-2-one (Compound 133)
5-chloro-3-(4-bromo-benzylidene)-1,3-dihydro-indol-2-one (Compound 134)
5-chloro-3-(4-methyl-benzylidene)-1,3-dihydro-indol-2-one (Compound 135)
3-anthracen-9-ylmethylene-5-chloro-1,3-dihydro-indol-2-one (Compound 136)
5-chloro-3-naphtalen-2-ylmethylene-1,3-dihydro-indol-2-one (Compound 137)
5-chloro-3-(2,4-dimethoxy-benzylidene)-1,3-dihydro-indol-2-one (Compound 138)
5-chloro-3-(2,4-dichloro-benzylidene)-1,3-dihydro-indol-2-one (Compound 139)
5-chloro-3-(3,4-dimethoxy-benzylidene)-1,3-dihydro-indol-2-one (Compound 140)
5-Chloro-3-(3,5-dimethoxy-benzylidene)-1,3-dihydro-indol-2-one (Compound 141)
5-chloro-3-(3,5-di-tert-butyl-4-hydroxy-benzylidene)-1,3-dihydro-indol-2-one (Compound 142)
5-chloro-3-(3,4,5-trimethoxy-benzylidene)-1,3-dihydro-indol-2-one (Compound 143)
3-benzylidene-5-Chloro-1,3-dihydro-indol-2-one (Compound 144)
5-chloro-3-(3-hydroxy-benzylidene)-1,3-dihydro-indol-2-one (Compound 145)
5-chloro-3-(2-methoxy-benzylidene)-1,3-dihydro-indol-2-one (Compound 146)
5-chloro-3-(2-methyl-benzylidene)-1,3-dihydro-indol-2-one (Compound 147)
3-(3,5-dibromo-4-hydroxy-benzylidene)-1,3-dihydro-indol-2-one (Compound 148)
3-(3,4-difluoro-benzylidene)-1,3-dihydro-indol-2-one (Compound 149)
3-(2-hydroxy-naphtalen-1-ylmethylene)-1,3-dihydro-indol-2-one (Compound 150)
3-(4-methyl-benzylidene)-1,3-dihydro-indol-2-one (Compound 151)
3-(3,4-dimethoxy-benzylidene)-1,3-dihydro-indol-2-one (Compound 152)
3-(3-methoxy-benzylidene)-1,3-dihydro-indol-2-one (Compound 153)
3-(2-chloro-benzylidene)-1,3-dihydro-indol-2-one (Compound 154)
3-(3-chloro-benzylidene)-1,3-dihydro-indol-2-one (Compound 155)
3-(2,4-dichloro-benzylidene)-1,3-dihydro-indol-2-one (Compound 156)
3-(3,4-dichloro-benzylidene)-1,3-dihydro-indol-2-one (Compound 157)
3-(3-bromo-benzylidene)-1,3-dihydro-indol-2-one (Compound 158)
3-(4-hydroxy-benzylidene)-1,3-dihydro-indol-2-one (Compound 159)
3-(3-hydroxy-benzylidene)-1,3-dihydro-indol-2-one (Compound 160)
3-(2,4-dihydroxy-benzylidene)-1,3-dihydro-indol-2-one (Compound 161)
5-chloro-3-(2,5-dimethoxy-benzylidene)-1,3-dihydro-indol-2-one (Compound 162)
3-(3,4,5-trimethoxy-benzylidene)-1,3-dihydro-indol-2-one (Compound 163)
3-(3,5-dimethoxy-benzylidene)-1,3-dihydro-indol-2-one (Compound 164)
3-(2,3-dimethoxy-benzylidene)-1,3-dihydro-indol-2-one (Compound 165)
3-(2-methoxy-naphtalen-1-ylmethylene)-1,3-dihydro-indol-2-one (Compound 166)
3-(2-methoxy-benzylidene)-1,3-dihydro-indol-2-one (Compound 167)
3-(4-hydroxy-3-methoxy-benzylidene)-1,3-dihydro-indol-2-one (Compound 168)
3-(3-hydroxy-4-methoxy-benzylidene)-1,3-dihydro-indol-2-one (Compound 169)
5-bromo-3-(2,5-dimethoxy-benzylidene)-1,3-dihydro-indol-2-one (Compound 170)
6-bromo-3-(2,5-dimethoxy-benzylidene)-1,3-dihydro-indol-2-one (Compound 171)
7-bromo-3-(2,5-dimethoxy-benzylidene)-1,3-dihydro-indol-2-one (Compound 172)
3-(2,5-dimethoxy-benzylidene)-6-fluoro-1,3-dihydro-indol-2-one (Compound 173)
3-(2,5-dimethoxy-benzylidene)-5-trifluoromethyl-1,3-dihydro-indol-2-one (Compound 174)
5-amino-3-(2,5-dimethoxy-benzylidene)-1,3-dihydro-indol-2-one (Compound 175)
6-chloro-5-(2-chloro-acetyl)-3-(2,5-dimethoxy-benzylidene)-1,3-dihydro-indol-2-one (Compound 176)
3-(2,5-dimethoxy-benzylidene)-5-hydroxy-1,3-dihydro-indol-2-one (Compound 177)
3-(2,5-dimethoxy-benzylidene)-2-oxo-2,3-dihydro-1H-indole-5-carboxylic acid methyl ester (Compound 178)
3-(9-ethyl-9H-carbazol-3-ylmethylene)-1,3-dihydro-indol-2-one (Compound 179)
3-(2-hydroxy-3-methoxy-benzylidene)-1,3-dihydro-indol-2-one (Compound 180)
3-(2,5-dimethoxy-benzylidene)-4,5-difluoro-1,3-dihydro-indol-2-one (Compound 181)
3-(3,5-dichloro-2-hydroxy-benzylidene)-1,3-dihydro-indol-2-one (Compound 182)
3-(2,5-diethoxy-benzylidene)-1,3-dihydro-indol-2-one (Compound 183)
3-(2,5-dihydroxy-benzylidene)-1,3-dihydro-indol-2-one (Compound 184)
3-(2,4,5-trimethoxy-benzylidene)-1,3-dihydro-indol-2-one (Compound 185)
3-(9-methyl-9H-carbazol-3-ylmethylene)-1,3-dihydro-indol-2-one (Compound 186)
3-(2-hydroxy-5-trifluoromethoxy-benzylidene)-1,3-dihydro-indol-2-one (Compound 187)
3-(1H-indol-5-ylmethylene)-1,3-dihydro-indol-2-one (Compound 188)
3-(1H-indol-4-ylmethylene)-1,3-dihydro-indol-2-one (Compound 189)
3-(1H-indol-7-ylmethylene)-1,3-dihydro-indol-2-one (Compound 190)
3-(1,4-dimethyl-9H-carbazol-2-ylmethylene)-1,3-dihydro-indol-2-one (Compound 191)
3-(2-benzyloxy-4,5-dimethoxy-benzylidene)-1,3-dihydro-indol-2-one (Compound 192)
3-(2,5-dichloro-benzylidene)-1,3-dihydro-indol-2-one (Compound 193)
3-(2,5-dimethoxy-benzylidene)-2-oxo-2,3-dihydro-1H-indole-7-carbonitrile (Compound 194)
3-(2,5-dimethoxy-benzylidene)-6-methanesulfonyl-1,3-dihydro-indol-2-one (Compound 195)
3-(2,5-dimethoxy-benzylidene)-2-oxo-2,3-dihydro-1H-indole-5-carbonitrile (Compound 196)
3-(2,5-dimethoxy-benzylidene)-6-trifluoromethyl-1,3-dihydro-indol-2-one (Compound 197)
3-(2,5-dimethoxy-benzylidene)-7-fluoro-1,3-dihydro-indol-2-one (Compound 198)
3-(2,5-dimethoxy-benzylidene)-2-oxo-2,3-dihydro-1H-indole-6-carbonitrile (Compound 199)
6-chloro-3-(2,5-dimethoxy-benzylidene)-1,3-dihydro-indol-2-one (Compound 200)
3-(2,5-dibromo-benzylidene)-1,3-dihydro-indol-2-one (Compound 201)
3-(5-bromo-2-ethoxy-benzylidene)-1,3-dihydro-indol-2-one (Compound 202)
3-(5-bromo-2-methoxy-benzylidene)-1,3-dihydro-indol-2-one (Compound 203)
3-(2-fluoro-5-methoxy-benzylidene)-1,3-dihydro-indol-2-one (Compound 204)
3-(2,5-difluoro-benzylidene)-1,3-dihydro-indol-2-one (Compound 205)
3-(2-chloro-5-nitro-benzylidene)-1,3-dihydro-indol-2-one (Compound 206)
3-(2,5-bis-trifluoromethyl-benzylidene)-1,3-dihydro-indol-2-one (Compound 207)
3-(2,4-dichloro-benzylidene)-1,3-dihydro-indol-2-one (Compound 208)
3-(2-hydroxy-5-methoxy-benzylidene)-1,3-dihydro-indol-2-one (Compound 209)
3-(1H-indol-6-ylmethylene)-1,3-dihydro-indol-2-one (Compound 210)
3-(2,5-dimethoxy-benzylidene)-5-fluoro-1,3-dihydro-indol-2-one (Compound 211)
3-[4-(quinolin-2-ylmethoxy)-benzylidene]-1,3-dihydro-indol-2-one (Compound 212)
3-[4-(naphthalen-2-ylmethoxy)-benzylidene]-1,3-dihydro-indol-2-one (Compound 213)
3-[3,5-dichloro-2-(quinolin-2-ylmethoxy)-benzylidene]-1,3-dihydro-indol-2-one (Compound 214)
2-[4-(2-oxo-1,2-dihydro-indol-3-ylidenemethyl)-phenoxy]-propionic acid (Compound
215)
2-benzyl-3-butylamino-5-(2-oxo-1,2-dihydro-indol-3-ylidenemethyl)-benzenesulfonamide (Compound 216)
2-benzyl-3-benzylamino-5-(2-oxo-1,2-dihydro-indol-3-ylidenemethyl)-benzenesulfonamide (Compound 217)
3-[(furan-2-ylmethyl)-amino]-5-(2-oxo-1,2-dihydro-indol-3-ylidenemethyl)-2-phenoxy-benzenesulfonamide (Compound 218)
3-methylamino-5-(2-oxo-1,2-dihydro-indol-3-ylidenemethyl)-2-phenoxy-benzenesulfonamide (Compound 219)
2-benzyl-3-ethoxy-5-(2-oxo-1,2-dihydro-indol-3-ylidenemethyl)-benzenesulfonamide (Compound 220)
[2-(2-oxo-1,2-dihydro-indol-3-ylidenemethyl)-phenoxy]-acetic acid (Compound 221)
3-[4-(6-methyl-pyridin-2-ylmethoxy)-benzylidene]-1,3-dihydro-indol-2-one (Compound 222)
4-[4-(5-chloro-2-oxo-1,2-dihydro-indol-3-ylidenemethyl)-phenyl]-piperazine-1-carbaldehyde (Compound 223)
5-chloro-3-(4-isopropyl-benzylidene)-1,3-dihydro-indol-2-one (Compound 224)
4-[4-(2-oxo-1,2-dihydro-indol-3-ylidenemethyl)-phenyl]-piperazine-1-carbaldehyde (Compound 225)
3-[5-methoxy-2-(2-morpholin-4-yl-ethoxy)-benzylidene]-1,3-dihydro-indol-2-one; hydrochloride (Compound 258)
3-[5-methoxy-2-(2-piperidin-1-yl-ethoxy)-benzylidene]-1,3-dihydro-indol-2-one; hydrochloride (Compound 259)
3-(2,5-dimethoxy-benzylidene)-5,7-difluoro-1,3-dihydro-indol-2-one (Compound 260)
3-[4-(1-quinolin-4-yl-ethoxy)-benzylidene]-1,3-dihydro-indol-2-one (Compound 261)
3-[4-(pyridin-4-ylmethoxy)-benzylidene]-1,3-dihydro-indol-2-one (Compound 262) and
5-amino-3-(2,5-dimethoxy-benzylidene)-1,3-dihydro-indol-2-one; methanesulfonic acid (Compound 265)

57. The method of claim 32 wherein the compound is a compound of general formula IV 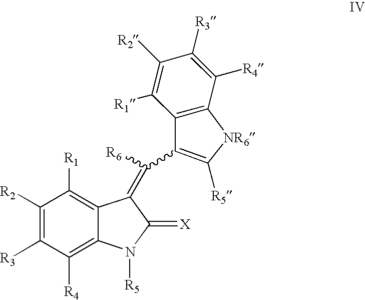
wherein R₁, R₂, R₃, R₄, R₅, R₆ and X are as indicated in claim 1,
R₁″, R₂″, R₃″, R₄″ and R₅″ are the same or different and independently selected from the group consisting of hydrogen, halogen, trihalomethyl, C₁₋₁₂-alkyl, C₂₋₁₂-alkenyl, C₄₋₁₂-alkadienyl, C₆₋₁₂-alkatrienyl, C₂₋₁₂-alkynyl, hydroxy, carboxy, formyl, aryl, heteroaryl, carbocyclyl, heterocyclyl, amino, carbamoyl, cyano, guanidino, carbamido, —OR₁₀, —C(O)R₁₀, —C(O)OR₁₀, OC(O)R₁₀, —NR₁₀R₁₁, —C(O)NR₁₀R₁₁, —NHC(O)R₁₀, —SR₁₀, —S(O)R₁₀, —S(O)₂R₁₀, —S(O)₂NR₁₀R₁₁ and —S(O)OR₁₀, wherein R₁₀ and R₁₁ are the same or different and independently selected from the group consisting of hydrogen, C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl, or wherein R₁₀ and R₁₁, together with the nitrogen atom to which they are attached form a heterocyclic or heteroaryl ring, each of C₁₋₁₂-alkyl, C₂₋₁₂-alkenyl, C₄₋₁₂-alkadienyl, C₆₋₁₂-alkatrienyl, C₂₋₁₂-alkynyl, aryl, heteroaryl, carbocyclyl and heterocyclyl being optionally substituted with one or more, same or different substituents selected from the group consisting of hydrogen, halogen, trihalomethyl, C₁₋₆-alkyl, C₂₋₆-alkenyl, C₄₋₆-alkadienyl, C₂₋₆-alkynyl, hydroxy, carboxy, formyl, aryl, heteroaryl, carbocyclyl, heterocyclyl, amino, carbamoyl, cyano, guanidino, carbamido, —OR₁₂, —C(O)R₁₂, —C(O)OR₁₂, —OC(O)R₁₂, —NR₁₂R₁₃, —C(O)NR₁₂R₁₃, —NHC(O)R₁₂, —SR₁₂, —S(O)R₁₂, —S(O)₂R₁₂, —S(O)₂NR₁₂R₁₃ and —S(O)OR₁₂, wherein R₁₂ and R₁₃ are the same or different and independently selected from the group consisting of hydrogen, C₁₋₆-alkyl, C₂₋₆-alkenyl, C₄₋₆-alkadienyl, C₂₋₆-alkynyl, aryl, heteroaryl, carbocyclyl and heterocyclyl, or wherein R₁₂ and R₁₃, together with the nitrogen atom to which they are attached form a heterocyclic or heteroaryl ring, each C₁₋₆-alkyl, C₂₋₆-alkenyl, C₄₋₆-alkadienyl, C₂₋₆-alkynyl, aryl, heteroaryl, carbocyclyl and heterocyclyl substituent being optionally substituted with one or more, same or different substituents selected from the group consisting of hydrogen, hydroxy, C₁₋₄alkyl, C₁₋₄alkoxy, nitro, cyano, amino, oxo, halogen, trihalomethyl, C₁₋₄alkylthio, C₁₋₄alkylamino, C₁₋₄alkoxycarbonyl, carboxy, —CONH₂, S(O)NH₂, aryl, heteroaryl, heterocyclyl or carbocyclyl, said aryl, heteroaryl, heterocyclyl or carbocyclyl being optionally substituted with one or more of hydrogen, hydroxy, C₁₋₄ alkyl, C₁₋₄ alkoxy, nitro, cyano, amino, oxo, halogen, trihalomethyl, C₁₋₄ alkylthio, C₁₋₄ alkylamino, C₁₋₄alkoxycarbonyl, carboxy, —CONH₂ or —S(O)NH₂; and
R₆″ is hydrogen, heterocyclyl, heteroaryl, —C(O)R₂₃, —S(O)₂R₂₃, —C(O)OR₂₃ or C₁₋₆alkyl optionally substituted with heterocyclyl, heteroaryl or —C(O)OR₂₃, wherein R₂₃ is hydrogen, C₁₋₆alkyl, aryl, heteroaryl or heterocyclyl.

58. The method of claim 57 wherein, in the compound of formula IV, R₂, R₃, R₄, R₅, R₆ and X are as indicated in claim 31, and R₁″, R₂″, R₃″, R₄″ and R₅″ are the same or different and independently selected from the group consisting of with C₁₋₁₀alkyl, C₁₋₁₀alkoxy, aryl, heteroaryl, aryloxy, C₁₋₁₀alkylaryl, C₁₋₁₀alkylaryloxy, halogen, trihalomethyl, a sugar residue, S(O)R₁₈, S(O)₂R₁₈, S(O)₂NR₁₈R₁₉, S(O)₃R₁₈, SR₁₈, NO₂, NR₁₈R₁₉, OR₁₈, CN, CH₂OH, C(O)R₁₈, C(O)OR₁₈, OC(O)R₁₈, NHC(O)R₁₈, (CH₂)ₙC(O)₂R₁₈ and C(O)NR₁₈R₁₉, wherein R₁₈ is hydrogen, C₁₋₆alkyl, heteroaryl or aryl, said C₁₋₆ alkyl, heteroaryl or aryl being optionally substituted with hydroxy or NR₂₆R₂₇, wherein R₂₆ and R₂₇ are independently hydrogen or C₁₋₆ alkyl or, together with the nitrogen atom to which they are attached, form a heteroaryl or heterocyclic ring, R₁₉ is hydrogen, C₁₋₆alkyl or aryl, and n is 0-3; and R₆″ is hydrogen, C₁₋₆ alkyl, heteroaryl, heteroaryl-C₁₋₆ alkyl, C(O)R₁₈, C(O)OR₁₈ or S(O)₂R₁₈, wherein R₁₈ is as indicated above.

59. The method of claim 57 wherein, in the compound of formula IV, R₅″ is hydrogen or C₁₋₆ alkyl.

60. The method of claim 54 wherein, in the compound of formula IV, R₆″ is hydrogen or C₁₋₆ alkyl.

61. The method of claim 57 wherein, in the compound of formula IV, R₅ is hydrogen, hydroxy, C(O)R₁₄ or C(O)OR₁₄, wherein R₁₄ is as defined in claim 32.

62. The method of claim 57 wherein the compound is selected from the group consisting of
3-(1H-indol-3-ylmethylene)-1,3-dihydro-indol-2-one (Compound 57)
[3-(2-oxo-1,2-dihydro-indol-3-ylidenemethyl)-indol-1-yl]-acetic acid methyl ester (Compound 24)
[3-(2-oxo-1,2-dihydro-indol-3-ylidenemethyl)-indol-1-yl]-acetic acid ethyl ester (Compound 25)
[3-(2-oxo-1,2-dihydro-indol-3-ylidenemethyl)-indol-1-yl]-acetic acid (Compound 26)
3-[3-(2-oxo-1,2-dihydro-indol-3-ylidenemethyl)-indol-1-yl]-propionic acid ethyl ester (Compound 27)
3-[3-(2-oxo-1,2-dihydro-indol-3-ylidenemethyl)-indol-1-yl]-propionic acid (Compound 28)
3-[1-(2-chloro-thiazol-5-ylmethyl)-1H-indol-3-ylmethylene]-1,3-dihydro-indol-2-one (Compound 29)
3-(1-methyl-1H-indol-3-ylmethylene)-1,3-dihydro-indol-2-one (Compound 30)
3-(1-propyl-1H-indol-3-ylmethylene)-1,3-dihydro-indol-2-one (Compound 31)
3-(1H-indol-3-ylmethylene)-2-oxo-2,3-dihydro-indole-1-carboxylic acid tert-butyl ester (Compound 40)
1-hydroxy-3-(1H-indol-3-ylmethylene)-1,3-dihydro-indol-2-one (Compound 47)
(1-Methyl-1H-indol-3-yl)-(2-oxo-1,2-dihydro-indol-3-ylidene)-acetic acid (Compound 56)
3-(2-phenyl-1H-indol-3-ylmethylene)-1,3-dihydro-indol-2-one (Compound 58)
3-(1-methyl-2-phenyl-1H-indol-3-ylmethylene)-1,3-dihydro-indol-2-one (Compound 59)
3-[2-(4-chloro-phenyl)-1H-indol-3-ylmethylene]-1,3-dihydro-indol-2-one (Compound 60)
3-(2-naphthalen-2-yl-1H-indol-3-ylmethylene)-1,3-dihydro-indol-2-one (Compound 61)
5-chloro-3-(1H-indol-3-ylmethylene)-1,3-dihydro-indol-2-one (Compound 62)
3-(5-methyl-1H-indol-3-ylmethylene)-1,3-dihydro-indol-2-one (Compound 63)
5,7-difluoro-3-(1H-indol-3-ylmethylene)-1,3-dihydro-indol-2-one (Compound 64)
5-bromo-3-(1H-indol-3-ylmethylene)-1,3-dihydro-indol-2-one (Compound 65)
6-fluoro-3-(1H-indol-3-ylmethylene)-1,3-dihydro-indol-2-one (Compound 66)
6-bromo-3-(1H-indol-3-ylmethylene)-1,3-dihydro-indol-2-one (Compound 67)
5-hydroxy-3-(1H-indol-3-ylmethylene)-1,3-dihydro-indol-2-one (Compound 68)
3-(4,5,6,7-tetrafluoro-1H-indol-3-ylmethylene)-1,3-dihydro-indol-2-one (Compound 69)
3-(6-fluoro-1H-indol-3-ylmethylene)-1,3-dihydro-indol-2-one (Compound 70)
3-[2-(4-chloro-phenyl)-5-nitro-1H-indol-3-ylmethylene)-1,3-dihydro-indol-2-one (Compound 71)
7-bromo-3-(1H-indol-3-ylmethylene)-1,3-dihydro-indol-2-one (Compound 72)
3-(6-methyl-1H-indol-3-ylmethylene)-1,3-dihydro-indol-2-one (Compound 73)
3-(7-methyl-1H-indol-3-ylmethylene)-1,3-dihydro-indol-2-one (Compound 74)
3-(2-methyl-1H-indol-3-ylmethylene)-1,3-dihydro-indol-2-one (Compound 75)
3-(5-fluoro-1-methyl-1H-indol-3-ylmethylene)-1,3-dihydro-indol-2-one (Compound 76)
3-(5-fluoro-1H-indol-3-ylmethylene)-1,3-dihydro-indol-2-one (Compound 77)
3-(5-methoxy-1H-indol-3-ylmethylene)-1,3-dihydro-indol-2-one (Compound 78)
3-(5-benzyloxy-1H-indol-3-ylmethylene)-1,3-dihydro-indol-2-one (Compound 79)
3-(6-methoxy-1H-indol-3-ylmethylene)-1,3-dihydro-indol-2-one (Compound 80)
3-(5-methoxy-1-methyl-1H-indol-3-ylmethylene)-1,3-dihydro-indol-2-one (Compound 81)
3-(6-methoxy-1-methyl-1H-indol-3-ylmethylene)-1,3-dihydro-indol-2-one (Compound 82)
3-(4-benzyloxy-1H-indol-3-ylmethylene)-1,3-dihydro-indol-2-one (Compound 83)
3-(1H-indol-3-ylmethylene)-2-oxo-2,3-dihydro-1H-indol-6-carbonitrile (Compound 84)
3-(1H-indol-3-ylmethylene)-2-oxo-2,3-dihydro-1H-indol-7-carbonitrile (Compound 85)
3-(1H-indol-3-ylmethylene)-2-oxo-2,3-dihydro-1H-indol-5-carbonitrile (Compound 86)
7-fluoro-3-(1H-indol-3-ylmethylene)-1,3-dihydro-indol-2-one (Compound 87)
3-(1H-indol-3-ylmethylene)-6-trifluoromethyl-1,3-dihydro-indol-2-one (Compound 88)
3-(1H-indol-3-ylmethylene)-6-methanesulfonyl-1,3-dihydro-indol-2-one (Compound 89)
3-(1H-indol-3-ylmethylene)-5-trifluoromethyl-1,3-dihydro-indol-2-one (Compound 90)
3-(1H-indol-3-ylmethylene)-5,6-dimethoxy-1,3-dihydro-indol-2-one (Compound 91)
4,5-difluoro-3-(1H-indol-3-ylmethylene)-1,3-dihydro-indol-2-one (Compound 92)
3-(1H-indol-3-ylmethylene)-5-methoxy-1,3-dihydro-indol-2-one (Compound 92A)
6-chloro-3-(1H-indol-3-ylmethylene)-1,3-dihydro-indol-2-one (Compound 93) and
3-[1-Methyl-2-(4-methyl-piperazin-1-yl)-1H-indol-3-ylmethylene]-1,3-dihydro-indol-2-one (Compound 94)
